SEQUENCE LISTING (177 sequences)

SEQ ID NO: 1 - protein, 775 aa, synthetic construct
  REGION 1..775: note = source = /note="Description of Artificial Sequence: Syntheticpolypeptide"
MAQVSKQTSKKRELSIDEYQGARKWCFTIAFNKALVNRDKNDGLFVESLLRHEKYSKHDWYDEDTRALIKCSTQAANAKAEALRNYFSHYRHSPGCLTFTAEDELRTIMERAYERAIFECRRRETEVIIEFPSLFEGDRITTAGVVFFVSFFVERRVLDRLYGAVSGLKKNEGQYKLTRKALSMYCLKDSRFTKAWDKRVLLFRDILAQLGRIPAEAYEYYHGEQGDKKRANDNEGTNPKRHKDKFIEFALHYLEAQHSEICFGRRHIVREEAGAGDEHKKHRTKGKVVVDFSKKDEDQSYYISKNNVIVRIDKNAGPRSYRMGLNELKYLVLLSLQGKGDDAIAKLYRYRQHVENILDVVKVTDKDNHVFLPRFVLEQHGIGRKAFKQRIDGRVKHVRGVWEKKKAATNEMTLHEKARDILQYVNENCTRSFNPGEYNRLLVCLVGKDVENFQAGLKRLQLAERIDGRVYSIFAQTSTINEMHQVVCDQILNRLCRIGDQKLYDYVGLGKKDEIDYKQKVAWFKEHISIRRGFLRKKFWYDSKKGFAKLVEEHLESGGGQRDVGLDKKYYHIDAIGRFEGANPALYETLARDRLCLMMAQYFLGSVRKELGNKIVWSNDSIELPVEGSVGNEKSIVFSVSDYGKLYVLDDAEFLGRICEYFMPHEKGKIRYHTVYEKGFRAYNDLQKKCVEAVLAFEEKVVKAKKMSEKEGAHYIDFREILAQTMCKEAEKTAVNKVRRAFFHHHLKFVIDEFGLFSDVMKKYGIEKEWKFPVK

SEQ ID NO: 2 - protein, 805 aa, synthetic construct
  REGION 1..805: note = source = /note="Description of Artificial Sequence: Syntheticpolypeptide"
MKVENIKEKSKKAMYLINHYEGPKKWCFAIVLNRACDNYEDNPHLFSKSLLEFEKTSRKDWFDEETRELVEQADTEIQPNPNLKPNTTANRKLKDIRNYFSHHYHKNECLYFKNDDPIRCIMEAAYEKSKIYIKGKQIEQSDIPLPELFESSGWITPAGILLLASFFVERGILHRLMGNIGGFKDNRGEYGLTHDIFTTYCLKGSYSIRAQDHDAVMFRDILGYLSRVPTESFQRIKQPQIRKEGQLSERKTDKFITFALNYLEDYGLKDLEGCKACFARSKIVREQENVESINDKEYKPHENKKKVEIHFDQSKEDRFYINRNNVILKIQKKDGHSNIVRMGVYELKYLVLMSLVGKAKEAVEKIDNYIQDLRDQLPYIEGKNKEEIKEYVRFFPRFIRSHLGLLQINDEEKIKARLDYVKTKWLDKKEKSKELELHKKGRDILRYINERCDRELNRNVYNRILELLVSKDLTGFYRELEELKRTRRIDKNIVQNLSGQKTINALHEKVCDLVLKEIESLDTENLRKYLGLIPKEEKEVTFKEKVDRILKQPVIYKGFLRYQFFKDDKKSFVLLVEDALKEKGGGCDVPLGKEYYKIVSLDKYDKENKTLCETLAMDRLCLMMARQYYLSLNAKLAQEAQQIEWKKEDSIELIIFTLKNPDQSKQSFSIRFSVRDFTKLYVTDDPEFLARLCSYFFPVEKEIEYHKLYSEGINKYTNLQKEGIEAILELEKKLIERNRIQSAKNYLSFNEIMNKSGYNKDEQDDLKKVRNSLLHYKLIFEKEHLKKFYEVMRGEGIEKKWSLIV

SEQ ID NO: 3 - protein, 790 aa, synthetic construct
  REGION 1..790: note = source = /note="Description of Artificial Sequence: Syntheticpolypeptide"
MNGIELKKEEAAFYFNQAELNLKAIEDNIFDKERRKTLLNNPQILAKMENFIFNFRDVTKNAKGEIDCLLLKLRELRNFYSHYVHKRDVRELSKGEKPILEKYYQFAIESTGSENVKLEIIENDAWLADAGVLFFLCIFLKKSQANKLISGISGFKRNDDTGQPRRNLFTYFSIREGYKVVPEMQKHFLLFSLVNHLSNQDDYIEKAHQPYDIGEGLFFHRIASTFLNISGILRNMKFYTYQSKRLVEQRGELKREKDIFAWEEPFQGNSYFEINGHKGVIGEDELKELCYAFLIGNQDANKVEGRITQFLEKFRNANSVQQVKDDEMLKPEYFPANYFAESGVGRIKDRVLNRLNKAIKSNKAKKGEIIAYDKMREVMAFINNSLPVDEKLKPKDYKRYLGMVRFWDREKDNIKREFETKEWSKYLPSNFWTAKNLERVYGLAREKNAELFNKLKADVEKMDERELEKYQKINDAKDLANLRRLASDFGVKWEEKDWDEYSGQIKKQITDSQKLTIMKQRITAGLKKKHGIENLNLRITIDINKSRKAVLNRIAIPRGFVKRHILGWQESEKVSKKIREAECEILLSKEYEELSKQFFQSKDYDKMTRINGLYEKNKLIALMAVYLMGQLRILFKEHTKLDDITKTTVDFKISDKVTVKIPFSNYPSLVYTMSSKYVDNIGNYGFSNKDKDKPILGKIDVIEKQRMEFIKEVLGFEKYLFDDKIIDKSKFADTATHISFAEIVEELVEKGWDKDRLTKLKDARNKALHGEILTGTSFDETKSLINELKK

SEQ ID NO: 4 - protein, 792 aa, synthetic construct
  REGION 1..792: note = source = /note="Description of Artificial Sequence: Syntheticpolypeptide"
MSPDFIKLEKQEAAFYFNQTELNLKAIESNILDKQQRMILLNNPRILAKVGNFIFNFRDVTKNAKGEIDCLLFKLEELRNFYSHYVHTDNVKELSNGEKPLLERYYQIAIQATRSEDVKFELFETRNENKITDAGVLFFLCMFLKKSQANKLISGISGFKRNDPTGQPRRNLFTYFSAREGYKALPDMQKHFLLFTLVNYLSNQDEYISELKQYGEIGQGAFFNRIASTFLNISGISGNTKFYSYQSKRIKEQRGELNSEKDSFEWIEPFQGNSYFEINGHKGVIGEDELKELCYALLVAKQDINAVEGKIMQFLKKFRNTGNLQQVKDDEMLEIEYFPASYFNESKKEDIKKEILGRLDKKIRSCSAKAEKAYDKMKEVMEFINNSLPAEEKLKRKDYRRYLKMVRFWSREKGNIEREFRTKEWSKYFSSDFWRKNNLEDVYKLATQKNAELFKNLKAAAEKMGETEFEKYQQINDVKDLASLRRLTQDFGLKWEEKDWEEYSEQIKKQITDRQKLTIMKQRVTAELKKKHGIENLNLRITIDSNKSRKAVLNRIAIPRGFVKKHILGWQGSEKISKNIREAECKILLSKKYEELSRQFFEAGNFDKLTQINGLYEKNKLTAFMSVYLMGRLNIQLNKHTELGNLKKTEVDFKISDKVTEKIPFSQYPSLVYAMSRKYVDNVDKYKFSHQDKKKPFLGKIDSIEKERIEFIKEVLDFEEYLFKNKVIDKSKFSDTATHISFKEICDEMGKKGCNRNKLTELNNARNAALHGEIPSETSFREAKPLINELKK

SEQ ID NO: 5 - protein, 792 aa, synthetic construct
  REGION 1..792: note = source = /note="Description of Artificial Sequence: Syntheticpolypeptide"
MSPDFIKLEKQEAAFYFNQTELNLKAIESNIFDKQQRVILLNNPQILAKVGDFIFNFRDVTKNAKGEIDCLLLKLRELRNFYSHYVYTDDVKILSNGERPLLEKYYQFAIEATGSENVKLEIIESNNRLTEAGVLFFLCMFLKKSQANKLISGISGFKRNDPTGQPRRNLFTYFSVREGYKVVPDMQKHFLLFVLVNHLSGQDDYIEKAQKPYDIGEGLFFHRIASTFLNISGILRNMEFYIYQSKRLKEQQGELKREKDIFPWIEPFQGNSYFEINGNKGIIGEDELKELCYALLVAGKDVRAVEGKITQFLEKFKNADNAQQVEKDEMLDRNNFPANYFAESNIGSIKEKILNRLGKTDDSYNKTGTKIKPYDMMKEVMEFINNSLPADEKLKRKDYRRYLKMVRIWDSEKDNIKREFESKEWSKYFSSDFWMAKNLERVYGLAREKNAELFNKLKAVVEKMDEREFEKYRLINSAEDLASLRRLAKDFGLKWEEKDWQEYSGQIKKQISDRQKLTIMKQRITAELKKKHGIENLNLRITIDSNKSRKAVLNRIAVPRGFVKEHILGWQGSEKVSKKTREAKCKILLSKEYEELSKQFFQTRNYDKMTQVNGLYEKNKLLAFMVVYLMERLNILLNKPTELNELEKAEVDFKISDKVMAKIPFSQYPSLVYAMSSKYADSVGSYKFENDEKNKPFLGKIDTIEKQRMEFIKEVLGFEEYLFEKKIIDKSEFADTATHISFDEICNELIKKGWDKDKLTKLKDARNAALHGEIPAETSFREAKPLINGLKK

SEQ ID NO: 6 - protein, 799 aa, synthetic construct
  REGION 1..799: note = source = /note="Description of Artificial Sequence: Syntheticpolypeptide"
MNIIKLKKEEAAFYFNQTILNLSGLDEIIEKQIPHIISNKENAKKVIDKIFNNRLLLKSVENYIYNFKDVAKNARTEIEAILLKLVELRNFYSHYVHNDTVKILSNGEKPILEKYYQIAIEATGSKNVKLVIIENNNCLTDSGVLFLLCMFLKKSQANKLISSVSGFKRNDKEGQPRRNLFTYYSVREGYKVVPDMQKHFLLFALVNHLSEQDDHIEKQQQSDELGKGLFFHRIASTFLNESGIFNKMQFYTYQSNRLKEKRGELKHEKDTFTWIEPFQGNSYFTLNGHKGVISEDQLKELCYTILIEKQNVDSLEGKIIQFLKKFQNVSSKQQVDEDELLKREYFPANYFGRAGTGTLKEKILNRLDKRMDPTSKVTDKAYDKMIEVMEFINMCLPSDEKLRQKDYRRYLKMVRFWNKEKHNIKREFDSKKWTRFLPTELWNKRNLEEAYQLARKENKKKLEDMRNQVRSLKENDLEKYQQINYVNDLENLRLLSQELGVKWQEKDWVEYSGQIKKQISDNQKLTIMKQRITAELKKMHGIENLNLRISIDTNKSRQTVMNRIALPKGFVKNHIQQNSSEKISKRIREDYCKIELSGKYEELSRQFFDKKNFDKMTLINGLCEKNKLIAFMVIYLLERLGFELKEKTKLGELKQTRMTYKISDKVKEDIPLSYYPKLVYAMNRKYVDNIDSYAFAAYESKKAILDKVDIIEKQRMEFIKQVLCFEEYIFENRIIEKSKFNDEETHISFTQIHDELIKKGRDTEKLSKLKHARNKALHGEIPDGTSFEKAKLLINEIKK

SEQ ID NO: 7 - protein, 803 aa, synthetic construct
  REGION 1..803: note = source = /note="Description of Artificial Sequence: Syntheticpolypeptide"
MNAIELKKEEAAFYFNQARLNISGLDEIIEKQLPHIGSNRENAKKTVDMILDNPEVLKKMENYVFNSRDIAKNARGELEALLLKLVELRNFYSHYVHKDDVKTLSYGEKPLLDKYYEIAIEATGSKDVRLEIIDDKNKLTDAGVLFLLCMFLKKSEANKLISSIRGFKRNDKEGQPRRNLFTYYSVREGYKVVPDMQKHFLLFTLVNHLSNQDEYISNLRPNQEIGQGGFFHRIASKFLSDSGILHSMKFYTYRSKRLTEQRGELKPKKDHFTWIEPFQGNSYFSVQGQKGVIGEEQLKELCYVLLVAREDFRAVEGKVTQFLKKFQNANNVQQVEKDEVLEKEYFPANYFENRDVGRVKDKILNRLKKITESYKAKGREVKAYDKMKEVMEFINNCLPTDENLKLKDYRRYLKMVRFWGREKENIKREFDSKKWERFLPRELWQKRNLEDAYQLAKEKNTELFNKLKTTVERMNELEFEKYQQINDAKDLANLRQLARDFGVKWEEKDWQEYSGQIKKQITDRQKLTIMKQRITAALKKKQGIENLNLRITTDTNKSRKVVLNRIALPKGFVRKHILKTDIKISKQIRQSQCPIILSNNYMKLAKEFFEERNFDKMTQINGLFEKNVLIAFMIVYLMEQLNLRLGKNTELSNLKKTEVNFTITDKVTEKVQISQYPSLVFAINREYVDGISGYKLPPKKPKEPPYTFFEKIDAIEKERMEFIKQVLGFEEHLFEKNVIDKTRFTDTATHISFNEICDELIKKGWDENKIIKLKDARNAALHGKIPEDTSFDEAKVLINELKK

SEQ ID NO: 8 - DNA, 36 bp, synthetic construct
  misc_feature 1..36: note = source = /note="Description of Artificial Sequence: Syntheticoligonucleotide"
  source 1..36: mol_type = other DNA
gctggagcagcccccgatttgtggggtgattacagc

SEQ ID NO: 9 - DNA, 36 bp, synthetic construct
  misc_feature 1..36: note = source = /note="Description of Artificial Sequence: Syntheticoligonucleotide"
  source 1..36: mol_type = other DNA
gctgaagaagcctccgatttgagaggtgattacagc

SEQ ID NO: 10 - DNA, 36 bp, synthetic construct
  misc_feature 1..36: note = source = /note="Description of Artificial Sequence: Syntheticoligonucleotide"
  source 1..36: mol_type = other DNA
gctgtgatagacctcgatttgtggggtagtaacagc

SEQ ID NO: 11 - DNA, 36 bp, synthetic construct; identical to SEQ ID NO: 10
  misc_feature 1..36: note = source = /note="Description of Artificial Sequence: Syntheticoligonucleotide"
  source 1..36: mol_type = other DNA

SEQ ID NO: 12 - DNA, 36 bp, synthetic construct; identical to SEQ ID NO: 10
  misc_feature 1..36: note = source = /note="Description of Artificial Sequence: Syntheticoligonucleotide"
  source 1..36: mol_type = other DNA

SEQ ID NO: 13 - DNA, 36 bp, synthetic construct
  misc_feature 1..36: note = source = /note="Description of Artificial Sequence: Syntheticoligonucleotide"
  source 1..36: mol_type = other DNA
gctgtgatgggcctcaatttgtggggaagtaacagc

SEQ ID NO: 14 - DNA, 36 bp, synthetic construct
  misc_feature 1..36: note = source = /note="Description of Artificial Sequence: Syntheticoligonucleotide"
  source 1..36: mol_type = other DNA
gctgtgataggcctcgatttgtggggtagtaacagc

SEQ ID NO: 15 - DNA, 2328 bp, unidentified
  misc_feature 1..2328: note = source = /note="Description of Unknown:Cas13e.1 sequence"
  source 1..2328: mol_type = other DNA
atggcgcaagtgtcaaagcagacttcgaaaaagagagagttgtctatcgatgaatatcaaggtgctcggaaatggtgttttacgattgccttcaacaaggctcttgtgaatcgagataagaacgacgggctttttgtcgagtcgctgttacgccatgaaaagtattcaaagcacgactggtacgatgaggatacacgcgctttgatcaagtgtagcacacaagcggccaatgcgaaggccgaggcgttaagaaactatttctcccactatcgacattcgcccgggtgtctgacatttacagcagaagatgagttgcggacaatcatggaaagggcgtatgagcgggcgatctttgaatgcaggagacgcgaaactgaagtgatcatcgagtttcccagcctgttcgaaggcgaccggatcactacggcgggggttgtgtttttcgtttcgttctttgttgaacggcgggtgctggatcgtttgtacggtgcggtaagtgggcttaagaaaaacgaaggacagtacaagctgactcggaaggcgctttcgatgtattgcctgaaagacagtcgtttcacgaaggcgtgggacaaacgcgtgctgcttttcagggatatactcgcgcagcttggacgcatccctgcggaggcgtatgaatactaccacggagagcagggcgacaagaaaagagcaaacgacaatgaggggacgaatccgaaacgccataaagacaagttcatcgagtttgcactgcattatctggaggcgcaacacagtgagatatgcttcgggcggcgacacattgtcagggaggaggccggggcaggcgacgaacacaaaaagcacaggaccaaaggcaaggtagttgtcgacttttcaaaaaaagacgaagatcagtcatactatatcagtaagaacaatgttatcgtcaggattgataagaatgccgggcctcggagttatcgcatggggcttaacgaattgaaataccttgtattgcttagccttcagggaaagggcgacgatgcgattgcaaaactgtacaggtatcggcagcatgtggagaacattctggatgtagtgaaggtcacagataaggataatcacgtcttcctgccgcgatttgtgctggagcaacatgggattggcaggaaagcttttaagcaaagaatagacggcagagtaaagcatgttcgaggggtgtgggaaaagaagaaggcggcgaccaacgagatgacacttcacgagaaggcgcgggacattcttcaatacgtaaatgaaaattgcacgaggtctttcaatcccggcgagtacaaccggctgctggtgtgtctggttggcaaggatgttgagaattttcaggcgggactgaaacgcctgcaactggccgagcgaatcgacgggcgggtatattcaatttttgcgcagacctccacaataaacgagatgcatcaggtggtgtgtgatcagattctcaacagactttgccgaatcggcgatcagaagctctacgattatgtggggcttgggaagaaggatgaaatagattacaagcagaaggttgcatggttcaaggagcatatttctatccgcaggggtttcttgcgcaagaagttctggtatgacagcaagaagggattcgcgaagcttgtggaagagcatttggaaagcggcggcggacagagggacgttgggctggataaaaagtattatcatattgatgcgattgggcgattcgagggtgctaatccagccttgtatgaaacgctggcgcgagaccgtttgtgtctgatgatggcgcaatacttcctggggagtgtacgcaaggaattgggtaataaaattgtgtggtcgaatgatagcatcgagttgcccgtggagggctcagtgggtaacgaaaaaagcatcgtcttctcagtgagtgattacggcaagttatatgtgttggatgacgctgagtttcttgggcggatatgtgagtactttatgccgcacgaaaaagggaagatacggtatcatacagtttacgaaaaagggtttagggcatataatgatctgcagaagaaatgtgtcgaggcggtgctggcgtttgaagagaaggttgtcaaagccaaaaagatgagcgagaaggaaggggcgcattatattgattttcgtgagatactggcacaaacaatgtgtaaagaggcggagaagaccgccgtgaataaggtgcgtagagcgtttttccatcatcatttaaagtttgtgatagatgaatttgggttgtttagtgatgttatgaagaaatatggaattgaaaaggagtggaagtttcctgttaaatga

SEQ ID NO: 16 - DNA, 2418 bp, unidentified
  misc_feature 1..2418: note = source = /note="Description of Unknown:Cas13e.2 sequence"
  source 1..2418: mol_type = other DNA
atgaaggttgaaaatattaaagaaaaaagcaaaaaagcaatgtatttaatcaaccattatgagggacccaaaaaatggtgttttgcaatagttctgaatagggcatgtgataattacgaggacaatccacacttgttttccaaatcacttttggaatttgaaaaaacaagtcgaaaagattggtttgacgaagaaacacgagagcttgttgagcaagcagatacagaaatacagccaaatcctaacctgaaacctaatacaacagctaaccgaaaactcaaagatataagaaactatttttcgcatcattatcacaagaacgaatgcctgtattttaagaacgatgatcccatacgctgcattatggaagcggcgtatgaaaaatctaaaatttatatcaaaggaaagcagattgagcaaagcgatataccattgcccgaattgtttgaaagcagcggttggattacaccggcggggattttgttactggcatccttttttgttgaacgagggattctacatcgcttgatgggaaatatcggaggatttaaagataatcgaggcgaatacggtcttacacacgatatttttaccacctattgtcttaagggtagttattcaattcgggcgcaggatcatgatgcggtaatgttcagagatattctcggctatctgtcacgagttcccactgagtcatttcagcgtatcaagcaacctcaaatacgaaaagaaggccaattaagtgaaagaaagacggacaaatttataacatttgcactaaattatcttgaggattatgggctgaaagatttggaaggctgcaaagcctgttttgccagaagtaaaattgtaagggaacaagaaaatgttgaaagcataaatgataaggaatacaaacctcacgagaacaaaaagaaagttgaaattcacttcgatcagagcaaagaagaccgattttatattaatcgcaataacgttattttgaagattcagaagaaagatggacattccaacatagttaggatgggagtatatgaacttaaatatctcgttcttatgagtttagtgggaaaagcaaaagaagcagttgaaaaaattgacaactatatccaggatttgcgagaccagttgccttacatagaggggaaaaataaggaagagattaaagaatacgtcaggttctttccacgatttatacgttctcacctcggtttactacagattaacgatgaagaaaagataaaagctcgattagattatgttaagaccaagtggttagataaaaaggaaaaatcgaaagagcttgaacttcataaaaaaggacgggacatcctcaggtatatcaacgagcgatgtgatagagagcttaacaggaatgtatataaccgtattttagagctcctggtcagcaaagacctcactggtttttatcgtgagcttgaagaactaaaaagaacaaggcggatagataaaaatattgtccagaatctttctgggcaaaaaaccattaatgcactgcatgaaaaggtctgtgatctggtgctgaaggaaatcgaaagtctcgatacagaaaatctcaggaaatatcttggattgatacccaaagaagaaaaagaggtcactttcaaagaaaaggtcgataggattttgaaacagccagttatttacaaagggtttctgagataccaattcttcaaagatgacaaaaagagttttgtcttacttgttgaagacgcattgaaggaaaaaggaggaggttgtgatgttcctcttgggaaagagtattataaaatcgtgtcacttgataagtatgataaagaaaataaaaccctgtgtgaaactctggcgatggataggctttgccttatgatggcaagacaatattatctcagtctgaatgcaaaacttgcacaggaagctcagcaaatcgaatggaagaaagaagatagtatagaattgattattttcaccttaaaaaatcccgatcaatcaaagcagagtttttctatacggttttcggtcagagattttacgaagttgtatgtaacggatgatcctgaatttctggcccggctttgttcctactttttcccagttgaaaaagagattgaatatcacaagctctattcagaagggataaataaatacacaaacctgcaaaaagagggaatcgaagcaatactcgagcttgaaaaaaagcttattgaacgaaatcggattcaatctgcaaaaaattatctctcatttaatgagataatgaataaaagcggttataataaagatgagcaggatgatctaaagaaggtgcgaaattctcttttgcattataagcttatctttgagaaagaacatctcaagaagttctatgaggttatgagaggagaagggatagagaaaaagtggtctttaatagtatga

SEQ ID NO: 17 - DNA, 2373 bp, unidentified
  misc_feature 1..2373: note = source = /note="Description of Unknown:Cas13f.1 sequence"
  source 1..2373: mol_type = other DNA
atgaatggcattgaattaaaaaaagaagaagcagcattttattttaatcaggcagagcttaatttaaaagccatagaagacaatatttttgataaagaaagacgaaagactctgcttaataatccacagatacttgccaaaatggaaaatttcattttcaatttcagagatgtaacaaaaaatgcaaaaggggaaattgactgcttgctgttgaaactaagagagctgagaaacttttactcgcattatgtccacaaacgagatgtaagagaattaagcaagggcgagaaacctatacttgaaaagtattaccaatttgcgattgaatcaaccggaagtgaaaatgttaaacttgagataatagaaaacgacgcgtggcttgcagatgccggtgtgttgtttttcttatgtatttttttgaagaaatctcaggcaaataagcttataagcggtatcagcggttttaaaagaaacgatgataccggtcagccgagaaggaatttatttacctatttcagtataagggagggatacaaggttgttccggaaatgcagaaacatttccttttgttttctcttgttaatcatctctctaatcaagatgattatattgaaaaagcgcatcagccatacgatataggcgagggtttattttttcatcgaatagcttctacatttcttaatataagtgggattttaagaaatatgaaattctatacctatcagagtaaaaggttagtagagcagcggggagaactcaaacgagaaaaggatatttttgcgtgggaagaaccgtttcaaggaaatagttattttgaaataaatggtcataaaggagtaatcggtgaagatgaattgaaggaactatgttatgcatttctgattggcaatcaagatgctaataaagtggaaggcaggattacacaatttctagaaaagtttagaaatgcgaacagtgtgcaacaagttaaagatgatgaaatgctaaaaccagagtattttcctgcaaattattttgctgaatcaggcgtcggaagaataaaggatagagtgcttaatcgtttgaataaagcgattaaaagcaataaggccaagaaaggagagattatagcatacgataagatgagagaggttatggcgttcataaataattctctgccggtagatgaaaaattgaaaccaaaagattacaaacgatatctgggaatggttcgtttctgggacagggaaaaagataacataaagcgggagttcgagacaaaagaatggtctaaatatcttccatctaatttctggacggcaaaaaaccttgaaagggtctatggtctggcaagagagaaaaacgcagaattattcaataaactaaaagcggatgtagaaaaaatggacgaacgggaacttgagaagtatcagaagataaatgatgcaaaggatttggcaaatttacgccggcttgcaagcgactttggtgtgaagtgggaagaaaaagactgggatgagtattcaggacagataaaaaaacaaattacagacagccagaaactaacaataatgaagcagcggataaccgcaggactaaagaaaaagcacggcatagaaaatcttaacctgagaataactatcgacatcaataaaagcagaaaggcagttttgaacagaattgcgattccgaggggttttgtaaaaaggcatattttaggatggcaagagtctgagaaggtatcgaaaaagataagagaggcagaatgcgaaattctgctgtcgaaagaatacgaagaactatcgaaacaatttttccaaagcaaagattatgacaaaatgacacggataaatggcctttatgaaaaaaacaaacttatagccctgatggcagtttatctaatggggcaattgagaatcctgtttaaagaacacacaaaacttgacgatattacgaaaacaactgtggatttcaaaatatctgataaggtgacggtaaaaatccccttttcaaattatccttcgctcgtttatacaatgtccagtaagtatgttgataatatagggaattatggattttccaacaaagataaagacaagccgattttaggtaagattgatgtaatagaaaaacagcgaatggaatttataaaagaggttcttggttttgaaaaatatctttttgatgataaaataatagataaaagcaaatttgctgatacagcgactcatataagttttgcagaaatagttgaggagcttgttgaaaaaggatgggacaaagacagactgacaaaacttaaagatgcaagaaataaagccctgcatggtgaaatactgacgggaaccagctttgatgaaacaaaatcattgataaacgaattaaaaaaatga

SEQ ID NO: 18 - DNA, 2379 bp, unidentified
  misc_feature 1..2379: note = source = /note="Description of Unknown:Cas13f.2 sequence"
  source 1..2379: mol_type = other DNA
atgtccccagatttcatcaaattagaaaaacaggaagcagctttttactttaatcagacagagcttaatttaaaagccatagaaagcaatattttagacaaacaacagcgaatgattctgcttaataatccacggatacttgccaaagtaggaaatttcattttcaatttcagagatgtaacaaaaaatgcaaaaggagaaatagactgtctgctatttaaactggaagagctaagaaacttttactcgcattatgttcataccgacaatgtaaaggaattgagtaacggagaaaaacccctactggaaagatattatcaaatcgctattcaggcaaccaggagtgaggatgttaagttcgaattgtttgaaacaagaaacgagaataagattacggatgccggtgtattgtttttcttatgtatgtttttaaaaaaatcacaggcaaacaagcttataagcggtatcagcggcttcaaaagaaatgatccaacaggccagccgagaagaaacttatttacctatttcagtgcaagagaaggatataaggctttgcctgatatgcagaaacattttcttctttttactctggttaattatttgtcgaatcaggatgagtatatcagcgagcttaaacaatatggagagattggtcaaggagccttttttaatcgaatagcttcaacatttttgaatatcagcgggatttcaggaaatacgaaattctattcgtatcaaagtaaaaggataaaagagcagcgaggcgaactcaatagcgaaaaggacagctttgaatggatagagcctttccaaggaaacagctattttgaaataaatgggcataaaggagtaatcggcgaagacgaattaaaagaactttgttatgcattgttggttgccaagcaagatattaatgccgttgaaggcaaaattatgcaattcctgaaaaagtttagaaatactggcaatttgcagcaagttaaagatgatgaaatgctggaaatagaatattttcccgcaagttattttaatgaatcaaaaaaagaggacataaagaaagagattcttggccggctggataaaaagattcgctcctgctctgcaaaggcagaaaaagcctatgataagatgaaagaggtgatggagtttataaataattctctgccggcagaggaaaaattgaaacgcaaagattatagaagatatctaaagatggttcgtttctggagcagagaaaaaggcaatatagagcgggaatttagaacaaaggaatggtcaaaatatttttcatctgatttttggcggaagaacaatcttgaagatgtgtacaaactggcaacacaaaaaaacgctgaactgttcaaaaatctaaaagcggcagcagagaaaatgggtgaaacggaatttgaaaagtatcagcagataaacgatgtaaaggatttggcaagtttaaggcggcttacgcaagattttggtttgaagtgggaagaaaaggactgggaggagtattccgagcagataaaaaaacaaattacggacaggcagaaactgacaataatgaaacaaagggttacggctgaactaaagaaaaagcacggcatagaaaatcttaatctgagaataaccatcgacagcaataaaagcagaaaggcggttttgaacagaatagcaattccaagaggatttgtaaaaaaacatattttaggctggcagggatctgagaagatatcgaaaaatataagggaagcagaatgcaaaattctgctatcgaaaaaatatgaagagttatcaaggcagttttttgaagccggtaatttcgataagctgacgcagataaatggtctttatgaaaagaataaacttacagcttttatgtcagtatatttgatgggtcggttgaatattcagcttaataagcacacagaacttggaaatcttaaaaaaacagaggtggattttaagatatctgataaggtgactgaaaaaataccgttttctcagtatccttcgcttgtctatgcgatgtctcgcaaatatgttgacaatgtggataaatataaattttctcatcaagataaaaagaagccatttttaggtaaaattgattcaattgaaaaagaacgtattgaattcataaaagaggttctcgattttgaagagtatctttttaaaaataaggtaatagataaaagcaaattttccgatacagcgactcatattagctttaaggaaatatgtgatgaaatgggtaaaaaaggatgtaaccgaaacaaactaaccgaacttaacaacgcaaggaacgcagccctgcatggtgaaataccgtcggagacctcttttcgtgaagcaaaaccgttgataaatgaattgaaaaaatga

SEQ ID NO: 19 - DNA, 2379 bp, unidentified
  misc_feature 1..2379: note = source = /note="Description of Unknown:Cas13f.3 sequence"
  source 1..2379: mol_type = other DNA
atgtccccagatttcatcaaattagaaaaacaagaagcagctttttactttaatcagacagagcttaatttaaaagccatagaaagcaatattttcgacaaacaacagcgagtgattctgcttaataatccacagatacttgccaaagtaggagattttattttcaatttcagagatgtaacaaaaaacgcaaaaggagaaatagactgtttgctattgaaactaagagagctgagaaacttttactcacactatgtctataccgatgacgtgaagatattgagtaacggcgaaagacctctgctggaaaaatattatcaatttgcgattgaagcaaccggaagtgaaaatgttaaacttgaaataatagaaagcaacaaccgacttacggaagcgggcgtgctgtttttcttgtgtatgtttttgaaaaagtctcaggcaaataagcttataagcggtatcagcggttttaaaagaaatgacccgacaggtcagccgagaaggaatttatttacctacttcagtgtaagggagggatacaaggttgtgccggatatgcagaaacattttcttttgtttgttcttgtcaatcatctctctggtcaggatgattatattgaaaaggcgcaaaagccatacgatataggcgagggtttattttttcatcgaatagcttctacatttcttaatatcagtgggattttaagaaatatggaattctatatttaccagagcaaaagactaaaggagcagcaaggagagctcaaacgtgaaaaggatatttttccatggatagagcctttccagggaaatagttattttgaaataaatggtaataaaggaataatcggcgaagatgaattgaaagagctttgttatgcgttgctggttgcaggaaaagatgtcagagccgtcgaaggtaaaataacacaatttttggaaaagtttaaaaatgcggacaatgctcagcaagttgaaaaagatgaaatgctggacagaaacaattttcccgccaattatttcgccgaatcgaacatcggcagcataaaggaaaaaatacttaatcgtttgggaaaaactgatgatagttataataagacggggacaaagattaaaccatacgacatgatgaaagaggtaatggagtttataaataattctcttccggcagatgaaaaattgaaacgcaaagattacagaagatatctaaagatggttcgtatctgggacagtgagaaagataatataaagcgggagtttgaaagcaaagaatggtcaaaatatttttcatctgatttctggatggcaaaaaatcttgaaagggtctatgggttggcaagagagaaaaacgccgaattattcaataagctaaaagcggttgtggagaaaatggacgagcgggaatttgagaagtatcggctgataaatagcgcagaggatttggcaagtttaagacggcttgcgaaagattttggcctgaagtgggaagaaaaggactggcaagagtattctgggcagataaaaaaacaaatttctgacaggcagaaactgacaataatgaaacaaaggattacggctgaactaaagaaaaagcacggcatagaaaatctcaatcttagaataaccatcgacagcaataaaagcagaaaggcagttttgaacagaatcgcagttccaagaggttttgtgaaagagcatattttaggatggcaggggtctgagaaggtatcgaaaaagacaagagaagcaaagtgcaaaattctgctctcgaaagaatatgaagaattatcaaagcaatttttccaaaccagaaattacgacaagatgacgcaggtaaacggtctttacgaaaagaataaactcttagcatttatggtcgtttatcttatggagcggttgaatatcctgcttaataagcccacagaacttaatgaacttgaaaaagcagaggtggatttcaagatatctgataaggtgatggccaaaatcccgttttcacagtatccttcgcttgtgtacgcgatgtccagcaaatatgctgatagtgtaggcagttataaatttgagaatgatgaaaaaaacaagccgtttttaggcaagatcgatacaatagaaaaacaacgaatggagtttataaaagaagtccttggttttgaagagtatctttttgaaaagaagataatagataaaagcgaatttgccgacacagcgactcatataagttttgatgaaatatgtaatgagcttattaaaaaaggatgggataaagacaaactaaccaaacttaaagatgccaggaacgcggccctgcatggcgaaataccggcggagacctcttttcgtgaagcaaaaccgttgataaatggattgaaaaaatga

SEQ ID NO: 20 - DNA, 2400 bp, unidentified
  misc_feature 1..2400: note = source = /note="Description of Unknown:Cas13f.4 sequence"
  source 1..2400: mol_type = other DNA
atgaacatcattaaattaaaaaaagaagaagctgcgttttattttaatcagacgatcctcaatctttcagggcttgatgaaattattgaaaaacaaattccgcacataatcagcaacaaggaaaatgcaaagaaagtgattgataagattttcaataaccgcttattattaaaaagtgtggagaattatatctacaactttaaagatgtggctaaaaacgcaagaactgaaattgaggctatattgttgaaattagtagagctacgtaatttttactcacattacgttcataatgataccgtcaagatactaagtaacggtgaaaaacctatactggaaaaatattatcaaattgctatagaagcaaccggaagtaaaaatgttaaacttgtaatcatagaaaacaacaactgtctcacggattctggcgtgctgtttttgctgtgtatgttcttaaaaaaatcacaggcaaacaagcttataagttccgttagtggttttaaaaggaatgataaagaaggacaaccgagaagaaatctattcacttattatagtgtgagggagggatataaggttgtgcctgatatgcagaagcatttccttctattcgctctggtcaatcatctatctgagcaggatgatcatattgagaagcagcagcagtcagacgagctcggtaagggtttgtttttccatcgtatagcttcgacttttttaaacgagagcggcatcttcaataaaatgcaattttatacatatcagagcaacaggctaaaagagaaaagaggagaactcaaacacgaaaaggatacctttacatggatagagccttttcaaggcaatagttattttacgttaaatggacataagggagtgattagtgaagatcaattgaaggagctttgttacacaattttaattgagaagcaaaacgttgattccttggaaggtaaaattatacaatttctcaaaaaatttcagaatgtcagcagcaagcagcaagttgacgaagatgaattgcttaaaagagaatatttccctgcaaattactttggccgggcaggaacagggaccctaaaagaaaagattctaaaccggcttgataagaggatggatcctacatctaaagtgacggataaagcttatgacaaaatgattgaagtgatggaatttatcaatatgtgccttccgtctgatgagaagttgaggcaaaaggattatagacgatacttaaagatggttcgtttctggaataaggaaaagcataacattaagcgcgagtttgacagtaaaaaatggacgaggtttttgccgacggaattgtggaataaaagaaatctagaagaagcctatcaattagcacggaaagagaacaaaaagaaacttgaagatatgagaaatcaagtacgaagccttaaagaaaatgaccttgaaaaatatcagcagattaattacgttaatgacctggagaatttaaggcttctgtcacaggagttaggtgtgaaatggcaggaaaaggactgggttgaatattccgggcagataaagaagcagatatcagacaatcagaaacttacaatcatgaaacaaaggattaccgctgaactaaagaaaatgcacggcatcgagaatcttaatcttagaataagcattgacacgaataaaagcaggcagacggttatgaacaggatagctttgcccaaaggttttgtgaagaatcatatccagcaaaattcgtctgagaaaatatcgaaaagaataagagaggattattgtaaaattgagctatcgggaaaatatgaagaactttcaaggcaattttttgataaaaagaatttcgataagatgacactgataaacggcctttgtgaaaagaacaaacttatcgcatttatggttatctatcttttggagcggcttggatttgaattaaaggagaaaacaaaattaggcgagcttaaacaaacaaggatgacatataaaatatccgataaggtaaaagaagatatcccgctttcctattaccccaagcttgtgtatgcaatgaaccgaaaatatgttgacaatatcgatagttatgcatttgcggcttacgaatccaaaaaagctattttggataaagtggatatcatagaaaagcaacgtatggaatttatcaaacaagttctctgttttgaggaatatattttcgaaaataggattatcgaaaaaagcaaatttaatgacgaggagactcatataagttttacacaaatacatgatgagcttattaaaaaaggacgggacacagaaaaactctctaaactcaaacatgcaaggaataaagccttgcacggcgagattcctgatgggacttcttttgaaaaagcaaagctattgataaatgaaatcaaaaaatga

SEQ ID NO: 21 - DNA, 2412 bp, unidentified
  misc_feature 1..2412: note = source = /note="Description of Unknown:Cas13f.5 sequence"
  source 1..2412: mol_type = other DNA
atgaatgctatcgaactaaaaaaagaggaagcagcattttattttaatcaggcaagactcaacatttcaggacttgatgaaattattgaaaagcagttaccacatataggtagtaacagggagaatgcgaaaaaaactgttgatatgattttggataatcccgaagtcttgaagaagatggaaaattatgtctttaactcacgagatatagcaaagaacgcaagaggtgaacttgaagcattgttgttgaaattagtagaactgcgtaatttttattcacattatgttcataaagatgatgttaagacattgagttacggagaaaaacctttactggataaatattatgaaattgcgattgaagcgaccggaagtaaagatgtcagacttgagataatagatgataaaaataagcttacagatgccggtgtgctttttttattgtgtatgtttttgaaaaaatcagaggcaaacaaacttatcagttcaatcaggggctttaaaagaaacgataaagaaggccagccgagaagaaatctattcacttactacagtgtcagagagggatataaggttgtgcctgatatgcagaaacattttcttttattcacactggttaaccatttgtcaaatcaggatgaatacatcagtaatcttaggccgaatcaagaaatcggccaagggggatttttccatagaatagcatcaaaatttttgagcgatagcgggattttacatagtatgaaattctacacctaccggagtaaaagactaacagaacaacggggggagcttaagccgaaaaaagatcattttacatggatagagccttttcagggaaacagttatttttcagtgcagggccaaaaaggagtaattggtgaagagcaattaaaggagctttgttatgtattgctggttgccagagaagattttagggccgttgagggcaaagttacacaatttctgaaaaagtttcagaatgctaataacgtacagcaagttgaaaaagatgaagtgctggaaaaagaatattttcctgcaaattattttgaaaatcgagacgtaggcagagtaaaggataagatacttaatcgtttgaaaaaaatcactgaaagctataaagctaaagggagggaggttaaagcctatgacaagatgaaagaggtaatggagtttataaataattgcctgccaacagatgaaaatttgaaactcaaagattacagaagatatctgaaaatggttcgtttctggggcagggaaaaggaaaatataaagcgggaatttgacagtaaaaaatgggagaggtttttgccaagagaactctggcagaaaagaaacctcgaagatgcgtatcaactggcaaaagagaaaaacaccgagttattcaataaattgaaaacaactgttgagagaatgaacgaactggaattcgaaaagtatcagcagataaacgacgcaaaagatttggcaaatttaaggcaactggcgcgggacttcggcgtgaagtgggaagaaaaggactggcaagagtattcggggcagataaaaaaacaaattacagacaggcaaaaacttacaataatgaaacaaaggattactgctgcattgaagaaaaagcaaggcatagaaaatcttaatcttaggataacaaccgacaccaataaaagcagaaaggtggtattgaacagaatagcgctacctaaaggttttgtaaggaagcatatcttaaaaacagatataaagatatcaaagcaaataaggcaatcacaatgtcctattatactgtcaaacaattatatgaagctggcaaaggaattctttgaggagagaaattttgataagatgacgcagataaacgggctatttgagaaaaatgtacttatagcgtttatgatagtttatctgatggaacaactgaatcttcgacttggtaagaatacggaacttagcaatcttaaaaaaacggaggttaattttacgataaccgacaaggtaacggaaaaagtccagatttcgcagtatccatcgcttgttttcgccataaacagagaatatgttgatggaatcagcggttataagttaccgcccaaaaaaccgaaagagcctccgtatactttcttcgagaaaatagacgcaatagaaaaagaacgaatggaattcataaaacaggtcctcggtttcgaagaacatctttttgagaagaatgtaatagacaaaactcgctttactgatactgcgactcatataagttttaatgaaatatgtgatgagcttataaaaaaaggatgggacgaaaacaaaataataaaacttaaagatgcgaggaatgcagcattgcatggtaagataccggaggatacgtcttttgatgaagcgaaagtactgataaatgaattaaaaaaatga

SEQ ID NO: 22 - DNA, 2328 bp, synthetic construct
  misc_feature 1..2328: note = source = /note="Description of Artificial Sequence: Syntheticpolynucleotide"
  source 1..2328: mol_type = other DNA
atggcccaggtgagcaagcagacctccaagaagagggagctgagcatcgacgagtaccagggcgcccggaagtggtgcttcaccattgccttcaacaaggccctggtgaaccgggacaagaacgacggcctgttcgtggaaagcctgctgagacacgagaagtacagcaagcacgactggtacgacgaagatacccgggccctgatcaagtgcagcacccaggccgccaacgccaaggctgaagccctgcggaactacttcagtcactaccggcatagccctggctgcctgaccttcaccgccgaggacgaactgcggaccatcatggagagagcctatgagcgggccatcttcgagtgcagaagaagagagacagaggtgatcatcgagtttcccagcctgttcgagggcgaccggatcaccaccgccggcgtggtgtttttcgtgagctttttcgtggaaagaagagtgctggatcggctgtatggagccgtgtccggcctgaagaagaatgagggacagtacaagctgacccggaaggccctgagcatgtactgcctgaaggacagcagattcaccaaggcctgggataagcgggtgctgctgttcagagacatcctggcccagctgggaagaatccccgccgaggcctacgagtactaccacggcgagcagggtgataagaagagagctaacgacaatgagggcacaaatcccaagcggcacaaggacaagttcatcgaatttgcactgcactacctggaagcccagcacagcgagatctgcttcggcagacgccacatcgtgcgggaagaggccggcgccggcgatgagcacaagaagcaccggaccaagggaaaggtggtggtggacttcagcaagaaggacgaggaccagagctactatatctccaagaacaacgtgatcgtgcggatcgacaagaacgccggccctagaagctaccggatgggcctgaacgagctgaagtacctcgtgctgctgagcctgcaggggaagggcgacgatgccatcgccaagctgtacagatacagacagcacgtggagaacatcctggatgtggtgaaggtgaccgataaggataaccacgtgttcctgccccgcttcgtgctggagcagcacggcatcggcagaaaggccttcaagcagcggatcgatggacgggtgaagcacgtgcggggcgtgtgggagaagaagaaggccgccaccaatgaaatgaccctgcacgagaaggccagagacatcctgcagtacgtgaacgaaaactgcacccggtccttcaaccctggcgaatacaacagactgctggtgtgcctggtgggcaaggacgtggagaactttcaggccggcctgaagcggctgcagctggccgaaaggatcgatggccgggtgtactccatcttcgcccagaccagcaccatcaatgagatgcaccaggtggtgtgcgaccagatcctgaaccggctgtgcagaatcggcgaccagaagctgtacgattacgtgggactgggcaagaaggacgaaatcgactacaagcagaaggtggcctggttcaaggagcacatcagcatccggagaggattcctgagaaagaagttctggtacgatagcaagaagggattcgcaaagctggtggaggaacacctggagtccggcggcggccagcgcgacgtgggcctggacaagaagtactaccacatcgacgccatcggcagattcgagggcgccaaccccgccctgtacgagaccctggccagagatcggctgtgcctcatgatggcccagtacttcctgggcagcgtgagaaaggaactgggcaacaagattgtgtggagcaacgacagcatcgaactgcctgtggaaggctctgtgggaaatgagaagagcatcgtgttctccgtgtctgactacggcaagctgtacgtgctggacgatgccgaattcctgggccggatctgcgaatacttcatgccccacgaaaagggcaagatccggtaccacacagtgtacgaaaagggctttagagcatacaacgacctgcagaagaagtgcgtggaggccgtgctggctttcgaagagaaggtggtgaaggccaagaagatgagcgagaaggaaggcgcccactacatcgacttccgggagatcctggcccagaccatgtgcaaggaggccgagaagaccgcagtgaacaaggtgagacgcgccttcttccaccaccacctgaagttcgtgattgacgagttcggcctgttcagcgacgtgatgaagaagtacggcatcgagaaggaatggaagttccctgtcaagtaa

SEQ ID NO: 23 - DNA, 2418 bp, synthetic construct
  misc_feature 1..2418: note = source = /note="Description of Artificial Sequence: Syntheticpolynucleotide"
  source 1..2418: mol_type = other DNA
atgaaggtggagaacatcaaggaaaagtccaagaaggctatgtatctgatcaaccactatgaaggccctaagaagtggtgcttcgccatcgtgctgaatagggcctgcgacaactatgaggataacccccacctgttcagcaagagcctgctggaatttgaaaagaccagcagaaaggactggttcgacgaggagaccagggaactggtggagcaggccgacaccgagatccagcccaaccccaacctgaagcctaacaccaccgccaacagaaagctgaaggacatccggaactacttcagccaccactaccacaagaatgagtgcctgtacttcaagaacgacgaccctatccggtgcatcatggaggcagcctacgagaagtccaagatctacatcaagggcaagcagattgagcagtccgacatccccctccctgagctgtttgagtctagcggctggatcaccccagccggcatcctgctgctggccagcttctttgtggagagaggcattctgcacagactgatgggcaacatcggcggcttcaaggacaaccggggcgaatacggactgacccacgatatcttcaccacctactgcctgaagggcagctactccatcagagcccaggaccacgacgccgtgatgttcagagacatcctgggctacctgagcagagtgccgaccgagagctttcagcgcatcaagcagccacagatcagaaaggaggggcagctgagcgagcggaagacagacaagtttatcaccttcgccctgaactacctggaagattatggactgaaggatctggaaggctgcaaggcctgcttcgcccggagcaagatcgtgagagagcaggagaacgtggaaagcatcaatgacaaggagtacaagcctcacgaaaacaagaagaaggtggaaatccacttcgatcagtctaaggaagaccggttctacatcaaccggaacaacgtgatcctgaagatccagaagaaggacggccacagcaacatcgtgagaatgggcgtgtacgagctgaagtatctggtgctgatgtccctggtgggcaaggccaaggaagccgtggagaagatcgacaactacatccaggatctgagagaccagctgccctacatcgagggcaagaacaaggaagaaatcaaggagtacgtgagattcttccccagattcatcagatcccacctgggcctgctgcagattaacgatgaggagaagatcaaggcccggctggactatgtgaagacaaagtggctggacaagaaggagaagtccaaggagctggagctgcacaagaagggccgggatatcctgcggtacatcaacgagcggtgcgaccgggagctgaaccggaacgtgtacaaccggatcctggagctgctggtgagcaaggacctgaccggcttctaccgggagctggaggagctgaagcggaccagacggatcgataagaacattgtgcagaacctgtccggccagaagaccatcaacgccctgcacgaaaaggtgtgcgatctcgtgctgaaggagatcgagagcctggacaccgagaacctgcggaagtacctgggcctgatccccaaggaggagaaggaagtgacctttaaggagaaggtggacaggatcctgaagcagccggtgatctacaagggcttcctgcggtaccagttcttcaaggacgacaagaagagcttcgtgctgctggtggaagacgccctgaaggagaagggaggcggctgcgacgtgcccctgggcaaggagtactacaagatcgtgtccctggacaagtatgacaaggaaaataagaccctgtgcgagaccctggcaatggatagactgtgcctgatgatggcccggcagtattacctgagcctgaacgccaagctggcccaggaggcccagcagatcgaatggaagaaggaggatagcattgagctgatcatcttcacactgaagaatcctgaccagtccaagcagagcttctccatccggttcagcgtgcgggacttcaccaagctgtacgtgaccgacgaccccgaattcctggcccggctgtgcagctacttcttccccgtggagaaggagatcgaataccacaagctgtactctgaaggcattaacaagtacaccaacctgcagaaggaggggatcgaagccatcctggagctggagaagaagctgatcgaaagaaaccggatccagtccgccaagaactacctgagctttaacgaaatcatgaacaagagcggctacaacaaggatgagcaggatgacctgaagaaggtgaggaactccctgctgcactacaagctgatcttcgaaaaggagcacctgaagaagttctatgaagtgatgcggggcgagggaatcgagaagaagtggtccctgatcgtgtaa

SEQ ID NO: 24 - DNA, 2373 bp, synthetic construct
  misc_feature 1..2373: note = source = /note="Description of Artificial Sequence: Syntheticpolynucleotide"
  source 1..2373: mol_type = other DNA
atgaatggcatcgagctgaagaaggaagaagccgccttctacttcaatcaggccgagctgaacctgaaggccattgaggacaacatcttcgacaaggagagacggaagacactgctgaacaacccccagatcctggccaagatggagaactttatcttcaatttccgggacgtgaccaagaacgccaagggcgaaatcgactgcctgctgctgaagctgagagagctgcggaacttttacagccactacgtgcacaagcgggacgtcagagaactgagcaagggcgagaagccgatcctggagaagtactaccagttcgccatcgaatccaccggctctgagaacgtgaagctcgaaatcatcgaaaacgacgcctggctggccgacgccggcgtgctgttcttcctgtgcatcttcctgaagaagagccaggcaaacaagctgatcagcggcatcagcggcttcaagagaaacgacgacaccggccagcctcggagaaacctgttcacctacttctccatccgggagggctacaaggtggtgcccgaaatgcagaagcacttcctgctgttctccctggtgaaccacctgagcaaccaggacgattatatcgaaaaggcccaccagccctacgacatcggcgagggcctcttcttccaccggattgccagcaccttcctgaacatctccggaatcctgagaaacatgaagttctacacctatcagagcaagagactggtggagcagagaggcgagctgaagcgggaaaaggacatcttcgcctgggaagaaccgtttcagggcaattcctactttgagatcaacggccacaagggcgtgattggcgaagacgagctgaaggagctgtgctacgccttcctgatcggcaaccaggacgccaacaaggtggagggccggatcacccagttcctggagaagttcagaaacgccaacagcgtgcagcaggtgaaggacgacgagatgctgaagcctgaatatttccccgccaactactttgccgagagcggcgtgggccggatcaaggaccgggtgctgaacagactgaacaaggccatcaagagcaacaaggccaagaagggcgagatcatcgcctatgacaagatgagagaagtgatggctttcatcaataactctctgcccgtggacgagaagctgaagcccaaggattacaagagatacctgggcatggtgagattctgggatagagaaaaggacaatatcaagcgcgagttcgaaacgaaggagtggagcaagtatctgccctccaacttctggaccgccaagaacctggagagagtgtacggactggcccgggaaaagaacgcagagctgtttaacaagctgaaggccgacgtggagaagatggacgaaagagagctggaaaagtatcagaagatcaacgacgccaaggatctggccaacctgcggcggctggccagcgacttcggagtgaagtgggaggagaaggattgggacgagtactccggccagatcaagaagcagatcacagattcccagaagctgaccatcatgaagcagagaatcacagccggcctgaagaagaagcacggcatcgaaaacctgaacctgaggatcaccatcgacatcaacaagtccagaaaggccgtgctgaatcggatcgccatccccagaggatttgtgaagcggcacatcctgggctggcaggaatccgagaaggtgagcaagaagatcagagaagccgaatgcgagattctgctgagcaaggagtacgaggagctgagcaagcagttctttcagagcaaggactacgacaagatgacccgcatcaacggcctgtacgagaagaataagctgatcgccctgatggccgtgtatctgatggggcagctgagaatcctgttcaaggagcacaccaagctggacgacatcaccaagaccaccgtggatttcaagatcagcgacaaggtgaccgtgaagatccccttctccaactatccctccctggtgtacaccatgagcagcaagtacgtggacaatatcggcaactacggcttcagcaacaaggacaaggataagcccattctgggcaagatcgacgtgatcgagaagcagcggatggagtttatcaaggaggtgctgggattcgagaagtacctgtttgacgataagatcatcgacaagagcaagttcgccgacaccgccacccacatcagctttgccgaaatcgtggaagaactggtggagaagggctgggacaaggaccggctgacgaagctgaaggatgcccggaacaaggccctgcacggcgagatcctgaccggcaccagcttcgacgagacaaagtccctgatcaacgagctgaagaagtaa

SEQ ID NO: 25 - DNA, 2379 bp, synthetic construct
  misc_feature 1..2379: note = source = /note="Description of Artificial Sequence: Syntheticpolynucleotide"
  source 1..2379: mol_type = other DNA
atgagccctgatttcatcaagctggagaagcaggaagcagccttctactttaaccagaccgagctgaacctgaaggccatcgaatccaatatcctggataagcagcagagaatgatcctgctgaacaaccccagaatcctggccaaggtgggcaacttcatcttcaatttccgggacgtgaccaagaacgcaaagggcgaaatcgactgcctgctgttcaagctggaggaactgcggaacttctacagccactacgtgcacaccgataacgtgaaggaactgtccaacggagagaagcctctgctggagcggtactaccagatcgccatccaggccacaagaagcgaggacgtgaagttcgagctgttcgagaccaggaacgagaacaagatcaccgacgcaggcgtgctgttcttcctgtgcatgttcctgaagaagagccaggctaataagctgatttccggcatcagcggcttcaagcggaacgaccccaccggccagcccagacggaacctctttacctacttctctgcccgggagggctacaaggccctgcctgacatgcagaagcacttcctgctgttcaccctggtgaactacctgagcaaccaggacgagtacatctccgagctgaagcagtacggagagatcggacagggagccttcttcaacagaatcgccagcaccttcctgaacatcagcggcatcagcggcaacaccaagttctacagctaccagagcaagagaatcaaggagcagcggggcgaactgaacagcgaaaaggacagcttcgagtggatcgagccctttcagggcaactcttattttgagatcaacggccacaagggcgtgatcggcgaagacgagctgaaggagctgtgctacgccctgctggtggccaagcaggacatcaatgccgtggagggaaagatcatgcagttcctgaagaagttcaggaacaccggcaacctgcagcaggtgaaggacgacgagatgctggaaatcgagtactttcccgccagctacttcaacgagagcaagaaggaggacatcaagaaggagatcctgggcagactggacaagaagatccggtcctgcagcgccaaggccgagaaggcctacgacaagatgaaggaggtgatggagtttatcaataacagcctgcccgccgaggagaagctgaagaggaaggactaccgcagatacctgaagatggtgagattctggtccagagaaaagggcaacatcgagagagagttcagaaccaaggagtggtccaagtacttcagcagcgacttctggagaaagaacaatctggaggatgtgtacaagctggccacccagaagaacgccgagctgttcaagaatctgaaggccgccgccgagaagatgggcgaaacagaattcgaaaagtaccagcagatcaacgatgtgaaggacctggccagcctgagacggctgacccaggatttcggcctgaagtgggaggagaaggattgggaggagtacagcgaacagatcaagaagcagatcaccgaccggcagaagctgacaatcatgaagcagcgggtgaccgccgagctgaagaagaagcacggcatcgagaatctgaacctcagaattaccatcgattccaacaagagcagaaaggccgtgctgaacagaatcgccattccccggggcttcgtgaagaagcacattctgggctggcagggcagcgaaaagatcagcaagaatatccgggaggccgagtgcaagatcctgctgtccaagaagtatgaggagctgtctcggcagttctttgaggctggcaacttcgacaagctgacccagatcaacggcctgtacgaaaagaataagctgaccgccttcatgtccgtctacctgatgggcagactgaacatccagctgaacaagcacacggagctgggaaatctgaagaagaccgaggtggacttcaagatttccgacaaggtgacagaaaagatccccttctcccagtaccctagcctggtgtacgctatgagccggaagtacgtggacaacgtggacaagtacaagttcagccaccaggacaagaagaagcccttcctgggcaagatcgacagcatcgaaaaggagagaatcgaattcatcaaggaggtgctggacttcgaagagtacctgtttaagaacaaggtgatcgacaagagcaagttcagcgataccgccacccatatctctttcaaggaaatctgcgacgagatgggcaagaagggctgcaaccgcaacaagctgaccgagctgaataacgctagaaacgccgcactgcacggagaaatccccagcgagaccagcttccgggaggccaagcccctgatcaacgaactgaagaagtaa

SEQ ID NO: 26 - DNA, 2379 bp, synthetic construct
  misc_feature 1..2379: note = source = /note="Description of Artificial Sequence: Syntheticpolynucleotide"
  source 1..2379: mol_type = other DNA
atgagccctgacttcatcaagctggaaaagcaggaagccgccttctactttaatcagaccgagctgaacctgaaggccatcgagagcaacatcttcgacaagcagcagcgggtgatcctgctgaataacccccagatcctggccaaggtgggcgacttcatcttcaacttccgggacgtgaccaagaacgccaagggagaaatcgactgcctgctgctgaagctgcgggagctgagaaacttctacagccactatgtgtacaccgacgacgtgaagatcctgagcaacggcgagaggcccctgctggagaagtactaccagtttgccatcgaggccaccggatctgagaatgtgaagctggagatcatcgagagcaacaaccggctgaccgaagcgggcgtgctgttcttcctgtgcatgttcctgaagaagagccaggccaacaagctgatttccggcatctccggattcaagcgcaacgaccctaccggacagcctcggcggaacctgttcacctactttagcgtgcgggagggctacaaggtggtgcccgacatgcagaagcacttcctgctgttcgtgctggtgaaccacctgtccggccaggatgactatattgagaaggcccagaagccctacgacatcggcgaaggcctgttcttccacagaatcgccagcacctttctcaacatcagcggcatcctgagaaacatggaattctacatctaccagagcaagcggctgaaggagcagcagggagagctgaagagagagaaggacatcttcccttggatcgagcctttccagggcaacagctactttgagatcaacggaaacaagggcatcatcggcgaggacgaactgaaggaactgtgctacgccctgctggtggccggcaaggacgtgagagccgtggaaggaaagatcacccagttcctggagaagttcaagaacgccgataacgcccagcaggtggagaaggatgaaatgctggaccggaacaacttccctgccaattactttgccgaaagcaacatcggcagcatcaaggaaaagatcctgaatagactgggcaagaccgacgactcctacaacaagaccggcaccaagatcaagccctacgacatgatgaaggaggtgatggagttcatcaataattctctgcccgccgatgagaagctgaagcggaaggactaccggagatacctgaagatggtccggatctgggacagcgaaaaggacaatatcaagcgggagtttgagagcaaggaatggagcaagtatttcagcagcgacttctggatggccaagaacctggaaagagtgtacggcctggccagggaaaagaacgccgagctgtttaacaagctgaaggccgtggtggagaagatggacgagcgggagttcgaaaagtaccggctgatcaacagcgccgaagacctggccagcctgcggagactggccaaggacttcggcctgaagtgggaggagaaggactggcaggagtattctggccagatcaagaagcagatctccgacagacagaagctgacaattatgaagcagcggatcacagccgaactgaagaagaagcacggaatcgagaacctgaatctgcggatcaccatcgacagcaacaagtccagaaaggccgtgctgaaccggatcgccgtgccccggggcttcgtgaaggaacacatcctgggctggcaaggctctgaaaaggtgagcaagaagaccagagaagccaagtgcaagatcctgctgagcaaggagtacgaggaactgagcaagcagttctttcagacacggaattacgacaagatgacccaggtgaacggcctgtacgagaagaacaagctgctggccttcatggtggtgtacctgatggagagactgaacatcctgctgaacaagcccacagagctgaacgaactggaaaaggccgaagtggacttcaagatctccgacaaggtgatggccaagatccctttctctcagtaccccagcctggtgtatgcaatgagctccaagtacgccgacagcgtgggctcttacaagttcgaaaacgacgagaagaacaagccctttctgggcaagatcgacacaatcgagaagcagagaatggagttcatcaaggaggtgctgggcttcgaggaatacctgttcgagaagaagatcatcgataagagcgaattcgccgacaccgccacccacatcagcttcgacgagatctgcaacgagctgatcaagaagggctgggacaaggacaagctgaccaagctgaaggacgcccggaacgccgccctgcacggcgagatccccgccgagaccagcttccgggaggccaagcccctgattaacggcctgaagaagtaa

SEQ ID NO: 27 - DNA, 2400 bp, synthetic construct
  misc_feature 1..2400: note = source = /note="Description of Artificial Sequence: Syntheticpolynucleotide"
  source 1..2400: mol_type = other DNA
atgaacatcatcaagctgaagaaggaggaagccgccttttactttaaccagacaatcctgaatctgagcggcctggacgagatcatcgagaagcagatcccccacatcatctccaataaggaaaacgccaagaaggtgattgataagatcttcaataacagactgctgctgaagagcgtggaaaactatatctacaacttcaaggacgtggccaagaacgcccggaccgaaatcgaagccatcctgctgaagctggtggagctgagaaacttctactcccactacgtgcacaacgacaccgtgaagatcctgtccaatggcgagaagcccatcctggaaaagtactaccagatcgccatcgaagccaccggctctaagaacgtgaagctggtcattatcgaaaacaacaactgcctgaccgactccggcgtgctgttcctgctgtgcatgttcctgaagaagagccaggccaacaagctgattagcagcgtgagcggctttaagcggaacgacaaggaaggccagcccagaaggaacctctttacttactatagcgtgagggaaggctacaaggtggtgccagacatgcagaagcacttcctgctgttcgccctggtcaaccacctgtccgagcaggacgaccacatcgagaagcagcagcagagcgacgagctgggcaagggcctgttcttccacagaatcgccagcacattcctgaatgaaagcggcatcttcaacaagatgcagttttacacctaccagagcaatcggctgaaggagaagcggggcgagctgaagcacgagaaggacaccttcacctggatcgagcctttccagggaaacagctacttcaccctgaacgggcacaagggcgtgatcagcgaggatcagctgaaggaactgtgctacacaatcctgatcgagaagcagaacgtggacagcctggagggcaagatcattcagttcctgaagaagtttcagaacgtgtctagcaagcagcaggtggatgaggacgagctgctgaagcgggaatacttccccgccaactacttcggccgggccggcaccggcaccctgaaggagaagatcctgaaccggctggacaagcggatggaccccaccagcaaggtgaccgacaaggcctatgacaagatgatcgaggtgatggagttcatcaacatgtgcctgcccagcgacgagaagctgcggcagaaggattaccggagatatctgaagatggtcagattctggaacaaggagaagcacaacatcaagagagaattcgacagcaagaagtggaccagattcctgcccaccgagctgtggaataagcggaacctggaggaagcctaccagctggcccggaaggagaacaagaagaagctggaggacatgaggaatcaggtgaggagcctgaaggagaacgacctggagaagtaccagcagatcaactatgtgaacgacctggaaaacctgcggctgctgtcccaagagctgggcgtgaagtggcaggagaaggactgggtggaatacagcggccagatcaagaagcagatcagcgataaccagaagctgacaatcatgaagcagagaatcaccgccgagctgaagaagatgcacggcatcgagaacctgaacctgagaatcagcatcgacaccaacaagtcccggcagactgtgatgaacagaattgccctgcccaagggcttcgtgaagaaccacattcagcagaacagcagcgagaagatcagcaagagaatcagagaggactactgcaagatcgagctgtccggcaagtacgaagagctgagcagacagtttttcgacaagaagaactttgacaagatgaccctgatcaacggactgtgcgagaagaataagctcatcgccttcatggtgatttacctgctggagcggctgggcttcgagctgaaggagaagaccaagctgggcgagctgaagcagacccggatgacatataagatcagcgacaaggtgaaggaggacatccccctctcctactaccccaagctggtgtacgccatgaatcggaagtatgtggacaacatcgatagctacgccttcgccgcctacgagtctaagaaggccatcctggacaaggtggacatcattgagaagcagagaatggaattcatcaagcaggtgctgtgcttcgaggaatacatcttcgagaacagaatcatcgagaagagcaagttcaacgatgaggagacccacatcagcttcacccagatccacgacgaactgatcaagaagggcagagataccgaaaagctgagcaagctgaagcacgccagaaacaaggccctgcacggcgagatccccgacgggaccagctttgagaaggccaagctgctgatcaacgaaatcaagaagtaa

SEQ ID NO: 28 - DNA, 2412 bp, synthetic construct
  misc_feature 1..2412: note = source = /note="Description of Artificial Sequence: Syntheticpolynucleotide"
  source 1..2412: mol_type = other DNA
atgaacgccatcgagctgaagaaggaagaggccgccttctacttcaaccaggccagactgaacatctctggcctggacgaaatcatcgagaagcaactgccacacatcggctctaacagagagaacgccaagaagactgtggacatgatcctggataaccccgaggtgctgaagaagatggaaaactacgtgttcaactcccgcgatattgccaagaatgcccggggcgagctggaggccctgctgctgaagctggtcgagctgagaaacttctatagccactacgtgcacaaggacgacgtcaagacactgagctacggtgagaagcctctgctggataagtactacgagatcgccatcgaagccaccggatccaaggacgtgcggctggagatcattgacgacaagaataagctgaccgacgccggagtgctgttcctgctgtgcatgttcctgaagaagagcgaggctaacaagctgatttccagcatccggggcttcaagaggaacgacaaggagggccagcctagaagaaacctgttcacctactacagcgtgagagagggctataaggtggtgcccgacatgcagaagcactttctgctgttcaccctggtgaaccacctgtccaatcaggacgagtacatctccaacctgcgcccaaaccaggaaatcggccagggcggatttttccaccggatcgccagcaagttcctgagcgacagcggaatcctgcacagcatgaagttctacacatacagatccaagcggctgaccgagcagcggggagagctgaagcccaagaaggaccactttacatggatcgagcctttccagggcaattcctacttcagcgtgcagggccagaagggcgtgatcggagaggagcagctcaaggagctgtgctacgtgctgctggtggcccgggaggacttcagagccgtggagggcaaggtgacccagttcctgaagaagttccagaatgccaataacgtgcagcaggtggagaaggacgaggtgctggaaaaggagtacttccccgccaactactttgagaaccgggacgtgggaagagtcaaggacaagatcctgaacagactgaagaagatcaccgagagttataaggccaagggtagagaggtgaaggcctacgacaagatgaaggaagtgatggagttcatcaacaactgcctgcccaccgatgaaaacctgaagctgaaggactaccggcggtacctgaagatggtgagattctggggcagagagaaggaaaacatcaagcgggagttcgactccaagaagtgggagcgctttctcccccgggagctgtggcagaagagaaacctggaggacgcctaccagctcgccaaggagaagaacacagagctgttcaacaagctgaagaccaccgtggagagaatgaacgaactggagttcgagaagtaccagcagatcaatgacgccaaggacctggccaacctgagacagctggccagagactttggagtgaagtgggaggaaaaggactggcaggaatactctggacagatcaagaagcagatcaccgaccggcagaagctgaccatcatgaagcagcggatcaccgccgccctgaagaagaagcagggaatcgaaaacctgaacctgagaatcacaacagatacgaataagagcaggaaggtggtgctgaaccggatcgcactgcccaagggattcgtcagaaagcacatcctgaagaccgacatcaagatcagcaagcagatccggcagagccagtgccctatcatcctgtctaacaactacatgaagctggccaaggagttctttgaagagcggaacttcgataagatgacccagatcaatggcctgttcgagaagaacgtgctgatcgccttcatgatcgtgtacctgatggagcagctgaacctgagactgggcaagaacaccgagctgtccaacctgaagaagaccgaggtgaactttaccatcaccgacaaggtgaccgagaaggtgcaaatctcccagtaccccagcctggtgttcgccattaaccgggagtacgtggacggcatcagcggctacaagctgccccccaagaagcccaaggaacctccctacaccttcttcgaaaagatcgacgccatcgaaaaggagcggatggaattcatcaagcaggtgctgggcttcgaggagcacctcttcgaaaagaacgtgatcgacaagacccggtttaccgacaccgccacccacatcagcttcaatgagatctgcgatgagctgatcaagaagggctgggacgaaaacaagatcatcaagctgaaggatgcacggaacgctgccctgcacggcaagatccctgaagatacctcctttgacgaagccaaggtgctgatcaacgaactgaagaagtaa

SEQ ID NO: 29 - protein, 7 aa, synthetic construct
  REGION 1..7: note = source = /note="Description of Artificial Sequence: Syntheticpeptide"
GSGGGGS

SEQ ID NO: 30 - protein, 15 aa, synthetic construct
  REGION 1..15: note = source = /note="Description of Artificial Sequence: Syntheticpeptide"
GGGGSGGGGSGGGGS

SEQ ID NO: 31 - protein, 774 aa, synthetic construct
  REGION 1..774: note = source = /note="Description of Artificial Sequence: Syntheticpolypeptide"
AQVSKQTSKKRELSIDEYQGARKWCFTIAFNKALVNRDKNDGLFVESLLRHEKYSKHDWYDEDTRALIKCSTQAANAKAEALANYFSAYRHSPGCLTFTAEDELRTIMERAYERAIFECRRRETEVIIEFPSLFEGDRITTAGVVFFVSFFVERRVLDRLYGAVSGLKKNEGQYKLTRKALSMYCLKDSRFTKAWDKRVLLFRDILAQLGRIPAEAYEYYHGEQGDKKRANDNEGTNPKRHKDKFIEFALHYLEAQHSEICFGRRHIVREEAGAGDEHKKHRTKGKVVVDFSKKDEDQSYYISKNNVIVRIDKNAGPRSYRMGLNELKYLVLLSLQGKGDDAIAKLYRYRQHVENILDVVKVTDKDNHVFLPRFVLEQHGIGRKAFKQRIDGRVKHVRGVWEKKKAATNEMTLHEKARDILQYVNENCTRSFNPGEYNRLLVCLVGKDVENFQAGLKRLQLAERIDGRVYSIFAQTSTINEMHQVVCDQILNRLCRIGDQKLYDYVGLGKKDEIDYKQKVAWFKEHISIRRGFLRKKFWYDSKKGFAKLVEEHLESGGGQRDVGLDKKYYHIDAIGRFEGANPALYETLARDRLCLMMAQYFLGSVRKELGNKIVWSNDSIELPVEGSVGNEKSIVFSVSDYGKLYVLDDAEFLGRICEYFMPHEKGKIRYHTVYEKGFRAYNDLQKKCVEAVLAFEEKVVKAKKMSEKEGAHYIDFREILAQTMCKEAEKTAVNKVAAAFFAAHLKFVIDEFGLFSDVMKKYGIEKEWKFPVK

SEQ ID NO: 32 - protein, 444 aa, synthetic construct
  REGION 1..444: note = source = /note="Description of Artificial Sequence: Syntheticpolypeptide"
LSMYCLKDSRFTKAWDKRVLLFRDILAQLGRIPAEAYEYYHGEQGDKKRANDNEGTNPKRHKDKFIEFALHYLEAQHSEICFGRRHIVREEAGAGDEHKKHRTKGKVVVDFSKKDEDQSYYISKNNVIVRIDKNAGPRSYRMGLNELKYLVLLSLQGKGDDAIAKLYRYRQHVENILDVVKVTDKDNHVFLPRFVLEQHGIGRKAFKQRIDGRVKHVRGVWEKKKAATNEMTLHEKARDILQYVNENCTRSFNPGEYNRLLVCLVGKDVENFQAGLKRLQLAERIDGRVYSIFAQTSTINEMHQVVCDQILNRLCRIGDQKLYDYVGLGKKDEIDYKQKVAWFKEHISIRRGFLRKKFWYDSKKGFAKLVEEHLESGGGQRDVGLDKKYYHIDAIGRFEGANPALYETLARDRLCLMMAQYFLGSVRKELGNKIVWSNDSIELP

SEQ ID NO: 33 - protein, 21 aa, synthetic construct
  REGION 1..21: note = source = /note="Description of Artificial Sequence: Syntheticpeptide"
GGGGGSGGGGSGGGGSGGGGS

SEQ ID NO: 34 - protein, 385 aa, synthetic construct
  REGION 1..385: note = source = /note="Description of Artificial Sequence: Syntheticpolypeptide"
QLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 35 - protein, 7 aa, Macaca mulatta polyomavirus 1
PKKKRKV

SEQ ID NO: 36 - protein, 1194 aa, synthetic construct
  REGION 1..1194: note = source = /note="Description of Artificial Sequence: Syntheticpolypeptide"
PKKKRKVAQVSKQTSKKRELSIDEYQGARKWCFTIAFNKALVNRDKNDGLFVESLLRHEKYSKHDWYDEDTRALIKCSTQAANAKAEALANYFSAYRHSPGCLTFTAEDELRTIMERAYERAIFECRRRETEVIIEFPSLFEGDRITTAGVVFFVSFFVERRVLDRLYGAVSGLKKNEGQYKLTRKALSMYCLKDSRFTKAWDKRVLLFRDILAQLGRIPAEAYEYYHGEQGDKKRANDNEGTNPKRHKDKFIEFALHYLEAQHSEICFGRRHIVREEAGAGDEHKKHRTKGKVVVDFSKKDEDQSYYISKNNVIVRIDKNAGPRSYRMGLNELKYLVLLSLQGKGDDAIAKLYRYRQHVENILDVVKVTDKDNHVFLPRFVLEQHGIGRKAFKQRIDGRVKHVRGVWEKKKAATNEMTLHEKARDILQYVNENCTRSFNPGEYNRLLVCLVGKDVENFQAGLKRLQLAERIDGRVYSIFAQTSTINEMHQVVCDQILNRLCRIGDQKLYDYVGLGKKDEIDYKQKVAWFKEHISIRRGFLRKKFWYDSKKGFAKLVEEHLESGGGQRDVGLDKKYYHIDAIGRFEGANPALYETLARDRLCLMMAQYFLGSVRKELGNKIVWSNDSIELPVEGSVGNEKSIVFSVSDYGKLYVLDDAEFLGRICEYFMPHEKGKIRYHTVYEKGFRAYNDLQKKCVEAVLAFEEKVVKAKKMSEKEGAHYIDFREILAQTMCKEAEKTAVNKVAAAFFAAHLKFVIDEFGLFSDVMKKYGIEKEWKFPVKPKKKRKVGGGGGSGGGGSGGGGSGGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 37 - protein, 865 aa, synthetic construct
  REGION 1..865: note = source = /note="Description of Artificial Sequence: Syntheticpolypeptide"
MPKKKRKVLSMYCLKDSRFTKAWDKRVLLFRDILAQLGRIPAEAYEYYHGEQGDKKRANDNEGTNPKRHKDKFIEFALHYLEAQHSEICFGRRHIVREEAGAGDEHKKHRTKGKVVVDFSKKDEDQSYYISKNNVIVRIDKNAGPRSYRMGLNELKYLVLLSLQGKGDDAIAKLYRYRQHVENILDVVKVTDKDNHVFLPRFVLEQHGIGRKAFKQRIDGRVKHVRGVWEKKKAATNEMTLHEKARDILQYVNENCTRSFNPGEYNRLLVCLVGKDVENFQAGLKRLQLAERIDGRVYSIFAQTSTINEMHQVVCDQILNRLCRIGDQKLYDYVGLGKKDEIDYKQKVAWFKEHISIRRGFLRKKFWYDSKKGFAKLVEEHLESGGGQRDVGLDKKYYHIDAIGRFEGANPALYETLARDRLCLMMAQYFLGSVRKELGNKIVWSNDSIELPPKKKRKVGGGGGSGGGGSGGGGSGGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 38 - DNA, 47 bp, synthetic construct
  misc_feature 1..47: note = source = /note="Description of Artificial Sequence: Syntheticoligonucleotide"
  source 1..47: mol_type = other DNA
gctaccgaaccgtggggcagtagacgatgggccggatgggccccgac

SEQ ID NO: 39 - DNA, 50 bp, synthetic construct
  misc_feature 1..50: note = source = /note="Description of Artificial Sequence: Syntheticoligonucleotide"
  source 1..50: mol_type = other DNA
gtcggggcccatccggcccatcgtccactgccccacggttcggtagctag

SEQ ID NO: 40 - protein, 18 aa, synthetic construct
  REGION 1..18: note = source = /note="Description of Artificial Sequence: Syntheticpeptide"
EGRGSLLTCGDVEENPGP

SEQ ID NO: 41 - protein, 19 aa, synthetic construct
  REGION 1..19: note = source = /note="Description of Artificial Sequence: Syntheticpeptide"
ATNFSLLKQAGDVEENPGP

SEQ ID NO: 42 - protein, 1402 aa, synthetic construct
  REGION 1..1402: note = source = /note="Description of Artificial Sequence: Syntheticpolypeptide"
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDGSLQLPPLERLTLGGGGGSGGGGSGGGGSGGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 43 - DNA, 2555 bp, synthetic construct
  misc_feature 1..2555: note = source = /note="Description of Artificial Sequence: Syntheticpolynucleotide"
  source 1..2555: mol_type = other DNA
atgaggaagttagaagatctgaggtctgagtgggaggctgtaaaccatttacttcgggagctgaggacaaagcagcctgaccgtgcccctggactgagcactactggagcctgtaagtacattttgtcccaggtgttctcttttgctttagctgtttgttcaaaagcgtggctgggaggttatctctgagtgaaaggaaaattggtttctgattctgcataaaggagtaaacatttatgttatttattatgcaaaaggtctttgagtgtgttccttggatactgagagcattacaagctgtgttttctgaagattcaactgcaaggaaaagagggcagacgaggagaagccataatggtttaacctgatactttatcctgttccttagcattaaaactggatggctttgtggaggcacgttgtatctctaggagaaggtctctcctttgaatttctgcttcactggaagcagagtaatcgtggcagcagttttaaattaaaattaaaggtggttcttgataaggaaacccaacagaccaccacattactagcatctaaacacagatatcaaggtgaaagttcagtgacactggagactttaaaaacatgtgcgatttccctagggtccagcttcaaattaatttacttcctattcaagggaattttaaatcagaaagaagatcttatcccatcttgttttgcctttgttttttcttgaataaaaaaaaaataagtaaaatttatttccctggcaaggtctgaaaacttttgttttctttaccacttccacaatgtatatgattgttactgagaaggcttatttaacttaagttacttgtccaggcatgagaatgagcaaaatcgttttttaaaaaattgttaaatgtatattaatgaaaaggttgaatcttttcattttctaccatgtattgctaaacaaagtatccacattgttagaaaaagatatataatgtcatgaataagagtttggctcaaattgttactcttcaattaaatttgacttattgttattgaaattggctctttagcttgtgtttctaatttttctttttcttcttttttcctttttgcaaaaacccaaaatattttagctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggtatgagaaaaaatgataaaagttggcagaagtttttctttaaaatgaagattttccaccaatcactttactctcctagaccatttcccaccagttcttaggcaactgtttctctctcagcaaacacattactctcactattcagcctaagtataatcaaggatataaattaatgcaaataacaaaagtagccatacattaaaaaggaaatatacaaaaaaaaaaaaaaaaaaaaagcagaaaccttacaagaatagttgtctcagttaaatttactaaacaacctggtattttaaaaatctattttataccaaataagtcactcaactgagctatttacatttaaactgtttgttttggcactacgcagcccaacatattgcagaatcaaatataatagtctgggaattgattattatccactcttctaagttgtctgtgccaatttgccttctccaatgataaggataattgaaagagagctataacttaaaaagagaagagcacaaccgttggagtcagtcctcaggtgttttccaccatttctttgagacaatgtccctctttgaaacctcactaagtaagagagactagatacccaatgagctcatctgggcctgtctctgcttcccatcttgactctggttttggaacatccaaacacagatccttatgcccacaagtaagtgctgaggtaatagagccaagccctctagtacctttctaataaataattgttatttagtgtcagagtctaaagttgaatttatatttctaaacatggcaccaatattgtagtttatttcaatgcaagtaatttaatagaaagtcaaatttgtcacctgaagaaatgattttgttaattattttacctatatcactcatagcaccttggatatatttaatgagaaatatacatgtgcaatgacgtttagattctaaatttccactgtcttctcttgagtaataattactgttctttattcttatttttattccagttgaaagaattcagattcagtgggatgaggttcaagaacagctgcagaacaggagacaacagttgaatgaattaaaggattcaacacaatggctggaagctaaggaagaagccgaacaggtcataggacaggtcagaggcaagcttgactcatggaaagaaggtcctcacacagtagatgcaatccaaaagaagatcacagaaaccaag

SEQ ID NO: 44 - DNA, 60 bp, synthetic construct
  misc_feature 1..60: note = source = /note="Description of Artificial Sequence: Syntheticoligonucleotide"
  source 1..60: mol_type = other DNA
gctttacaaagttctctgcaagagcaataaagtggcctatactatctcagcaccactgtg

SEQ ID NO: 45 - protein, 614 aa, synthetic construct
  REGION 1..614: note = source = /note="Description of Artificial Sequence: Syntheticpolypeptide"
MYLSKVIIARAWSRDLYQLLQGLWHLFPNRPDAARDFLFHVEKRNTPEGCHVLLQSAQMPVSTAVATVIKTKQVEFQLQVGVPLYFRLRANPIKTILDNQKRLDSKGNIKRCRVPLIKEAEQIAWLQRKLGNAARVEDVHPISERPQYFSGDGKSGKIQTVCFEGVLTINDAPALIDLVQQGIGPAKSMGCGLLSLAPLLPPLERLTLGGGGGSGGGGSGGGGSGGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 46 - protein, 614 aa, synthetic construct
  REGION 1..614: note = source = /note="Description of Artificial Sequence: Syntheticpolypeptide"
MYLSKVIIARAWSRDLYQLLQGLWHLFPNRPDAARDFLFHVEKRNTPEGCHVLLQSAQMPVSTAVATVIKTKQVEFQLQVGVPLYFRLRANPIKTILDNQKRLDSKGNIKRCRVPLIKEAEQIAWLQRKLGNAARVEDVHPISERPQYFSGDGKSGKIQTVCFEGVLTINDAPALIDLVQQGIGPAKSMGCGLLSLAPLLPPLERLTLGGGGGSGGGGSGGGGSGGGGSQLHLPQVLADAVSRLVLGKFGDLTDNFSSPHARRKVLAGVVMTTGTDVKDAKVISVSTGGKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 47 - DNA, 29 bp, synthetic construct
  misc_feature 1..29: note = source = /note="Description of Artificial Sequence: Syntheticoligonucleotide"
  source 1..29: mol_type = other DNA
gagttccccgcgccagcggggataaaccg

SEQ ID NO: 48 - protein, 9 aa, synthetic construct
  REGION 1..9: note = source = /note="Description of Artificial Sequence: Syntheticpeptide"
LPPLERLTL

SEQ ID NO: 49 - DNA, 78 bp, synthetic construct
  misc_feature 1..78: note = source = /note="Description of Artificial Sequence: Syntheticoligonucleotide"
  source 1..78: mol_type = other DNA
agaaaagtccacatgataacagagaatatcaatgcctcttagagaagcattcataaaaggtatgaattacattatttc

SEQ ID NO: 50 - DNA, 4611 bp, synthetic construct
  misc_feature 1..4611: note = source = /note="Description of Artificial Sequence: Syntheticpolynucleotide"
  source 1..4611: mol_type = other DNA
atgattgaacaccctgctggtggctacaagaaactatttgaaactgtggaggaactgtcctcaccactaacagctcatgtcacaggttggtctcattgatcttagcttgcttattgattgtacattctaaactggcatcttgcagtatagcctaggttgatataaaccatgacacctttcttgctttatcctgagtactgggttcaagctaacagaacctaactcttcagaaagctttcttttctgtcaagatcatcctgtatcagacatcaagtaagcagtagacaggagccactgttagtatcatcaactctgtccaaagacctcatgtgactcatgctttgctatgtgtaacaggatgctatgtgaaatgtcagcagttgggcaaatcttttctctgttttctgctctataaaaggcaaggagagcagtgaacactcttcaactagatggtggtttagtatcaaacattagctttcagtcaaagcaaatattgttttctaatttttatttgatgtctgcttgatagttgaacttaatatgtaggcaacaatcgatttgttttaaaataaaaacctggtaaggattgagcaaacatgaacttttctggggatgagaggactgatcaaacctcattgtgaaaaatggaagtccccaatgacaatcttggtgccattcatttttctttttttatgaggaaacaaaagctcttagagaaagcaggaggacattctacatatgaatccaggtcctgacacttatccctgtatcttatctgccacaaagggtgaatttcataaagcgtgctcatttatatcccaagataaatgggagttttagattttaaaaactcagccaacattcttgctgcttcatataaaaggttctttctacatgagtagagtagtgcacatctttaatttcagcattcaaaacacagaggcagaagtgtgaggccagcttggtctgtttaactgagtatagaccatcaaaaaaccctatcttaaaaaatgcattttttaaaaaattgacacctagttttaatattttgatccatgctcctaaagtctatccttgtactttttaaaccaccaaattgcataagaaaaactgatgttctgtctataggtaagctgacaaataacaaataggcacatagaaaatctagtaagtagtaccacctatagctctagattttaaagagccaggattcctgttttttcatttaaatagtcttttttctatagacacatctttttaaaatacatttattctcactcttaatccatacttctactcctcactccactaaaatatatcaagaaatttgaaaatcttcttaaaaatgagatggcaaaaggcaaatggattggtgtttggggaaatctattaggtggagagtcccaatcctctcaagatgcttctccctcactccttccaaccctttatccacactctggggcatctccacatttcccaaggtgtgatctcctagtgaaggtgagtcaagtcagcagacattttctaagtgctgtccagcgcgagggctggaaatgaaaatcactgccagctctatgaggaagaagctgcccaatcaggctgctgatatactctgccttaccaaggacaagcctagcccaaggcagggataagaagcaatgttctgtctccccttcatcacaggcaggatccccctctggctcaccggcagtctccttcgatgtgggccaggactctttgaagttggatctgagccattttaccacctgtttgatgggtaagccctcctgcacaagtttgactttaaagaaggacatgtcacataccacagaaggtaaagcagcactccatgccactcagagtagagcctatctcagttcttctcctgtttgaactttcaccttcatgaacccagagaaaacctctaaacacatttctttcttctttcttcatggtctagccatgtctgtatatttgcacagccacctgagaatgttaaaatgagataatggggttcaagagctctgagacaaacatggctatcttcaaatcctgaataaaccatgcaccattttgtcttaaatgatggcatctggtagggaattattggcctctaattactttgtcaactgtagaatgaaagaaatggtagaatctactctatagagtctacccatgactaaatgagaataaaatatgtcactcattgtaataatgcagtaaaaacaaaaactgctagtacatctgtgattactagactattgggttatctaaatttctttcattttttatcatcagaaaggtctaaaaaagaagctggtaaaatgactcagaaggtgtgggtacatgccattaagccttaccctaatccaatccacatataaactacctctagcaagttttcctctgaactctgtatgcattccatagctcctgcacatgtgtgtgtgtgtgcgcgcacacacacacacactaatgtttaaaaaattatagggacttgaagagagttccacaaaagggggggagggacaaaatatgtcacagaaacactagatacagatgtgaagaagagtgagagaagggagaaagacagagggatacaaagagaaaatttttagccagttatttgctaaagggttagagcaatgactcagcatccagagaacccaggattcaattctgagcaccatggcagctgctgctcacaactgtctacaactctagaccaaggggatccgatgtactttctagcctctatgggtaacaggcacacatgtagtacacagacatatgtgcaagcagaacacacatacatataatattcgtaataataattttattaatagtaacaacaataaagagttattactaaagccatcattccaagaagcccccataacaacacagtaaataccctgattcctatttgaatattcaggcttgttagtggtatgattaccataaatttcaagcattttgaagggacatcaaaggaagagtaaggggaaactggaaataggatttgaactgtgacaaaagagaggaagggactgacaagcataagtttttctaggaaagcaattttggatttcagacacagattttatataacttctaatgtgtactttcatatacattcctttttataaaagcacaaaatcaaattttactctgccttccatatggcaagcctcaagtagcattttccaggtggtctaggaagtgactcacagataaatcagcctgtgaaagagataaaattcaataaaaactaaattttaaaatatataatacattttagaattaaagagaaacacaaggtcaaacagatgaaaaaaatataatcaatcacactatgtcctataatgtgatgagctacatgtgtgacatagccacatgttctagtttgttttctacttctgtgataaacatcatgaccaagaacaagttgagtaggaaagatgtttatttcatctttacaattcatcaccaggggaagccaaggcaggaaggaatcagaacatggaacacagaccatgaaggaatattgtttactgcctaatggtttgtgtcttcttcttcttcttcttcttcttcttcttcttcttcttcttcttcttcttcttcttcttcttcttcttcttcttcttcttcttcttcttcttctccttttcttcttcttcttcttcttcttctttcttcttctttcttcttctttcttcttcttctttcttctttcttcttcttcttcatcttcttctttcttcttcttcttcaccttcctcctcctcctcctcttccttctcctccactgctgccactgctgccaccacctccttcttctctttacctctctctcctttcctaaccctttcttttaaaaatgttttggaaatatttcatgtatatgttgcctgcatgtatatctgtgcaccacattcatgcagtacctgcagaaactggaagatcatgtcacatctcctggaagtggagttatagatagatggttgtgagcccaaaacatggatgctgggaattgaatccaagtcttctggaagagcagtcaatgatctcaatggctgagccaccacttcagccttcagcctgttttctgaagtggtacctgtccagtggtaccatacacagtgggctggtcttgcctgtatcaatcattaatttgttaaaatgtcctcatggacttacctacagaaaatctgatggaggcatcttttcaatgaaagttttcttttcctccaaagaattctagtttgtgtcaagttgaaaaaatctacacaccatgacgtggggcactttcctgttttctaactctgatcaatgtcacaatcacatatatctactcatctttcggagcctcagtctctctatcaccggcttggggagattcatgattccaatttgatgaaaacacactgactctcagtttcatgtgtctagaatgtatagcctacttgtatcctttatccatttctcaatattcttcagattcatccgcactgatgcttatgttcgagcaatgactgagaagaggattgtcataacagaatttggcacctgtgctttcccagacccctgcaagaatatattttccag

SEQ ID NO: 51 - protein, 198 aa, synthetic construct
  REGION 1..198: note = source = /note="Description of Artificial Sequence: Syntheticpolypeptide"
YLSKVIIARAWSRDLYQLLQGLWHLFPNRPDAARDFLFHVEKRNTPEGCHVLLQSAQMPVSTAVATVIKTKQVEFQLQVGVPLYFRLRANPIKTILDNQKRLDSKGNIKRCRVPLIKEAEQIAWLQRKLGNAARVEDVHPISERPQYFSGDGKSGKIQTVCFEGVLTINDAPALIDLVQQGIGPAKSMGCGLLSLAPL

SEQ ID NO: 52 - protein, 280 aa, synthetic construct
  REGION 1..280: note = source = /note="Description of Artificial Sequence: Syntheticpolypeptide"
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKKLSHGFPPEVEEQDDGTLPMSCAQESGMDRHPAACASARINV

SEQ ID NO: 53 - protein, 147 aa, synthetic construct
  REGION 1..147: note = source = /note="Description of Artificial Sequence: Syntheticpolypeptide"
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMG

SEQ ID NO: 54 - DNA, 30 bp, synthetic construct
  misc_feature 1..30: note = source = /note="Description of Artificial Sequence: Syntheticoligonucleotide"
  source 1..30: mol_type = other DNA
ggaggaggcctcccagcccatggtcttctt

SEQ ID NO: 55 - protein, 198 aa, unidentified
  REGION 1..198: note = source = /note="Description of Unknown:Cas6e sequence"
YLSKVIIARAWSRDLYQLHQGLWHLFPNRPDAARDFLFHVEKRNTPEGCHVLLQSAQMPVSTAVATVIKTKQVEFQLQVGVPLYFRLRANPIKTILDNQKRLDSKGNIKRCRVPLIKEAEQIAWLQRKLGNAARVEDVHPISERPQYFSGDGKSGKIQTVCFEGVLTINDAPALIDLVQQGIGPAKSMGCGLLSLAPL

SEQ ID NO: 56 - protein, 385 aa, synthetic construct
  REGION 1..385: note = source = /note="Description of Artificial Sequence: Syntheticpolypeptide"
QLHLPQVLADAVSRLVIGKFGDLTDNFSSPHARRIGLAGVVMTTGTDVKDAKVICVSTGAKCINGEYLSDRGLALNDCHAEIVSRRSLLRFLYTQLELYLNNEDDQKRSIFQKSERGGFRLKENIQFHLYISTSPCGDARIFSPHEAILEEPADRHPNRKARGQLRTKIEAGQGTIPVRNNASIQTWDGVLQGERLLTMSCSDKIARWNVVGIQGSLLSIFVEPIYFSSIILGSLYHGDHLSRAMYQRISNIEDLPPLYTLNKPLLTGISNAEARQPGKAPIFSVNWTVGDSAIEVINATTGKGELGRASRLCKHALYCRWMRVHGKVPSHLLRSKITKPNVYHETKLAAKEYQAAKARLFTAFIKAGLGAWVEKPTEQDQFSLT

SEQ ID NO: 57 - protein, 336 aa, unidentified
  REGION 1..336: note = source = /note="Description of Unknown:ADAR1DD sequence"
SLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNF

SEQ ID NO: 58 - protein, 336 aa, synthetic construct
  REGION 1..336: note = source = /note="Description of Artificial Sequence: Syntheticpolypeptide"
SLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGQGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNF

SEQ ID NO: 59 - protein, 385 aa, unidentified
  REGION 1..385: note = source = /note="Description of Unknown:ADAR2DD sequence"
QLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 60 - protein, 198 aa, synthetic construct [sequence omitted]

SEQ ID NO: 61 - protein, 208 aa, synthetic construct
  REGION 1..208: note = source = /note="Description of Artificial Sequence: Syntheticpolypeptide"
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 62 - protein, 229 aa, synthetic construct [sequence omitted]

SEQ ID NO: 63 - protein, 4 aa, synthetic construct
  REGION 1..4: note = source = /note="Description of Artificial Sequence: Syntheticpeptide"
GGGS

SEQ ID NO: 64 - protein, 16 aa, unidentified
  REGION 1..16: note = source = /note="Description of Unknown:Nucleoplasmin bipartite NLS sequence"
KRPAATKKAGQAKKKK

SEQ ID NO: 65 - protein, 9 aa, unidentified
  REGION 1..9: note = source = /note="Description of Unknown:C-myc NLS sequence"
PAAKRVKLD

SEQ ID NO: 66 - protein, 11 aa, unidentified
  REGION 1..11: note = source = /note="Description of Unknown:C-myc NLS sequence"
RQRRNELKRSP

SEQ ID NO: 67 - protein, 38 aa, Homo sapiens
NQSSNFGPMKGGNFGGRSSGPYGGGGQYFAKPRNQGGY

SEQ ID NO: 68 - protein, 42 aa, unidentified
  REGION 1..42: note = source = /note="Description of Unknown:IBB domain from importin-alpha sequence"
RMRIZFKNKGKDTAELRRRRVEVSVELRKAKKDEQILKRRNV

SEQ ID NO: 69 - protein, 8 aa, unidentified
  REGION 1..8: note = source = /note="Description of Unknown:Myoma T protein sequence"
VSRKRPRP

SEQ ID NO: 70 - protein, 8 aa, unidentified
  REGION 1..8: note = source = /note="Description of Unknown:Myoma T protein sequence"
PPKKARED

SEQ ID NO: 71 - protein, 8 aa, Homo sapiens
PQPKKKPL

SEQ ID NO: 72 - protein, 12 aa, Mus musculus
SALIKKKKKMAP

SEQ ID NO: 73 - protein, 5 aa, Influenza A virus
DRLRR

SEQ ID NO: 74 - protein, 7 aa, Influenza A virus
PKQKKRK

SEQ ID NO: 75 - protein, 10 aa, Hepatovirus D
RKLKKKIKKL

SEQ ID NO: 76 - protein, 10 aa, Mus musculus
REKKKFLKRR

SEQ ID NO: 77 - protein, 20 aa, Homo sapiens
KRKGDEVDGVDEVAKKKSKK

SEQ ID NO: 78 - protein, 17 aa, Homo sapiens
RKCLQAGMNLEARKTKK

SEQ ID NO: 79 - RNA, 36 nt, synthetic construct
  misc_feature 1..36: note = source = /note="Description of Artificial Sequence: Syntheticoligonucleotide"
  source 1..36: mol_type = other RNA
ggcccaacatgaggatcacccatgtctgcaggggcc

SEQ ID NO: 80 - RNA, 29 nt, synthetic construct
  misc_feature 1..29: note = source = /note="Description of Artificial Sequence: Syntheticoligonucleotide"
  source 1..29: mol_type = other RNA
ggcccatgctgtctaagacagcatgggcc

SEQ ID NO: 81 - RNA, 34 nt, synthetic construct
  misc_feature 1..34: note = source = /note="Description of Artificial Sequence: Syntheticoligonucleotide"
  source 1..34: mol_type = other RNA
ggccctaagggtttatatggaaacccttagggcc

SEQ ID NO: 82 - protein, 130 aa, synthetic construct
  REGION 1..130: note = source = /note="Description of Artificial Sequence: Syntheticpolypeptide"
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 83 - protein, 133 aa, synthetic construct
  REGION 1..133: note = source = /note="Description of Artificial Sequence: Syntheticpolypeptide"
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 84 - protein, 128 aa, synthetic construct
  REGION 1..128: note = source = /note="Description of Artificial Sequence: Syntheticpolypeptide"
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVVQATSEDLVVNLVPLGR

SEQ ID NO: 85 - protein, 30 aa, synthetic construct
  SITE 1..30: note = /note="This sequence may encompass 2-15 'Gly Ser'repeating units"
  REGION 1..30: note = source = /note="Description of Artificial Sequence: Syntheticpolypeptide"
GSGSGSGSGSGSGSGSGSGSGSGSGSGSGS

SEQ ID NO: 86 - protein, 28 aa, synthetic construct
  SITE 1..28: note = /note="This sequence may encompass 2-4 'Gly Ser Gly Gly GlyGly Ser' repeating units"
  REGION 1..28: note = source = /note="Description of Artificial Sequence: Syntheticpeptide"
GSGGGGSGSGGGGSGSGGGGSGSGGGGS

SEQ ID NO: 87 - protein, 30 aa, synthetic construct
  SITE 1..30: note = /note="This sequence may encompass 5-10 'Gly Gly Ser'repeating units"
  REGION 1..30: note = source = /note="Description of Artificial Sequence: Syntheticpolypeptide"
GGSGGSGGSGGSGGSGGSGGSGGSGGSGGS

SEQ ID NO: 88 - protein, 28 aa, synthetic construct
  SITE 1..28: note = /note="This sequence may encompass 3-7 'Gly Gly Gly Ser'repeating units"
  REGION 1..28: note = source = /note="Description of Artificial Sequence: Syntheticpeptide"
GGGSGGGSGGGSGGGSGGGSGGGSGGGS

SEQ ID NO: 89 - protein, 25 aa, synthetic construct
  SITE 1..25: note = /note="This sequence may encompass 3-5 'Gly Gly Gly Gly Ser'repeating units"
  REGION 1..25: note = source = /note="Description of Artificial Sequence: Syntheticpeptide"
GGGGSGGGGSGGGGSGGGGSGGGGS

SEQ ID NO: 90 - protein, 24 aa, synthetic construct
  SITE 1..24: note = /note="This sequence may encompass 3-4 'Gly Gly Gly GlyGly Ser' repeating units"
  REGION 1..24: note = source = /note="Description of Artificial Sequence: Syntheticpeptide"
GGGGGSGGGGGSGGGGGSGGGGGS

SEQ ID NO: 91 - protein, 12 aa, synthetic construct
  REGION 1..12: note = source = /note="Description of Artificial Sequence: Syntheticpeptide"
GGGSGGGSGGGS

SEQ ID NO: 92 - protein, 88 aa, synthetic construct
  REGION 1..88: note = source = /note="Description of Artificial Sequence: Syntheticpolypeptide"
EASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 93 - protein, 5 aa, synthetic construct
  REGION 1..5: note = source = /note="Description of Artificial Sequence: Syntheticpeptide"
GGGGS

SEQ ID NO: 94 - protein, 6 aa, synthetic construct
  REGION 1..6: note = source = /note="Description of Artificial Sequence: Syntheticpeptide"
GGGGGS

SEQ ID NO: 95 - protein, 30 aa, synthetic construct; identical to SEQ ID NO: 87
  SITE 1..30: note = /note="This sequence may encompass 1-10 'Gly Gly Ser'repeating units"
  REGION 1..30: note = source = /note="Description of Artificial Sequence: Syntheticpolypeptide"

SEQ ID NO: 96 - DNA, 51 bp, synthetic construct
  misc_feature 1..51: note = source = /note="Description of Artificial Sequence: Syntheticoligonucleotide"
  source 1..51: mol_type = other DNA
gctagctaccgaaccgtggggcagtagacgatgggccggatgggccccgac

SEQ ID NO: 97 - DNA, 50 bp, synthetic construct; identical to SEQ ID NO: 39
  misc_feature 1..50: note = source = /note="Description of Artificial Sequence: Syntheticoligonucleotide"
  source 1..50: mol_type = other DNA

SEQ ID NO: 98 - DNA, 57 bp, synthetic construct
  misc_feature 1..57: note = source = /note="Description of Artificial Sequence: Syntheticoligonucleotide"
  source 1..57: mol_type = other DNA
gctttacaaagttctctgcaagagcaataaagtggcctatactatctcagcaccact

SEQ ID NO: 99 - DNA, 50 bp, synthetic construct
  misc_feature 1..50: note = source = /note="Description of Artificial Sequence: Syntheticoligonucleotide"
  source 1..50: mol_type = other DNA
gtgctgagatagtataggccactccattgctcttgcagagaactttgtaa

SEQ ID NO: 100 - DNA, 63 bp, synthetic construct
  misc_feature 1..63: note = source = /note="Description of Artificial Sequence: Syntheticoligonucleotide"
  source 1..63: mol_type = other DNA
tccacatgataacagagaatatcaatgcctcttagagaagcattcataaaaggtatgaattac

SEQ ID NO: 101 - DNA, 50 bp, synthetic construct
  misc_feature 1..50: note = source = /note="Description of Artificial Sequence: Syntheticoligonucleotide"
  source 1..50: mol_type = other DNA
tcataccttttatgaatgcttctccaagaggcattgatattctctgttat

SEQ ID NO: 102 - DNA, 71 bp, synthetic construct
  misc_feature 1..71: note = source = /note="Description of Artificial Sequence: Syntheticoligonucleotide"
  source 1..71: mol_type = other DNA
ggatctgagccattttaccacctgtttgatgggtaagccctcctgcacaagtttgactttaaagaaggaca

SEQ ID NO: 103 - DNA, 50 bp, synthetic construct
  misc_feature 1..50: note = source = /note="Description of Artificial Sequence: Syntheticoligonucleotide"
  source 1..50: mol_type = other DNA
tcaaacttgtgcaggagggcccacccatcaaacaggtggtaaaatggctc

SEQ ID NO: 104 - RNA, 36 nt, synthetic construct
  misc_feature 1..36: note = source = /note="Description of Artificial Sequence: Syntheticoligonucleotide"
  source 1..36: mol_type = other RNA
gctggagcagcccccgatttgtggggtgattacagc

SEQ ID NO: 105 - RNA, 36 nt, synthetic construct
  misc_feature 1..36: note = source = /note="Description of Artificial Sequence: Syntheticoligonucleotide"
  source 1..36: mol_type = other RNA
gctgaagaagcctccgatttgagaggtgattacagc

SEQ ID NO: 106 - RNA, 36 nt, synthetic construct
  misc_feature 1..36: note = source = /note="Description of Artificial Sequence: Syntheticoligonucleotide"
  source 1..36: mol_type = other RNA
gctgtgatagacctcgatttgtggggtagtaacagc

SEQ ID NO: 107 - RNA, 36 nt, synthetic construct; identical to SEQ ID NO: 106
  misc_feature 1..36: note = source = /note="Description of Artificial Sequence: Syntheticoligonucleotide"
  source 1..36: mol_type = other RNA

SEQ ID NO: 108 - RNA, 36 nt, synthetic construct; identical to SEQ ID NO: 106
  misc_feature 1..36: note = source = /note="Description of Artificial Sequence: Syntheticoligonucleotide"
  source 1..36: mol_type = other RNA

SEQ ID NO: 109 - RNA, 36 nt, synthetic construct
  misc_feature 1..36: note = source = /note="Description of Artificial Sequence: Syntheticoligonucleotide"
  source 1..36: mol_type = other RNA
gctgtgatgggcctcaatttgtggggaagtaacagc

SEQ ID NO: 110 - RNA, 36 nt, synthetic construct
  misc_feature 1..36: note = source = /note="Description of Artificial Sequence: Syntheticoligonucleotide"
  source 1..36: mol_type = other RNA
gctgtgataggcctcgatttgtggggtagtaacagc

SEQ ID NO: 111 - protein, 866 aa, synthetic construct
  REGION 1..866: note = Synthetic: Cas13e.3 amino acid sequence
MAVDYSLKNEWYREINKSCFTVALNVAYDNCKAKGHENLLREAQRSKGGITNEQIKNVQTEIKTRLEDIRSHFSHFYHDEKSLIFEKDNIVKDFLESAYEKAQSSVIGSTRQSDYKGVVPPLFEPHDGMITAAGVVFLASFFCHRSNVYRMLGAVKGFKHTGKEELSDGAKRDYGFTRRLMAHYSLRDSYVIKAEETKSFRDLLGYLSRVPQQAVDWLNEHNQLSEDEKKEFLNQKPSDEESQEQSKTENTDRQADRMPRRSLRKTDKFILFAAKFIEDWAQKEKMDVTFARYQKTVTEDENKNQDGKQVRDVQLKYEKDTKKLNPDFDYKWTYYIRNNHAIIQIKPDEYKQAVSARISENELKYLVLLIFQGKGWEAIKKIGDYIFHIGNKIKIGRFDHNEERRMPSFLKNPPADIIGEMVENRLKYIRDELNKVIETIKKEEPQNNKWLLYKGKKISIILKFISDSISDIKKRPDVNEYNTLRDMLQKLDFDNFYERLKSYVSEGRIEQTLYDEIKGIKDISTLCIKICELRLAALEELEKEGGDDLNKYIGLAVQEKHKNYDDSNTPQKKAERFLESQFSVGKNFLRETFYDEYIKNRKSLYEIIKEKITGITPLNENRWYLMDKNPKEFESKDSKIIRGLCNIYIQDILCMKIALWYYENLSPSYKNKLKWDFIGQGFGYDRYKLSYKTDCGITIEFKLADLNRLDIIEKPKMIENICHSFILEKDVKKQTISWHEFRQDGIAKYRKLQKEVVEAVFEFENSLKIPDKNWLTQGYVPFNKNKRFEDKGFSTFILEEAVRKGKIKSDDKEPLRKVRTDFFHEQFDSTDAERRIFDKYMPAKHDGKNKGGKMQEKQEKSYTRRI

SEQ ID NO: 112 - protein, 798 aa, synthetic construct
  REGION 1..798: note = Synthetic: Cas13e.4 amino acid sequence
MEKEQGLYSIDRYQGAGKWCFAIGANRAWDNYNERPKLFSESLLRYEKATRRDWFDEETRGLIKKSDVRQRLRKIRCYFSHYCHDNTCLGFDPDDDLRKIMEKAYERAIFEQRKHLSTETDIETPALFEPHGRITAAGVVFFCSFFVERRILNRLMGRIPGFKKTEGEYGATRQMFSKYCLRDSYSIRASDSNAVLFRDILGYLSRAPSQYYRHNKDQCDKDGHPERKKDKFINLALRYLESFVPARLRNHTLSVGRKEVVRMETNAVAEGEGEYRPYPPKAKVKVVFTEDDPERPYYITHNTVILQTAKKEEDIHHCKVGVNELKYLVLLCLQGKAEKAVAGIEGYVRRIQGRFADHTNKVARDDDERLVRGLPEFVRVASGIETPDEVRELKSRLDHIRKKWQTKKAESAEAQLHRKARDVLRHINWESQRPLGIEQYNRLLELLVNRDLESFAAEMKELKRRGLISEELLKSVEGIRNLNTLHVKVCNLVLTRLEHLVENDPEELKRHIGIVPREEKEGPSYEEKVRAFVQQPMMYRGFLRNSFFKGSGKSFAKLVEEELHKKGCPDVPLGTDYYLVRDLERDERKNRFHNDNAALYETLALDRLCVLMARDCLVRLNRNLEKHATRISWEATDAGDTICLELPRRDRDHESFRLSFGVRDYPKLYVMDDPVFLCGLMKHFFPDNQAIQYHELYSEGINKYTAMQAEGIAATLKLEEKTIKEKNMQIPATGYIRFCEIVSQSDFAPGEKRVLKNVRNGLLHYHLEFEPTEWAEFREIMKREGFDTAKKRKSTRKK

SEQ ID NO: 113 - protein, 804 aa, synthetic construct
  REGION 1..804: note = Synthetic: Cas13e.5 amino acid sequence
MKIKNENTDKKTELYSIDKYKGRDKWCFAIVLNKAQTNLTENPDLFEQTITKYDRIRKEGWFDEETKKLIYIQENEHKIKGEIKTLAREVLKNLRNYFSHHFYKQDCLIFPKDNIVRIIMGRAYERSEYEIKKNLKEDISIELPALFEPEGKITTAGIVFFTSFFVERRFLHRLMGSVQGFTKTEGEYKITRDVFAKYCLRDSYSVKAQDNDAVMFRDIIGYLSRVPTESFQHIKNPKKQNESQLSERKTDKFISFALKYLKDYGFEDLKGHYTAFFARSEIKKEKEDIEIKDDKKHKPHRMKSKIEIHFDKTKEDRFYIERNNVILKIQRKGGRANILRMGIYELKYLVLLCLSGKAREAINRIDDYLNDLRNKIPHIENMNKEGIGEQIRSLPGFVRSQLGFVQIDDEKKKENRLDYVEKKWEKKRAESKELKLNRKGRDILRYINERCKKPLTIDRYNRILELLVEKNIEGFYHELEELRKTGRIEKNITQALVGEKNINALHIKICKLVQDELKSLEKEDLKKYIGLTPKEEKVVSFEEKLGRILDKPVIYKGFLRYQFFKNDKKSFARLVEEIIKEKTGGLDVPIETEYYSISTLGRFDKANKTLYETLAMDRLCMMMARRYFLSLNKILAKRAQNIEWKKESGKEFIVFKFNMPQDTGKSISIRFSPKDYTKLYVKNDSEFLARLCQYFFPNEKAIDYHKLYSHGINKYTNFQKEGIEAILELEEKIIKKRKINSPENYLSFEEILNQSIYNDEEKNTLIQIRHSLLHYQILFSKNDLTKFYNVMKREGIEKIWSLVI

SEQ ID NO: 114 - protein, 878 aa, synthetic construct
  REGION 1..878: note = Synthetic: Cas13e.6 amino acid sequence
MAVNYSLREKWYRGVNKCCFTVALNIAVDNCKSKGCETLLKEAEHSKGGITDEQIQQSYTEVEKRLNDIRNYFSHFYHGDECLIFKKDDIVKRFMESVFATAVSNVVGGTKESDYKGVVPPLFEQSNEDYMITAAGVIFLASFFCHRSNVYRMLGAVKGFKHTGKEELSDGQKRDHGFTHRLLAHYSLRDSYSVKIEETKSFRDLLGYLSRVPQQAVDWLNERNELSEDEKKEFLNQKSSEEESPEQPEPENAEWRTEKTSRRSLRKTEKFILFAAKFIEDRAEKEKQDVTFARYQKTVTKEENKNQDGKQARVVRLKYEEDKKDDEKPREHFNLEWMYYIRNEHAIIQIKPKDKEAVAARISENELKYLVLLIFEGKGGDAFNKLSDYIFRMTQKIKSGQINPNEARLPSFLKNPVKNITDKMVRNRLDYIRGQIKDVLEKINMEEPQNNKWLIYKGKKISLVLKFISDGISDIKKRPNVKEYDTLRDTLQKLDFNRFYERLKSYVSDGRLAAALYDKIKGIDDISELCKKVCELMFARLAELEKKGGFELYRYIGMEVQEKDEKYDEWNSPQKKAERFLESQFSIGENFLRESFYSEYCQKQECIDKEISLNTSVKNRKSLVYIVKEKLKDIMPLHNDRWYLIDRNPKDFERKDSKVIKGLCNTYVQDVLCMKMARWYYGQLNPALKNNIKWDETGQGHGYDRYKLSYRTNFGITIEFKLADFTRLDIIEKSDMIENICRSFIKPNRTISWYDFKQDGVEEYRKRQYKAVRAVFAFEESLIIPGRDWLSQGFVPFIKNEEYVKKGFSLFVLDEAVRQLKIKGSDKDAMRQVRNDFFHEQFQAKDEQWKVFEGYLSCFMIDRPKGEKNKKRYNGNKK

SEQ ID NO: 115 - protein, 871 aa, synthetic construct
  REGION 1..871: note = Synthetic: Cas13e.7 amino acid sequence
MEKYLIKNFEGINKSKFTVALNIANDNCKNKGIQELLKEAQRSKGGITDTQITEVQEHIKERLNSVRNYFSHCYHEKKPLYFEANDPVKIFLEETFAKAVENLQGRFLSDKYKLTVPPLFEPNQNNTITAAGVIFLASFFCHRSYVYRMLGGIPGFKRSDKKKWGDGQKIDYGFTRKLMSFYSLRDSYSVNVQENKELTAFRDILGYLARVPGQAIDWLIEKGKLTKEEGKQFYLGEQSEEREEKAKKEEIKYALRKTDKFMLFAVRFIEDWAEQERIKVEFARYEKMTIVNENKKQDEKEERKVKFVSDEPTAAGWTYYIRNNHAIIKIIPDDKKKKAVSARISENELKYLVLTIIDGNGKNAIAYIGDYIFRTARQIENKSYNAESEKYAPAFVRGGQKKSVDKRIKYIRDEIQQVINDIEAEQEKQKNEQDAPAENRTWLIYKGKKISIILRYVNDNIAEYKKRLSVTEYNELRGYLQQLDFINFHRKLAEYQHHGRLPNGFAESINKFQDLSKLCIEVCERQKKKLQEMAAKGGIELEQYIGLAPKEENQEQNKYATKANNFIKVWLSIPENFLRQKFYDKFCKQQECKNKGSDKPDNTSVPQRKYFIAIIREKNIRPIHADKYYLLGQNPKDYERPDGKIIRQLCDVYCKDGLCMAMAKWYYENRLGKFKDLIEWQTGDDKQQHGYAGHTLEYQATEKIKIRFKLADFTRLDIIEPPERVKNICRQWETELLKKTRDGTISWYDFKLNGLEPYRQWQGYAVADIFWFEESLKINETQWQGRTHMPFNFEKDKPLWCNILDEAVKQNKIEKQDTQALRRVRHDCFHEEFLANYEQLKIFKNLISDKAKDAKPKDKKSRKNEQKYGKR

SEQ ID NO: 116 - protein, 813 aa, synthetic construct
  REGION 1..813: note = Synthetic: Cas13e.8 amino acid sequence
METTEEKKEIYSIDKYQGAGKWCFAIILNRARDNYEGNPHLLSESLLRFEAVNRRDWFDEETRELIIQMDAETKTDSNLKPSTPAYKILIGIRNYFSHFYHTEECLYFKEDDPIRIILENAYKKAKQHHIERLEKETDIEFPALFESNDRITSAGIIFFSSLFVERRILNRLMGYVGGFKKTKGEYNITREIFSTYCLRDSYSIMAADSNAVIFRDILGYLSRVPSEYYQHNKEKCEKENKPKRKTKKFIYFALRYLEKFVLKEIKNQKVSIARMEVVREKSKEAEGEDEQHKPYPDKAKVKIVFDSIGKELPYYINHKTVIMKIQRNGEAVNFCKIGINELKYLVLLCLQGKSVDAIAKINGYIDRIKKRLENPKTRIDIDSERDNEFIKGLPEFVKIQSGRTPDEEREKKSRIDYIRKKWEKKKEESPKTELHRKGRDILRYINWHCEPPLGSEEYNHLLSLLVNKDLDGFENELKELKRTEQISKKLLEMLKGFSNLNELHLKVCSIVPVELVYLEKNDPEKLAEYIGLIPQKTDKEPPSYEEKVKNFIKQPMIYKGFLRDTFFSSGKTFAKLVEETFLHKYPHSDVPLGRDYYHVTTLDRFHKDNSILYETLALDRLCVVMARKFHETLNQELAKESKQIVWENNTIILELPRSKTSSSDTFQICFDIKHYMKLYVMDDVEFLGGLMRHFFQKEKTIEYYNLYSFGINKYTEMQRNGIEAILRLEEKIIREKNIKRKPDKNYISFYEIMEASNYPDNDKKTLNKVRRALLHYHLKFEPADYNKFVAIMKREGLERKKKTGKATRRWVKK

SEQ ID NO: 117 - protein, 789 aa, synthetic construct
  REGION 1..789: note = Synthetic: Cas13f.6 amino acid sequence
MENIKLEKQKAAFYFNQAELNLKAIEGNIFDKGRRKTLFDNPKILSKVENFIFNFKDVTKNAKGEIDCLLSKLMELRNFYSHYVHKPDVKELSKGEKPLLERYYQIAIEATGSENVKLEIIENDKWLTDAGVLLFLCMFLKKSQANKLISGISGFKRNDTFGQPRRNLFNYFSVRERYKVVPDMQKHFLLFVLVNHLSEQDDYIEKAQQPYNIGEGLFFHRIASTFLNVSGILRNMEFYTYQSKRLKEQRGELKREKDIFTWEEPFQGNSYFEINGHKGVIGEDELKELCYALLSYNKSKYAVEQIEKFLKGFGEVKSEQEIRDSDILNESYFPTNYFAESNIGSIKEKILNRLGKTDDSYKKTGTKIKPYDMMKEVMEFINNSLPADEKLKRKDYRRYLKMVRIWDSEKDNIKREFESKEWSKYFSSNFWMAKNLERVYGLAREKNAELFNKLKAVVEKMDEREFEKYRQINSAEDLASLRRLANDYGVKWEEKDWQEYSGQIKKQISDRQKLTIMKQRITAELKKKHGIENLNLRITIDSNKSRKAVLNRIAVPRGFVKEHILGWQGSEKVSKKTREAKCKILLSKEYEELSKQFFQTRNYDKMTQVNSLYEKNKLIAFMAVYLMGQLNIRFDKPTRLNELEKAEVDFKISDKVTAKIPFSQYPSLVYAMSSKYADSVGSYKFENDEKNKPFLGKIDIIEKQRMEFIKEVLGFEEYLFEKKIIDKSKFADTATHISFREICDELIQKGWDENKLTNLKDARNAALHGEIPAETSFREAKPLINGLKK

SEQ ID NO: 118 - protein, 799 aa, synthetic construct
  REGION 1..799: note = Synthetic: Cas13f.7 amino acid sequence
MINIELKKEEAAFYFNQANLNISGLDEVIEKQLPHIGSKKENAKKAIDKIFDNITVLKKVENFVFYFKDVAKNERVELDALLLKLIDLRNFYSHYVHNDNVKILSDGEETLLEKYYQIAIEATGSKDVKLEIIDNEKKLTDAGILFLLCMFLKKSQANKLISSISGFKRNDKEWQPRRNLFTYYSLREGYKVVPDMEKHFLLFTLVNHLSTQDENIENTQPSDDIGRGLFFHRIASTFLNISGIFNNMEFYPYQSNRLKERRGDIAPDKDSFAWIEPFQGNSYFKINGYKGVIGENELKELCFAVLLHNKSKYAVEQIEKFLKCFKEVQSKQEIIECDILDECYFPANYLNQPETKSLKEKLLSRITGKINYSFDTAEKAFDKMKDVMEFINGCLPSDEKLKRKDYSRYLKMVRFWGGEKDNIKREFEGKKWTRFFPSELWHKRTLEDVYKFALKKNKKRLEELKVKIEGLNEDDLLKYQKVNNIKNLENLRLLAHDLDLSWREKDWGEYSGQIKKQISDNQKLTIMKQRVIAELKKKHGIENINLRISLDSNKSIQAVLNRIAIPKGFIKRHVLHLQENEKTSRKIREAKCKILLSKKYEYLSRKFLDEKNFDKLTQINGLYEKNRLIAFMVIYLLKQLGLELKNETKLIELKKTRVKYKISDKVAEDIPLSHYPSLVYAMSRKYVDNIDNYEFPDEYAKKAILDKVDIIENQRMEFIKQVLGFEKYLFDNNIIDKSKFTDVETHISFVKIHDELIEKGWDTEKLSKLKHARNKALHGEIPGGTSFEKAKLLINELKK

SEQ ID NO: 119 - protein, 877 aa, synthetic construct
  REGION 1..877: note = Synthetic: Cas13d.1 amino acid sequence
MKKQKSKKTVSKTSGLKEALSVQGTVIMTSFGKGNMANLSYKIPSSQKPQNLNSSAGLKNVEVSGKKIKFQGRHPKIATTDNPLFKPQPGMDLLCLKDKLEMHYFGKTFDDNIHIQLIYQILDIEKILAVHVNNIVFTLDNVLHPQKEELTEDFIGAGGWRINLDYQTLRGQTNKYDRFKNYIKRKELLYFGEAFYHENERRYEEDIFAILTLLSALRQFCFHSDLSSDESDHVNSFWLYQLEDQLSDEFKETLSILWEEVTERIDSEFLKTNTVNLHILCHVFPKESKETIVRAYYEFLIKKSFKNMGFSIKKLREIMLEQSDLKSFKEDKYNSVRAKLYKLFDFIITYYYDHHAFEKEALVSSLRSSLTEENKEEIYIKTARTLASALGADFKKAAADVNAKNIRDYQKKANDYRISFEDIKIGNTGIGYFSELIYMLTLLLDGKEINDLLTTLINKFDNIISFIDILKKLNLEFKFKPEYADFFNMTNCRYTLEELRVINSIARMQKPSADARKIMYRDALRILGMDNRPDEEIDRELERTMPVGADGKFIKGKQGFRNFIASNVIESSRFHYLVRYNNPHKTRTLVKNPNVVKFVLEGIPETQIKRYFDVCKGQEIPPTSDKSAQIDVLARIISSVDYKIFEDVPQSAKINKDDPSRNFSDALKKQRYQAIVSLYLTVMYLITKNLVYVNSRYVIAFHCLERDAFLHGVTLPKMNKKIVYSQLTTHLLTDKNYTTYGHLKNQKGHRKWYVLVKNNLQNSDITAVSSFRNIVAHISVVRNSNEYISGIGELHSYFELYHYLVQSMIAKNNWYDTSHQPKTAEYLNNLKKHHTYCKDFVKAYCIPFGYVVPRYKNLTINELFDRNNPNPEPKEEV

SEQ ID NO: 120 - protein, 864 aa, synthetic construct
  REGION 1..864: note = Synthetic: Cas13d.2 amino acid sequence
MKKSKVKLNGVKAVYHISPDVRVIAAFGRGNNSVLDKHIENGSVEELQNHSDIEVNISRKTYSFRKKSLKKAAGQFSVPDNTNDQLGIREELEKEIFGRKFDDNIHIQAAYAVNDIIKMLAVAANLAETAINGLDRENTENDMIGFCIIPHITYQTYADDKRSKFEGFIEKVKTQGTFSYFPDILPKFKKEESEEESDNEKLYYIMCIISLIRNSATHSKSSNSDTTDYIFGEFNSVNKEALTTTADNLIKSKIDFINKEGFSKNQKNNIYRLLKAKADTPENTARLIRRLYAFTIRKQDKNLGFSLKKLRECAIRSIDRSIEYMKYLPSKKYDTVRSKLYTLMDFVVYSYLKYHKDGKKFSKEMVEQLRAAESDKVKDDIYRDEAEKLYNIEIISRTINALISDIKSDFDQPKHGNECYQPINDGMKEAEKDFITTDQLSLFTKFIYVLCQFLDGKNINILLSSLISKFQQIEAFNGDIRKLNLNIRDDGKIGYDSKKYSIFEKSGQIADDLDKLRGVIKMDINDLNAYETMIKDALRVIGVDESDIESIYQTHFKTQDKKDSVSGFFRNNIINSRRFRYIIKYINPSDAYRIIQNENVRNYVLGRMNDAIIDRYAHSVGIEDKVHDKRKVLSDILSKVKFDNFTKLTYINPKDKNKGEKAKEREKPKAILGLYLTIVYLVVKSLVRINSQYVMAVYHLERDSRLCPGVSSNNLLSMTNHYCDRDNHLLKEKHIVKLERYKKTQKTQKTICTAYRNAIAHLSAVRKGVKYIGDIQKADSYFGIYHYCMQKLIYSANGQPFAEFVRSIFGDEKELDKLRNGSYSQAILRALNYPFGYNPARYKNLSYEKIFIRAWQDEDTNKKT

SEQ ID NO: 121 - protein, 880 aa, synthetic construct
  REGION 1..880: note = Synthetic: Cas13d.3 amino acid sequence
MNKIHKKQGKTTAKSLGLKSVLKIENDLVVTTFGKKDNPMVVEQSINKASGEKELYVDEDQVKFDSSLIKEKNILSLDSIQHSNHQIIVNIDQKDASEIGMDYLRLKPELEKEFFGKTFYDNVHIQIAYNLLDLKKIIGLHIGNAIQALENLGRDGSDLVGICDATKPLNYLDDVKQKADIGFMNRLKPYFMYFDGVLKLDNSKNKNGELNQLDIENWDVIRILSLIRQGCAHAGAYSSLLYTAQNNKVYADLINKALSIFSDDLDKFNKSFLKQSKMNLFILFDLYNCRFDRSLQEKIIKEYYRYVLYKDNKNLGFSLKNVRNLIIEGKYDEQERSGKLQTIRSKLNTLLDFYLYGYYQKNPTFVENIVAKLRESKNDEDKEKVYEEEYHRLLSENNYLVDKKCSDIVYRINEAVKNRKIFVNANINAVVEKVSCSCFPSLIYVLCKFLDGKEVNELTTAIINKLENIASLINALVTLKSYGGFSEQYKIFDYPNINGLIDDFRMVKNLTSTKRKLKKASGGEDRIGRQLYADAINIFKEDSFVSANDEKGTGLDQYVNKFFSKDDLGARKVRNLLLNNIIKNRRFVYLIKYIDPKDCYKLVHNEKIVRFALGQYDESQMPLNQLQKYYDAVIENREGFRKCNDRKKIIDTLVSEINRVSIDGILDIGNRLVNRGNNDYINHQKQIISLYLTIAYLIVKGVVHTNSLYFIAWHAYERDNNFKFGNDGKDYLALTKEYLTNKKKRVKQLLDHNIEEANNSLDSKYFSAYRNKVVHLNFCNIFVNYLDGIGDIHSYYDIYQYVIQKWSIAERSKDFIDPQYLTKLSNDLKQYRTYQRNFLKIINLPFAYNLARYKNLTIGDLFNDKYPLPKETVKEFYNEE

SEQ ID NO: 122 - protein, 880 aa, synthetic construct
  REGION 1..880: note = Synthetic: Cas13d.4 amino acid sequence
MAKKKRMSAKERKQQQINLRIKKATEDSTKKVNTTVAVNNKPISKEIKKSKAKLAGVKWVIKANDDVAYISSFGKGNNSVLEKRIIGDVSSDVNKDSHMYVNPKYTKKNYEIKNGFSSGSSLTTHPNKPDKNSGMDALCLKTYFEKEIFKDKFNDNMHIQAIYNIFDIEKTLAKHITNIIYAVNSLDRSYIQSGNDTIGFGLNFNIPYAEYGGGKDSNGKPENKSAWEKRESFIKFYNNAKDRFGYFESVFYQNGKQISEEKFYIYLNILNFVRNSTFHYNNTSSHLYKERYCKINPKNNLKTDFEFVSYLNEFVKNKFKNVNKNFISNEKNNLYIILNAYGEDIEDVEVVKKYSKELYKLSVLKTNKNLGVNVKKLRESAIEYGYCPLPYDKEKEVAKLSSIKHKLYKTYDFVITHYLNSNDKLLLEIVEALRLSKNDDKKENVYKIYAEKIFKAEYVINPIKTISNLFAEKGDKLFNEKVSISEEYVEDIRIDKNIHNFTKVIFFLTCFLDGKEINDLLTNIISKLQVIEDHNNVIKAIANNNDAVYKDYSDKYAVFKNSGKIATELEAIKSIARMENKINKAFKEPLLKDAMLALGVSPNDLDEKYEKYFKTDVDADKDHQKVSTFLMNNVINNSRFKYVVKYINPADINRLAKNKHLVKFVLDQIPHKQIDSYYNSVSTVEEPSYKGKIQLLTKKITGLNFYSLFENCKIPNVEKEKKKAVITLYFTIIYILVKNLVNINGLYTLALYFVERDGFFYKKICEKKDKKKTNKDVDYLLLPEIFSGSKYREETKNLKLPKEKDREIMKKYLPNDEDRKEYNKFFKQYRNNIVHLNIIANLSKLTSTIDKEINSYFEIFHYCAQRVMFDYCKNNNKVVL

SEQ ID NO: 123 - protein, 895 aa, synthetic construct
  REGION 1..895: note = Synthetic: Cas13d.5 amino acid sequence
MKKNSNDKTNAKRMGIKSFIKNGDERFITTSIKNEFPVELKLDVIKKTCEPAHEPVSFDYDPKKIDFEKPVLKEKLTSGQSGQKLSTRLFIQKDRDICGIRRKYLEKIFNSNFIEEKKDSNLPMQIVAKVLSTEKVFSNALNKIISQFLSMPRGGVTDNHGEYEIIGNIINHKSLQELNKEKKTKRIKKYLQSVIKNQSYLYNKQFLLSLDESKGSRNDIDENELYDYIRFLAILRNGIAHVFYEKNEPETAKESLFRLVDFIKNDKKLEGAFAKIKIQVNTLYKCRKEEYIKKSGKNFEIIRKIYQNDKPDEKVKDWIRYDFDKSYKYIGLSVAKLGNYTSWAKDIDNLRDKSNPDSGYAGIMHRLNEFSVYLKVKALSTEEKDKYLKNLISKENCEEKDKYYKNIAQFFCSSDLKFANVLQMVKEIKKNKGCTSEDKNCKLCVDERKFNDLSVIVYFISCFLDNKDQNIFLSDLINRFGALSDLLRIQNKILGAGNKYNENYSFLKNERYVTEIKMELETIFALVKVSYKKEDKAFNRLLEDGLVMFGFSKDEAGMKVAGLKEIKEKKEGHYKNKSRSFLINSIVNSRKFAYLAKSIDPQKVPAIIKNEHIVRYILGRINKTNPGQIGRYWRYIMSQNHAGTDKVDDLTNEIIKINIKNILNDAGGWQKSKLNDNNNKKKLKYQQLIGLYLTVAYIFVKNMLECNARYFSAFAQIEKDYLIYTNSDEFYYIDKNKKNLVTERYLKLVKDIIEKNKNTVRKDKIFRKKRQRKHLADISKSIIEFEKLPCCIFTLLRNITEHLNVASNIDIIEGYGKRAGKYHKNAPASYFIFYHYIIQKILADKICTRNLLNIINTYGEPSISFIKIIYVPFAYNLPRYLNLTDARIFCNMDDK

SEQ ID NO: 124 - protein, 857 aa, synthetic construct
  REGION 1..857: note = Synthetic: Cas13c.1 amino acid sequence
MKKLKNPSNRNSLPSIIISKFDSSKIYEIKVKYEKLARLDRLEIGDMSLDENLNILFKKVNFNGIDLEILNPLLLDFDSYTISGKLQKNSTNKTILTLKKDGKIIKYNVLEKDNKYFKNGKEFVIPKDVKEEGKRLVNDKFLLTIEDKKREENSLPKKRKKETQRDILKDETIEIYKRISSNSNIKSEDIYRIKRYMLFRSDMMFFYTFIDNFFYCLYKNKNEQLWNTNFKEKENLGKFIEFTLNDTLKNPRNGILKSYSKDLKVVQEDFVKIKDIFEKIRHALAHFDFTFIDNLLSNNIEFDFNIKLLNIVIEDSQDLYYEAKKEFIEDEKMDILDEKDISIKKLYTFYSKIDIKKPAFNKLINSFLIKDGVENSKLKEYIKEKYNCHYFIDIHDNKEYKKIYNEHKKLISENQNLQLNSKENGQKIKINNDRLEELKGKMNELTKANSLKRLEFKLRLAFGFIKVEYNIFKDFKNNFSEDIKKDMNIDLEKIKSYLDTSYSNNQFFNYKVYNKKTKQKDIDKDIFDDIEKETLKELVENDSLLKIILLFYIFTPKELKGEFLGFIKKFYHDTKNIDKDTKDKEEPLEQIKQEVPLKLKILEKNLTILTIFNYSISLNIEYDKNNNSFYERGNKFKKIYKDLKISHNQEEFDKSLLAPLLKYYMNLYKLLNDFEIYLLLKYKNKDNLNKESLNKLINDEQLKHNDHYNFTTLLSEYFNFDPKKNKKYETLTILRNSISHQKIDNLIYNLDKNKILEQRVKIVELIKEQRDIKETLKFDPINDFTMKTVQLLKSLENQSEKRDKIEEILKQQDLSANDFYNIYKLKGVESIKKELFIRLGKTKIEEKIQEDIAKGSI

SEQ ID NO: 125 - protein, 839 aa, synthetic construct
  REGION 1..839: note = Synthetic: Cas13c.2 amino acid sequence
MNSIEKIKKPSNRNSIPSIIISDYDENKIKEIKVKYLKLARLDKITIQDMEIRDNIVEFKKILLNGIEHTIKDNQKIEFDNYEITAYVRASKQRRDGKITQAKYVVTITDKYLRDNEKEKRFKSTERELPNDTLLMRYKQISGFDTLTSKDIYKIKRYIDFKNEMLFYFQFIEEFFSPLLPKGTNFYSLNIEQNKDKVVKYIVYRLNDDFKNQSLNQFIKKTDTIKYDFLKIQKILSDFRHALAHFDFDFIQKFFDDELDKNRFDISTISLIKTMLQEKEEKYYQEKNNYIEDSDTLTLFDEKESNFSKIHNFYIKISQKKPAFNKLINSFLSKDGVPNEELKSYLATKKIDFFEDIHSNKEYKKIYIKHKNLVVEKQKEESQEKPNGQKLKNYNDELQKLKDEMNKITKQNSLNRLEVKLRLAFGFIANEYNYNFKNFNDKFTLDVKKEQKIKVFKNSSNEKLKEYFESTFIEKRFFHFCVKFFNKKTKKEETKQKNIFNLIENETLEELVKESPLLQIITLLYLFIPKELQGEFVGFILKIYHHTKNITNDTKEDEKSIEDTQNSFSLKLKILAKNLRGLQLFNYSLSHNTLYNTKEHFFYEKGNRWQSVYKSLEISHNQDEFDIHLVIPVIKYYINLNKLIGDFEIYALLTYADKNSITEKLSDITKRDDLKFRGYYNFSTLLFKTFMINTNYEQNQKSTQYIKQTRNDIAHQNIENMLKAFENNEIFAQREEIVNYLQKEHKMQEILHYNPINDFTMKTVQYLKSLNIHSQKESKIADIHKKESLVPNDYYLIYKLKVIELLKQKVIEAIGETKDEEKIKNAIAKEEQIKKGYNK

SEQ ID NO: 126 - DNA, 36 bp, synthetic construct
  misc_feature 1..36: note = Synthetic: Cas13e.3 DR coding sequence
  source 1..36: mol_type = other DNA
gctggagcagccctcgatttgctgggtaatcacagc

SEQ ID NO: 127 - DNA, 36 bp, synthetic construct
  misc_feature 1..36: note = Synthetic: Cas13e.4 DR coding sequence
  source 1..36: mol_type = other DNA
gctgaagcaaccctggttttgcggggtgattacagc

SEQ ID NO: 128 - DNA, 36 bp, synthetic construct
  misc_feature 1..36: note = Synthetic: Cas13e.5 DR coding sequence
  source 1..36: mol_type = other DNA
gctgtagaagcctccgatttgtgaggtgatgacagc

SEQ ID NO: 129 - DNA, 36 bp, synthetic construct
  misc_feature 1..36: note = Synthetic: Cas13e.6 DR coding sequence
  source 1..36: mol_type = other DNA
gctggagcagccctcgatttgcagggtaatcacagc

SEQ ID NO: 130 - DNA, 36 bp, synthetic construct
  misc_feature 1..36: note = Synthetic: Cas13e.7 DR coding sequence
  source 1..36: mol_type = other DNA
gctggagcagccctcgatttgcagggttatcacagc

SEQ ID NO: 131 - DNA, 36 bp, synthetic construct
  misc_feature 1..36: note = Synthetic: Cas13e.8 DR coding sequence
  source 1..36: mol_type = other DNA
gttggagtagccccggatttgcggggtgattacagc

SEQ ID NO: 132 - DNA, 36 bp, synthetic construct; identical to SEQ ID NO: 10
  misc_feature 1..36: note = Synthetic: Cas13f.6 DR coding sequence
  source 1..36: mol_type = other DNA

SEQ ID NO: 133 - DNA, 36 bp, synthetic construct
  misc_feature 1..36: note = Synthetic: Cas13f.7 DR coding sequence
  source 1..36: mol_type = other DNA
gctgtgatggacctcgatttgtggggtagtaacagc

SEQ ID NO: 134 - DNA, 36 bp, synthetic construct
  misc_feature 1..36: note = Synthetic: Cas13d.1 DR coding sequence
  source 1..36: mol_type = other DNA
caactacaaccccgtaaaaatacggggttctgaaac

SEQ ID NO: 135 - DNA, 36 bp, synthetic construct
  misc_feature 1..36: note = Synthetic: Cas13d.2 DR coding sequence
  source 1..36: mol_type = other DNA
gttaaataccacctaagaatgaggaggttctataac

SEQ ID NO: 136 - DNA, 36 bp, synthetic construct
  misc_feature 1..36: note = Synthetic: Cas13d.3 DR coding sequence
  source 1..36: mol_type = other DNA
gaacgatagcctgctgaaatatgcaggttctaagac

SEQ ID NO: 137 - DNA, 36 bp, synthetic construct
  misc_feature 1..36: note = Synthetic: Cas13d.4 DR coding sequence
  source 1..36: mol_type = other DNA
gattgaaagctatgcgaatttgcacagtcttaaaac

SEQ ID NO: 138 - DNA, 36 bp, synthetic construct
  misc_feature 1..36: note = Synthetic: Cas13d.5 DR coding sequence
  source 1..36: mol_type = other DNA
gagatagacccttgttaactcgtaaggttctgtgac

SEQ ID NO: 139 - DNA, 36 bp, synthetic construct
  misc_feature 1..36: note = Synthetic: Cas13c.1 DR coding sequence
  source 1..36: mol_type = other DNA
attggatatacccctaatttgagaggggaataaaac

SEQ ID NO: 140 - DNA, 36 bp, synthetic construct
  misc_feature 1..36: note = Synthetic: Cas13c.2 DR coding sequence
  source 1..36: mol_type = other DNA
gttggactataccctcgtttgtagggggaataaaac

SEQ ID NO: 141 - protein, 289 aa, synthetic construct
  REGION 1..289: note = Synthetic: SsoCas6 amino acid sequence
MPLIFKIGYNVIPLQDVILPTPSSKVLKYLIQSGKLIPSLKDLITSRDKYKPIFISHLGFNQRRIFQTNGNLKTITKGSRLSSIIAFSTQANVLSEVADEGIFETVYGKFHIMIESIEIVEVEKLKEEVEKHMNDNIRVRFVSPTLLSSKVLLPPSLSERYKKIHAGYSTLPSVGLIVAYAYNVYCNLIGKKEVEVRAFKFGILSNALSRIIGYDLHPVTVAIGEDSKGNLRKARGVMGWIEFDIPDERLKRRALNYLLTSSYLGIGRSRGIGFGEIRLEFRKIEEKEG

SEQ ID NO: 142 - protein, 218 aa, synthetic construct
  REGION 1..218: note = Synthetic: MmCas6 amino acid sequence
MDLEYMHISYPNILLNMRDGSKLRGYFAKKYIDEEIVHNHRDNAFVYKYPQIQFKIIDRSPLIIGIGSLGINFLESKRIFFEKELIISNDTNDITEVNVHKDMDHFGTTDKILKYQFKTPWMALNAKNSEIYKNSDEIDREEFLKRVLIGNILSMSKSLGYTIEEKLKVKINLKEVPVKFKNQNMVGFRGEFYINFDIPQYLGIGRNVSRGFGTVVKV

SEQ ID NO: 143 - protein, 242 aa, synthetic construct
  REGION 1..242: note = Synthetic: SpCas5d amino acid sequence
MYRSRDFYVRVSGQRALFTNPATKGGSERSSYSVPTRQALNGIVDAIYYKPTFTNIVTEVKVINQIQTELQGVRALLHDYSADLSYVSYLSDVVYLIKFHFVWNEDRKDLNSDRLPAKHEAIMERSIRKGGRRDVFLGTRECLGLLDDISQEEYETTVSYYNGVNIDLGIMFHSFAYPKDKKTPLKSYFTKTVMKNGVITFKAQSECDIVNTLSSYAFKAPEEIKSVNDECMEYDAMEKGEN

SEQ ID NO: 144 - protein, 236 aa, synthetic construct
  REGION 1..236: note = Synthetic: BhCas5d amino acid sequence
MRNEVQFELFGDYALFTDPLTKIGGEKLSYSVPTYQALKGIAESIYWKPTIVFVIDELRVMKPIQMESKGVRPIEYGGGNTLAHYTYLKDVHYQVKAHFEFNLHRPDLAFDRNEGKHYSILQRSLKAGGRRDIFLGARECQGYVAPCEFGSGDGFYDGQGKYHLGTMVHGFNYPDETGQHQLDVRLWSAVMENGYIQFPRPEDCPIVRPVKEMEPKIFNPDNVQSAEQLLHDLGGE

SEQ ID NO: 145 - protein, 260 aa, synthetic construct
  REGION 1..260: note = Synthetic: SaCas6 amino acid sequence
MPNDPYSLYSIVIELGAAEKGFPTGILGRSLHSQVLQWFKQDNPFLATELHQSQISPFSISPLMGKRHAKLTKAGDRLFFRICLLRGDLLQPLLNGIEQTVNQSVCLDKFRFRLCQTHILPGSHPLAGASHYSLISQTPVSSKITLDFKSSTSFKVDRKIIQVFPLGEHVFNSLLRRWNNFAPEDLHFSQVDWSIPIAAFDVKTIPIHLKKVEIGAQGWVTYIFPNTEQAKIASVLSEFAFFSGVGRKTTMGMGQVQVRS

SEQ ID NO: 146 - protein, 199 aa, synthetic construct
  REGION 1..199: note = Synthetic: EcCas6e amino acid sequence
MYLSKVIIARAWSRDLYQLHQGLWHLFPNRPDAARDFLFHVEKRNTPEGCHVLLQSAQMPVSTAVATVIKTKQVEFQLQVGVPLYFRLRANPIKTILDNQKRLDSKGNIKRCRVPLIKEAEQIAWLQRKLGNAARVEDVHPISERPQYFSGDGKSGKIQTVCFEGVLTINDAPALIDLVQQGIGPAKSMGCGLLSLAPL

SEQ ID NO: 147 - protein, 187 aa, synthetic construct
  REGION 1..187: note = Synthetic: PaCas6f amino acid sequence
MDHYLDIRLRPDPEFPPAQLMSVLFGKLHQALVAQGGDRIGVSFPDLDESRSRLGERLRIHASADDLRALLARPWLEGLRDHLQFGEPAVVPHPTPYRQVSRVQAKSNPERLRRRLMRRHDLSEEEARKRIPDTVARALDLPFVTLRSQSTGQHFRLFIRHGPLQVTAEEGGFTCYGLSKGGFVPWF

SEQ ID NO: 148 - protein, 314 aa, synthetic construct
  REGION 1..314: note = Synthetic: MtCas6 amino acid sequence
MAARRGGIRRTDLLRRSGQPRGRHRASAAESGLTWISPTLILVGFSHRGDRRMTEHLSRLTLTLEVDAPLERARVATLGPHLHGVLMESIPADYVQTLHTVPVNPYSQYALARSTTSLEWKISTLTNEARQQIVGPINDAAFAGFRLRASGIATQVTSRSLEQNPLSQFARIFYARPETRKFRVEFLTPTAFKQSGEYVFWPDPRLVFQSLAQKYGAIVDGEEPDPGLIAEFGQSVRLSAFRVASAPFAVGAARVPGFTGSATFTVRGVDTFASYIAALLWFGEFSGCGIKASMGMGAIRVQPLAPREKCVPKP

SEQ ID NO: 149 - protein, 264 aa, synthetic construct
  REGION 1..264: note = Synthetic: PfCas6 amino acid sequence
MRFLIRLVPEDKDRAFKVPYNHQYYLQGLIYNAIKSSNPKLATYLHEVKGPKLFTYSLFMAEKREHPKGLPYFLGYKKGFFYFSTCVPEIAEALVNGLLMNPEVRLWDERFYLHEIKVLREPKKFNGSTFVTLSPIAVTVVRKGKSYDVPPMEKEFYSIIKDDLQDKYVMAYGDKPPSEFEMEVLIAKPKRFRIKPGIYQTAWHLVFRAYGNDDLLKVGYEVGFGEKNSLGFGMVKVEGNKTTKEAEEQEKITFNSREELKTGV

SEQ ID NO: 150 - protein, 236 aa, synthetic construct
  REGION 1..236: note = Synthetic: PaCsf5 amino acid sequence
MFVTQVIFNIGERTYPDRARAMVAELMDGVQPGLVATLMNYIPGTSTSRTEFPTVQFGGASDGFCLLGFGDGGGAIVRDAVPLIHAALARRMPDRIIQVEHKEHSLSAEARPYVLSYTVPRMVVQKKQRHAERLLHEAEGKAHLEGLFLRSLQRQAAAVGLPLPENLEVEFKGAVGDFAAKHNPNSKVAYRGLRGAVFDVNARLGGIWTAGFMLSKGYGQFNATHQLSGAVNALSE

SEQ ID NO: 151 - protein, 259 aa, synthetic construct
  REGION 1..259: note = Synthetic: MtCsf5 amino acid sequence
MHQTLIRINWPKGFKCPPAEFREKLAKSEMFPPEFFHYGTELAVWDKQTAEVEGKIKTVSKEKIIKTFDKPIPLNGRAPVRVIGGQAWAGVIADPEMEGMLIPHLGSILKVASSAAGCAVKIELEQRKFGISYTEYPVKYNLRELVLKRRCEDARSTDIESLIADRIWGGVSGESYYGIDGTCAKFGFEPPSREQLELRIFPMKNIGLHMKSSDGLSKEYMSLIDAEVWMNAKLEGVWQVGNLISRGYGRFIKSIGAQS

SEQ ID NO: 152 - DNA, 24 bp, synthetic construct
  misc_feature 1..24: note = Synthetic: SsoCas6 DR coding sequence
  source 1..24: mol_type = other DNA
gataatctcttatagaattgaaag

SEQ ID NO: 153 - DNA, 37 bp, synthetic construct
  misc_feature 1..37: note = Synthetic: MmCas6 DR coding sequence
  source 1..37: mol_type = other DNA
ctaaaagaataacttgcaaaataacaagcattgaaac

SEQ ID NO: 154 - DNA, 32 bp, synthetic construct
  misc_feature 1..32: note = Synthetic: SpCas5d DR coding sequence
  source 1..32: mol_type = other DNA
gtctcacccttcatgggtgagtggattgaaat

SEQ ID NO: 155 - DNA, 32 bp, synthetic construct
  misc_feature 1..32: note = Synthetic: BhCas5d DR coding sequence
  source 1..32: mol_type = other DNA
gtcgcactcttcatgggtgcgtggattgaaat

SEQ ID NO: 156 - DNA, 37 bp, synthetic construct
  misc_feature 1..37: note = Synthetic: SaCas6 DR coding sequence
  source 1..37: mol_type = other DNA
gtttcagtcccgtagtcgggatttagtggttggaaag

SEQ ID NO: 157 - DNA, 29 bp, synthetic construct
  misc_feature 1..29: note = Synthetic: EcCas6e DR coding sequence
  source 1..29: mol_type = other DNA
gagttccccgcgccagcggggataaaccg

SEQ ID NO: 158 - DNA, 28 bp, synthetic construct
  misc_feature 1..28: note = Synthetic: PaCas6f DR coding sequence
  source 1..28: mol_type = other DNA
gttcactgccgtataggcagctaagaaa

SEQ ID NO: 159 - DNA, 36 bp, synthetic construct
  misc_feature 1..36: note = Synthetic: MtCas6 DR coding sequence
  source 1..36: mol_type = other DNA
gtcgtcagacccaaaaccccgagaggggacggaaac

SEQ ID NO: 160 - DNA, 30 bp, synthetic construct
  misc_feature 1..30: note = Synthetic: PfCas6 DR coding sequence
  source 1..30: mol_type = other DNA
gttacaataagactaaaatagaattgaaag

SEQ ID NO: 161 - DNA, 29 bp, synthetic construct
  misc_feature 1..29: note = Synthetic: PaCsf5 DR coding sequence
  source 1..29: mol_type = other DNA
gtatttcccgcgtgcgcgggggtgagcgg

SEQ ID NO: 162 - DNA, 37 bp, synthetic construct
  misc_feature 1..37: note = Synthetic: MtCsf5 DR coding sequence
  source 1..37: mol_type = other DNA
tattggatacacccactcattggtgggtggttagaac

SEQ ID NO: 163 - protein, 385 aa, synthetic construct
  REGION 1..385: note = Synthetic: hADAR2DD-E488Q/T375G (ADARv2)
QLHLPQVLADAVSRLVLGKFGDLTDNFSSPHARRKVLAGVVMTTGTDVKDAKVISVSTGGKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 164 - DNA, 30 bp, synthetic construct
  misc_feature 1..30: note = Synthetic: >spacer coding sequence-5mis/30nt targeting mCherry *
  source 1..30: mol_type = other DNA
ctcccagcccatggttttcttctgcattac

SEQ ID NO: 165 - DNA, 2217 bp, synthetic construct
  misc_feature 1..2217: note = Synthetic: >The reporter plasmid (pCMV-BFP-P2A-mCherry(W148X)-ployA)
  source 1..2217: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcatgagcgagctgattaaggagaacatgcacatgaagctgtatatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaggacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacactgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacatacgtcgagcagcacgaggtggcagtggccagatactgcgacctccctagcaaactggggcacaagctgaatggcgccactaacttctccctgttgaaacaagcaggggatgtcgaagagaatcccgggccaatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaaaaccatgggctaggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaatcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 166 - DNA, 50 bp, synthetic construct
  misc_feature 1..50: note = Synthetic: > spacer coding sequence targeting mCherry (W148X)
  source 1..50: mol_type = other DNA
tacatccgctcggaggaggcctcccagcccatggttttcttctgcattac

SEQ ID NO: 167 - DNA, 30 bp, synthetic construct
  misc_feature 1..30: note = Synthetic: > NT-spacer coding sequence
  source 1..30: mol_type = other DNA
ggtcttcgatattcaagcgtcggaagacct

SEQ ID NO: 168 - protein, 505 aa, synthetic construct
  REGION 1..505: note = Synthetic: > dCas13e.2-N150+C150 amino acid sequence
MSGWITPAGILLLASFFVERGILHRLMGNIGGFKDNRGEYGLTHDIFTTYCLKGSYSIRAQDHDAVMFRDILGYLSRVPTESFQRIKQPQIRKEGQLSERKTDKFITFALNYLEDYGLKDLEGCKACFARSKIVREQENVESINDKEYKPHENKKKVEIHFDQSKEDRFYINRNNVILKIQKKDGHSNIVRMGVYELKYLVLMSLVGKAKEAVEKIDNYIQDLRDQLPYIEGKNKEEIKEYVRFFPRFIRSHLGLLQINDEEKIKARLDYVKTKWLDKKEKSKELELHKKGRDILRYINERCDRELNRNVYNRILELLVSKDLTGFYRELEELKRTRRIDKNIVQNLSGQKTINALHEKVCDLVLKEIESLDTENLRKYLGLIPKEEKEVTFKEKVDRILKQPVIYKGFLRYQFFKDDKKSFVLLVEDALKEKGGGCDVPLGKEYYKIVSLDKYDKENKTLCETLAMDRLCLMMARQYYLSLNAKLAQEAQQIEWKKEDSIELII

SEQ ID NO: 169 - protein, 445 aa, synthetic construct
  REGION 1..445: note = Synthetic: > dCas13e.2-N180+C180 amino acid sequence
MGFKDNRGEYGLTHDIFTTYCLKGSYSIRAQDHDAVMFRDILGYLSRVPTESFQRIKQPQIRKEGQLSERKTDKFITFALNYLEDYGLKDLEGCKACFARSKIVREQENVESINDKEYKPHENKKKVEIHFDQSKEDRFYINRNNVILKIQKKDGHSNIVRMGVYELKYLVLMSLVGKAKEAVEKIDNYIQDLRDQLPYIEGKNKEEIKEYVRFFPRFIRSHLGLLQINDEEKIKARLDYVKTKWLDKKEKSKELELHKKGRDILRYINERCDRELNRNVYNRILELLVSKDLTGFYRELEELKRTRRIDKNIVQNLSGQKTINALHEKVCDLVLKEIESLDTENLRKYLGLIPKEEKEVTFKEKVDRILKQPVIYKGFLRYQFFKDDKKSFVLLVEDALKEKGGGCDVPLGKEYYKIVSLDKYDKENKTLCETLAMDRLCLMMA

SEQ ID NO: 170 - protein, 506 aa, synthetic construct
  REGION 1..506: note = Synthetic: > dCas13e.3-N180+C180 amino acid sequence
MAHYSLRDSYVIKAEETKSFRDLLGYLSRVPQQAVDWLNEHNQLSEDEKKEFLNQKPSDEESQEQSKTENTDRQADRMPRRSLRKTDKFILFAAKFIEDWAQKEKMDVTFARYQKTVTEDENKNQDGKQVRDVQLKYEKDTKKLNPDFDYKWTYYIRNNHAIIQIKPDEYKQAVSARISENELKYLVLLIFQGKGWEAIKKIGDYIFHIGNKIKIGRFDHNEERRMPSFLKNPPADIIGEMVENRLKYIRDELNKVIETIKKEEPQNNKWLLYKGKKISIILKFISDSISDIKKRPDVNEYNTLRDMLQKLDFDNFYERLKSYVSEGRIEQTLYDEIKGIKDISTLCIKICELRLAALEELEKEGGDDLNKYIGLAVQEKHKNYDDSNTPQKKAERFLESQFSVGKNFLRETFYDEYIKNRKSLYEIIKEKITGITPLNENRWYLMDKNPKEFESKDSKIIRGLCNIYIQDILCMKIALWYYENLSPSYKNKLKWDFIGQGFGYDR

SEQ ID NO: 171 - protein, 571 aa, synthetic construct
  REGION 1..571: note = Synthetic: > dCas13e.7-N150+C150 amino acid sequence
MGIPGFKRSDKKKWGDGQKIDYGFTRKLMSFYSLRDSYSVNVQENKELTAFRDILGYLARVPGQAIDWLIEKGKLTKEEGKQFYLGEQSEEREEKAKKEEIKYALRKTDKFMLFAVRFIEDWAEQERIKVEFARYEKMTIVNENKKQDEKEERKVKFVSDEPTAAGWTYYIRNNHAIIKIIPDDKKKKAVSARISENELKYLVLTIIDGNGKNAIAYIGDYIFRTARQIENKSYNAESEKYAPAFVRGGQKKSVDKRIKYIRDEIQQVINDIEAEQEKQKNEQDAPAENRTWLIYKGKKISIILRYVNDNIAEYKKRLSVTEYNELRGYLQQLDFINFHRKLAEYQHHGRLPNGFAESINKFQDLSKLCIEVCERQKKKLQEMAAKGGIELEQYIGLAPKEENQEQNKYATKANNFIKVWLSIPENFLRQKFYDKFCKQQECKNKGSDKPDNTSVPQRKYFIAIIREKNIRPIHADKYYLLGQNPKDYERPDGKIIRQLCDVYCKDGLCMAMAKWYYENRLGKFKDLIEWQTGDDKQQHGYAGHTLEYQATEKIKIRFKLADFTRLDIIEP

SEQ ID NO: 172 - protein, 492 aa, synthetic construct
  REGION 1..492: note = Synthetic: > dCas13f.2-N150+C150 amino acid sequence
MLISGISGFKRNDPTGQPRRNLFTYFSAREGYKALPDMQKHFLLFTLVNYLSNQDEYISELKQYGEIGQGAFFNRIASTFLNISGISGNTKFYSYQSKRIKEQRGELNSEKDSFEWIEPFQGNSYFEINGHKGVIGEDELKELCYALLVAKQDINAVEGKIMQFLKKFRNTGNLQQVKDDEMLEIEYFPASYFNESKKEDIKKEILGRLDKKIRSCSAKAEKAYDKMKEVMEFINNSLPAEEKLKRKDYRRYLKMVRFWSREKGNIEREFRTKEWSKYFSSDFWRKNNLEDVYKLATQKNAELFKNLKAAAEKMGETEFEKYQQINDVKDLASLRRLTQDFGLKWEEKDWEEYSEQIKKQITDRQKLTIMKQRVTAELKKKHGIENLNLRITIDSNKSRKAVLNRIAIPRGFVKKHILGWQGSEKISKNIREAECKILLSKKYEELSRQFFEAGNFDKLTQINGLYEKNKLTAFMSVYLMGRLNIQLNKHTE

SEQ ID NO: 173 - DNA, 36 bp, synthetic construct
  source 1..36: mol_type = other DNA
gttgtggaaggtccagttttgaggggctattacaac

SEQ ID NO: 174 - protein, 983 aa, synthetic construct
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKED

SEQ ID NO: 175 - protein, 1402 aa, synthetic construct; identical to SEQ ID NO: 42

SEQ ID NO: 176 - protein, 983 aa, synthetic construct
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKAAATCIDGQTRVAVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKED

SEQ ID NO: 177 - protein, 1402 aa, synthetic construct
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKAAATCIDGQTRVAVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDGSLQLPPLERLTLGGGGGSGGGGSGGGGSGGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

(19) United States
(12) Patent Application Publication
(10) Pub. No.: US 2023/0086489 A1
(43) Pub. Date: Mar. 23, 2023

(54) NOVEL DESIGN OF GUIDE RNA AND USES THEREOF

(71) Applicant: HuiGene Therapeutics Co., Ltd., Shanghai (CN)

(72) Inventors: Xing Wang, Shanghai (CN); Linyu Shi, Shanghai (CN); Xuan Yao, Shanghai (CN)

(73) Assignee: HuiGene Therapeutics Co., Ltd., Shanghai (CN)

(21) Appl. No.: 17/930,510

(22) Filed: Sep. 8, 2022

Related U.S. Application Data
(63) Continuation of application No. PCT/CN2022/115961, filed on Aug. 30, 2022.

(30) Foreign Application Priority Data
Aug. 30, 2021 (CN) PCT/CN2021/115423

(51) Int. Cl. C12N 9/22; C12N 9/78; C12N 15/11; C12N 15/62; C12N 15/90

(52) U.S. Cl. CPC C12N 9/22; C12N 9/78; C12Y 305/04004; C12N 15/11; C12N 15/62; C12N 15/907; C12N 2310/20; C12N 2800/80; C07K 2319/80; A61K 48/00

(57) ABSTRACT

The disclosure provides a novel design of guide RNA and uses thereof. In one aspect, the guide RNA comprises a spacer sequence flanked by a direct repeat sequence at both the 5′ end and the 3′ end of the spacer sequence.

### REFERENCE TO RELATED APPLICATIONS

This application is a continuation application of International Patent Application No. PCT/CN2022/115961, filed on Aug. 30, 2022, which claims foreign priority under 35 USC 365(b) to International Patent Application No. PCT/CN2021/115423, filed on Aug. 30, 2021, the entire contents of each of the above referenced applications, including any sequence listing and drawings, are incorporated herein by reference in its entirety.

### SEQUENCE LISTING

The instant application contains a Sequence Listing which has been submitted electronically in XML format and is hereby incorporated by reference in its entirety. Said XML copy, created on Sep. 20, 2022, is named 132045-01001_SL.xml and is 309,778 bytes in size.

### BACKGROUND

Since the development of RNA base editors by the team of Professor ZHANG Feng, the CRISPR RNA (crRNA) binding property of Class 2, Type VI (Cas13) effector proteins and CRISPR-associated Protein for Class 1 pre-crRNA processing (CasPR, e.g., Cas6) has been utilized in combination with a heterologous function domain (e.g., an adenine deamination domain) associated with such a Cas protein and a guide RNA to constitute a CRISPR-Cas system for various purposes (e.g., A-to-I base editing) based on the function of the heterologous function domain (e.g., an adenine deamination domain). The guide RNA comprises a direct repeat sequence capable of forming a complex with the Cas protein associated with the heterologous function domain and a spacer sequence capable of hybridizing to a target RNA, thereby targeting or recruiting the Cas protein and the associated heterologous function domain (e.g., an adenine deamination domain) to the target RNA. However, the efficiency of such a CRISPR-Cas system may limit its use in practices, such as, the commercial development of therapeutic products.

It would be desirable in the art to further improve the efficiency of such a CRISPR-Cas system.

### SUMMARY

One aspect of the disclosure provides a CRISPR-Cas system, comprising:

(a) a CRISPR RNA (crRNA) binding polypeptide or a polynucleotide coding sequence (e.g., a DNA coding sequence or an RNA coding sequence) thereof, the crRNA binding polypeptide comprising, consisting essentially of, or consisting of a crRNA binding domain of a Cas effector protein,

(b) a heterologous functional domain or a polynucleotide coding sequence thereof (e.g., a DNA coding sequence or an RNA coding sequence), and

(c) a guide RNA (gRNA) or a polynucleotide coding sequence (e.g., a DNA coding sequence or an RNA coding sequence) thereof, the gRNA comprising:

(i) a 5′ direct repeat (DR) sequence and a 3′ direct repeat (DR) sequence, each capable of forming a complex with the crRNA binding domain; and

(ii) a spacer sequence capable of hybridizing to a target RNA, and guiding or recruiting the complex to the target RNA,

wherein the crRNA binding polypeptide:

(1) is linked (e.g., fused) to the heterologous functional domain, and

(2) substantially lacks the ability (e.g., having no more than 50%, 40%, 30%, 20%, 10%, 5%, 2%, or 1% of that of the Cas effector protein) to process or cleave DR sequence on the gRNA, and

wherein the spacer sequence is flanked by the 5′ and 3′ DR sequences at the 5′ end and the 3′ end of the spacer sequence, respectively; optionally, the 5′ and 3′ DR sequences are identical.

Another aspect of the disclosure provides a guide RNA (gRNA) comprising:

a) a 5′ direct repeat (DR) sequence and a 3′ direct repeat (DR) sequence, each capable of forming a complex with a CRISPR RNA (crRNA) binding polypeptide comprising, consisting essentially of, or consisting of a crRNA binding domain of a Cas effector protein; and

b) a spacer sequence capable of hybridizing to a target RNA, and guiding or recruiting the complex to the target RNA,

wherein the spacer sequence is flanked by the 5′ and 3′ DR sequences at the 5′ end and the 3′ end of the spacer sequence, respectively; optionally, the 5′ and 3′ DR sequences are identical.

In some embodiments, the crRNA binding polypeptide substantially lacks the ability (e.g., having no more than 50%, 40%, 30%, 20%, 10%, 5%, 2%, or 1% of that of the Cas effector protein) to process or cleave DR sequence on the gRNA.

In some embodiments, the crRNA binding polypeptide is linked (e.g., fused) to a heterologous functional domain.

Another aspect of the disclosure provides a modified Cas13 protein with both HEPN1 and HEPN2 domains substantially removed from a parental or wild-type Cas13 effector protein (e.g., substantially lacking both the HEPN1 and HEPN2 domains of the parental or wild-type Cas13 effector protein), with the proviso that the modified Cas13 protein is not minidCas13e.1-N180+C150.

In some embodiments, the modified Cas13 protein has a first deletion of or comprising the HEPN1 domain, and a second deletion of or comprising the HEPN2 domain, and substantially lacking the ability (e.g., having no more than 50%, 40%, 30%, 20%, 10%, 5%, 2%, or 1% of that of the parental or wild-type Cas13 effector protein) to process or cleave a direct repeat (DR) sequence capable of forming a complex with the modified Cas13 protein in a guide RNA (gRNA) comprising:

a) the direct repeat sequence; and

b) a spacer sequence capable of hybridizing to a target RNA, and guiding or recruiting the complex to the target RNA.

In some embodiments, (1) the first deletion is no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues larger than the HEPN1 domain of the parental or wild-type Cas13 effector protein, and is no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues smaller than the HEPN1 domain of the parental or wild-type Cas13 effector protein; and (2) the second deletion is no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues larger than the HEPN2 domain of the parental or wild-type Cas13 effector protein, and is no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues smaller than the HEPN2 domain of the parental or wild-type Cas13 effector protein.

In some embodiments, the parental or wild-type Cas13 effector protein is a Cas13a effector protein, a Cas13b effector protein, a Cas13c effector protein, a Cas13d effector protein, a Cas13e effector protein, or a Cas13f effector protein.

Another aspect of the disclosure provides a fusion protein comprising:

a) the modified Cas13 protein as described herein; and,

b) a heterologous functional domain, e.g., a deaminase domain.

Another aspect of the disclosure provides a CRISPR-Cas13 system comprising:

a) the modified Cas13 protein as described herein or the fusion protein as described herein or a polynucleotide coding sequence (e.g., a DNA coding sequence or an RNA coding sequence) thereof; and

b) a guide RNA (gRNA) or a polynucleotide coding sequence (e.g., a DNA coding sequence or an RNA coding sequence) thereof, the gRNA comprising:

i. a direct repeat (DR) sequence capable of forming a complex with the modified Cas13 protein of the fusion protein; and,

ii. a spacer sequence capable of hybridizing to a target RNA, and guiding or recruiting the complex to the target RNA.

In some embodiments, the gRNA comprises

a) a 5′ direct repeat (DR) sequence and a 3′ direct repeat (DR) sequence, each capable of forming a complex with the modified Cas protein of the fusion protein; and

b) a spacer sequence capable of hybridizing to a target RNA, and guiding or recruiting the complex to the target RNA,

wherein the spacer sequence is flanked by the 5′ and 3′ DR sequences at the 5′ end and the 3′ end of the spacer sequence, respectively; optionally, the 5′ and 3′ DR sequences are identical.

In some embodiments, the Cas effector protein is a Class 2, Type VI (Cas13) effector protein.

In some embodiments, the crRNA binding domain substantially lacks the HEPN1 domain and/or the HEPN2 domain of the Cas effector protein.

In some embodiments, the crRNA binding domain substantially lacks both the HEPN1 and HEPN2 domains of the Cas effector protein.

In some embodiments, the crRNA binding domain has a first deletion of or comprising the HEPN1 domain, and a second deletion of or comprising the HEPN2 domain.

In some embodiments, (1) the first deletion is no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues larger than the HEPN1 domain of the Cas13 effector protein, and is no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues smaller than the HEPN1 domain of the Cas13 effector protein; and (2) the second deletion is no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues larger than the HEPN2 domain of the Cas13 effector protein, and is no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues smaller than the HEPN2 domain of the Cas13 effector protein.

In some embodiments, the Cas13 effector protein is a Cas13a effector protein, a Cas13b effector protein, a Cas13c effector protein, a Cas13d effector protein, a Cas13e effector protein, or a Cas13f effector protein.

In some embodiments, the Cas effector protein comprises an amino acid sequence (1) of any one of SEQ ID NOs: 1-7, 111-125, and 173, or (2) having a sequence identity of at least 80%, 85%, 90%, 91%, 92%, 93%, 94%, 95%, 96%, 97%, 98%, or 99% to the amino acid sequence of any one of SEQ ID NOs: 1-7, 111-125, and 173.

In some embodiments, the DR sequence or the 5′ and/or the 3′ DR sequences each has substantially the same secondary structure as the secondary structure of any one of SEQ ID NOs: 8-14 and 126-140.

In some embodiments, the DR sequence or the 5′ and/or the 3′ DR sequences each is encoded by or comprises any one of SEQ ID NOs: 8-14 and 126-140.

In some embodiments, the Cas effector protein is a Class 2, Type VI-E (Cas13e) Cas effector protein (e.g., SEQ ID NO: 1), and wherein the crRNA binding domain lacks about 180 (e.g., 170, 171, 172, 173, 174, 175, 176, 177, 178, 179, 180, 181, 182, 183, 184, 185, 186, 187, 188, 189, or 190) N-terminal residues, and lacks about 150 (e.g., 140, 141, 142, 143, 144, 145, 146, 147, 148, 149, 150, 151, 152, 153, 154, 155, 156, 157, 158, 159, or 160) C-terminal residues of the Cas13e effector protein (e.g., SEQ ID NO: 1).

In some embodiments, the crRNA binding polypeptide comprises an amino acid sequence of any one of SEQ ID NOs: 32, 168-172, and 174.

In some embodiments, the Cas effector protein is a CasPR (CRISPR-associated Protein for Class 1 pre-crRNA processing).

In some embodiments, the CasPR is Cas5d, Cas6 (e.g., Cas6e), or Csf5.

In some embodiments, the CasPR comprises an amino acid sequence (1) of any one of SEQ ID NOs: 141-151, or (2) having a sequence identity of at least 80%, 85%, 90%, 91%, 92%, 93%, 94%, 95%, 96%, 97%, 98%, or 99% to the amino acid sequence of any one of SEQ ID NOs: 141-151.

In some embodiments, the DR sequence or the 5′ and/or the 3′ DR sequences each has substantially the same secondary structure as the secondary structure of any one of SEQ ID NOs: 47 and 152-162.

In some embodiments, the DR sequence or the 5′ and/or the 3′ DR sequences each is encoded by or comprises any one of SEQ ID NOs: 47 and 152-162.

In some embodiments, the CasPR is EcCas6e; optionally, the crRNA binding polypeptide comprises the amino acid sequence of SEQ ID NO: 51 (EcCas6e-H20L).

In some embodiments, the gRNA comprises, from 5′ to 3′, a first DR sequence, a first spacer sequence, a second DR sequence, a second spacer sequence, and a third DR sequence, whereby the first spacer sequence is flanked by the first and second DR sequences at the 5′ end and the 3′ end of the first spacer sequence, respectively, and the second spacer sequence is flanked by the second and third DR sequences at the 5′ end and the 3′ end of the second spacer sequence, respectively;

wherein the first spacer sequence and the second spacer sequence are each capable of hybridizing to a first target RNA and a second target RNA, respectively, and guiding or recruiting the complex to the first target RNA and the second target RNA, respectively, and wherein the first and the second target RNA are the same or different.

In some embodiments, the target RNA is encoded by a eukaryotic DNA.

In some embodiments, the eukaryotic DNA is a non-human mammalian DNA, a non-human primate DNA, a human DNA, a plant DNA, an insect DNA, a bird DNA, a reptile DNA, a rodent DNA, a fish DNA, a worm/nematode DNA, or a yeast DNA.

In some embodiments, the target RNA is an mRNA.

In some embodiments, the spacer sequence is between 15-100 nucleotides, 15-80 nucleotides, 15-60 nucleotides, between 25-50 nucleotides, between 30-50 nucleotides, about 100 nucleotides, about 80 nucleotides, about 60 nucleotides, about 55 nucleotides, about 50 nucleotides, about 45 nucleotides, about 40 nucleotides, about 35 nucleotides, about 30 nucleotides, about 20 nucleotides, or about 15 nucleotides in length.

In some embodiments, the spacer sequence is 90-100% complementary to the target RNA, and/or contains no more than 1, 2, 3, 4, or 5 consecutive or non-consecutive mismatches to the target RNA.

In some embodiments, the heterologous functional domain comprises: a reporter protein or a detection label (e.g., GST, HRP, CAT, GFP, HcRed, DsRed, CFP, YFP, BFP), a protein targeting moiety, a DNA binding domain (e.g., MBP, Lex A DBD, Gal4 DBD), an epitope tag (e.g., His, myc, V5, FLAG, HA, VSV-G, Trx, etc), a transcription activation domain (e.g., VP64 or VPR), a transcription inhibition domain (e.g., KRAB moiety or SID moiety), a nuclease domain (e.g., FokI), a deaminase domain (e.g., ADAR1, ADAR2, APOBEC, AID, or TAD), a methylation domain, a demethylation domain (e.g., FTO, ALKBH5), a methyltransferase domain, a methylation reader domain, a transcription release factor, an HDAC, a moiety having ssRNA cleavage activity, a moiety having dsRNA cleavage activity, a moiety having ssDNA cleavage activity, a moiety having dsDNA cleavage activity, a DNA or RNA ligase domain, or any combination thereof.

In some embodiments, the heterologous functional domain comprises a deaminase domain, for example, an adenosine deaminase domain, such as a double-stranded RNA-specific adenosine deaminase (e.g., Adenosine deaminase acting on RNA (ADAR), such as, ADAR1 or ADAR2), apolipoprotein B mRNA editing enzyme, catalytic polypeptide-like (APOBEC), activation-induced cytidine deaminase (AID), or a functional fragment thereof; or a cytidine deaminase domain, such as, RescueS (SEQ ID NO: 56), or a functional fragment thereof.

In some embodiments, the ADAR2 or a functional fragment thereof comprising ADAR2DD comprises E488Q mutation or a E-to-Q substitution mutation at a position corresponding to E488 of human ADAR2, and optionally further comprises T375G mutation or a T-to-G substitution mutation at a position corresponding to T375 of human ADAR2.

In some embodiments, the deaminase domain is hADAR2DD-E488Q (SEQ ID NO: 34), hADAR2DD-E488Q/T375G (SEQ ID NO: 163), or RescueS (SEQ ID NO: 56).

In some embodiments, the heterologous functional domain deaminates an adenosine (A) in the target RNA to an inosine (I) and/or deaminates a cytidine (C) in the target RNA to an uridine (U).

In some embodiments, the spacer sequence comprises a cystine (C) mismatch opposite to the adenosine (A) in the target RNA and/or an adenosine (A) mismatch opposite to the cytidine (C) in the target RNA.

In some embodiments, the cystine or adenosine mismatch is about 5, 6, 7, 8, 9, 10, 11, 12, 13, 14, 15, 16, 17, 18, 19, 20, 21, 22, 23, 24, 25, 26, 27, 28, 29, or 30 nucleotides (e.g., about 15-25 nucleotides) from the 5′ or 3′ DR sequence.

In some embodiments, the heterologous functional domain comprises a m6A-associated regulation domain, such as, a m6A-associated methyltransferase domain (e.g., METTL3, METTL14, WTAP, KIAA1429, or a functional fragment thereof), a m6A-associated demethylation domain (e.g., Fat mass and obesity-associated protein (FTO), ALKBH5, or a functional fragment thereof), or a combination thereof.

In some embodiments, the heterologous functional domain is fused or conjugated N-terminally, C-terminally, or internally to the crRNA binding polypeptide.

In some embodiments, the heterologous functional domain is fused C-terminally to the crRNA binding polypeptide.

In some embodiments, the crRNA binding polypeptide and the heterologous functional domain are linked via a linker.

In some embodiments, the linker comprises GS or 2-15 repeats thereof (SEQ ID NO: 85), GSGGGGS (SEQ ID NO: 29) or 2-4 repeats thereof (SEQ ID NO: 86), GGS or 5-10 repeats thereof (SEQ ID NO: 87), GGGS (G₃S) (SEQ ID NO: 63) or 3-7 repeats thereof (SEQ ID NO: 88), GGGGS (G₄S) (SEQ ID NO: 93) or 3-5 repeats thereof (SEQ ID NO: 89), GGGGGS (G₅S) (SEQ ID NO: 94) or 3-4 repeats thereof (SEQ ID NO: 90), or a mixture thereof, or SEQ ID NO: 33; optionally, the length of the linker is about 15, 16, 17, 18, 19, 20, 21, 22, 23, 24, 25, 26, or 27 residues.

In some embodiments, the crRNA binding polypeptide and/or the heterologous functional domain are/is linked to a nuclear localization signal (NLS) sequence or a nuclear export signal (NES).

In some embodiments, the crRNA binding polypeptide and/or the heterologous functional domain is linked to 2 or 3 NLS, such as SEQ ID NO: 35.

In some embodiments, the CRISPR-Cas system, the gRNA, the modified Cas13 protein, the fusion protein, or the CRISPR-Cas13 system as described herein comprises one each of NLS fused N- and C-terminally to the crRNA binding polypeptide.

Another aspect of the disclosure provides a polynucleotide comprising a first and a second polynucleotides encoding the protein component and the gRNA component of the CRISPR-Cas system, the gRNA, the modified Cas13 protein, the fusion protein, or the CRISPR-Cas13 system as described herein, respectively.

In some embodiments, the transcription of the protein component and the transcription of the gRNA are under the control of separate or independent promoters and/or enhancers.

In some embodiments, the transcription of the protein component is under the control of a constitutive promoter, an inducible promoter, a ubiquitous promoter, or a tissue specific promoter.

In some embodiments, the constitutive promoter is an RNA Pol II promoter, such as a CMV promoter, a CB promoter, a Cbh promoter, an EFS promoter, or a CAG promoter.

In some embodiments, the transcription of the gRNA component is under the control of an RNA Pol III promoter, such as a U6 promoter.

In some embodiments, the first polynucleotide is codon-optimized for expression in a cell, such as a eukaryotic cell, or a mammalian (e.g., human) cell.

Another aspect of the disclosure provides a vector comprising the polynucleotide as described herein.

In some embodiments, the vector is a plasmid.

In some embodiments, the vector is a retroviral vector, a phage vector, an adenoviral vector, a herpes simplex viral (HSV) vector, an AAV vector, or a lentiviral vector.

In some embodiments, the vector is an AAV vector comprising the polynucleotide as described herein flanked by a 5′ ITR (such as an AAV2 5′ ITR) and a 3′ ITR (such as an AAV2 3′ ITR).

In some embodiments, the polynucleotide as described herein further comprises an intron and/or an exon that promotes the transcription of the protein component.

In some embodiments, the vector further comprises a coding sequence for a polyA signal sequence operably linked to the first polynucleotide encoding the protein component.

In some embodiments, the vector further comprises a 5′ UTR and/or a 3′ UTR coding sequence in the first polynucleotide encoding the protein component.

In some embodiments, the vector further comprises a WPRE sequence.

Another aspect of the disclosure provides a recombinant AAV (rAAV) viral particle comprising the AAV vector as described herein, encapsidated within a capsid of the serotype of AAV1, AAV2, AAV3A, AAV3B, AAV4, AAV5, AAV6, AAV7, AAVrh74, AAV8, AAV9, AAV10, AAV11, AAV12, AAV13, AAV.DJ, AAV.PHP.eB, or a mutant thereof.

Another aspect of the disclosure provides a delivery system comprising (1) a delivery vehicle, and (2) the CRISPR-Cas system, the gRNA, the modified Cas13 protein, the fusion protein, or the CRISPR-Cas13 system as described herein, the polynucleotide as described herein, the vector as described herein, or the rAAV viral particle as described herein.

In some embodiments, the delivery vehicle is a nanoparticle (such as, a lipid nanoparticle), a liposome, an exosome, a microvesicle, or a gene-gun.

Another aspect of the disclosure provides a cell or a progeny thereof, comprising the CRISPR-Cas system, the gRNA, the modified Cas13 protein, the fusion protein, or the CRISPR-Cas13 system as described herein, the polynucleotide as described herein, the vector as described herein, the rAAV viral particle as described herein, or the delivery system as described herein.

In some embodiments, the cell or progeny thereof is a eukaryotic cell (e.g., a non-human mammalian cell, a non-human primate cell, a human cell, or a plant cell) or a prokaryotic cell (e.g., a bacteria cell).

Another aspect of the disclosure provides a non-human multicellular eukaryote comprising the cell or a progeny thereof as described herein.

In some embodiments, the non-human multicellular eukaryote is an animal (e.g., rodent or primate) model for a human genetic disorder.

Another aspect of the disclosure provides a pharmaceutical composition comprising:

(i) the CRISPR-Cas system, the gRNA, the modified Cas13 protein, the fusion protein, or the CRISPR-Cas13 system as described herein, the polynucleotide as described herein, the vector as described herein, the rAAV viral particle as described herein, the delivery system as described herein, or the cell or a progeny thereof as described herein; and

(ii) a pharmaceutically acceptable excipient.

Another aspect of the disclosure provides a kit comprising:

(i) the CRISPR-Cas system, the gRNA, the modified Cas13 protein, the fusion protein, or the CRISPR-Cas13 system as described herein, the polynucleotide as described herein, the vector as described herein, the rAAV viral particle as described herein, the delivery system as described herein, the cell or a progeny thereof as described herein, or the pharmaceutical composition as described herein; and

(ii) an instruction for using component (i).

Another aspect of the disclosure provides a method of modifying a target RNA, the method comprising contacting the target RNA with the modified Cas13 protein, the fusion protein, or the CRISPR-Cas13 system as described herein, the polynucleotide as described herein, the vector as described herein, the rAAV viral particle as described herein, the delivery system as described herein, the cell or a progeny thereof as described herein, the pharmaceutical composition as described herein, or the kit as described herein, wherein the spacer sequence is substantially complementary to at least 15 contiguous nucleotides of the target RNA; wherein the crRNA binding polypeptide associates with the gRNA to form a complex; wherein the complex binds to the target RNA;

and wherein upon binding of the complex to the target RNA, the complex modifies the target RNA (e.g., deaminates a target ribonucleotide base (e.g., A or C) in the target RNA).

In some embodiments, the target RNA is an mRNA, a tRNA, an rRNA, a non-coding RNA, a lncRNA, or a nuclear RNA.

In some embodiments, the target RNA has a mutation associated with a genetic disease or disorder or has or lacks a modification associated with epigenetics.

In some embodiments, the method as described herein causes one or more of: (i) in vitro or in vivo induction of cellular senescence; (ii) in vitro or in vivo cell cycle arrest; (iii) in vitro or in vivo cell growth inhibition; (iv) in vitro or in vivo induction of anergy; (v) in vitro or in vivo induction of apoptosis; and (vi) in vitro or in vivo induction of necrosis.

Another aspect of the disclosure provides a method of treating a condition or disease in a subject in need thereof, the method comprising administering to the subject the modified Cas13 protein, the fusion protein, or the CRISPR-Cas13 system as described herein, the polynucleotide as described herein, the vector as described herein, the rAAV viral particle as described herein, the delivery system as described herein, the cell or a progeny thereof as described herein, the pharmaceutical composition as described herein, or the kit as described herein, wherein the spacer sequence is substantially complementary to at least 15 contiguous nucleotides of a target RNA associated with the condition or disease; wherein the crRNA binding polypeptide associates with the gRNA to form a complex; wherein the complex binds to the target RNA; and wherein upon binding of the complex to the target RNA, the complex modifies the target RNA (e.g., deaminates a target ribonucleotide base (e.g., A or C) in the target RNA), thereby treating the condition or disease in the subject.

In some embodiments, the condition or disease is a genetic or epigenetic disease or disorder.

In some embodiments, the method is an in vitro method, an in vivo method, or an ex vivo method.

It should be understood that any one embodiment of the disclosure described herein, including those described only in the examples or claims, or only in one aspects/sections below, can be combined with any 5 other one or more embodiments of the disclosure, unless explicitly disclaimed or improper.

### BRIEF DESCRIPTION OF DRAWINGS

An understanding of the features and advantages of the disclosure will be obtained by reference to the following detailed description that sets forth illustrative embodiments, in which the principles of the disclosure may be utilized, and the accompanying drawings of which:

FIG. 1 is a schematic (not to scale) illustration of the genomic loci of the representative Cas13e and Cas13f family members. The Cas coding sequences (long bars with pointed end), followed by the multiple nearby direct repeat (DR) (short bars) and spacer sequences (diamonds), are shown.

FIG. 2 shows putative secondary structures of the DR sequences associated with the respective Cas13e and Cas13f proteins. Their coding sequences, from left to right, are represented by SEQ ID NOs: 104-110, respectively.

FIG. 3 shows the domain structures for the representative Cas13a-Cas13f proteins. The overall sizes, and the locations of the two RXXXXH motifs on each representative member of the Cas proteins are indicated.

FIG. 4 is a schematic (not to scale) drawing showing the series of progressive C-terminal deletion constructs for dCas13e.1 fused to hADAR2_{DD}-E488Q/T375G RNA base editor (shown as “ADAR2DD”), as well as other transcriptional control elements.

FIG. 5 is a schematic (not to scale) drawing showing the series of progressive C-terminal and optional N-terminal deletion constructs for dCas13e.1.

FIG. 6 shows the percentage RNA base editing activities of the fusion proteins comprising the same hADAR2_{DD}-E488Q/T375G and the indicated truncated dCas13e.1, represented by the percentage results of mCherry mutant conversion back to wild-type mCherry, in comparison with a control where the full length dCas13e.1 mutant (full length dCas13e.1-R84A,H89A,R739A,R740A,H744A,H745A mutant, SEQ ID NO: 139) was used in place of those truncated dCas13e.1. For negative control, a non-targeting spacer sequence (“NT”) was used in place of the targeting spacer sequence of the guide RNA (in configuration of 5′-spacer-DR-3′) targeting the mCherry reporter gene.

FIG. 7 shows schematic diagrams of hADAR2_{DD}-E488Q-based base editors with or without full length dCas13e.1 or minidCas13e.1.

FIG. 8 shows the results of transcriptome-wide A-to-I off-target base editing by the base editors in FIG. 7 based on RNAseq analysis.

FIG. 9 shows a schematic diagram of off-target RNA base editing detection using a fluorescence reporting system, including a reporter construct and one of the base editor expression constructs. In order to better explore the off-target system, an additional spacer sequence designed for the off-target site 1 was also provided without a DR sequence. FIG. 9 discloses SEQ ID NOS 96-97, respectively, in order of appearance.

FIG. 10 is a flow chart of the off-target RNA base editing detection experiment. The reporter construct was co-transferred into HEK293T cells with a respective base editor expression construct, and the transfected and cultured BFP and mCherry double positive cells were sorted at 72 hours. RNA was extracted, Sanger sequencing was performed after RT-PCR, and the off-target based editing efficiency/extent was analyzed.

FIG. 11 shows RT-PCR detection of A-to-I off-target RNA base editing by the indicated RNA base editors. According to the results of Sanger sequencing, the off-target base editing efficiency of the indicated RNA base editors was analyzed.

FIG. 12 is a schematic diagram of a DMD exon 52 deletion mini gene reporter system. The treatment of DMD Exon51 disease site can be monitored by EGFP reporter expression by RNA base editing changing A in the premature stop codon to I (G).

FIG. 13 shows the A-to-I base editing efficiency of minidCas13e.1-ADARv1 with single DR and dual DR gRNAs and four NES/NLS strategies.

FIG. 14 is a schematic diagram of a reporter system for use with an exemplary base editor system of the disclosure. The treatment of DMD Exon23X disease site can be realized by effecting the change of TAA>TGG to eliminate a premature stop codon. EGFP on the reporter cannot be expressed without eliminating the premature stop codon. FIG. 14 discloses SEQ ID NOS 98, 99 and 99, respectively, in order of appearance.

FIG. 15 shows the A-to-I base editing efficiency of the base editors in FIG. 14 with single DR and dual DR guide RNAs. Flow cytometry analysis of EGFP/(BFP⁺ & mCherry⁺) ratio after 48 h was conducted. The results showed that all dual DR (dDR) based editing systems achieved higher EGFP fluorescence ratios (i.e., higher A-I editing efficiency) compared to the corresponding single DR (sDR) based editing systems.

FIG. 16 is a schematic diagram of a reporter system for use with an exemplary base editor system of the disclosure. The treatment of DMD Exon54X disease site can be realized by effecting the change of TAG>TGG to eliminate a premature stop codon. EGFP on the reporter cannot be expressed without eliminating the premature stop codon. FIG. 16 discloses SEQ ID NOS 100, 101 and 101, respectively, in order of appearance.

FIG. 17 shows the A-to-I base editing efficiency of the base editor in FIG. 16 with single DR and dual DR guide RNAs. Flow cytometry analysis of EGFP/(BFP⁺ & mCherry⁺) ratio after 48 h was conducted. The results showed that the dual DR (dDR) based editing system achieved a higher EGFP fluorescence ratio (i.e., a higher A-I editing efficiency) compared to the corresponding single DR (sDR) based editing system.

FIG. 18 is a schematic diagram of a reporter system for use with an exemplary base editor system of the disclosure. The schematic diagram of the reporter and the base editor system mainly explored the base editing differences between dual DR (dDR) and single DR (sDR) under different nuclear sequences. Through TAA>TGG base editing, the selective splicing of pre-RNA was normalized, and the full-length protein was translated, thereby realizing the treatment of Rpe65 Q64X associated diseases. FIG. 18 discloses SEQ ID NOS 102, 103 and 103, respectively, in order of appearance.

FIG. 19 shows the result of analyzing the base editing efficiency associated with different base editing systems based on the results of Sanger sequencing. The results showed that under the combination of different nuclear sequence, higher A-to-I base editing efficiency was achieved for all the double DR (dDR) gRNA base editing systems than the corresponding single DR (sDR) gRNA base editing systems. A1/A2 (TA1A2>TGG) show the base editing at the two A bases, respectively.

FIGS. 20A and 20B show a gel image of RT-PCR gel electrophoresis and the analysis of the proportion of full-length mRNA. The results showed that, under different combinations of nuclear sequences (especially for 2xNLS and 3xNLS), the percentages of full-length mRNA (correctly processed mRNA) achieved by the double DR (dDR) gRNA base editing systems are higher than or comparable to that by the single DR (sDR) gRNA base editing systems.

FIG. 21 is a schematic diagram showing the reporter and base editor systems used in Example 8.

FIG. 22 shows that EcCas6e (“Cas6e”) has high DR processing activity, as reflected by the near zero level of EGFP expression, while the H20L mutation abolished the DR processing activity of EcCas6e, resulting in the high expression of EGFP.

FIG. 23 shows that the H20L mutant of EcCas6e retained substantially the same ability as EcCas6e to support RESCUES-mediated base editing at the mCherry target site. That is, the H20L mutant has almost no DR processing function, but it still retains a high applicability for base editing.

FIG. 24A shows the schematic constructs of exemplary reporter and expression plasmids for the evaluation of DR sequence-processing ability of Cas proteins (full length Cas13e.1 and mnidCas13e.1). FIG. 24B is a histogram showing the DR sequence-processing ability of the tested Cas proteins, represented by the percentage proportion of EGFP positive cells in BFP positive cells. Negative control: Reporter, indicating that only the reporter plasmid was transfected to host cells. All values are presented as mean±s.d. (n=3).

FIG. 25 shows the functional domain structures of Cas13e.1, Cas13e.2, Cas13e.3, Cas13e.7 and Cas13f.2. The RxxxxH motifs defining the catalytic site of Cas13e.1 is indicated as the region between R84-H89 (inclusive) and R739-H745 (inclusive), while corresponding motifs in Cas13e.2, Cas13e.3, Cas13e.7 and Cas13f.2 are not separately illustrated.

FIG. 26A shows the schematic constructs of exemplary reporter and expression plasmids for the evaluation of RNA base editing efficiency of base editors each comprising a truncated Cas13 protein (dCas13e.2-N150+C150, dCas13e.2-N180+C180, dCas13e.3-N180+C180, dCas13e.7-N150+C150, dCas13f.2-N150+C150, and as a positive control, the minidCas13e.1-N180+C150) and the same human ADAR2_{DD}-E488Q deaminase domain. FIG. 26B is a histogram showing the RNA base editing efficiency of the tested base editors, represented by the ratio of the number of mCherry-positive cells to the number of BFP and EGFP dual-positive cells. Negative control: minidCas13e.1-N180+C150 with non-targeting (NT) spacer sequence. Positive control: minidCas13e.1-N180+C150 with targeting spacer sequence. All values are presented as mean±s.d. (n=3).

FIG. 27 shows the schematic constructs of exemplary reporter and expression plasmids for the evaluation of DR sequence-processing ability of dPspCas13b and ddPspCas13b and A-to-I base editing efficiency of ddPspCas13b-based base editor with dual or single DR gRNA configuration.

FIG. 28 is a histogram showing the DR sequence-processing ability of the tested Cas proteins, represented by the percentage proportion of EGFP positive cells in BFP positive cells. All values are presented as mean±s.d. (n=3).

FIG. 29 is a histogram showing the A-to-I base editing efficiency of ddPspCas13b-based base editor with sDR or dDR gRNA configuration, represented by the percentage proportion of mCherry positive cells in BFP positive cells. Negative control: Reporter, indicating that only the reporter plasmid was transfected to host cells. All values are presented as mean±s.d. (n=3).

The figures herein are for illustrative purposes only and are not necessarily drawn to scale.

### DETAILED DESCRIPTION

Unless defined otherwise, all technical and scientific terms used herein have the same meaning as is commonly understood by one of ordinary skill in the art to which this disclosure belongs.

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The recitation of numerical ranges by endpoints herein includes all numbers and fractions subsumed within the respective ranges, as well as the recited endpoints.

The recitation of numbers herein except in the Examples includes the recitation of numerical ranges constituted with any two of the recited numbers as the endpoints of the numerical ranges.

The term “about X-Y” used herein has the same meaning as “about X to about Y.”

The term “about” or “approximately” in relation to a reference numerical value and its grammatical equivalents as used herein can include the numerical value itself and a range of values plus or minus 10% from that numerical value. For example, the amount “about 10” or “approximately 10” includes 10 and any amounts from 9 to 11. For example, the term “about” or “approximately” in relation to a reference numerical value can also include a range of values plus or minus 10%, 9%, 8%, 7%, 6%, 5%, 4%, 3%, 2%, or 1% from that value.

As used herein, reference to “not” a value or parameter generally means and describes “other than” a value or parameter. For example, the method is not used to treat cancer of type X means the method is used to treat cancer of types other than X.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, a “biological sample” may contain whole cells and/or live cells and/or cell debris. The biological sample may contain (or be derived from) a “bodily fluid”. The disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

A protein or nucleic acid derived from a species means that the protein or nucleic acid has a sequence identical to an endogenous protein or nucleic acid or a portion thereof in the species. The protein or nucleic acid derived from the species may be directly obtained from an organism of the species (e.g., by isolation), or may be produced, e.g., by recombination production or chemical synthesis.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or combinations thereof, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al, 1992; Milligan, 1993; WO 97/03211; WO 96/39154; Mata, 1997; Strauss-Soukup, 1997; and Samstag, 1996. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, “complementarity” refers to the ability of a nucleic acid to form hydrogen bond(s) with another nucleic acid by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid (e.g., about 5, 6, 7, 8, 9, 10 out of 10, being about 50%, 60%, 70%, 80%, 90%, and 100% complementary respectively). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least about 60%, 65%, 70%, 75%, 80%, 85%, 90%, 91%, 92%, 93%, 94%, 95%, 96%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N. Y. Where reference is made to a polynucleotide sequence, then complementary or partially complementary sequences are also envisaged. These are preferably capable of hybridizing to the reference sequence under highly stringent conditions. Generally, in order to maximize the hybridization rate, relatively low-stringency hybridization conditions are selected: about 20 to 25° C. lower than the thermal melting point (Tm). The Tm is the temperature at which 50% of specific target sequence hybridizes to a perfectly complementary probe in solution at a defined ionic strength and pH. Generally, in order to require 5 at least about 85% nucleotide complementarity of hybridized sequences, highly stringent washing conditions are selected to be about 5 to 15° C. lower than the Tm. A sequence capable of hybridizing with a given sequence is referred to as the “complement” of the given sequence.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. A sequence capable of hybridizing with a given sequence is referred to as the “complement” of the given sequence.

As described herein, sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences.

The terms “polypeptide”, “peptide”, and “protein” are used interchangeably herein to refer to polymers of amino acids of any length. The polymer may be linear or branched, it may comprise modified amino acids, and it may be interrupted by non-amino acids. A protein may have one or more polypeptides. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics. As used herein, the term “domain” or “protein domain” refers to a part of a protein sequence that may exist and function independently of the rest of the protein chain.

As used herein the term “variant” should be taken to mean the exhibition of qualities that have a pattern that deviates from what occurs in nature. As used herein, a polynucleotide or polypeptide “variant” is interpreted to mean a polynucleotide or polypeptide that differs from a reference polynucleotide or polypeptide, respectively. A typical variant of a polynucleotide differs in nucleic acid sequence from another reference polynucleotide. Changes in the nucleic acid sequence of the variant may or may not alter the amino acid sequence of a polypeptide encoded by the reference polynucleotide. Nucleotide changes may result in amino acid substitutions, insertions, and/or deletions in the polypeptide encoded by the reference sequence, as discussed below. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more substitutions, insertions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

As used herein the term “wild type” is a term of the art understood by skilled persons and means the typical form of an organism, strain, gene or characteristic as it occurs in nature as distinguished from mutant or variant forms. A “wild type” can be a base line. It can be isolated from sources in nature and not intentionally modified.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

As used herein, the term “genomic locus” or “locus” (plural loci) is the specific location of a gene or DNA sequence on a chromosome. A “gene” refers to stretches of DNA that encode a polypeptide or an RNA chain that has functional role to play in an organism and hence is the molecular unit of heredity in living organisms. For the purpose of this disclosure, it may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. As used herein, “expression of a genomic locus” or “gene expression” is the process by which information from a gene is used in the synthesis of a functional gene product. The products of gene expression are often proteins, but in non-protein coding genes such as rRNA genes or tRNA genes, the product is functional RNA. The process of gene expression is used by all known life—eukaryotes (including multicellular organisms), prokaryotes (bacteria and archaea) and viruses to generate functional products to survive. As used herein “expression” of a gene or nucleic acid encompasses not only cellular gene expression, but also the transcription and translation of nucleic acid(s) in cloning systems and in any other context. As used herein, “expression” also refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product”. If the polynucleotide is derived from genomic DNA, expression may include splicing of the mRNA in a eukaryotic cell.

A “cell” as used herein, is understood to refer not only to the particular individual cell, but to the progeny or potential progeny of the cell. Because certain modifications may occur in succeeding generations due to either mutation or environmental influences, such progeny may not, in fact, be identical to the parent cell, but are still included within the scope of the term as used herein.

The term “transduction” and “transfection” as used herein include all methods known in the art using an infectious agent (such as a virus) or other means to introduce DNA into cells for expression of a protein or molecule of interest. Besides a virus or virus like agent, there are chemical-based transfection methods, such as those using calcium phosphate, dendrimers, liposomes, or cationic polymers (e.g., DEAE-dextran or polyethylenimine); non-chemical methods, such as electroporation, cell squeezing, sonoporation, optical transfection, impalefection, protoplast fusion, delivery of plasmids, or transposons; particle-based methods, such as using a gene gun, magnectofection or magnet assisted transfection, particle bombardment; and hybrid methods, such as nucleofection.

The term “transfected” or “transformed” or “transduced” as used herein refers to a process by which exogenous nucleic acid is transferred or introduced into a target cell. A “transfected” or “transformed” or “transduced” cell is one, which has been transfected, transformed, or transduced with exogenous nucleic acid.

The term “in vivo” refers to inside the body of the organism from which the cell is obtained. “Ex vivo” or “in vitro” means outside the body of the organism from which the cell is obtained.

As used herein, “treatment” or “treating” is an approach for obtaining beneficial or desired results including clinical results. For purposes of this disclosure, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, reducing recurrence rate of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of a disease (such as cancer). The methods of the disclosure contemplate any one or more of these aspects of treatment.

As used herein, the term “parental”, when referring to in a context of obtaining a changed protein by changing an original protein, refers to the original protein from which the changed protein is derived. For example, a truncated Cas13e.1 protein can be derived from wild type Cas13e.1 by truncating the N-terminal and/or C-terminal residues of the wild type Cas13e.1, then the wild type Cas13e.1 is the parental protein of the truncated Cas13e.1 protein.

As used herein, the phrase “substantially removed” when referring to the substantial removal of both HEPN1 and HEPN2 domains of a Cas13 effector protein means that (1) no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid of each of HEPN1 and HEPN2 domains is not removed but retained on the Cas13 effector protein; AND (2) no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid of the functional domain immediately adjacent to HEPN1 or HEPN2 domain is removed.

It is understood that embodiments of the disclosure described herein include “consisting of” and/or “consisting essentially of” embodiments.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the disclosure. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### 1. Overview

RNA base editing, as one example of practical applications taking advantage of the CRISPR RNA (crRNA) binding property of CRISPR-associated (Cas) effector proteins, can be achieved by linking an RNA base editor to a targeting domain that brings the RNA base editor to a specific RNA target site. One of the frequently used targeting domains is a variant of a CRISPR-Cas system effector enzyme that has been modified to lose guide RNA-directed target RNA cleavage/RNase activity, such as the so-called dead Cas (dCas) having point mutations in the RNase catalytic domain. Such modified Cas can still bind to its guide RNA, which brings the Cas-RNA base editor to a specific target RNA site by hybridizing with the target RNA through the spacer sequence in the guide RNA, thus allowing the RNA base editor to modify (e.g., deaminate) a target ribonucleotide at the target RNA to effect base editing.

There are at least two important characteristics for such targeted RNA base editor—targeting efficiency and off-target activity. Targeting efficiency relates to the desired activity—how efficiently the targeted RNA base editor is brought to the target RNA and deaminates the target ribonucleotide at the target site. Off-target activity relates to the undesired activity—how often the targeted RNA base editor deaminates an unintended ribonucleotide, e.g., at an off-target location.

The disclosure described herein is partly based on the surprising discovery that enhanced applicability of CRISPR-Cas system (e.g., up to 200% enhanced targeting efficiency) can be achieved by using a transcribed guide RNA having a spacer sequence flanked by two (rather than one) DR sequences and a modified Cas protein capable of maintaining such a DR configuration of the guide RNA (in other words, not destroying such a DR configuration by processing or cleaving the DR sequence of the guide RNA).

While not wishing to be bound by any particular theory, it is believed that these Cas effector proteins (e.g., Cas13 or CasPR) can be modified to delete a substantial portion of the N- and/or C-terminal regions encompassing part or all of the HEPN domains (not just rendered their RNase activity deficient by inactivating catalytic activity in the RxxxxH motif by point mutations) or introduce an amino acid mutation, thus substantially reducing or eliminating the ability of these Cas effector proteins to process DR sequences in the primary transcript, and be able to work with transcribed guide RNA having a spacer sequence flanked by two DR sequences.

The disclosure described herein is further based on the surprising discovery that the same Cas effector proteins modified the same way, when linked to an RNA base editor, substantially reduces the inherent off-target activity of the base editor, based on transcriptome-wide assessment of off-target base editing efficiency. Furthermore, Cas effector proteins so modified surprisingly eliminated about 99% of the off-target activity of a corresponding dCas-based targeted RNA base editor, thus achieving 2 orders of magnitude better (lower) off-target base editing by the traditional dCas-based targeted RNA base editor.

The disclosure described herein is additionally based on the surprising discovery that the targeting efficiency of the subject targeted RNA base editor (based on modified Cas effector proteins) can be further enhanced by fusing 2-3 nuclear localization sequences (NLS) to the targeted RNA base editor, such as by fusing one NLS at both ends of the modified Cas effector enzyme used as the targeting domain.

Thus, in one aspect, the disclosure provides a CRISPR-Cas system, comprising:

(a) a CRISPR RNA (crRNA) binding polypeptide or a polynucleotide coding sequence (e.g., a DNA coding sequence or an RNA coding sequence) thereof, the crRNA binding polypeptide comprising, consisting essentially of, or consisting of a crRNA binding domain of a Cas effector protein,

(b) a heterologous functional domain or a polynucleotide coding sequence thereof (e.g., a DNA coding sequence or an RNA coding sequence), and

(c) a guide RNA (gRNA) or a polynucleotide coding sequence (e.g., a DNA coding sequence or an RNA coding sequence) thereof, the gRNA comprising:

(i) a 5′ direct repeat (DR) sequence and a 3′ direct repeat (DR) sequence, each capable of forming a complex with the crRNA binding domain; and

(ii) a spacer sequence capable of hybridizing to a target RNA, and guiding or recruiting the complex to the target RNA,

wherein the crRNA binding polypeptide:

(1) is linked (e.g., fused) to the heterologous functional domain, and

(2) substantially lacks the ability (e.g., having no more than 50%, 40%, 30%, 20%, 10%, 5%, 2%, or 1% of that of the Cas effector protein) to process or cleave DR sequence on the gRNA, and

wherein the spacer sequence is flanked by the 5′ and 3′ DR sequences at the 5′ end and the 3′ end of the spacer sequence, respectively; optionally, the 5′ and 3′ DR sequences are identical.

In another aspect, the disclosure provides a guide RNA (gRNA) comprising:

a) a 5′ direct repeat (DR) sequence and a 3′ direct repeat (DR) sequence, each capable of forming a complex with a CRISPR RNA (crRNA) binding polypeptide comprising, consisting essentially of, or consisting of a crRNA binding domain of a Cas effector protein; and

b) a spacer sequence capable of hybridizing to a target RNA, and guiding or recruiting the complex to the target RNA,

wherein the spacer sequence is flanked by the 5′ and 3′ DR sequences at the 5′ end and the 3′ end of the spacer sequence, respectively; optionally, the 5′ and 3′ DR sequences are identical.

In some embodiments, the crRNA binding polypeptide substantially lacks the ability (e.g., having no more than 50%, 40%, 30%, 20%, 10%, 5%, 2%, or 1% of that of the Cas effector protein) to process or cleave DR sequence on the gRNA.

In some embodiments, the crRNA binding polypeptide is linked (e.g., fused) to a heterologous functional domain.

In another aspect, the disclosure provides a modified Cas13 protein with both HEPN1 and HEPN2 domains substantially removed from a parental or wild-type Cas13 effector protein (e.g., substantially lacking both the HEPN1 and HEPN2 domains of the parental or wild-type Cas13 effector protein), with the proviso that the modified Cas13 protein is not minidCas13e.1-N180+C150.

In some embodiments, the modified Cas13 protein has a first deletion of or comprising the HEPN1 domain, and a second deletion of or comprising the HEPN2 domain, and substantially lacking the ability (e.g., having no more than 50%, 40%, 30%, 20%, 10%, 5%, 2%, or 1% of that of the parental or wild-type Cas13 effector protein) to process or cleave a direct repeat (DR) sequence capable of forming a complex with the modified Cas13 protein in a guide RNA (gRNA) comprising:

a) the direct repeat sequence; and

b) a spacer sequence capable of hybridizing to a target RNA, and guiding or recruiting the complex to the target RNA.

In some embodiments, (1) the first deletion is no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues larger than the HEPN1 domain of the parental or wild-type Cas13 effector protein, and is no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues smaller than the HEPN1 domain of the parental or wild-type Cas13 effector protein; and (2) the second deletion is no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues larger than the HEPN2 domain of the parental or wild-type Cas13 effector protein, and is no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues smaller than the HEPN2 domain of the parental or wild-type Cas13 effector protein.

In some embodiments, the parental or wild-type Cas13 effector protein is a Cas13a effector protein, a Cas13b effector protein, a Cas13c effector protein, a Cas13d effector protein, a Cas13e effector protein, or a Cas13f effector protein.

In another aspect, the disclosure provides a fusion protein comprising:

a) the modified Cas13 protein as described herein; and,

b) a heterologous functional domain, e.g., a deaminase domain.

In another aspect, the disclosure provides a CRISPR-Cas13 system comprising:

a) the modified Cas13 protein as described herein or the fusion protein as described herein or a polynucleotide coding sequence (e.g., a DNA coding sequence or an RNA coding sequence) thereof; and

b) a guide RNA (gRNA) or a polynucleotide coding sequence (e.g., a DNA coding sequence or an RNA coding sequence) thereof, the gRNA comprising:

i. a direct repeat (DR) sequence capable of forming a complex with the modified Cas13 protein of the fusion protein; and,

ii. a spacer sequence capable of hybridizing to a target RNA, and guiding or recruiting the complex to the target RNA.

In some embodiments, the gRNA comprises

a) a 5′ direct repeat (DR) sequence and a 3′ direct repeat (DR) sequence, each capable of forming a complex with the modified Cas protein of the fusion protein; and

b) a spacer sequence capable of hybridizing to a target RNA, and guiding or recruiting the complex to the target RNA,

wherein the spacer sequence is flanked by the 5′ and 3′ DR sequences at the 5′ end and the 3′ end of the spacer sequence, respectively; optionally, the 5′ and 3′ DR sequences are identical.

In certain embodiments, the CRISPR-Cas system (e.g., CRISPR-Cas13 system) of the disclosure further comprises, or is conjugated to, a heterologous functional domain. The heterologous functional domain may be another covalently or non-covalently linked protein or polypeptide or other molecules (such as detection reagents or drug/chemical moieties). Such other proteins/polypeptides/other molecules can be linked through, for example, chemical coupling, gene fusion, or other non-covalent linkage (such as biotin-streptavidin binding). Such derived proteins do not affect the function of the original protein, such as the ability to bind a guide RNA/crRNA of the disclosure to form a complex, and the ability to bind to a target RNA at a specific site, under the guidance of the crRNA that is at least partially complementary to the target RNA.

In certain embodiments, the heterologous functional domain comprises a nuclear localization signal (NLS, such as SV40 large T antigen NLS) to enhance the ability of the subject modified Cas effector protein or subject polypeptide of the disclosure (e.g., Cas13e and Cas13f-based crRNA binding domain) to enter cell nucleus. Such derivation can also be used to add a targeting molecule or moiety for specific cellular or subcellular locations. Such derivation can also be used to add a detectable label to facilitate the detection, monitoring, or purification of the subject CRISPR-Cas systems.

The derivation can be through adding any of the additional moieties at the N- or C-terminal of the subject CRISPR-Cas systems, or internally (e.g., internal fusion or linkage through side chains of internal amino acids), such as between the polypeptide of the disclosure comprising the crRNA binding domain and the RNA base editor.

In a related second aspect, the disclosure also provides conjugates of the subject crRNA binding polypeptide, which are conjugated with the RNA base editor, and optionally moieties such as other proteins or polypeptides, detectable labels, or combinations thereof. Such conjugated moieties may include, without limitation, localization signals, reporter genes (e.g., GST, HRP, CAT, GFP, HcRed, DsRed, CFP, YFP, BFP), labels (e.g., fluorescent dye such as FITC, or DAPI), NLS, targeting moieties, DNA binding domains (e.g., MBP, Lex A DBD, Gal4 DBD), epitope tags (e.g., His, myc, V5, FLAG, HA, VSV-G, Trx, etc), transcription activation domains (e.g., VP64 or VPR), transcription inhibition domains (e.g., KRAB moiety or SID moiety), nucleases (e.g., FokI), deamination domain (e.g., ADAR1, ADAR2, APOBEC, AID, or TAD), methylase, demethylase, transcription release factor, HDAC, ssRNA cleavage activity, dsRNA cleavage activity, ssDNA cleavage activity, dsDNA cleavage activity, DNA or RNA ligase, any combination thereof, etc.

In certain embodiments, the conjugate may include one or more (e.g., 2 or 3) NLSs, which can be located at or near N-terminal, C-terminal, internally, or combination thereof. The linkage can be through amino acids (such as D or E, or S or T), amino acid derivatives (such as Ahx, β-Ala, GABA or Ava), or PEG linkage.

In certain embodiments, conjugations do not affect the function of the original protein, such as the ability to bind a guide RNA/crRNA of the disclosure (described herein below) to form a complex, and the ability to bind to a target RNA at a specific site, under the guidance of the crRNA that is at least partially complementary to the target RNA.

With the disclosures generally described herein above, more detailed descriptions for the various aspects of the disclosure are provided in separate sections below. However, it should be understood that, for simplicity and to reduce redundancy, certain embodiments of the disclosure are only described under one section or only described in the claims or examples. Thus, it should also be understood that any one embodiment of the disclosure, including those described only under one aspect, section, or only in the claims or examples, can be combined with any other embodiment of the disclosure, unless specifically disclaimed or the combination is improper.

### 2. Exemplary Class 2, Type VI CRISPR RNA-Guided RNases, and Derivatives Thereof

The disclosure described herein provides Cas13 effector proteins that can be modified (e.g., by N- and/or C-terminal deletion) to eliminate not only the guide RNA-mediated RNase activity, but also substantially all (e.g., all) ability to process the initial long CRISPR sequence (the single long transcript encompassing much of the CRISPR array) to generate crRNAs with direct repeat (DR) sequences. Thus, while not wishing to be bound by any particular theory, the crRNA binding domain-containing polypeptide of the disclosure can work/complex with guide RNA with a spacer flanked by two DR sequences—one at each end of the spacer, without cleaving off one of the DR sequences.

In certain embodiments, the Cas effector enzyme is a Class 2, Type VI-A (Cas13a or C2c2), Type VI-B (Cas13b), Type VI-C(Cas13c), Type VI-D (Cas13d), Type VI-E (Cas13e), or Type VI-F (Cas13f) effector protein.

The Class 2, Type VI-E and Type-VI-F effector proteins, referred herein as Cas13e and Cas13f, are much smaller than the other Cas13 effector proteins (e.g., Cas13a-Cas13d), such that they can be more easily packaged with their crRNA coding sequences into small capacity gene therapy vectors, such as the AAV vectors. Further, the Cas13e and Cas13f effector proteins are more potent in knocking down RNA target sequences, and more efficient in RNA single base editing, as compared to the Cas13a, Cas13b, and Cas13d effector proteins. Thus, these new Cas proteins are more ideally suited for gene therapy.

Thus, in certain embodiments, the Cas effector protein is a Class 2, Type VI-E (Cas13e), or Type VI-F (Cas13f) Cas effector protein.

In certain embodiments, the Cas effector protein comprises an amino acid sequence of any one of SEQ ID NOs: 1-7, 111-125, and 173, or orthologs, homologs, the various derivatives (described herein below), wherein said orthologs, homologs, derivatives have maintained at least one function of any one of the proteins of SEQ ID NOs: 1-7, 111-125, and 173. Such functions include, but are not limited to, the ability to bind a guide RNA/crRNA of the disclosure to form a complex, and the ability to bind to a target RNA at a specific site, under the guidance of the crRNA that is at least partially complementary to the target RNA.

In certain embodiments, the Cas13 effector proteins of the disclosure can be: (i) any one of SEQ ID NOs: 1-7, 111-125, and 173; (ii) a derivative having one or more amino acids (e.g., 1, 2, 3, 4, 5, 6, 7, 8, 9, or 10 residues) of addition, deletion, and/or substitution (e.g., conserved substitution) of any one of SEQ ID NOs: 1-7, 111-125, and 173; or (iii) a derivative having amino acid sequence identity of at least about 80%, 85%, 90%, 91%, 92%, 93%, 94%, 95%, 96%, 97%, 98%, or 99% compared to any one of SEQ ID NOs: 1-7, 111-125, and 173.

In some embodiments, the Cas13 effector protein comprises an amino acid sequence (1) of any one of SEQ ID NOs: 1-7, 111-125, and 173, or (2) having a sequence identity of at least 80%, 85%, 90%, 91%, 92%, 93%, 94%, 95%, 96%, 97%, 98%, or 99% to the amino acid sequence of any one of SEQ ID NOs: 1-7, 111-125, and 173.

In certain embodiments, the Cas13 effector proteins, orthologs, homologs, derivatives are not naturally existing, e.g., having at least one amino acid difference compared to a naturally existing sequence.

In certain embodiments, the crRNA binding domain-containing polypeptide of the disclosure substantially lacks the N-terminal HEPN domain (e.g., RxxxxH domain) and/or the C-terminal HEPN domain (e.g., RxxxxH domain).

In certain embodiments, the Cas effector protein is a CRISPR Class 2, type VI effector having two strictly conserved Rx4-6H (N-terminal amino acid R and C-terminal amino acid H interposed with 4 to 6 amino acid) (RxxxxH to RxxxxxxH to) motifs, characteristic of Higher Eukaryotes and Prokaryotes Nucleotide-binding (HEPN) domains. Similar CRISPR Class 2, Type VI effector proteins that contain two HEPN domains have been previously characterized and include, for example, CRISPR Cas13a (C2c2), Cas13b, Cas13c, and Cas13d. As used herein, the N-terminal HEPN domain is also termed “HEPN1” domain, and the C-terminal HEPN domain is also termed as “HEPN2” domain.

HEPN domains have been shown to be RNase domains and confer the ability to bind to and cleave target RNA molecule. The target RNA may be any suitable form of RNA, including but not limited to mRNA, tRNA, ribosomal RNA, non-coding RNA, lncRNA (long non-coding RNA), and nuclear RNA. For example, in some embodiments, the Cas proteins recognize and cleave RNA targets located on the coding strand of open reading frames (ORFs).

Any of the Cas13 effector proteins, orthologs, homologs, derivatives thereof can be modified to delete the N- and/or C-terminal HEPN domains, leaving substantially only the crRNA binding domain in the internal part of the Cas effector proteins, orthologs, homologs, derivatives thereof.

In certain embodiments, the modified Cas13 effector proteins, orthologs, homologs, derivatives thereof substantially lack the N-terminal HEPN domain (e.g., RxxxxH domain) and/or the C-terminal HEPN domain (e.g., RxxxxH domain).

In some embodiments, the modified Cas13 effector proteins, orthologs, homologs, derivatives thereof substantially lack the HEPN1 domain (e.g., RxxxxH domain and/or the HEPN2 domain (e.g., RxxxxH domain of the Cas effector protein.

In some embodiments, the modified Cas13 effector proteins, orthologs, homologs, derivatives thereof substantially lack both the HEPN1 and HEPN2 domains of the Cas effector protein.

In some embodiments, the modified Cas13 effector proteins, orthologs, homologs, derivatives thereof have a first deletion of or comprising the HEPN1 domain, and a second deletion of or comprising the HEPN2 domain.

In some embodiments, (1) the first deletion is no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues larger than the HEPN1 domain of the Cas13 effector protein, and is no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues smaller than the HEPN1 domain of the Cas13 effector protein; and (2) the second deletion is no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues larger than the HEPN2 domain of the Cas13 effector protein, and is no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues smaller than the HEPN2 domain of the Cas13 effector protein.

In certain embodiments, the Cas effector protein is a Class 2, Type VI-E (Cas13e) Cas effector protein (e.g., SEQ ID NO: 1), and wherein said polypeptide lacks about 180 (e.g., 170, 171, 172, 173, 174, 175, 176, 177, 178, 179, 180, 181, 182, 183, 184, 185, 186, 187, 188, 189, or 190) N-terminal residues, and lacks about 150 (e.g., 140, 141, 142, 143, 144, 145, 146, 147, 148, 149, 150, 151, 152, 153, 154, 155, 156, 157, 158, 159, or 160) C-terminal residues of said Cas13e effector protein (e.g., SEQ ID NO: 1).

In some embodiments, the crRNA binding polypeptide comprises an amino acid sequence of any one of SEQ ID NOs: 32, 168-172, and 174.

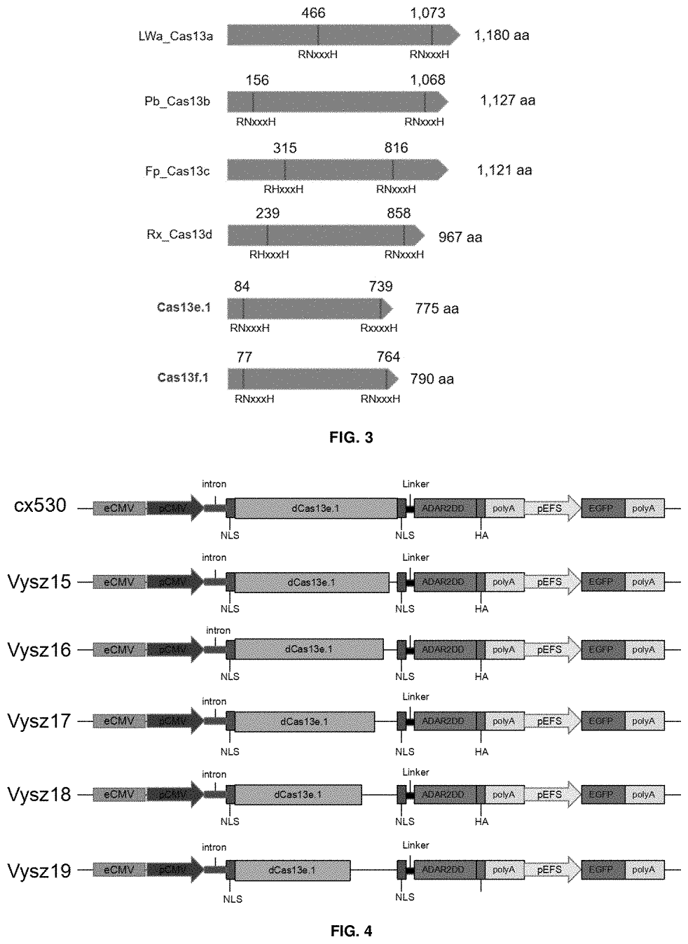

In one embodiment, the CRISPR Class 2, type VI effector is a Type VI-E and VI-F CRISPR-Cas effector protein, Cas13e or Cas13f. Direct comparison of the Type VI-E and VI-F CRISPR-Cas effector proteins with the effector of these other systems shows that Type VI-E and VI-F CRISPR-Cas effector proteins are significantly smaller (e.g., about 20% fewer amino acids) than even the smallest previously identified Type VI-D/Cas13d effectors (see FIG. 3), and have less than 30% sequence similarity in one to one sequence alignments to other previously described effector proteins, including the phylogenetically closest relatives Cas13b.

These two families of CRISPR Class 2, type VI effectors are particularly suitable for therapeutic applications since they are significantly smaller than other effectors (e.g., CRISPR Cas13a, Cas13b, Cas13c, and Cas13d effectors) which allows for the packaging of the nucleic acids encoding the effectors and their guide RNA coding sequences into delivery systems having size limitations, such as the AAV vectors.

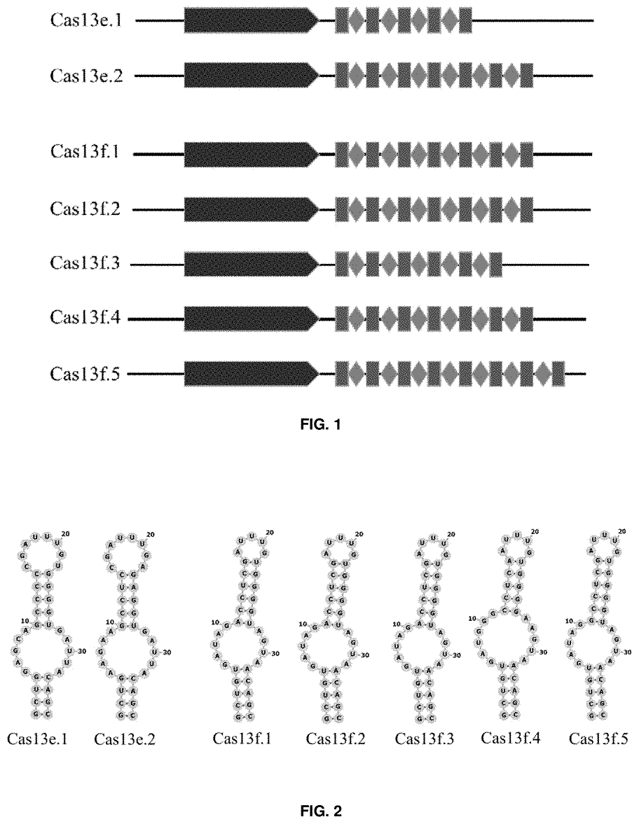

In bacteria, the Type VI-E and VI-F CRISPR-Cas systems include a single effector (approximately 775 residues and 790 residues, respectively) within close proximity to a CRISPR array (see FIG. 1). The CRISPR array includes direct repeat (DR) sequences typically 36 nucleotides in length, which are generally well conserved, both in sequences and secondary structures (see FIG. 2).

The crRNAs for the Type VI-E and -F effectors are processed from the 5′-end, such that the DR sequences normally end up at the 3′-end of the mature crRNA.

The spacers contained in the Cas13e and Cas13f CRISPR arrays are most commonly 30 nucleotides in length, with the majority of variation in length contained in the range of 29 to 30 nucleotides. However, a wide range of spacer length may be tolerated. For example, for use in a functional Cas13e or Cas13f effector protein, or homologs, orthologs, derivatives, fusions, conjugates, or functional fragment thereof, the spacer can be between 10-60 nucleotides, 20-50 nucleotides, 25-45 nucleotides, 25-35 nucleotides, or about 27, 28, 29, 30, 31, 32, or 33 nucleotides. For use in dCas version of any of the above, however, the spacer can be between 10-200 nucleotides, 20-150 nucleotides, 25-100 nucleotides, 25-85 nucleotides, 35-75 nucleotides, 45-60 nucleotides, or about 46, 47, 48, 49, 50, 51, 52, 53, 54, or 55 nucleotides; or 15-100 nucleotides, 15-80 nucleotides, 15-60 nucleotides, between 25-50 nucleotides, between 30-50 nucleotides, about 100 nucleotides, about 80 nucleotides, about 60 nucleotides, about 55 nucleotides, about 50 nucleotides, about 45 nucleotides, about 40 nucleotides, about 35 nucleotides, about 30 nucleotides, about 20 nucleotides, or about 15 nucleotides in length.

Exemplary Type VI CRISPR-Cas effector proteins are set forth in SEQ ID NO: 1-7, 111-125, and 173.

In Cas13e.1, the C-terminal motif may have two possibilities due to the RR and HH sequences flanking the motif. Mutations at one or both such domains may create an RNase dead version (or “dCas) of the Cas13 effector proteins, homologs, orthologs, fusions, conjugates, derivatives, or functional fragments thereof, while substantially maintaining their ability to bind the guide RNA and the target RNA complementary to the guide RNA.

The corresponding DR coding sequences for the Cas effector proteins are set forth in SEQ ID NO: 8-14 and 126-140.

Natural (wild-type) DNA coding sequences for Cas13e.1, Cas13e.2, Cas13f.1, Cas13f.2, Cas13f3, Cas13f.4, and Cas13f. 5 proteins are set forth in SEQ ID NOs: 15-21, respectively.

Human codon-optimized coding sequences for the seven Cas13e and Cas13f proteins (i.e., Cas13e.1, Cas13e.2, Cas13f.1, Cas13f.2, Cas13f.3, Cas13f.4 and Cas13f.5), generated for further functional experiments, are set forth in SEQ ID NOs: 22-28, respectively.

The seven CRISPR-Cas13e and Cas13f loci structures were shown in FIG. 1.

Further analysis of RNA secondary structures for the seven DR sequences in the pre-crRNA was conducted using RNAfold. The results were shown in FIG. 2. It is apparent that all shared very conserved secondary structure.

For example, in the Cas13e family, each DR sequence forms a secondary structure consisting of a 4-base pair stem (5′-GCUG-3′), followed by a symmetrical bulge of 5+5 nucleotides (excluding the 4 stem nucleotides), further followed by a 5-base pair stem (5′-GCC C/U C-3′), and a terminal 8-base loop (5′-CGAUUUGU-3′, excluding the 2 stem nucleotides).

Likewise, in the Cas13f family, with one exception (Cas13f.4), each DR sequence forms a secondary structure consisting of a 5-base pair stem (5′GCUGU3′), followed by a nearly symmetrical bulge of 5+4 nucleotides (excluding the 4 stem nucleotides), further followed by a 6-base pair stem (5′A/G CCUCG3′), and a terminal 5-base loop (5′AUUUG3′, excluding the 2 stem nucleotides). The only exception being the DR for Cas13f.4, in which the second step is 1 base pair shorter, and 2 additional bases were added to the first bulge to form a largely symmetrical 6+5 bulge.

Since the secondary structures of the DR sequences, including the location and size of the step, bulge, and loop structures, are likely more important than the specific nucleotide sequences that form such secondary structures, alternative or derivative DR sequences can also be used in the systems and methods of the disclosure, so long as these derivative or alternative DR sequences have a secondary structure that substantially resembles the secondary structure of an RNA encoded by any one of SEQ ID NO: 8-14 and 126-140. For example, the derivative DR sequence may have ±1 or 2 base pair(s) in one or both stems (see FIG. 2), have ±1, 2, or 3 bases in either or both of the single strands in the bulge, and/or have ±1, 2, 3, or 4 bases in the loop region.

In some embodiments, Class 2, Type VI CRISPR-Cas effector proteins include a “derivative” having an amino acid sequence with at least about 80% sequence identity to the amino acid sequence of any one of SEQ ID NOs: 1-7, 111-125, and 173 (e.g., 81%, 82%, 83%, 84%, 85%, 86%, 87% 88%, 89%, 90%, 91%, 92%, 93%, 94%, 95%, 96%, 97%, 98%, 99%, or 100%). Such derivative Cas effector proteins sharing significant protein sequence identity to any one of SEQ ID NOs: 1-7, 111-125, and 173 have retained at least one of the functions of the Cas of SEQ ID NOs: 1-7, 111-125, and 173, such as the ability to bind to and form a complex with a crRNA comprising at least one of the DR sequences of SEQ ID NOs: 8-14 and 126-140. For example, a Cas13e.1 derivative may share 85% amino acid sequence identity to SEQ ID NO: 1, 2, 3, 4, 5, 6, or 7, respectively, and retains the ability to bind to and form a complex with a crRNA having a DR sequence of SEQ ID NO: 8, 9, 10, 11, 12, 13, or 14, respectively.

Such derivative Cas proteins can be modified similarly as the corresponding wild-type Cas proteins, such as wild-type Cas13e.1, by, for example, N- and/or C-terminal deletions, in order to substantially eliminate all ability to process DR sequence native to the wild type Cas (e.g., Cas13e.1), yet substantially retain the ability to bind DR sequence/guide RNA to enable RNA base editing through the linked RNA base editor.

In some embodiments, the derivative comprises conserved amino acid residue substitutions compared to the corresponding wild-type Cas. In some embodiments, the derivative comprises only conserved amino acid residue substitutions (i.e., all amino acid substitutions in the derivative are conserved substitutions, and there is no substitution that is not conserved).

In some embodiments, the derivative comprises no more than 1, 2, 3, 4, 5, 6, 7, 8, 9, or 10 amino acid insertions or deletions into any one of the wild-type sequences of SEQ ID NOs: 1-7, 111-125, and 173. The insertion and/or deletion maybe clustered together, or separated throughout the entire length of the sequences, so long as at least one of the functions of the wild-type sequence is preserved. Such functions may include the ability to bind the guide/crRNA, the RNase activity, the ability to bind to and/or cleave the target RNA complementary to the guide/crRNA. In some embodiments, the insertions and/or deletions are not present in the Rx4-6H motifs, or within 5, 10, 15, or 20 residues from the Rx4-6H motifs.

In some embodiments, the derivative has retained the ability to bind guide RNA/crRNA.

In some embodiments, the derivative has retained the guide/crRNA-activated RNase activity.

In some embodiments, the derivative has retained the ability to bind target RNA and/or cleave the target RNA in the presence of the bound guide/crRNA that is complementary in sequence to at least a portion of the target RNA.

In other embodiments, the derivative has completely or partially lost the guide/crRNA-activated RNase activity, due to, for example, mutations in one or more catalytic residues of the RNA-guided RNase. Such derivatives are sometimes referred to as dCas, such as dCas13e.1, etc.

Thus, in certain embodiments, the derivative may be modified to have diminished nuclease/RNase activity, e.g., nuclease inactivation of at least 50%, at least 60%, at least 70%, at least 80%, at least 90%, at least 95%, at least 97%, or 100% as compared with the counterpart wild type proteins. The nuclease activity can be diminished by several methods known in the art, e.g., introducing mutations into the nuclease (catalytic) domains of the proteins. In some embodiments, catalytic residues for the nuclease activities are identified, and these amino acid residues can be substituted by different amino acid residues (e.g., glycine or alanine) to diminish the nuclease activity. In some embodiments, the amino acid substitution is a conservative amino acid substitution. In some embodiments, the amino acid substitution is a non-conservative amino acid substitution.

In some embodiments, the modification comprises one or more mutations (e.g., amino acid deletions, insertions, or substitutions) in at least one HEPN domain. In some embodiments, there is one, two, three, four, five, six, seven, eight, nine, or more amino acid substitutions in at least one HEPN domain. For example, in some embodiments, the one or more mutations comprise a substitution (e.g., an alanine substitution) at an amino acid residue corresponding to R84, H89, R739, H744, R740, H745 of SEQ ID NO: 1, or R97, H102, R770, H775 of SEQ ID NO: 2, or R77, H82, R764, H769 of SEQ ID NO: 3, or R79, H84, R766A, H771 of SEQ ID NO: 4, or R79, H84, R766, H771 of SEQ ID NO: 5, or R89, H94, R773, H778 of SEQ ID NO: 6, or R89, H94, R777, H782 of SEQ ID NO: 7.

In certain embodiments, the one or more mutations or the two or more mutations may be in a catalytically active domain of the effector protein comprising a HEPN domain, or a catalytically active domain which is homologous to a HEPN domain. In certain embodiments, the effector protein comprises one or more of the following mutations: R84A, H89A, R739A, H744A, R740A, H745A (wherein amino acid positions correspond to amino acid positions of Cas13e.1). The skilled person will understand that corresponding amino acid positions 5 in different Cas13e and Cas13f proteins may be mutated to the same effect. In certain embodiments, one or more mutations abolish catalytic activity of the protein completely or partially (e.g. altered cleavage rate, altered specificity, etc.).

Other exemplary (catalytic) residue mutations include: R97A, H102A, R770A, H775A of Cas13e.2, or R77A, H82A, R764A, H769A of Cas13f.1, or R79A, H84A, R766A, H771A of Cas13f.2, or R79A, H84A, R766A, H771A of Cas13f.3, or R89A, H94A, R773A, H778A of Cas13f.4, or R89A, H94A, R777A, H782A of Cas13f.5. In certain embodiments, any of the R and/or H residues herein may be replaced not be A but by G, V, or I.

The presence of at least one of these mutations results in a derivative having reduced or diminished RNase activity as compared to the corresponding wild-type protein lacking the mutations.

In certain embodiments, the effector protein as described herein is a “dead” effector protein, such as a dead Cas13e or Cas13f effector protein (i.e., dCas13e and dCas13f). In certain embodiments, the effector protein has one or more mutations or deletions in HEPN domain 1 (N-terminal). In certain embodiments, the effector protein has one or more mutations or deletions in HEPN domain 2 (C-terminal). In certain embodiments, the effector protein has one or more mutations or deletions in HEPN domain 1 and HEPN domain 2.

In some embodiments, a Type VI CRISPR-Cas effector proteins includes the amino acid sequence of any one of SEQ ID NOs: 1-7, 111-125, and 173. In some embodiments, the Type VI CRISPR-Cas effector proteins or derivatives thereof or functional fragments thereof recognizes and cleaves the target RNA without any additional requirements adjacent to or flanking the protospacer (i.e., protospacer adjacent motif “PAM” or protospacer flanking sequence “PFS” requirements).

In some embodiments, the crRNA binding domain-containing polypeptide of the disclosure is a “functional fragment” of the full-length parental or wild-type (SEQ ID NOs: 1-7, 111-125, and 173) or derivative Type VI Cas effector proteins.

A “functional fragment,” as used herein, refers to a fragment of a parental or wild-type protein of any one of SEQ ID NOs: 1-7, 111-125, and 173, or a derivative thereof, that has less-than full-length sequence.

The deleted residues in the functional fragment can be at the N-terminus, the C-terminus, and/or internally. The functional fragment retains at least one function of the parental or wild-type VI Cas effector protein, or at least one function of its derivative. Thus, a functional fragment is defined specifically with respect to the function at issue. For example, a functional fragment, wherein the function is the ability to bind crRNA and target RNA, may not be a functional fragment with respect to the RNase function, because losing the Rx4-6H motifs at both ends of the Cas may not affect its ability to bind a crRNA and target RNA, but may eliminate destroy the RNase activity.

In certain embodiments, the retained function includes the ability to form a complex with the guide RNA through binding to the DR sequence, yet the ability to process DR sequence is substantially lost.

In some embodiments, compared to full-length sequences SEQ ID NOs: 1-7, 111-125, and 173, the Type VI CRISPR-Cas effector proteins or derivatives thereof or functional fragments thereof lack about 30, 60, 90, 120, 150, or about 180 residues from the N-terminus. In certain embodiments, the Type VI CRISPR-Cas effector proteins or derivatives thereof or functional fragments thereof lack about 180 (e.g., 170, 171, 172, 173, 174, 175, 176, 177, 178, 179, 180, 181, 182, 183, 184, 185, 186, 187, 188, 189, or 190) N-terminal residues of the parental or wt Cas, such as wt Cas13e.1 (e.g., SEQ ID NO: 1).

In some embodiments, compared to full-length sequences SEQ ID NOs: 1-7, 111-125, and 173, the Type VI CRISPR-Cas effector proteins or derivatives thereof or functional fragments thereof lack about 30, 60, 90, 120, or about 150 residues from the C-terminus. In certain embodiments, the Type VI CRISPR-Cas effector proteins or derivatives thereof or functional fragments thereof lack about 150 (e.g., 140, 141, 142, 143, 144, 145, 146, 147, 148, 149, 150, 151, 152, 153, 154, 155, 156, 157, 158, 159, or 160) C-terminal residues of said Cas13e effector protein (e.g., SEQ ID NO: 1).

In some embodiments, the crRNA binding polypeptide comprises an amino acid sequence of any one of SEQ ID NOs: 32, 168-172, and 174.

In some embodiments, compared to full-length sequences SEQ ID NOs: 1-7, 111-125, and 173, the Type VI CRISPR-Cas effector proteins or derivatives thereof or functional fragments thereof lack about 30, 60, 90, 120, 150, or about 180 residues (e.g., 170, 171, 172, 173, 174, 175, 176, 177, 178, 179, 180, 181, 182, 183, 184, 185, 186, 187, 188, 189, or 190) from the N-terminus, and lack about 30, 60, 90, 120, or about 150 residues (e.g., 140, 141, 142, 143, 144, 145, 146, 147, 148, 149, 150, 151, 152, 153, 154, 155, 156, 157, 158, 159, or 160) from the C-terminus.

The inactivated Cas or derivative or functional fragment thereof can be fused, conjugated (e.g., through chemical linkage), or otherwise associated with one or more heterologous/functional domains (e.g., via fusion protein, linker peptides, “GS” linkers, etc.). These functional domains can have various activities, e.g., methylase activity, demethylase activity (e.g., Fat mass and obesity-associated protein (FTO), ALKBH5), methyltransferase activity (e.g., METTL3, METTL14, WTAP, KIAA1429), transcription activation activity, transcription repression/inhibition activity, transcription release factor activity, histone modification activity, RNA cleavage activity, DNA cleavage activity, nucleic acid binding activity, base-editing activity, and switch activity (e.g., light inducible). In some embodiments, the functional domains are Krüppel associated box (KRAB), SID (e.g. SID4X), VP64, VPR, VP16, Fok1, P65, HSF1, MyoD1, Adenosine Deaminase Acting on RNA such as ADAR1, ADAR2, APOBEC, cytidine deaminase (AID), TAD, mini-SOG, APEX, and biotin-APEX, or functional deaminase domain thereof (such as ADAR1DD or ADAR2DD).

In some embodiments, the heterologous functional domain comprises a deaminase domain, for example, an adenosine deaminase domain, such as a double-stranded RNA-specific adenosine deaminase (e.g., Adenosine deaminase acting on RNA (ADAR), such as, ADAR1 or ADAR2), apolipoprotein B mRNA editing enzyme, catalytic polypeptide-like (APOBEC), activation-induced cytidine deaminase (AID), or a functional fragment thereof; or a cytidine deaminase domain, such as, RescueS (SEQ ID NO: 56), or a functional fragment thereof.

In some embodiments, the functional domain is a base editing domain or RNA base editor, e.g., ADAR1 (including wild-type or ADAR1_{DD} version thereof, with or without the E1008Q mutation), ADAR2 (including wild-type or ADAR2_{DD} version thereof, with or without the E488Q mutation and/or the T375G mutation, or RescueS (SEQ ID NO: 56)), APOBEC, or AID.

In some embodiments, the ADAR2 or a functional fragment thereof comprising ADAR2_{DD} comprises E488Q mutation or a E-to-Q substitution mutation at a position corresponding to E488 of human ADAR2, and optionally further comprises T375G mutation or a T-to-G substitution mutation at a position corresponding to T375 of human ADAR2.

In some embodiments, the deaminase domain is hADAR2DD-E488Q (SEQ ID NO: 34), hADAR2DD-E488Q/T375G (SEQ ID NO: 163), or RescueS (SEQ ID NO: 56).

In some embodiments, the heterologous functional domain deaminates an adenosine (A) in the target RNA to an inosine (I) and/or deaminates a cytidine (C) in the target RNA to an uridine (U).

In some embodiments, the heterologous functional domain comprises a m6A-associated regulation domain, such as, a m6A-associated methyltransferase domain (e.g., METTL3, METTL14, WTAP, KIAA1429, or a functional fragment thereof), a m6A-associated demethylation domain (e.g., Fat mass and obesity-associated protein (FTO), ALKBH5, or a functional fragment thereof), or a combination thereof.

In some embodiments, the functional domain may comprise one or more nuclear localization signal (NLS) domains or nuclear export sequence (NES). The one or more heterologous functional domains may comprise at least two or more NLS/NES domains. The one or more NLS/NES domain(s) may be positioned at or near or in proximity to a terminus of the effector protein (e.g., Cas13e/Cas13f effector proteins) and if two or more NLSs/NESs, each of the two may be positioned at or near or in proximity to a terminus of the effector protein (e.g., Cas13e/Cas13f effector proteins). In certain embodiments, a 3′ NLS may be located C terminal to the RNA base editor fused C terminal to the targeting Cas moiety.

In some embodiments, at least one or more heterologous functional domains may be at or near the amino-terminus of the effector protein and/or wherein at least one or more heterologous functional domains is at or near the carboxy-terminus of the effector protein. The one or more heterologous functional domains may be fused to the effector protein. The one or more heterologous functional domains may be tethered to the effector protein. The one or more heterologous functional domains may be linked to the effector protein by a linker moiety.

In some embodiments, multiple (e.g., two, three, four, five, six, seven, eight, or more) identical or different functional domains are present.

In some embodiments, the functional domain (e.g., a base editing domain) is further fused to an RNA-binding domain (e.g., MS2).

In some embodiments, the functional domain is associated to or fused via a linker sequence (e.g., a flexible linker sequence or a rigid linker sequence). Exemplary linker sequences and functional domain sequences are provided in the table at the end of the specification.

### Amino Acid Sequences of Motifs and Functional Domains in Engineered Variants of Type VI CRISPR Cas Effector Proteins

In certain embodiments, the heterologous functional domain is fused or conjugated N-terminally, C-terminally, or internally to the crRNA binding polypeptide.

In certain embodiments, the heterologous functional domain is fused C-terminal to the crRNA binding polypeptide.

In certain embodiments, the crRNA binding polypeptide and the heterologous functional domain are linked via a linker.

In certain embodiments, the linker comprises GS or 2-15 repeats thereof (SEQ ID NO: 85), GSGGGGS (SEQ ID NO: 29) or 2-4 repeats thereof (SEQ ID NO: 86), GGS or 5-10 repeats thereof (SEQ ID NO: 87), GGGS (G₃S) (SEQ ID NO: 63) or 3-7 repeats thereof (SEQ ID NO: 88), GGGGS (G₄S) (SEQ ID NO: 93) or 3-5 repeats thereof (SEQ ID NO: 89), GGGGGS (G₅S) (SEQ ID NO: 94) or 3-4 repeats thereof (SEQ ID NO: 90), or a mixture thereof, or SEQ ID NO: 33; optionally, the length of the linker is about 15, 16, 17, 18, 19, 20, 21, 22, 23, 24, 25, 26, or 27 residues.

The positioning of the one or more functional domains on the inactivated Cas proteins is one that allows for correct spatial orientation for the functional domain to affect the target with the attributed functional effect. The positioning can be adjusted by using one or more GS linkers, such as those listed in the table above.

In certain embodiments, the crRNA binding domain-containing polypeptide of the disclosure and/or the heterologous functional domain is linked to 2 or 3 NLS, such as SEQ ID NO: 35.

In certain embodiments, the crRNA binding domain-containing polypeptide of the disclosure is fused N- and C-terminally with one each of NLS.

In some embodiments, the functional domain (e.g., NLS or NES) is positioned at the N-terminus of the Cas/dCas. In some embodiments, the functional domain is positioned at the C-terminus of the Cas/dCas. In some embodiments, the inactivated CRISPR-associated protein (dCas) is modified to comprise a first functional domain at the N-terminus and a second functional domain at the C-terminus.

Various examples of inactivated CRISPR-associated proteins fused with one or more functional domains and methods of using the same are described, e.g., in International Publication No. WO 2017/219027, which is incorporated herein by reference in its entirety, and in particular with respect to the features described herein.

In some embodiments, the RNA modifying activity of the CRISPR-Cas system (e.g., CRISPR-Cas13 system) of the disclosure can be modulated through endogenous RNA signatures (e.g., miRNA) in mammalian cells. A switch can be made by using a miRNA-complementary sequence in the 5′-UTR of mRNA encoding the CRISPR-Cas system (e.g., CRISPR-Cas13 system) of the disclosure. The switches selectively and efficiently respond to miRNA in the target cells. Thus, the switches can differentially control the genome editing by sensing endogenous miRNA activities within a heterogeneous cell population. Therefore, the switch systems can provide a framework for cell-type selective genome editing and cell engineering based on intracellular miRNA information (see, e.g., Hirosawa et al., Nucl. Acids Res. 45(13): e118, 2017).

The CRISPR-Cas system (e.g., CRISPR-Cas13 system) of the disclosure (e.g., those based on Class 2, Type VI CRISPR-Cas effector proteins) can be inducibly expressed, e.g., their expression can be light-induced or chemically-induced. This mechanism allows for activation of the functional domain in the CRISPR-associated proteins. Light inducibility can be achieved by various methods known in the art, e.g., by designing a fusion complex wherein CRY2 PHR/CIBN pairing is used in split CRISPR-associated proteins (see, e.g., Konermann et al., “Optical control of mammalian endogenous transcription and epigenetic states,” Nature 500:7463, 2013.

Chemical inducibility can be achieved, e.g., by designing a fusion complex wherein FKBP/FRB (FK506 binding protein/FKBP rapamycin binding domain) pairing is used in split CRISPR-associated proteins. Rapamycin is required for forming the fusion complex, thereby activating the CRISPR-associated proteins (see, e.g., Zetsche et al., “A split-Cas9 architecture for inducible genome editing and transcription modulation,” Nature Biotech. 33:2:139-42, 2015).

Furthermore, the expression of the CRISPR-Cas system (e.g., CRISPR-Cas13 system) of the disclosure can be modulated by inducible promoters, e.g., tetracycline or doxycycline controlled transcriptional activation (Tet-On and Tet-Off expression system), hormone inducible gene expression system (e.g., an ecdysone inducible gene expression system), and an arabinose-inducible gene expression system. When delivered as RNA, expression of the RNA targeting effector protein can be modulated via a riboswitch, which can sense a small molecule like tetracycline (see, e.g., Goldfless et al., “Direct and specific chemical control of eukaryotic translation with a synthetic RNA-protein interaction,” Nucl. Acids Res. 40:9: e64-e64, 2012).

In some embodiments, the crRNA binding domain-containing polypeptide of the disclosure includes at 5 least one (e.g., 1, 2, 3, 4, or 5) Nuclear Localization Signal (NLS) attached to the N-terminal or C-terminal of the protein. Non-limiting examples of NLSs include an NLS sequence derived from: the NLS of the SV40 virus large T-antigen, having the amino acid sequence PKKKRKV (SEQ ID NO: 35); the NLS from nucleoplasmin (e.g., the nucleoplasmin bipartite NLS with the sequence KRPAATKKAGQAKKKK, SEQ ID NO: 64); the c-myc NLS having the amino acid sequence PAAKRVKLD (SEQ ID NO: 65) or RQRRNELKRSP (SEQ ID NO: 66); the hRNPA1 M9 NLS having the sequence NQSSNFGPMKGGNFGGRSSGPYGGGG QYFAKPRNQGGY (SEQ ID NO: 67); the sequence RMRIZFKNKGKDTAELRRRRVEVSVE LRKAKKDEQILKRRNV (SEQ ID NO: 68) of the IBB domain from importin-alpha; the sequences VSRKRPRP (SEQ ID NO: 69) and PPKKARED (SEQ ID NO: 70) of the myoma T protein; the sequence PQPKKKPL (SEQ ID NO: 71) of human p53; the sequence SALIKKKKKMAP (SEQ ID NO: 72) of mouse c-abl IV; the sequences DRLRR (SEQ ID NO: 73) and PKQKKRK (SEQ ID NO: 74) of the influenza virus NS1; the sequence RKLKKKIKKL (SEQ ID NO: 75) of the Hepatitis virus delta antigen; the sequence REKKKFLKRR (SEQ ID NO: 76) of the mouse Mx1 protein; the sequence KRKGDEVDGVDEVAKKKSKK (SEQ ID NO: 77) of the human poly(ADP-ribose) polymerase; and the sequence RKCLQAGMNLEARK TKK (SEQ ID NO: 78) of the human glucocorticoid receptor. In some embodiments, the CRISPR-associated protein comprises at least one (e.g., 1, 2, 3, 4, 5, 6, 7, 8, 9, or 10) Nuclear Export Signal (NES) attached the N-terminal or C-terminal of the protein. In a preferred embodiment a C-terminal and/or N-terminal NLS or NES is attached for optimal expression and nuclear targeting in eukaryotic cells, e.g., human cells.

In some embodiments, the crRNA binding domain-containing polypeptide of the disclosure described herein are mutated at one or more amino acid residues to alter one or more functional activities. For example, in some embodiments, the crRNA binding domain-containing polypeptide of the disclosure is mutated at one or more amino acid residues to alter its helicase activity. In some embodiments, the crRNA binding domain-containing polypeptide of the disclosure is mutated at one or more amino acid residues to alter its nuclease activity (e.g., endonuclease activity or exonuclease activity). In some embodiments, the crRNA binding domain-containing polypeptide of the disclosure is mutated at one or more amino acid residues to alter its ability to functionally associate with a guide RNA. In some embodiments, the crRNA binding domain-containing polypeptide of the disclosure is mutated at one or more amino acid residues to alter its ability to functionally associate with a target nucleic acid.

In some embodiments, the crRNA binding domain-containing polypeptide of the disclosure described herein can be engineered to have a deletion in one or more amino acid residues to reduce the size of the enzyme while retaining one or more desired functional activities (e.g., nuclease activity and the ability to interact functionally with a guide RNA). The truncated CRISPR-associated protein can be advantageously used in combination with delivery systems having load limitations.

In some embodiments, the crRNA binding domain-containing polypeptide of the disclosure described herein can be fused to one or more peptide tags, including a His-tag, GST-tag, a V5-tag, FLAG-tag, HA-tag, VSV-G-tag, Trx-tag, or myc-tag.

In any of the embodiments herein, the linkage between the crRNA binding domain-containing polypeptide of the disclosure described herein and the other moiety (such as the RNA base editor) can be at the N- or C-terminal of the crRNA binding domain-containing polypeptide of the disclosure, and sometimes even internally via covalent chemical bonds. The linkage can be effected by any chemical linkage known in the art, such as peptide linkage, linkage through the side chain of amino acids such as D, E, S, T, or amino acid derivatives (Ahx, 13-Ala, GABA or Ava), or PEG linkage.

### 3. Exemplary CRISPR-Associated Proteins for Class 1 Pre-crRNA Processing (CasPR)

CRISPR clusters contain space sequences (or “spacers”) located between direct repeat (DR) sequences. The natural spacers in the CRISPR loci of bacteria are sequences complementary to antecedent mobile elements and target invading nucleic acids. CRISPR clusters are initially transcribed into long primary transcripts called pre-CRISPR RNAs (pre-crRNAs), which are subsequently processed into CRISPR RNAs (crRNAs) by sequence-specific CRISPR-associated (Cas) endonucleases that cleave the initial long primary transcripts (pre-crRNAs), usually at the base of the direct repeat hairpin RNA structures, into smaller, mature crRNAs. Such sequence-specific endonucleases are collectively referred to herein as “CasPRs” (CRISPR-associated Proteins for Class 1 pre-crRNA processing), “Cas pre-crRNA processing/maturation endonucleases”, or “pre-crRNA-processing Cas effector proteins”.

Most multi-subunit Class 1 systems process crRNAs with a CRISPR associated endonucleases called Cas6, which share conserved structural motifs that bind crRNAs. In general, Cas6 use a metal-ion-independent mechanism to cleave crRNAs on the 3′-side of stem-loops formed within the palindromic CRISPR repeat sequence. Cleavage is generally catalyzed by stabilizing nucleophilic attack from the 2′—OH group located upstream from the scissile phosphate. Although different Cas6 enzymes from different species tend to be diverse in sequence, this cleavage mechanism appears to be conserved, despite some structural and mechanistic differences. Often, a His residue is used to catalyze cleavage, though other residues, such as Lys, have been shown to catalyze the reaction when histidine is not present (e.g., in subtype I-A). In subtypes I-B, I-E, I-D and I-F, Cas6 makes structural and base specific interactions with the stable stem-loop formed by the palindromic CRISPR repeat and typically stays bound even after cleavage to form a component of the multi-subunit interference complex. In contrast, the repeats of subtypes I-A, III-A, and III-B are less stable, allowing Cas6 to dissociate from the processed crRNA and to perform multi-turnover crRNA cleavage.

Type IV CRISPR systems are also categorized as Class 1 as they are predicted to form multi-subunit crRNA-guided complexes. Distinct Type IV-A systems contain diverse cas6 gene sequences, including genes designated as cas6e and cas6f (cas6 sequences observed in subtypes I-E and I-F, also generally referred hereto as Cas6), and a Type IV-specific Cas6-like Csf5. The presence of Cas6 homologs suggests that Type IV-A systems process crRNAs through a Cas6-mediated mechanism. Indeed, although various mechanisms exist, Cas6-mediated metal-independent processing of crRNA is a conserved process across diverse Class 1 systems, including in Type IV systems. Type IV crRNA is cleaved on the 3′ side of the predicted stem-loop structure, with nucleophilic attack on the scissile phosphate coming from the 2′ hydroxyl of base G22 of the repeat.

Cas5 family proteins are found in several type I CRISPR-Cas systems. It is report that Cas5d cleaves pre-crRNA into unit length by recognizing both the hairpin structure and the 3′ single stranded sequence in the CRISPR repeat region. It is further shown that after pre-crRNA processing, Cas5d assembles with crRNA, Csd1, and Csd2 proteins to form a multi-sub-unit interference complex similar to Escherichia coli Cascade (CRISPR-associated complex for antiviral defense) in architecture. The results suggest that formation of a crRNA-presenting Cascade-like complex is likely a common theme among type I CRISPR subtypes. (see Nam K H, Haitjema C, Liu X, Ding F, Wang H, DeLisa M P, Ke A. Cas5d protein processes pre-crRNA and assembles into a cascade-like interference complex in subtype I-C/Dvulg CRISPR-Cas system. Structure. 2012 Sep. 5; 20(9):1574-84. doi: 10.1016/j.str.2012.06.016. Epub 2012 Jul. 26. PMID: 22841292; PMCID: PMC3479641.)

The disclosure described herein provides CasPR that can be modified (e.g., by amino acid mutation) to eliminate substantially all (e.g., all) ability to process the initial long CRISPR sequence (the single long transcript encompassing much of the CRISPR array) to generate crRNAs with direct repeat (DR) sequences. Thus, while not wishing to be bound by any particular theory, the crRNA binding domain-containing polypeptide of the disclosure can work/complex with guide RNA with a spacer flanked by two DR sequences—one at each end of the spacer, without cleaving off one of the DR sequences.

In some embodiments, the Cas effector protein is a CasPR (CRISPR-associated Protein for Class 1 pre-crRNA processing).

In certain embodiments, the modified CasPR lacks the ability to process DR sequences. In certain embodiments, the modified CasPR comprise a mutation in its catalytic domain, that substantially eliminates its ability to process DR sequences, yet the modified CasPR substantially retains its ability to bind to a guide RNA having DR sequences.

In some embodiments, the CasPR is Cas5d, Cas6 (e.g., Cas6e), or Csf5.

In some embodiments, the CasPR comprises an amino acid sequence (1) of any one of SEQ ID NOs: 141-151, or (2) having a sequence identity of at least 80%, 85%, 90%, 91%, 92%, 93%, 94%, 95%, 96%, 97%, 98%, or 99% to the amino acid sequence of any one of SEQ ID NOs: 141-151.

In certain embodiments, the CasPR is a Cas6e or Cas6f effector protein. In certain embodiments, the modified Cas6e or Cas6f lacks the ability to process DR sequences. In certain embodiments, the modified Cas6e comprise a mutation in its catalytic domain, such as the H20L mutation, that substantially eliminates its ability to process DR sequences, yet the modified Cas6e substantially retains its ability to bind to a guide RNA having DR sequences. In certain embodiments, the modified Cas6e in the polypeptide of the disclosure comprises the amino acid sequence of SEQ ID NO: 51 (EcCas6e-H20L).

The following sections describe three prototypical CasPR that can be used in the methods and systems of the disclosure, though other related CasPRs, particularly those related to SEQ ID NOs: 142-151, are within the scope of the disclosure.

### Cas5d

The Cas5d Cas processing enzyme (CasPR) is a Class 1, Type I-C CasPR that processes pre-crRNA in crRNA. It has about 250 residues, including a conserved 43-residue N-terminal region. When processing pre-crRNA, Cas5d initiates an intramolecular attack of the 2′-hydroxyl group of G26 (the 3-′end base of the predicted hairpin stem) on the scissile phosphodiester, cutting the precursor 3′ to G26 residue, yielding 5′-hydroxyl and 2′ and/or 3′ ends lacking a hydroxyl group (perhaps a 2′/3′ cyclic phosphodiester). It is believed to require between 4 and 8 nt downstream of the cleavage site for both binding and cleavage of the pre-crRNA. Substitution with dG at this G26 position abolishes cleavage but not RNA binding.

The high-resolution X-ray structure of Cas5d from Mannheimia succiniciproducens has been published (see Garside et al., RNA 18(11):2020-2028, 2012). The M succiniciproducens Cas5d shares strong sequence similarity with the Cas5d family of Dvulg-type Cas proteins, and a Cas5d ortholog from Thermus thermophilus is also an RNA endonuclease that specifically binds and cleaves pre-crRNA. Comparison of Cas5d by structural alignment with the Class 1, Type I crRNA CasPR Cse3 suggested that there is a conserved mechanism of RNA recognition among diverse CRISPR RNA processing enzymes. In addition, primary sequence alignments revealed that the T. thermophilus Cas5d is ˜40% identical and ˜65% similar to that of M succiniciproducens Cas5d, indicating the known structure of the M. succiniciproducens Cas5d forms an excellent basis for homology modeling of the structure of the other Cas5d with at least about 25%, or about 35-40% sequence identity, and/or at least about 60% sequence similarity.

BLASTp search in the NCBI nr database using the SpCas5d (I-C1) protein sequence (SEQ ID NO: 143) retrieved, in addition to the Streptococcus pyogenes query sequence, at least 100 homologous sequences sharing at least 80% sequence identity over the entire length of the query sequence, all within the Streptococcus genus, and most with more than 90% sequence identity.

Similarly, BLASTp search in the NCBI nr database using the BhCas5d (I-C2) protein sequence (SEQ ID NO: 144) retrieved, in addition to the Bacillus halodurans C-125 query sequence, at least 100 homologous sequences sharing at least 69% sequence identity over the entire length of the query sequence.

Thus one aspect of the disclosure provide a wild-type Class 1, Type I-C or Cas5d type CasPR protein (e.g., homologs, orthologs, paralogs) that shares at least about 65%, 69%, 70%, 75%, 80%, 85%, 90%, 91%, 92%, 93%, 94%, 95%, 96%, 97%, 98%, 99% or more sequence identity to SEQ ID NOs: 143 or 144, such as those that are currently available in the NCBI nr database and can be readily retrieved using SEQ ID NO: 143 or 144 as protein query sequence.

The terms “homologue” and “homolog” are used interchangeably herein and are well known in the art. A “homologue” as used herein also includes a protein of the same species which performs the same or a similar function as the protein it is a homologue of. Homologous proteins may but need not be structurally related, or are only partially structurally related. Homolog also encompasses “orthologue”/“ortholog” and “paralogue”/“paralog,” which arise from speciation event and multiplication event, respectively. That is, an “orthologue” of a protein is a protein of a different species which performs the same or a similar function as the protein it is an orthologue of, a “paralogue” of a protein is a protein of the same species that originates from gene multiplication and which performs the same or a similar function as the protein it is a paralog of Orthologous/paralogous proteins may but need not be structurally related, or are only partially structurally related. In particular embodiments, the homologue or orthologue or paralogue of a CasPR protein as referred to herein (e.g., Cas5d, Cas6, or Csf5) has a sequence homology or identity of at least 40%, at least 50%, at least 60%, at least 70%, at least 80%, at least 85%, at least 90%, such as for instance at least 95% with a CasPR effector protein herein.

In a related aspect, the disclosure provides a Class 1, Type I-C or Cas5d type variant/derivative CasPR protein, including a functional fragment thereof (e.g., at least the N-terminal 120, 130, 140, 150, 160, 170, 180, 190, 200, 210 or 220 residues), that shares at least about 65%, 69%, 70%, 75%, 80%, 85%, 90%, 91%, 92%, 93%, 94%, 95%, 96%, 97%, 98%, 99% or more (e.g., 100%) sequence identity to any one of the wild-type Cas5d CasPR described above. In certain embodiments, the functional fragment thereof retains the ability to bind to the DR sequence bound by the respective wild-type Cas5d sequences. In certain embodiments, the functional fragments comprise up to 99%, 95%, 90%, 85%, 80%, 75%, 70%, 65%, 60%, 55% or 50% of the respective wild-type Cas5d sequences.

As used herein, a “variant” of a protein has qualities or characteristics that have a pattern that deviates from what occurs in nature. A “derivative” derives from a protein and may have similar function, different function, a partial function of the protein from which it derives from.

In a related aspect, the disclosure provides a Class 1 Type I-C or Cas5d type variant/derivative CasPR protein that contains up to 1, 2, 3, 4, 5, 6, 7, 8, 9, or 10 amino acid substitutions (e.g., conserved substitutions), additions, or deletions compared to any one of the wild-type Cas5d CasPR described above. When there is more than 1 substitutions (e.g., conserved substitutions), additions, or deletions, the substitutions (e.g., conserved substitutions), additions, or deletions can be on consecutive or non-consecutive residues.

In certain embodiments, the variant/derivative thereof at least preserves the RNA-binding ability of the wild-type Class 1, Type I-C or Cas5d protein from which the variant/derivative is derived, such as the ability to bind to a cognate DR sequence in crRNA. The Class 1, Type I-C or Cas5d type variant/derivative thereof does not include any naturally existing or wild-type Cas5d from which the variant/derivative is derived.

In certain embodiments, the variant/derivative thereof further preserves the ability of the wild-type Class 1, Type I-C or Cas5d from which the variant/derivative is derived, to process pre-crRNA to mature crRNA, e.g., the endonuclease activity.

In certain embodiments, the variant/derivative thereof retains the ability to bind, but not the ability to cleave (e.g., the endonuclease activity) pre-crRNA to mature crRNA, compared to the wild-type Class 1, Type I-C or Cas5d from which the variant/derivative is derived. Cas5d structure reveals a ferredoxin domain-based architecture and a catalytic triad formed by Y46, K116, and H117 residues. See Nam et al., Structure 20:1574-84, 2012. Thus Cas5d (from Bacillus halodurans) mutant lacking endonuclease activity (or “dCas5d”) can be produced by mutating any one or more of the three residues in the catalytic triad. Other dCas5d from different species can be produced based on catalytic triad mutations corresponding to that in Bacillus halodurans.

For example, the catalytic residues of BhCAs5d and SpCas5d are Y46/K116/H117 and Y48/K118/H119, respectively. Thus a dCas5d protein based on these CasPR can be: dead BhCas5d (Y46A, K116A and/or H117A), and dead SpCas5d (Y48A, K118A and/or H119A). In certain embodiments, one, two, or three residues of the catalytic triad residues is/are mutated to create the “dead” nucleases, and the mutations can be, but are not limited to Ala, so long as the side chain of the mutated residue is substantially different from the original Y, K or H residue(s).

The endonuclease activity or lack thereof can be tested using any art recognized method, such as the gel mobility shift assay as described in Garside et al., RNA 18(11):2020-2028, 2012 (incorporated herein by reference).

The DR coding sequences for the Cas5d of SEQ ID NOs: 143 and 144 are SEQ ID NOs: 154 and 155. The DR sequences of the other Class 1, Type I-C or Cas5d endonucleases can be obtained from the respective CRISPR locus from which the Cas5d sequences originate.

In certain embodiments, the Cas5d CasPR, the variant or derivative thereof (including dCas5d mutant), or the functional fragment thereof binds to not just the full length or the natural DR hairpin RNA structure of the CRISPR locus to which they belong, but also binds to a truncated version of the DR hairpin RNA structure. In certain embodiments, the truncated version comprises the stem of the natural DR hairpin RNA structure, and optionally at least 4-8 nts (e.g., 4, 5, 6, 7, or 8 nts) of single-stranded sequence 3′ to the stem.

The truncated DR with the single-stranded sequence can be processed by Cas5d, and is thus useful for multiplexing targeting when the pre-crRNA processing activity of Cas5d is used to process and release individual crRNAs in the pre-crRNA transcript. When the processing function of Cas5d is not needed, however, the truncated DR can comprise only the hairpin region sequence but not the single-stranded sequence yet still preserving the ability for Cas5d binding.

In a related aspect, the disclosure provides a polynucleotide encoding any one of the Class 1, Type I-C or Cas5d CasPR proteins herein, including wild-type, derivative/variant (including dCas5d mutant), or functional fragment thereof.

In another related aspect, the disclosure provides reverse complement sequence of the above polynucleotides encoding any one of the Class 1, Type I-C or Cas5d CasPR proteins herein, including wild-type, derivative/variant thereof (including dCas5d mutant), and functional fragment thereof.

In certain embodiments, the polynucleotide is not a naturally occurring polynucleotide that encodes a wild-type Class 1, Type I-C or Cas5d CasPR protein herein.

In certain embodiments, the polynucleotide is codon-optimized, such as codon-optimized for eukaryotic or mammalian expression, e.g., human expression. It will be appreciated that, while codon-optimization for human is routinely available, codon optimization for a host of other species other than human, or for codon optimization for specific organs is known. In some embodiments, an enzyme coding sequence encoding a CasPR is codon optimized for expression in particular cells, such as eukaryotic cells. The eukaryotic cells may be those of or derived from a particular organism, such as a mammal, including but not limited to human, or non-human eukaryote or animal or mammal as herein discussed, e.g., mouse, rat, rabbit, dog, livestock, or non-human mammal or primate.

In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura et al., “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, P A), are also available. In some embodiments, one or more codons (e.g. 1, 2, 3, 4, 5, 10, 15, 20, 25, 50, or more, or all codons) in a sequence encoding a CasPR correspond to the most frequently used codon for a particular amino acid.

### Cas6

Cas6 is one of the six highly conserved or core Cas proteins, and is among the most widely distributed Cas proteins found in numerous archaea and bacteria. It is an endoribonuclease that cleaves the primary transcripts of the CRISPR pre-crRNAs, within each of the direct repeat sequences, in a sequence-specific manner to release individual crRNAs encoded by the CRISPR locus. Cas6 interacts with a specific sequence motif in the 5′ region of the CRISPR repeat element (e.g., 20-30 nucleotides from the 5′ end of the DR sequence) and cleaves at a defined site within the 3′ region of the repeat (which is about 20-25 nucleotides form the 5′ end of the DR sequence). The Cas6 cleavage products then undergo further processing to generate smaller mature psiRNA species.

The 1.8 angstrom crystal structure of the Pyrococcus furiosus Cas6 reveals two ferredoxin-like folds that are found in other RNA-binding proteins. The predicted active site of the enzyme is similar to that of tRNA splicing endonucleases. Like the functionally similar Cse3 (CRISPR-Cas system) protein of E. coli, Cas6 is a member of the RAMP (repeat-associated mysterious protein) superfamily proteins which contain G-rich loops and are predicted to be RNA-binding proteins. Cas6 is distinguished from the many other RAMP family members by a conserved sequence motif within the predicted C-terminal G-rich loop (consensus GhGxxxxxGhG, where h is hydrophobic and xxxxx has at least one lysine or arginine).

Mutation of 2 nt spanning the cleavage site drastically reduced the cleavage activity of PfCas6 without preventing binding of Cas6 to the DR RNA based on RNA gel mobility shift assay. The Cas6 cleavage site is at a junction within a potential stem—loop structure that may form by base-pairing between weakly palindromic sequences commonly found at the 5′ and 3′ termini of CRISPR DR sequences.

The RNA sequence requirements of Cas6 binding and endonucleolytic cleavage have been elucidated. RNA gel mobility shift assay showed that sequences in the 5′ region of the CRISPR DR sequence, especially the 5′ most 12 nt, most importantly the first 8 nt, are important for PfCas6 binding. Meanwhile, cleavage by Cas6 appears to involve additional elements, because there are mutations that dramatically reduce cleavage efficiency without disrupting PfCas6 binding. Specifically, substitution of 2 nt at the cleavage site disrupts cleavage but not binding. Substitution of the last 8 nt of the DR, small (4-nt) insertions or deletions, or substitution of 6 nt between the PfCas6-binding site and cleavage site, specifically disrupted cleavage. No cleavage activity was observed with a DNA repeat sequence. These results suggest that cleavage depends upon sequence elements along the length of the repeat and perhaps upon the distance between the binding and cleavage sites, and are consistent with a requirement for a specific RNA fold such as the predicted hairpin structure.

BLASTp search in the NCBI nr database using the SsoCas6 (I-A) protein sequence (SEQ ID NO: 141) retrieved about 16 of the top 100 homologous sequences each sharing at least 80% sequence identity over the entire length of the query sequence, most over 95% identity.

BLASTp search in the NCBI nr database using the MmCas6 protein sequence (SEQ ID NO: 142) retrieved, in addition to the Methanococcus maripaludis query sequence, 3 other homologous sequences sharing at least 63-70% sequence identity over the entire length of the query sequence.

BLASTp search in the NCBI nr database using the SaCas6 protein sequence (SEQ ID NO: 145) retrieved, in addition to the Synechococcus a. query sequence, another homologous sequences sharing at least 70% sequence identity over the entire length of the query sequence.

BLASTp search in the NCBI nr database using the EcCas6e protein sequence (SEQ ID NO: 146) retrieved, in addition to the E. coli query sequence, 99 other homologous sequences sharing at least 97% sequence identity over the entire length of the query sequence.

BLASTp search in the NCBI nr database using the PaCas6f protein sequence (SEQ ID NO: 147) retrieved, in addition to the Pseudomonas aeruginosa query sequence, about 60 other homologous sequences sharing at least 97% sequence identity over the entire length of the query sequence.

BLASTp search in the NCBI nr database using the MtCas6 protein sequence (SEQ ID NO: 148) retrieved, in addition to the Mycobacterium tuberculosis query sequence, about 35 homologous sequences sharing at least 99% sequence identity over the entire length of the query sequence, and another 50 or so sharing at least 99% sequence identity over at least 50-85% of the query sequence.

BLASTp search in the NCBI nr database using the PfCas6 protein sequence (SEQ ID NO: 149) retrieved, in addition to the Pyrococcus furiosus query sequence, about 4 sequences sharing at least 99% sequence identity over the entire length of the query sequence, and another 50 or so sharing at least 60-70% sequence identity over at least 90% of the query sequence.

Thus one aspect of the disclosure provide a wild-type Class 1, Type I or Cas6 type CasPR protein (e.g., homologs, orthologs, paralogs) that shares at least about 65%, 69%, 70%, 75%, 80%, 85%, 90%, 91%, 92%, 93%, 94%, 95%, 96%, 97%, 98%, 99% or more sequence identity to SEQ ID NOs: 141, 142, 145, 146, 147, 148, or 149, such as those that are currently available in the NCBI nr database and can be readily retrieved using SEQ ID NO: 141, 142, 145, 146, 147, 148, or 149 as protein query sequence.

In a related aspect, the disclosure provides a Class 1, Type I or Cas6 type variant/derivative CasPR protein, including a functional fragment thereof (e.g., at least the N-terminal 100, 110, 120, 130, 140, 150, 160, 170, 180, 190, or 200 residues), that shares at least about 65%, 69%, 70%, 75%, 80%, 85%, 90%, 91%, 92%, 93%, 94%, 95%, 96%, 97%, 98%, 99% or more sequence identity to any one of the wild-type Cas6 CasPR described above. In certain embodiments, the functional fragment thereof retains the ability to bind to the DR sequence bound by the respective wild-type Cas6 sequences. In certain embodiments, the functional fragments comprise up to 99%, 95%, 90%, 85%, 80%, 75%, 70%, 65%, 60%, 55% or 50% of the respective wild-type Cas6 sequences.

In a related aspect, the disclosure provides a Class 1, Type I or Cas6 type variant/derivative CasPR protein that contains up to 1, 2, 3, 4, 5, 6, 7, 8, 9, or 10 amino acid substitutions (e.g., conserved substitutions), additions, or deletions compared to any one of the wild-type Cas6 CasPR described above. When there is more than 1 substitutions (e.g., conserved substitutions), additions, or deletions, the substitutions (e.g., conserved substitutions), additions, or deletions can be on consecutive or non-consecutive residues.

In certain embodiments, the variant/derivative thereof at least preserves the RNA-binding ability of the wild-type Class 1, Type I or Cas6 protein from which the variant/derivative is derived, such as the ability to bind to a cognate DR sequence in crRNA. The Class 1, Type I or Cas6 type variant/derivative thereof does not include any naturally existing or wild-type Cas6 from which the variant/derivative is derived.

In certain embodiments, the variant/derivative thereof further preserves the ability of the wild-type Class 1, Type I or Cas6 from which the variant/derivative is derived, to process pre-crRNA to mature crRNA, e.g., the endonuclease activity.

In certain embodiments, the variant/derivative thereof retains the ability to bind, but not the ability to cleave (e.g., the endonuclease activity) pre-crRNA to mature crRNA, compared to the wild-type Class 1, Type I or Cas6 from which the variant/derivative is derived.

Three conserved residues (Y31, H46, and K52) that are separated from one another in the primary sequences of Cas6 proteins from diverse organisms were found to cluster in the crystal structure of Cas6 from P. furiosus (Carte et al., Genes Dev 22:3489-3496, 2008), which structure was also found to be similar to the configuration of archaeal tRNA splicing endonuclease. Substitution of any of the three triad amino acids with Alanine led to a significant decrease in cleavage activity relative to wild-type Cas6. No cleavage activity was observed with the Y31A and H46A Cas6 mutants. The cleavage activity was reduced ˜40-fold at the highest tested concentration (500 nM) of K52A Cas6 mutant relative to wild-type Cas6. Meanwhile, based on gel mobility shift assay, Tyr31, His46, and Lys52 were found to be not required for binding to CRISPR repeat RNA (Carte et al., RNA 16(11):2181-2188, 2010). Thus these three conserved amino acids comprise a catalytic triad required for Cas6 cleavage of the CRISPR crRNA. Cas6 mutants lacking cleavage activity from P. furiosus and other species can be readily produced based on mutating the corresponding residues of Y31, H46, and K52 in P. furiosus.

The catalytic residues of four Cas6 include at least: MtCas6: Y29, K51; MmCas6: Y34, K56; EcCas6e: H18; and PaCas6f: Y31, H36, K52. Thus a dCas6 protein based on these CasPR can be: dead MtCas6 (Y29A and/or K51A); dead MmCas6 (Y34A and/or K56A); dead EcCas6e: H18A; and dead PaCas6f: Y31A, H36A, and/or K52A. In certain embodiments, one, two, or three residues of the catalytic residues is/are mutated to create the “dead” nucleases, and the mutations can be, but are not limited to Ala, so long as the side chain of the mutated residue is substantially different from the original (e.g., Y, K or H) residue(s).

The endonuclease activity or lack thereof can be tested using any art recognized method, such as the gel mobility shift assay as described in Carte et al., RNA 16(11):2181-2188, 2010 (incorporated herein by reference).

The DR coding sequences for the Cas6 of SEQ ID NOs: 141, 142, 145, 146, 147, 148, and 149 are SEQ ID NOs: 152, 153, 156, 157, 158, 159, or 160, respectively. The DR sequences of the other Class 1, Type I or Cas6 endonucleases can be obtained from the respective CRISPR locus from which the Cas6 sequences originate.

In certain embodiments, the Cas6 CasPR, the variant or derivative thereof (including dCas5d mutant), or the functional fragment thereof binds to not just the full length or the natural DR hairpin RNA structure of the CRISPR locus to which they belong, but also binds to a truncated version of the DR hairpin RNA structure. In certain embodiments, the truncated version comprises the most 5′ 8-12 nt (e.g., 8, 9, 10, 11, or 12 nts) of the cognate DR sequence for the respective Cas6, such as the most 5′ 22-25 nts of the cognate DR sequence for the respective Cas6.

In a related aspect, the disclosure provides a polynucleotide encoding any one of the Class 1, Type I or Cas6 CasPR proteins herein, including wild-type, derivative/variant (including dCas5d mutant), or functional fragment thereof.

In another related aspect, the disclosure provides reverse complement sequence of the above polynucleotides encoding any one of the Class 1, Type I or Cas6 CasPR proteins herein, including wild-type, derivative/variant thereof (including dCas5d mutant), and functional fragment thereof.

In certain embodiments, the polynucleotide is not a naturally occurring polynucleotide that encodes a wild-type Class 1, Type I or Cas6 CasPR protein herein.

In certain embodiments, the polynucleotide is codon-optimized for mammalian expression.

### Csf5

Csf5 is also known as the CRISPR-Cas type IV Cas6 crRNA endonuclease (see Ozcan et al., Nat Microbiol. 4(1):89-96, 2019). It processes CRISPR pre-crRNA into mature crRNAs that are specifically incorporated into type IV CRISPR-ribonucleoprotein (crRNP) complexes. Structures of RNA-bound Csf5 have been obtained and studied.

At least in M. australiensis Type IV CRISPR system (Ma Cas6-IV), the stem of the DR hairpin RNA structure may be recognized primarily through shape rather than base-specific interactions, because base switches at the base of the DR hairpin RNA stem would not disrupt base pairing and are acceptable for Ma Cas6-IV binding if both Watson Crick and G-U wobble base pairs are preserved. Other base switches in the arms and loop of the hairpin likewise suggest that those positions are recognized through shape, or are not necessary at all for binding.

Structural comparisons between the Ma Cas6-IV and Csf5 from Aromatoleum aromaticum (PDB 6H9I) reveal that they both contain the dual RRM domain scaffold generally observed in Cas6 proteins. The C-terminal RRM domains of both enzymes contain the motifs that bind crRNA (groove-binding element or GBE, β-hairpin, and G-loop), but the C-terminal domain of Csf5 differs from Ma Cas6-IV in that the second alpha helix (α2) of the canonical RRM fold is absent. In both Csf5 and Ma Cas6-IV, the al helices of the N-terminal RRM domains have been replaced with helix-turn-helix motifs that house putative active-site residues. However, instead of the small loop sequence observed in Ma Cas6-IV that connects the helix-loop-helix to β2, Csf5 has an insertion of ˜40 amino acids called the α-helical finger domain (α-HFD) that contains two additional helices. One of these helices interacts with the minor groove of the crRNA stem-loop, providing additional contacts for binding the crRNA that may provide additional specificity toward Type IV crRNA repeats.

BLASTp search in the NCBI nr database using the PaCsf5 protein sequence (SEQ ID NO: 150) retrieved, in addition to the Pseudomonas aeruginosa query sequence, about 6 homologous sequences sharing at least 80% sequence identity over the entire length of the query sequence, and 4 of which over 98% identical.

Thus one aspect of the disclosure provide a wild-type Class 1, Type IV or Csf5 type CasPR protein (e.g., homologs, orthologs, paralogs) that shares at least about 65%, 69%, 70%, 75%, 80%, 85%, 90%, 91%, 92%, 93%, 94%, 95%, 96%, 97%, 98%, 99% or more sequence identity to SEQ ID NO: 150 or 151, such as those that are currently available in the NCBI nr database and can be readily retrieved using SEQ ID NO: 150 or 151 as protein query sequence.

In a related aspect, the disclosure provides a Class 1, Type IV or Csf5 type variant/derivative CasPR protein, including a functional fragment thereof (e.g., at least the N-terminal 120, 130, 140, 150, 160, 170, 180, 190, 200, 210 or 220 residues), that shares at least about 65%, 69%, 70%, 75%, 80%, 85%, 90%, 91%, 92%, 93%, 94%, 95%, 96%, 97%, 98%, 99% or more sequence identity to any one of the wild-type Class 1, Type IV or Csf5 CasPR described above. In certain embodiments, the functional fragment thereof retains the ability to bind to the DR sequence bound by the respective wild-type Csf5 sequences. In certain embodiments, the functional fragments comprise up to 99%, 95%, 90%, 85%, 80%, 75%, 70%, 65%, 60%, 55% or 50% of the respective wild-type Csf5 sequences.

In a related aspect, the disclosure provides a Class 1, Type IV or Csf5 type variant/derivative CasPR protein that contains up to 1, 2, 3, 4, 5, 6, 7, 8, 9, or 10 amino acid substitutions (e.g., conserved substitutions), additions, or deletions compared to any one of the wild-type Class 1, Type IV or Csf5 CasPR described above. When there is more than 1 substitutions (e.g., conserved substitutions), additions, or deletions, the substitutions (e.g., conserved substitutions), additions, or deletions can be on consecutive or non-consecutive residues.

In certain embodiments, the variant/derivative thereof at least preserves the RNA-binding ability of the wild-type Class 1, Type IV or Csf5 protein from which the variant/derivative is derived, such as the ability to bind to a cognate DR sequence in crRNA. The Class 1, Type IV or Csf5 type variant/derivative thereof does not include any naturally existing or wild-type Class 1, Type IV or Csf5 from which the variant/derivative is derived.

In certain embodiments, the variant/derivative thereof further preserves the ability of the wild-type Class 1, Type IV or Csf5 from which the variant/derivative is derived, to process pre-crRNA to mature crRNA, e.g., the endonuclease activity.

In certain embodiments, the variant/derivative thereof retains the ability to bind, but not the ability to cleave (e.g., the endonuclease activity) pre-crRNA to mature crRNA, compared to the wild-type Class 1, Type IV or Csf5 from which the variant/derivative is derived.

Both Csf5 and Ma Cas6-IV contain a histidine in the N-terminal RRM at the same sequence position (H44), but the Csf5 H44 is within the 40 amino acid insert α-HFD and is several Ångstroms away from the scissile phosphate, and does not participate in nuclease activity. Rather, mutation of arginine residues located on the Csf5 helix-turn-helix and the G-loop (R23A, R38A, R242A) impaired cleavage. Notably several of these arginines are located in similar positions to the active site residues of Ma Cas6-IV (His44 and Tyr31), supporting the notion that these Type IV Cas proteins rely on similar structural themes to bind and cleave crRNA substrates despite their diverse sequences. See Taylor et al., RNA Biol. 16(10):1438-1447, 2019. Thus Csf5 mutant lacking endonuclease activity (or “dCsf5”) can be produced by mutating any one or more of the three residues corresponding to the catalytic triad (R23, R38, and R242) of Csf5 from Aromatoleum aromaticum (PDB 6H9I), including other dCsf5 from different species.

The endonuclease activity or lack thereof can be tested using any art recognized method, such as the gel mobility shift assay as described in Garside et al., RNA 18(11):2020-2028, 2012 (incorporated herein by reference).

The DR coding sequences for the Csf5 of SEQ ID NOs: 150 and 151 are SEQ ID NOs: 161 and 162, respectively. The DR sequences of the other Class 1, Type IV or Csf5 endonucleases can be obtained from the respective CRISPR locus from which the Csf5 sequences originate.

In certain embodiments, the Csf5 CasPR, the variant or derivative thereof (including dCsf5 mutant), or the functional fragment thereof binds to not just the full length or the natural DR hairpin RNA structure of the CRISPR locus to which they belong, but also binds to a truncated version of the DR hairpin RNA structure. In certain embodiments, the truncated version comprises at least the stem of the natural DR hairpin RNA structure. In certain embodiments, the Csf5 CasPR, the variant or derivative thereof (including dCsf5 mutant), or the functional fragment thereof binds to a variant DR hairpin RNA structure that preserves substantially all the structural features (e.g., stems, loops, bulges in the stem, etc.) but having different nucleotide sequences (e.g., at least 1, 2, 3, 4, 5, 6, 7, 8, 9, 10, 11, 12, 13, 14, 15, 16, 17, 18, 19, 20 or more nucleotide sequence differences compared to the wild-type DR sequence).

In a related aspect, the disclosure provides a polynucleotide encoding any one of the Class 1, Type IV or Csf5 CasPR proteins herein, including wild-type, derivative/variant (including dCsf5 mutant), or functional fragment thereof.

In another related aspect, the disclosure provides reverse complement sequence of the above polynucleotides encoding any one of the Class 1, Type IV or Csf5 CasPR proteins herein, including wild-type, derivative/variant (including dCsf5 mutant), or functional fragment thereof.

In certain embodiments, the polynucleotide is not a naturally occurring polynucleotide that encodes a wild-type Class 1, Type IV or Csf5 CasPR protein herein.

In certain embodiments, the polynucleotide is codon-optimized for mammalian expression.

### Functional Fragments

Functional fragments of the subject CasPRs (e.g., Cas5d, Cas6, and Csf5), including wild-type, variant, and derivative thereof, are also provided. The functional fragments of the disclosure preserve or maintain at least one function of the full-length protein from which they originate. For example, in some embodiments, the preserved function is binding to cognate crRNA particularly the DR sequence or structural elements therein responsible for CasPR binding. In other embodiments, the preserved function is catalytic activity towards pre-crRNA. In some embodiments, both binding to DR sequence and catalytic activity are preserved.

For example, in certain embodiments, to reduce the size of a fusion protein of the subject CasPR and the one or more functional domains (see below), the C-terminus of the CasPR (e.g., Cas5d, Cas6, and Csf5) can be truncated while still maintaining its RNA binding function. For example, at least or no more than 5 amino acids, 10 amino acids, 15 amino acids, 20 amino acids, 25 amino acids, 30 amino acids, 35 amino acids, 40 amino acids, 45 amino acids, 50 amino acids, 55 amino acids, 60 amino acids, 65 amino acids, 70 amino acids, 75 amino acids, 80 amino acids, 85 amino acids, 90 amino acids, or 100 amino acid, may be truncated at the C-terminus of the CasPR.

In some embodiments, the N-terminus of the CasPR (e.g., Cas5d, Cas6, and Csf5) may be truncated. For example, at least or no more than 5 amino acids, 10 amino acids, 15 amino acids, 20 amino acids, 25 amino acids, 30 amino acids, 35 amino acids, 40 amino acids, 45 amino acids, 50 amino acids, 55 amino acids, 60 amino acids, 65 amino acids, 70 amino acids, 75 amino acids, 80 amino acids, 85 amino acids, 90 amino acids, or 100 amino acid may be truncated at the N-terminus of the subject CasPR.

In some embodiments, both the N- and the C-termini of the subject CasPR may be truncated. Not specifically recited herein but are explicitly incorporated is a permutation and combination of each N- and each C-terminal deletions mentioned above, such as C-terminal deletion of at least/no more than 5 residues AND N-terminal deletions of at least/no more than 5, 10, 15, 20, 20, 25, 30, 35, 40, 45, 50, 55, 60, 65, 70, 75, 80, 85, 90, 95, or 100 residues; . . . and, C-terminal deletion of at least/no more than 100 residues AND N-terminal deletions of at least/no more than 5, 10, 15, 20, 20, 25, 30, 35, 40, 45, 50, 55, 60, 65, 70, 75, 80, 85, 90, 95, or 100 residues.

### Split Proteins

In certain embodiments, the functional fragment is a so-called “split protein,” in that it contains one of two parts of the full length CasPR enzyme—the RNA binding domain or the endonuclease domain, which together substantially comprise a functional CasPR. The split should always be so that the catalytic domain(s) are unaffected. The use of a split version of the CasPR may not only allow increased specificity but may also be advantageous for delivery (e.g., smaller size). Thus in certain embodiments, the split CasPR may function as a nuclease. In another embodiment, the split CasPR may be a nuclease dead-CasPR which is essentially an RNA-binding protein with very little or no catalytic activity, due to typically mutation(s) in its catalytic domains or the lack of the catalytic domain altogether. The nuclease dead-split CasPR can be fused to other heterologous functional domains described herein to target such heterologous functional domains to a specific site on a target RNA.

In certain embodiments, each half of the split CasPR may be fused to a dimerization partner, such as the rapamycin-sensitive dimerization domains, which allow the generation of a chemically inducible split CasPR temporal control of CasPR activity. For example, the split CasPR RNA binding domain may bind to the guide RNA at the target site, and the split CasPR nuclease domain (or nuclease-dead version of the nuclease domain) may be fused to a heterologous functional domain, such as a deaminase. Thus CasPR can be rendered chemically inducible by being split into two fragments and that rapamycin-sensitive dimerization domains may be used for controlled reassembly of the CasPR or fusion thereof.

### Conservative Substitutions

In certain embodiments, derivatives or variants of the CasPRs (e.g., Cas5d, Cas6, and Csf5) include proteins that differ from the wild-type sequence by one or more conservative substitutions, include substitutions inside or outside the RNA binding or catalytic domain. In certain embodiments, the substitution does not include substitution of the catalytic triad residues. In certain embodiments, the substitution includes substitution of the catalytic triad residues.

Such amino acid substitutions may be made based on the differences or similarities in amino acid properties, such as polarity, charge, solubility, hydrophobicity, hydrophilicity, and/or the amphipathic nature of the residues. For this purpose, amino acids have been grouped together based on the functional groups they carry, i.e., based on the properties of their side chains alone. Typically, a grouping as shown below can be used for conservative substitution.

| Set | | Sub-unit | |
|---|---|---|---|
| Hydrophbic | FWYHKMILVAGC | Aromatic | FWHY |
| | (SEQ ID NO: 240) | Aliphatic | ILV |
| Polar | WYHKREDCSTNQ | Charged | HKRED |
| | (SEQ ID NO: 241) | Positively charged | HKR |
| | | Negatively charged | HD |
| Small | VCAGSFTND | Tiny | AGS |
| | (SEQ ID NO: 242) | | |

### Homology Modeling

Numerous subject CasPR protein sequences have been described herein, including publically available database sequences incorporated herein that satisfy certain threshold sequence identity requirements to the subject CasPRs (e.g., SEQ ID NOs: 141-151). Homology modeling can be used to predict the structure of the related CasPRs, such as homologs, orthologs, paralogs, variants, derivatives, and functional fragments thereof, partly based on the known structures of certain CasPRs within a subfamily, and the sequence homology/identity between the related CasPRs.

For example, corresponding residues in other CasPR orthologs can be identified by the methods of Zhang et al. (Nature 490(7421):556-60, 2012, incorporated herein by reference) and Chen et al. (PLoS Comput Biol. 11(5):e1004248, 2015, incorporated herein by reference). The method involves taking a pair a query proteins and using structural alignment to identify structural representatives that correspond to either their experimentally determined structures or homology models. Structural alignment is further used to identify both close and remote structural neighbors by considering global and local geometric relationships. Whenever two neighbors of the structural representatives form a complex reported in the Protein Data Bank, this defines a template for modelling the interaction between the two query proteins. Models of a complex are created by superimposing the representative structures on their corresponding structural neighbor in the template. Also see Dey et al., Prot Sci. 22:359-66, 2013.

4. RNA Guides, Guide RNAs (gRNAs), or crRNAs

In some embodiments, the CRISPR-Cas system described herein include at least one RNA guide (e.g., a gRNA or a crRNA).

The architecture of multiple RNA guides is known in the art (see, e.g., International Publication Nos. WO 2014/093622 and WO 2015/070083, the entire contents of each of which are incorporated herein by reference).

In some embodiments, the CRISPR-Cas system described herein include multiple RNA guides (e.g., one, two, three, four, five, six, seven, eight, or more RNA guides). In certain embodiments, each guide RNA independently comprises a (different) spacer sequence capable of hybridizing to one or more target RNA, said spacer sequence is flanked by a direct repeat (DR) sequence (e.g., native to the Cas effector protein) at both the 5′ end and the 3′ end of the spacer sequence.

In some embodiments, the RNA guide includes a crRNA. In some embodiments, the RNA guide includes a crRNA but not a tracrRNA.

Sequences for guide RNAs from multiple CRISPR systems are generally known in the art, see, for example, Grissa et al. (Nucleic Acids Res. 35 (web server issue): W52-7, 2007; Grissa et al., BMC Bioinformatics 8:172, 2007; Grissa et al., Nucleic Acids Res. 36 (web server issue): W145-8, 2008; and Moller and Liang, PeerJ 5: e3788, 2017; the CRISPR database at: crispr.i2bc.paris-saclayfr/crispr/BLAST/CRISPRsBlast.php; and MetaCRAST available at: github.com/molleraj/MetaCRAST). All incorporated herein by reference.

In some embodiments, the crRNA includes a direct repeat (DR) sequence and a spacer sequence (e.g., the spacer sequence is flanked by one copy each of the DR sequence). In certain embodiments, the crRNA comprises, consists essentially of, or consists of a direct repeat sequence linked to a spacer sequence, both at the 5′ end and 3′ end of the spacer sequence.

In some embodiments, the crRNA includes a first direct repeat (DR) sequence, a first spacer sequence, a second DR sequence, a second spacer sequence, and a third DR sequence, wherein the first spacer sequence is flanked by the first and second DR sequences at both the 5′ end and 3′ end of the first spacer sequence, and the second spacer sequence is flanked by the second and third DR sequences at both the 5′ end and 3′ end of the second spacer sequence, wherein the first and second spacer sequences can be the same or different, and wherein the first, second, and third DR sequences can be the same or different.

In general, the Cas protein forms a complex with the mature crRNA, which spacer sequence directs the complex to a sequence-specific binding with the target RNA that is substantially complementary to the spacer sequence, and/or hybridizes to the spacer sequence. The resulting complex comprises the Cas protein and the mature crRNA bound to the target RNA.

The direct repeat sequences for the Cas13e and Cas13f systems are generally well conserved, especially at the ends, with a GCTG for Cas13e and GCTGT for Cas13f at the 5′-end, reverse complementary to a CAGC for Cas13e and ACAGC for Cas13f at the 3′ end. This conservation suggests strong base pairing for an RNA stem-loop structure that potentially interacts with the protein(s) in the locus.

In certain embodiments, each DR sequence in the guide RNA of the disclosure has substantially the same secondary structure as the secondary structure of any one of SEQ ID NOs: 8-14, 126-140, and 153-162, depending on the specific Cas effector protein compatible with the DR sequences.

In certain embodiments, each DR sequence is encoded by or comprises any one of SEQ ID NOs: 8-14, 126-140, and 153-162.

In some embodiments, the direct repeat sequence, when in RNA, comprises the general secondary structure of 5′-Sla-Ba-S2a-L-S2b-Bb-S1b-3′, wherein segments S1a and S1b are reverse complement sequences and form a first stem (S1) having 4 nucleotides in Cas13e and 5 nucleotides in Cas13f; segments Ba and Bb do not base pair with each other and form a symmetrical or nearly symmetrical bulge (B), and have 5 nucleotides each in Cas13e, and 5 (Ba) and 4 (Bb) or 6 (Ba) and 5 (Bb) nucleotides respectively in Cas13f; segments S2a and S2b are reverse complement sequences and form a second stem (S2) having 5 base pairs in Cas13e and either 6 or 5 base pairs in Cas13f; and L is an 8-nucleotide loop in Cas13e and a 5-nucleotide loop in Cas13f. See FIG. 2.

In certain embodiments, S1a has a sequence of GCUG in Cas13e and GCUGU in Cas13f.

In certain embodiments, S2a has a sequence of GCCCC in Cas13e and A/G CCUC G/A in Cas13f (wherein the first A or G may be absent).

In some embodiments, the direct repeat sequence comprises or consists of a nucleic acid sequence of SEQ ID NOs: 8-14, 126-140 and 152-162.

As used herein, “direct repeat sequence” may refer to the DNA coding sequence in the CRISPR locus, or to the RNA encoded by the same in crRNA. Thus, when any of SEQ ID NOs: 8-14, 126-140 and 152-162 is referred to in the context of an RNA molecule, such as crRNA, each T is understood to represent a U.

In some embodiments, the direct repeat sequence comprises or consists of a nucleic acid sequence having up to 1, 2, 3, 4, 5, 6, 7, or 8 nucleotides of deletion, insertion, or substitution of SEQ ID NOs: 8-14, 126-140 and 152-162. In some embodiments, the direct repeat sequence comprises or consists of a nucleic acid sequence having at least 80%, 85%, 90%, 95%, or 97% of sequence identity with SEQ ID NOs: 8-14, 126-140 and 152-162 (e.g., due to deletion, insertion, or substitution of nucleotides in SEQ ID NOs: 8-14, 126-140 and 152-162). In some embodiments, the direct repeat sequence comprises or consists of a nucleic acid sequence that is not identical to any one of SEQ ID NOs: 8-14, 126-140 and 152-162, but can hybridize with a complement of any one of SEQ ID NOs: 8-14, 126-140 and 152-162 under stringent hybridization conditions, or can bind to a complement of any one of SEQ ID NOs: 8-14, 126-140 and 152-162 under physiological conditions.

In certain embodiments, the deletion, insertion, or substitution does not change the overall secondary structure of that of SEQ ID NOs: 8-14, 126-140 and 152-162 (e.g., the relative locations and/or sizes of the stems and bulges and loop do not significantly deviate from that of the original stems, bulges, and loop). For example, the deletion, insert, or substitution may be in the bulge or loop region so that the overall symmetry of the bulge remains largely the same. The deletion, insertion, or substitution may be in the stems so that the length of the stems do not significantly deviate from that of the original stems (e.g., adding or deleting one base pair in each of the two stems correspond to 4 total base changes).

In certain embodiments, the deletion, insertion, or substitution results in a derivative DR sequence that may have ±1 or 2 base pair(s) in one or both stems (see FIG. 2), have ±1, 2, or 3 bases in either or both of the single strands in the bulge, and/or have ±1, 2, 3, or 4 bases in the loop region.

In certain embodiments, any of the above direct repeat sequences that is different from any one of SEQ ID NOs: 8-14, 126-140 and 152-162 retains the ability to function as a direct repeat sequence in the Cas13 proteins or CasPRs, as the DR sequence of SEQ ID NOs: 8-14, 126-140 and 152-162.

In some embodiments, the direct repeat sequence comprises or consists of a nucleic acid having a nucleic acid sequence of any one of SEQ ID NOs: 8-14, 126-140 and 152-162, with a truncation of the initial three, four, five, six, seven, or eight 3′ nucleotides.

In some embodiments, the Cas effector protein comprises the amino acid sequence of SEQ ID NO: 1 and the crRNA comprises a direct repeat sequence, wherein the direct repeat sequence comprises or consists of the nucleic acid sequence of SEQ ID NO: 8.

In some embodiments, the Cas effector protein comprises the amino acid sequence of SEQ ID NO: 2 and the crRNA comprises a direct repeat sequence, wherein the direct repeat sequence comprises or consists of the nucleic acid sequence of SEQ ID NO: 9.

In some embodiments, the Cas effector protein comprises the amino acid sequence of SEQ ID NO: 3 and the crRNA comprises a direct repeat sequence, wherein the direct repeat sequence comprises or consists of the nucleic acid sequence of SEQ ID NO: 10.

In some embodiments, the Cas effector protein comprises the amino acid sequence of SEQ ID NO: 4 and the crRNA comprises a direct repeat sequence, wherein the direct repeat sequence comprises or consists of the nucleic acid sequence of SEQ ID NO: 11.

In some embodiments, the Cas effector protein comprises the amino acid sequence of SEQ ID NO: 5 and the crRNA comprises a direct repeat sequence, wherein the direct repeat sequence comprises or consists of the nucleic acid sequence of SEQ ID NO: 12.

In some embodiments, the Cas effector protein comprises the amino acid sequence of SEQ ID NO: 6 and the crRNA comprises a direct repeat sequence, wherein the direct repeat sequence comprises or consists of the nucleic acid sequence of SEQ ID NO: 13.

In some embodiments, the Cas effector protein comprises the amino acid sequence of SEQ ID NO: 7 and the crRNA comprises a direct repeat sequence, wherein the direct repeat sequence comprises or consists of the nucleic acid sequence of SEQ ID NO: 14.

In some embodiments, the Cas effector protein comprises the amino acid sequence of SEQ ID NO: 111 and the crRNA comprises a direct repeat sequence, wherein the direct repeat sequence comprises or consists of the nucleic acid sequence of SEQ ID NO: 126.

In some embodiments, the Cas effector protein comprises the amino acid sequence of SEQ ID NO: 112 and the crRNA comprises a direct repeat sequence, wherein the direct repeat sequence comprises or consists of the nucleic acid sequence of SEQ ID NO: 127.

In some embodiments, the Cas effector protein comprises the amino acid sequence of SEQ ID NO: 113 and the crRNA comprises a direct repeat sequence, wherein the direct repeat sequence comprises or consists of the nucleic acid sequence of SEQ ID NO: 128.

In some embodiments, the Cas effector protein comprises the amino acid sequence of SEQ ID NO: 114 and the crRNA comprises a direct repeat sequence, wherein the direct repeat sequence comprises or consists of the nucleic acid sequence of SEQ ID NO: 129.

In some embodiments, the Cas effector protein comprises the amino acid sequence of SEQ ID NO: 115 and the crRNA comprises a direct repeat sequence, wherein the direct repeat sequence comprises or consists of the nucleic acid sequence of SEQ ID NO: 130.

In some embodiments, the Cas effector protein comprises the amino acid sequence of SEQ ID NO: 116 and the crRNA comprises a direct repeat sequence, wherein the direct repeat sequence comprises or consists of the nucleic acid sequence of SEQ ID NO: 131.

In some embodiments, the Cas effector protein comprises the amino acid sequence of SEQ ID NO: 117 and the crRNA comprises a direct repeat sequence, wherein the direct repeat sequence comprises or consists of the nucleic acid sequence of SEQ ID NO: 132.

In some embodiments, the Cas effector protein comprises the amino acid sequence of SEQ ID NO: 118 and the crRNA comprises a direct repeat sequence, wherein the direct repeat sequence comprises or consists of the nucleic acid sequence of SEQ ID NO: 133.

In some embodiments, the Cas effector protein comprises the amino acid sequence of SEQ ID NO: 119 and the crRNA comprises a direct repeat sequence, wherein the direct repeat sequence comprises or consists of the nucleic acid sequence of SEQ ID NO: 134.

In some embodiments, the Cas effector protein comprises the amino acid sequence of SEQ ID NO: 120 and the crRNA comprises a direct repeat sequence, wherein the direct repeat sequence comprises or consists of the nucleic acid sequence of SEQ ID NO: 135.

In some embodiments, the Cas effector protein comprises the amino acid sequence of SEQ ID NO: 121 and the crRNA comprises a direct repeat sequence, wherein the direct repeat sequence comprises or consists of the nucleic acid sequence of SEQ ID NO: 136.

In some embodiments, the Cas effector protein comprises the amino acid sequence of SEQ ID NO: 122 and the crRNA comprises a direct repeat sequence, wherein the direct repeat sequence comprises or consists of the nucleic acid sequence of SEQ ID NO: 137.

In some embodiments, the Cas effector protein comprises the amino acid sequence of SEQ ID NO: 123 and the crRNA comprises a direct repeat sequence, wherein the direct repeat sequence comprises or consists of the nucleic acid sequence of SEQ ID NO: 138.

In some embodiments, the Cas effector protein comprises the amino acid sequence of SEQ ID NO: 124 and the crRNA comprises a direct repeat sequence, wherein the direct repeat sequence comprises or consists of the nucleic acid sequence of SEQ ID NO: 139.

In some embodiments, the Cas effector protein comprises the amino acid sequence of SEQ ID NO: 125 and the crRNA comprises a direct repeat sequence, wherein the direct repeat sequence comprises or consists of the nucleic acid sequence of SEQ ID NO: 140.

In some embodiments, the Cas effector protein comprises the amino acid sequence of SEQ ID NO: 141 and the crRNA comprises a direct repeat sequence, wherein the direct repeat sequence comprises or consists of the nucleic acid sequence of SEQ ID NO: 152.

In some embodiments, the Cas effector protein comprises the amino acid sequence of SEQ ID NO: 142 and the crRNA comprises a direct repeat sequence, wherein the direct repeat sequence comprises or consists of the nucleic acid sequence of SEQ ID NO: 153.

In some embodiments, the Cas effector protein comprises the amino acid sequence of SEQ ID NO: 143 and the crRNA comprises a direct repeat sequence, wherein the direct repeat sequence comprises or consists of the nucleic acid sequence of SEQ ID NO: 154.

In some embodiments, the Cas effector protein comprises the amino acid sequence of SEQ ID NO: 144 and the crRNA comprises a direct repeat sequence, wherein the direct repeat sequence comprises or consists of the nucleic acid sequence of SEQ ID NO: 155.

In some embodiments, the Cas effector protein comprises the amino acid sequence of SEQ ID NO: 145 and the crRNA comprises a direct repeat sequence, wherein the direct repeat sequence comprises or consists of the nucleic acid sequence of SEQ ID NO: 156.

In some embodiments, the Cas effector protein comprises the amino acid sequence of SEQ ID NO: 146 and the crRNA comprises a direct repeat sequence, wherein the direct repeat sequence comprises or consists of the nucleic acid sequence of SEQ ID NO: 157.

In some embodiments, the Cas effector protein comprises the amino acid sequence of SEQ ID NO: 147 and the crRNA comprises a direct repeat sequence, wherein the direct repeat sequence comprises or consists of the nucleic acid sequence of SEQ ID NO: 158.

In some embodiments, the Cas effector protein comprises the amino acid sequence of SEQ ID NO: 148 and the crRNA comprises a direct repeat sequence, wherein the direct repeat sequence comprises or consists of the nucleic acid sequence of SEQ ID NO: 159.

In some embodiments, the Cas effector protein comprises the amino acid sequence of SEQ ID NO: 149 and the crRNA comprises a direct repeat sequence, wherein the direct repeat sequence comprises or consists of the nucleic acid sequence of SEQ ID NO: 160.

In some embodiments, the Cas effector protein comprises the amino acid sequence of SEQ ID NO: 150 and the crRNA comprises a direct repeat sequence, wherein the direct repeat sequence comprises or consists of the nucleic acid sequence of SEQ ID NO: 161.

In some embodiments, the Cas effector protein comprises the amino acid sequence of SEQ ID NO: 151 and the crRNA comprises a direct repeat sequence, wherein the direct repeat sequence comprises or consists of the nucleic acid sequence of SEQ ID NO: 162.

In classic CRISPR systems, the degree of complementarity between a guide sequence (e.g., a crRNA) and its corresponding target sequence can be about 50%, 60%, 75%, 80%, 85%, 90%, 95%, 97.5%, 99%, or 100%. In some embodiments, the degree of complementarity is 90-100%. In certain embodiments, the spacer sequence contains no more than 1, 2, 3, 4, or 5 consecutive or non-consecutive mismatches with the target RNA.

The guide RNAs can be about 5, 10, 11, 12, 13, 14, 15, 16, 17, 18, 19, 20, 21, 22, 23, 24, 25, 26, 27, 28, 29, 30, 35, 40, 45, 50, 75, 100, 125, 150, 175, 200 or more nucleotides in length. For example, for use in a functional Cas13e or Cas13f effector protein, or homologs, orthologs, derivatives, fusions, conjugates, or functional fragment thereof, the spacer can be between 10-60 nucleotides, 20-50 nucleotides, 25-45 nucleotides, 25-35 nucleotides, 15-60 nucleotides, 25-50 nucleotides, about 55 nucleotides, about 50 nucleotides, about 45 nucleotides, about 40 nucleotides, about 35 nucleotides, or about 30 nucleotides, or about 27, 28, 29, 30, 31, 32, or 33 nucleotides. For use in dCas version of any of the above, however, the spacer can be between 10-200 nucleotides, 20-150 nucleotides, 25-100 nucleotides, 25-85 nucleotides, 35-75 nucleotides, 45-60 nucleotides, or about 46, 47, 48, 49, 50, 51, 52, 53, 54, or 55 nucleotides; or between 15-100 nucleotides, 15-80 nucleotides, 15-60 nucleotides, between 25-50 nucleotides, between 30-50 nucleotides, about 100 nucleotides, about 80 nucleotides, about 60 nucleotides, about 55 nucleotides, about 50 nucleotides, about 45 nucleotides, about 40 nucleotides, about 35 nucleotides, about 30 nucleotides, about 20 nucleotides, or about 15 nucleotides in length.

In certain embodiments, the spacer sequence comprises a cystine (C) mismatch opposite to the adenosine (A) in the target RNA and/or an adenosine (A) mismatch opposite to the cytidine (C) in the target RNA.

In certain embodiments, the cystine or adenosine mismatch is about 5, 6, 7, 8, 9, 10, 11, 12, 13, 14, 15, 16, 17, 18, 19, 20, 21, 22, 23, 24, 25, 26, 27, 28, 29, or 30 nucleotides (e.g., about 15-25 nucleotides) from the 5′ or 3′ DR sequence.

To reduce off-target interactions, e.g., to reduce the guide interacting with a target sequence having low complementarity, mutations can be introduced to the CRISPR systems so that the CRISPR systems can distinguish between target and off-target sequences that have greater than 80%, 85%, 90%, or 95% complementarity. In some embodiments, the degree of complementarity is from 80% to 95%, e.g., about 83%, 84%, 85%, 86%, 87%, 88%, 89%, 90%, 91%, 92%, 93%, 94%, or 95% (for example, distinguishing between a target having 18 nucleotides from an off-target of 18 nucleotides having 1, 2, or 3 mismatches). Accordingly, in some embodiments, the degree of complementarity between a guide sequence and its corresponding target sequence is greater than 94.5%, 95%, 95.5%, 96%, 96.5%, 97%, 97.5%, 98%, 98.5%, 99%, 99.5%, or 99.9%. In some embodiments, the degree of complementarity is 100%.

It is known in the field that complete complementarity is not required, provided there is sufficient complementarity to be functional. Modulations of cleavage efficiency can be exploited by introduction of mismatches, e.g., one or more mismatches, such as 1 or 2 mismatches between spacer sequence and target sequence, including the position of the mismatch along the spacer/target. The more central (i.e., not at the 3′ or 5′-ends) a mismatch, e.g., a double mismatch, is located; the more cleavage efficiency is affected. Accordingly, by choosing mismatch positions along the spacer sequence, cleavage efficiency can be modulated. For example, if less than 100% cleavage of targets is desired (e.g., in a cell population), 1 or 2 mismatches between spacer and target sequence can be introduced in the spacer sequences.

Type VI CRISPR-Cas effector proteins have been demonstrated to employ more than one RNA guide, thus enabling the ability of these effectors, and systems and complexes that include them, to target multiple nucleic acids. In some embodiments, the CRISPR systems described herein include multiple RNA guides (e.g., two, three, four, five, six, seven, eight, nine, ten, fifteen, twenty, thirty, forty, or more) RNA guides. In some embodiments, the CRISPR systems described herein include a single RNA strand or a nucleic acid encoding a single RNA strand, wherein the RNA guides are arranged in tandem. The single RNA strand can include multiple copies of the same RNA guide, multiple copies of distinct RNA guides, or combinations thereof. The processing capability of the Class 1, Type VI CRISPR-Cas effector proteins described herein enables these effectors to be able to target multiple target nucleic acids (e.g., target RNAs) without a loss of activity. In some embodiments, the Class 1, Type VI CRISPR-Cas effector proteins may be delivered in complex with multiple RNA guides directed to different target RNA. In some embodiments, the Class 1, Type VI CRISPR-Cas effector proteins may be co-delivered with multiple RNA guides, each specific for a different target nucleic acid. Methods of multiplexing using CRISPR-associated proteins are described, for example, in U.S. Pat. No. 9,790,490 B2, and EP 3009511 B1, the entire contents of each of which are expressly incorporated herein by reference.

The spacer length of crRNAs can range from about 10-60 nucleotides, such as 15-50 nucleotides, 20-50 nucleotides, 25-50 nucleotide, or 19-50 nucleotides. In some embodiments, the spacer length of a guide RNA is at least 16 nucleotides, at least 17 nucleotides, at least 18 nucleotides, at least 19 nucleotides, at least 20 nucleotides, at least 21 nucleotides, or at least 22 nucleotides. In some embodiments, the spacer length is from 15 to 17 nucleotides (e.g., 15, 16, or 17 nucleotides), from 17 to 20 nucleotides (e.g., 17, 18, 19, or 20 nucleotides), from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 45 nucleotides (e.g., 30, 31, 32, 33, 34, 35, 36, 37, 38, 39, 40, 41, 42, 43, 44, or 45 nucleotides), from 30 or 35 to 40 nucleotides, from 41 to 45 nucleotides, from 45 to 50 nucleotides (e.g., 45, 46, 47, 48, 49, or 50 nucleotides), or longer. In some embodiments, the spacer length is from about 15 to about 42 nucleotides.

In some embodiments, the spacer sequence is between 15-100 nucleotides, 15-80 nucleotides, 15-60 nucleotides, between 25-50 nucleotides, between 30-50 nucleotides, about 100 nucleotides, about 80 nucleotides, about 60 nucleotides, about 55 nucleotides, about 50 nucleotides, about 45 nucleotides, about 40 nucleotides, about 35 nucleotides, about 30 nucleotides, about 20 nucleotides, or about 15 nucleotides in length.

In some embodiments, the direct repeat length of the guide RNA is 15-36 nucleotides, is at least 16 nucleotides, is from 16 to 20 nucleotides (e.g., 16, 17, 18, 19, or 20 nucleotides), is from 20-30 nucleotides (e.g., 20, 21, 22, 23, 24, 25, 26, 27, 28, 29, or 30 nucleotides), is from 30-40 nucleotides (e.g., 30, 31, 32, 33, 34, 35, 36, 37, 38, 39, or 40 nucleotides), or is about 36 nucleotides (e.g., 33, 34, 35, 36, 37, 38, or 39 nucleotides). In some embodiments, the direct repeat length of the guide RNA is 36 nucleotides.

In some embodiments, the overall length of the crRNA/guide RNA is about 36 nucleotides longer than any one of the spacer sequence lengths described herein above. For example, the overall length of the crRNA/guide RNA may be between 45-86 nucleotides, or 60-86 nucleotides, 62-86 nucleotides, or 63-86 nucleotides.

Guide RNAs (e.g., crRNAs) can be generated as components of inducible systems. The inducible nature of the systems allows for spatio-temporal control of gene editing or gene expression. In some embodiments, the stimuli for the inducible systems include, e.g., electromagnetic radiation, sound energy, chemical energy, and/or thermal energy.

In some embodiments, the transcription of guide RNA (e.g., crRNA) can be modulated by inducible promoters, e.g., tetracycline or doxycycline controlled transcriptional activation (Tet-On and Tet-Off expression systems), hormone inducible gene expression systems (e.g., ecdysone inducible gene expression systems), and arabinose-inducible gene expression systems. Other examples of inducible systems include, e.g., small molecule two-hybrid transcription activations systems (FKBP, ABA, etc.), light inducible systems (Phytochrome, LOV domains, or cryptochrome), or Light Inducible Transcriptional Effector (LITE). These inducible systems are described, e.g., in WO 2016205764 and U.S. Pat. No. 8,795,965, both of which are incorporated herein by reference in the entirety.

Chemical modifications can be applied to the crRNA's phosphate backbone, sugar, and/or base. Backbone modifications such as phosphorothioates modify the charge on the phosphate backbone and aid in the delivery and nuclease resistance of the oligonucleotide (see, e.g., Eckstein, “Phosphorothioates, essential components of therapeutic oligonucleotides,” Nucl. Acid Ther., 24, pp. 374-387, 2014); modifications of sugars, such as 2′-O-methyl (2′-OMe), 2′-F, and locked nucleic acid (LNA), enhance both base pairing and nuclease resistance (see, e.g., Allerson et al. “Fully 2′-modified oligonucleotide duplexes with improved in vitro potency and stability compared to unmodified small interfering RNA,” J. Med. Chem. 48.4: 901-904, 2005). Chemically modified bases such as 2-thiouridine or N6-methyladenosine, among others, can allow for either stronger or weaker base pairing (see, e.g., Bramsen et al., “Development of therapeutic-grade small interfering RNAs by chemical engineering,” Front. Genet., 2012 Aug. 20; 3:154). Additionally, RNA is amenable to both 5′ and 3′ end conjugations with a variety of functional moieties including fluorescent dyes, polyethylene glycol, or proteins.

A wide variety of modifications can be applied to chemically synthesized crRNA molecules. For example, modifying an oligonucleotide with a 2′-OMe to improve nuclease resistance can change the binding energy of Watson-Crick base pairing. Furthermore, a 2′-OMe modification can affect how the oligonucleotide interacts with transfection reagents, proteins or any other molecules in the cell. The effects of these modifications can be determined by empirical testing.

In some embodiments, the crRNA includes one or more phosphorothioate modifications. In some embodiments, the crRNA includes one or more locked nucleic acids for the purpose of enhancing base pairing and/or increasing nuclease resistance.

A summary of these chemical modifications can be found, e.g., in Kelley et al., “Versatility of chemically synthesized guide RNAs for CRISPR-Cas9 genome editing,” J. Biotechnol. 233:74-83, 2016; WO 2016205764; and U.S. Pat. No. 8,795,965 B2; each which is incorporated by reference in its entirety.

The sequences and the lengths of the RNA guides (e.g., crRNAs) described herein can be optimized. In some embodiments, the optimized length of an RNA guide can be determined by identifying the processed form of crRNA (i.e., a mature crRNA), or by empirical length studies for crRNA tetraloops.

The crRNAs can also include one or more aptamer sequences. Aptamers are oligonucleotide or peptide molecules have a specific three-dimensional structure and can bind to a specific target molecule. The aptamers can be specific to gene effectors, gene activators, or gene repressors. In some embodiments, the aptamers can be specific to a protein, which in turn is specific to and recruits and/or binds to specific gene effectors, gene activators, or gene repressors. The effectors, activators, or repressors can be present in the form of fusion proteins. In some embodiments, the guide RNA has two or more aptamer sequences that are specific to the same adaptor proteins. In some embodiments, the two or more aptamer sequences are specific to different adaptor proteins. The adaptor proteins can include, e.g., MS2, PP7, Qβ, F2, GA, fr, JP501, M12, R17, BZ13, JP34, JP500, KU1, M11, MX1, TW18, VK, SP, FI, ID2, NL95, TW19, AP205, ϕkCb5, ϕkCb8r, ϕkCb12r, ϕkCb23r, 7s, and PRR1. Accordingly, in some embodiments, the aptamer is selected from binding proteins specifically binding any one of the adaptor proteins as described herein. In some embodiments, the aptamer sequence is a MS2 binding loop (5′-ggcccAACAUGAGGAUCACCCAUGUCUGCAGgggcc-3′, SEQ ID NO: 79). In some embodiments, the aptamer sequence is a QBeta binding loop (5′-ggcccAUGCUGUCUAAGACA GCAUgggcc-3′, SEQ ID NO: 80). In some embodiments, the aptamer sequence is a PP7 binding loop (5′-ggcccUAAGGGUUUAUAUGGAAACCCUUAgggcc-3′ (SEQ ID NO: 81). A detailed description of aptamers can be found, e.g., in Nowak et al., “Guide RNA engineering for versatile Cas9 functionality,” Nucl. Acid. Res., 44(20):9555-9564, 2016; and WO 2016205764, which are incorporated herein by reference in their entirety.

In certain embodiments, the methods make use of chemically modified guide RNAs. Examples of guide RNA chemical modifications include, without limitation, incorporation of 2′-O-methyl (M), 2′-O-methyl 3′-phosphorothioate (MS), or 2′-O-methyl 3′-thioPACE (MSP) at one or more terminal nucleotides. Such chemically modified guide RNAs can comprise increased stability and increased activity as compared to unmodified guide RNAs, though on-target vs. off-target specificity is not predictable. See, Hendel, Nat Biotechnol. 33(9):985-9, 2015, incorporated by reference). Chemically modified guide RNAs may further include, without limitation, RNAs with phosphorothioate linkages and locked nucleic acid (LNA) nucleotides comprising a methylene bridge between the 2′ and 4′ carbons of the ribose ring.

The disclosure also encompasses methods for delivering multiple nucleic acid components, wherein each nucleic acid component is specific for a different target locus of interest thereby modifying multiple target loci of interest. The nucleic acid component of the complex may comprise one or more protein-binding RNA aptamers. The one or more aptamers may be capable of binding a bacteriophage coat protein. The bacteriophage coat protein may be selected from the group comprising Qβ, F2, GA, fr, JP501, MS2, M12, R17, BZ13, JP34, JP500, KU1, M11, MX1, TW18, VK, SP, FI, ID2, NL95, TW19, AP205, ϕCb5, ϕCb8r, ϕCb23r, 7s and PRR1. In certain embodiments, the bacteriophage coat protein is MS2.

### 5. Target RNA

The target RNA can be any RNA molecule of interest, including naturally-occurring and engineered RNA molecules.

In certain embodiments, the target RNA is encoded by a eukaryotic DNA. In certain embodiments, the eukaryotic DNA is a non-human mammalian DNA, a non-human primate DNA, a human DNA, a plant DNA, an insect DNA, a bird DNA, a reptile DNA, a rodent DNA, a fish DNA, a worm/nematode DNA, a yeast DNA.

In certain embodiments, the target RNA can be an mRNA, a tRNA, a ribosomal RNA (rRNA), a microRNA (miRNA), an interfering RNA (siRNA), a ribozyme, a riboswitch, a satellite RNA, a microswitch, a microzyme, or a viral RNA.

In certain embodiments, the target RNA is an mRNA.

In some embodiments, the target nucleic acid is associated with a condition or disease (e.g., an infectious disease, a genetic disease or disorder, or a cancer).

Thus, in some embodiments, the systems described herein can be used to treat a condition or disease by targeting these nucleic acids. For instance, the target nucleic acid associated with a condition or disease may be an RNA molecule that is overexpressed in a diseased cell (e.g., a cancer or tumor cell). The target nucleic acid may also be a toxic RNA and/or a mutated RNA (e.g., an mRNA molecule having a splicing defect or a mutation). The target nucleic acid may also be an RNA that is specific for a particular microorganism (e.g., a pathogenic bacteria).

### 6. Complex

In an aspect of the disclosure, it is provided a fusion/conjugate comprising a crRNA binding polypeptide of the disclosure (comprising a crRNA binding domain that retains the crRNA binding domain but substantially lacks ability to process DR sequence) linked to (e.g., fused with) an RNA base editor, which fusion/conjugate is in turn complexed with a guide RNA comprising a spacer sequence for hybridizing with a target RNA, wherein the spacer sequence is flanked by two DR sequences compatible with the crRNA binding domain.

In certain embodiments, the guide RNA comprises a spacer sequence designed to be at least partially complementary to a target RNA, and a DR sequence flanking both ends of the spacer sequence.

In certain embodiments, the complex further comprises the target RNA bound by the guide RNA.

In certain embodiments, the DR sequence is not naturally occurring/existing, i.e., not any one of SEQ ID NOs: 8-14, 126-140, and 152-162, due to, for example, addition, deletion, and/or substitution of at least one nucleotide base in the wild-type sequence. In certain embodiments, the spacer sequence is not naturally occurring, in that it is not present or encoded by any spacer sequences present in the wild-type CRISPR locus of a prokaryote in which the subject Cas13e or Cas13f exists. The spacer sequence may be not naturally existing when it is not 100% complementary to a naturally-occurring bacterialphage nucleic acid.

In a related aspect, the disclosure also provides a cell comprising any of the complex of the disclosure.

In certain embodiments, the cell is a prokaryote.

In certain embodiments, the cell is a eukaryote. When the cell is a eukaryote, the complex in the eukaryotic cell can be a naturally existing Cas13 or CasPR complex in a prokaryote from which the Cas13 or CasPR is isolated.

### 7. Polynucleotides and Vectors

In an aspect of the disclosure, it is provided nucleic acids or polynucleotides encoding the protein component (e.g., the fusion of the heterologous functional domain and the crRNA binding domain-containing polypeptide of the disclosure) and the guide RNA (e.g., crRNA) component described herein.

In some embodiments, the nucleic acid or polynucleotide is isolated.

In some embodiments, the nucleic acid is a synthetic nucleic acid. In some embodiments, the nucleic acid is a DNA molecule. In some embodiments, the nucleic acid is an RNA molecule (e.g., an mRNA molecule encoding the protein component). In some embodiments, the mRNA is capped, polyadenylated, substituted with 5-methyl cytidine, substituted with pseudouridine, or a combination thereof.

Thus, one aspect of the disclosure provides a polynucleotide comprising a first and a second polynucleotides encoding the protein component and the gRNA component of the CRISPR-Cas system, the gRNA, the modified Cas13 protein, the fusion protein, or the CRISPR-Cas13 system of the disclosure, respectively.

In certain embodiments, the transcription of the protein component and the transcription of the guide RNA are under the control of separate or independent promoters and/or enhancers.

In some embodiments, the first polynucleotide is operably linked to a regulatory element (e.g., a promoter and/or an enhancer). In some embodiments, the promoter is a constitutive promoter. In some embodiments, the promoter is an inducible promoter. In some embodiments, the promoter is a cell-specific promoter. In some embodiments, the promoter is an organism-specific promoter.

In certain embodiments, the transcription of the protein component is under the control of a constitutive promoter, an inducible promoter, a ubiquitous promoter, or a tissue specific promoter.

Suitable promoters are known in the art and include, for example, a pol I promoter, a pol II promoter, a pol III promoter, a T7 promoter, a U6 promoter, a H1 promoter, retroviral Rous sarcoma virus LTR promoter, a cytomegalovirus (CMV) promoter, a SV40 promoter, a dihydrofolate reductase promoter, and a β-actin promoter. For example, a U6 promoter can be used to regulate the expression of a guide RNA molecule described herein.

In certain embodiments, the constitutive promoter is an RNA Pol II promoter, such as a CMV promoter, a CB promoter, a Cbh promoter, an EFS promoter, or a CAG promoter.

In certain embodiments, the promoter is a ubiquitous, tissue-specific, cell-type specific, constitutive, or inducible promoter; optionally, wherein the promoter comprises a promoter selected from the group consisting of: a Cbh promoter, a Cba promoter, a pol I promoter, a pol II promoter, a pol III promoter, a T7 promoter, a U6 promoter, a H1 promoter, a retroviral Rous sarcoma virus LTR promoter, a cytomegalovirus (CMV) promoter, a SV40 promoter, a dihydrofolate reductase promoter, a β-actin promoter, an elongation factor 1a short (EFS) promoter, a β glucuronidase (GUSB) promoter, a cytomegalovirus (CMV) immediate-early (Ie) enhancer and/or promoter, a chicken β-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nβ2 promoter, a preproenkephalin (PPE) promoter, an enkephalin (Enk) promoter, an excitatory amino acid transporter 2 (EAAT2) promoter, a glial fibrillary acidic protein (GFAP) promoter, and a myelin basic protein (MBP) promoter.

In certain embodiments, the transcription of the gRNA component is under the control of an RNA Pol III promoter, such as a U6 promoter.

In certain embodiments, the promoter is a ubiquitous, tissue-specific, cell-type specific, constitutive, or inducible promoter; optionally selected from a group consisting of a Cbh promoter, a Cba promoter, a pol I promoter, a pol II promoter, a pol III promoter, a T7 promoter, a U6 promoter, a H1 promoter, a retroviral Rous sarcoma virus LTR promoter, a cytomegalovirus (CMV) promoter, a SV40 promoter, a dihydrofolate reductase promoter, a β-actin promoter, an elongation factor 1α short (EFS) promoter, a β glucuronidase (GUSB) promoter, a cytomegalovirus (CMV) immediate-early (Ie) enhancer and/or promoter, a chicken β-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nβ2 promoter, a preproenkephalin (PPE) promoter, an enkephalin (Enk) promoter, an excitatory amino acid transporter 2 (EAAT2) promoter, a glial fibrillary acidic protein (GFAP) promoter, and a myelin basic protein (MBP) promoter; optionally wherein the promoter is an RNA pol III promoter.

In certain embodiments, the RNA pol III promoter is U6, H1, 7SK, or a variant thereof.

In certain embodiments, the first polynucleotide is codon-optimized for expression in a cell, such as a eukaryotic cell, or a mammalian (e.g., human) cell.

In some embodiments, the nucleic acid(s) are present in a vector (e.g., a viral vector or a phage).

Thus, a related aspect of the disclosure provides a vector comprising the polynucleotide of the disclosure.

In certain embodiments, the vector is a cloning vector, or an expression vector. The vectors can be plasmids, phagemids, Cosmids, etc. The vectors may include one or more regulatory elements that allow for the propagation of the vector in a cell of interest (e.g., a bacterial cell or a mammalian cell). In some embodiments, the vector includes a nucleic acid encoding the CRISPR-Cas system described herein. In some embodiments, the vector includes multiple nucleic acids, each encoding a component of the CRISPR-Cas system described herein.

In one aspect, the present disclosure provides nucleic acid sequences that are at least 50%, 55%, 60%, 65%, 70%, 75%, 80%, 85%, 90%, 91%, 92%, 93%, 94%, 95%, 96%, 97%, 98%, 99%, or 100% identical to the nucleic acid sequences described herein, i.e., nucleic acid sequences encoding the Cas proteins, derivatives, functional fragments, or guide/crRNA, including the DR sequences of SEQ ID NOs: 8-14, 126-140, and 152-162.

In another aspect, the present disclosure also provides nucleic acid sequences encoding amino acid sequences that are at least 50%, 55%, 60%, 65%, 70%, 75%, 80%, 85%, 90%, 91%, 92%, 93%, 94%, 95%, 96%, 97%, 98%, 99%, or 100% identical to the amino acid sequences described herein, such as SEQ ID NOs: 1-7, 111-125, and 141-151, or any of the CRISPR-Cas system described herein.

In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as the sequences described herein. In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from the sequences described herein.

In related embodiments, the disclosure provides amino acid sequences having at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as the sequences described herein. In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from the sequences described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In general, the length of a reference sequence aligned for comparison purposes should be at least 80% of the length of the reference sequence, and in some embodiments is at least 90%, 95%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

The proteins described herein (e.g., CRISPR-Cas system) can be delivered or used as either nucleic acid molecules or polypeptides.

In certain embodiments, the nucleic acid molecule encoding the CRISPR-Cas system are codon-optimized for expression in a host cell or organism. The host cell may include established cell lines (such as 293T cells) or isolated primary cells. The nucleic acid can be codon optimized for use in any organism of interest, in particular human cells or bacteria. For example, the nucleic acid can be codon-optimized for any prokaryotes (such as E. coli), or any eukaryotes such as human and other non-human eukaryotes including yeast, worm, insect, plants and algae (including food crop, rice, corn, vegetables, fruits, trees, grasses), vertebrate, fish, non-human mammal (e.g., mice, rats, rabbits, dogs, birds (such as chicken), livestock (cow or cattle, pig, horse, sheep, goat etc.), or non-human primates). Codon usage tables are readily available, for example, at the “Codon Usage Database” available at www.kazusa.orjp/codon/, and these tables can be adapted in a number of ways. See Nakamura et al., Nucl. Acids Res. 28:292, 2000 (incorporated herein by reference in its entirety). Computer algorithms for codon optimizing a particular sequence for expression in a particular host cell are also available, such as Gene Forge (Aptagen; Jacobus, Pa.).

An example of a codon optimized sequence is in this instance a sequence optimized for expression in a eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a Cas correspond to the most frequently used codon for a particular amino acid.

In certain embodiments, the vector is a retroviral vector, a phage vector, an adenoviral vector, a herpes simplex viral (HSV) vector, an AAV vector, or a lentiviral vector.

In certain embodiments, the vector is an AAV vector comprising the polynucleotide of the disclosure flanked by a 5′ ITR (such as an AAV2 5′ ITR) and a 3′ ITR (such as an AAV2 3′ ITR).

In certain embodiments, in the vector of the disclosure, the polynucleotide of the disclosure further comprises an intron and/or an exon that promotes transcription of the protein component of the CRISPR-Cas system.

In certain embodiments, the vector of the disclosure further comprises a coding sequence for a polyA signal sequence operably linked to the first polynucleotide encoding the protein component of the CRISPR-Cas system.

In certain embodiments, the vector of the disclosure further comprises a 5′ UTR and/or a 3′ UTR coding sequence in the first polynucleotide encoding the protein component of the CRISPR-Cas system.

In certain embodiments, the vector of the disclosure further comprises a WPRE sequence.

In a related aspect, the disclosure also provides a recombinant AAV (rAAV) viral particle comprising the AAV vector of the disclosure, encapsidated within a capsid of the serotype of AAV1, AAV2, AAV3A, AAV3B, AAV4, AAV5, AAV6, AAV7, AAVrh74, AAV8, AAV9, AAV10, AAV11, AAV12, AAV13, AAV.DJ, AAV.PHP.eB, or a mutant thereof.

### 8. Delivery

Through this disclosure and the knowledge in the art, the CRISPR-Cas system described herein, or any of the components thereof described herein (Cas proteins, derivatives, functional fragments or the various fusions or adducts thereof, and guide RNA/crRNA), nucleic acid molecules thereof, and/or nucleic acid molecules encoding or providing components thereof, can be delivered by various delivery systems such as vectors, e.g., plasmids and viral delivery vectors, using any suitable means in the art. Such methods include (and are not limited to) electroporation, lipofection, microinjection, transfection, sonication, gene gun, etc.

In an aspect of the disclosure, it is provided a delivery system comprising (1) a delivery vehicle, and (2) the CRISPR-Cas system, the gRNA, the modified Cas13 protein, the fusion protein, or the CRISPR-Cas13 system of the disclosure, the polynucleotide of the disclosure, the vector of the disclosure, or the rAAV viral particle of the disclosure.

In certain embodiments, the CRISPR-Cas system and/or any of the RNAs (e.g., guide RNAs or crRNAs) and/or accessory proteins can be delivered using suitable vectors, e.g., plasmids or viral vectors, such as adeno-associated viruses (AAV), lentiviruses, adenoviruses, retroviral vectors, and other viral vectors, or combinations thereof. The proteins and one or more crRNAs can be packaged into one or more vectors, e.g., plasmids or viral vectors. For bacterial applications, the nucleic acids encoding any of the components of the CRISPR-Cas system described herein can be delivered to the bacteria using a phage. Exemplary phages, include, but are not limited to, T4 phage, Mu, λ, phage, T5 phage, T7 phage, T3 phage, Φ29, M13, MS2, Qβ, and ΦX174.

In some embodiments, the vectors, e.g., plasmids or viral vectors, are delivered to the tissue of interest by, e.g., intramuscular injection, intravenous administration, transdermal administration, intranasal administration, oral administration, or mucosal administration. Such delivery may be either via a single dose, or multiple doses. One skilled in the art understands that the actual dosage to be delivered herein may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

In certain embodiments, the delivery is via adenoviruses, which can be at a single dose containing at least 1×10⁵ particles (also referred to as particle units, pu) of adenoviruses. In some embodiments, the dose preferably is at least about 1×10⁶ particles, at least about 1×10⁷ particles, at least about 1×10⁸ particles, and at least about 1×10⁹ particles of the adenoviruses. The delivery methods and the doses are described, e.g., in WO 2016205764 A1 and U.S. Pat. No. 8,454,972 B2, both of which are incorporated herein by reference in the entirety.

In some embodiments, the delivery is via plasmids. The dosage can be a sufficient number of plasmids to elicit a response. In some cases, suitable quantities of plasmid DNA in plasmid compositions can be from about 0.1 to about 2 mg. Plasmids will generally include (i) a promoter; (ii) a sequence encoding a nucleic acid-targeting CRISPR-Cas system, operably linked to a promoter (e.g., the same promoter or a different promoter); (iii) a selectable marker; (iv) an origin of replication; and (v) a transcription terminator downstream of and operably linked to (ii). The plasmids can also encode the RNA components of the CRISPR-Cas system, but one or more of these may instead be encoded on different vectors. The frequency of administration is within the ambit of the medical or veterinary practitioner (e.g., physician, veterinarian), or a person skilled in the art.

In another embodiment, the delivery is via liposomes or lipofection formulations and the like, and can be prepared by methods known to those skilled in the art. Such methods are described, for example, in WO 2016205764 and U.S. Pat. Nos. 5,593,972; 5,589,466; and 5,580,859; each of which is incorporated herein by reference in its entirety.

In some embodiments, the delivery is via nanoparticles (e.g., lipid nanoparticle (LNP)) or exosomes. For example, exosomes have been shown to be particularly useful in delivery RNA.

Further means of introducing one or more components of the CRISPR-Cas system to the cell is by using cell penetrating peptides (CPP). In some embodiments, a cell penetrating peptide is linked to the CRISPR-Cas system. In some embodiments, the CRISPR-Cas system and/or guide RNAs are coupled to one or more CPPs to effectively transport them inside cells (e.g., plant protoplasts). In some embodiments, the CRISPR-Cas system and/or guide RNA(s) are encoded by one or more circular or non-circular DNA molecules that are coupled to one or more CPPs for cell delivery.

CPPs are short peptides of fewer than 35 amino acids derived either from proteins or from chimeric sequences capable of transporting biomolecules across cell membrane in a receptor independent manner. CPPs can be cationic peptides, peptides having hydrophobic sequences, amphipathic peptides, peptides having proline-rich and anti-microbial sequences, and chimeric or bipartite peptides. Examples of CPPs include, e.g., Tat (which is a nuclear transcriptional activator protein required for viral replication by HIV type 1), penetratin, Kaposi fibroblast growth factor (FGF) signal peptide sequence, integrin f33 signal peptide sequence, polyarginine peptide Args sequence, Guanine rich-molecular transporters, and sweet arrow peptide. CPPs and methods of using them are described, e.g., in Hällbrink et al., “Prediction of cell-penetrating peptides,” Methods Mol. Biol., 2015; 1324:39-58; Ramakrishna et al., “Gene disruption by cell-penetrating peptide-mediated delivery of Cas9 protein and guide RNA,” Genome Res., 2014 June; 24(6):1020-7; and WO 2016205764 A1; each of which is incorporated herein by reference in its entirety.

Various delivery methods for the CRISPR-Cas system described herein are also described, e.g., in U.S. Pat. No. 8,795,965, EP 3009511, WO 2016205764, and WO 2017070605; each of which is incorporated herein by reference in its entirety.

In some embodiments, the crRNA binding polypeptide and/or the heterologous functional domain and/or the gRNA as described herein is delivered in the form of a rAAV particle packaging a RNA encoding the crRNA binding polypeptide and/or the heterologous functional domain and/or the gRNA by means of a AAV packaging system capable of packaging an RNA as described in, for example, PCT/CN2022/075366, which is incorporated herein by reference in its entirety. In some embodiments, the polynucleotide coding sequence is an RNA coding sequence.

Instead of packaging a single strand (ss)DNA sequence as a vector genome of a AAV particle, systems and methods of packaging an RNA sequence as a vector genome into a AAV particle is recently developed and applicable herein. See PCT/CN2022/075366, which is incorporated herein by reference in its entirety.

When the vector genome is RNA as in, for example, PCT/CN2022/075366, for simplicity of description and claiming, sequence elements described herein for DNA vector genomes, when present in RNA vector genomes, should generally be considered to be applicable for the RNA vector genomes except that the deoxyribonucleotides in the DNA sequence are the corresponding ribonucleotides in the RNA sequence (e.g., dT is equivalent to U, and dA is equivalent to A) and/or the the element in the DNA sequence is replaced with the corresponding element with a corresponding function in the RNA sequence or omitted because its function is unnecessary in the RNA sequence and/or an additional element necessary for the RNA vector genome is introduced.

As used herein, a coding sequence, e.g., as a sequence element of AAV vector genomes herein, is construed, understood, and considered as covering and covers both a DNA coding sequence and an RNA coding sequence. When it is a DNA coding sequence, an RNA sequence can be transcribed from the DNA coding sequence, and optionally further a protein can be translated from the transcribed RNA sequence as necessary. When it is an RNA coding sequence, the RNA coding sequence per se can be an RNA sequence for use (although it seems that the RNA coding sequence does not encode something), or an RNA sequence can be produced from the RNA coding sequence, e.g., by RNA processing (although it seems that the RNA coding sequence does not encode something), or a protein can be translated from the RNA coding sequence.

For example, a (e.g., Cas13, NLS) coding sequence (encoding a (e.g., Cas13, NLS) polypeptide) covers either a (e.g., Cas13, NLS) DNA coding sequence from which a (e.g., Cas13, NLS) polypeptide is expressed (indirectly via transcription and translation) or a (e.g., Cas13, NLS) RNA coding sequence from which a (e.g., Cas13, NLS) polypeptide is translated (directly).

For example, a (e.g., sgRNA) coding sequence (encoding an RNA (e.g., a sgRNA) sequence) covers either a (e.g., sgRNA) DNA coding sequence from which an RNA sequence (e.g., a sgRNA sequence or array) is transcribed or a (e.g., sgRNA) RNA coding sequence (1) which per se is the RNA sequence (e.g., a sgRNA sequence or array) for use, or (2) from which a sgRNA sequence or array is produced, e.g., by RNA processing.

In some embodiments for RNA AAV vector genomes, 5′-ITR and/or 3′-ITR as DNA packaging signals would be unnecessary and can be omitted, while RNA packaging signals can be introduced.

In some embodiments for AAV RNA vector genomes, promoters to drive transcription of DNA sequences would be unnecessary and can be omitted at least partly.

In some embodiments for AAV RNA vector genomes, polyA signal sequence would be unnecessary and can be omitted, while a polyA tail can be introduced.

Similarly, other DNA elements of AAV DNA vector genomes can be either omitted or replaced with corresponding RNA elements and/or new RNA elements can be introduced, in order to adapt to the strategy of delivering an RNA vector genome by rAAV particles.

### 9. Cells and Progenies Thereof

In certain embodiments, the methods of the disclosure can be used to introduce the CRISPR-Cas system described herein into a cell, and cause the cell and/or its progeny to alter the production of one or more cellular produces, such as antibody, starch, ethanol, or any other desired products. Such cells and progenies thereof are within the scope of the disclosure.

In an aspect of the disclosure, it is provided a cell or a progeny thereof, comprising the CRISPR-Cas system, the gRNA, the modified Cas13 protein, the fusion protein, or the CRISPR-Cas13 system of the disclosure, the polynucleotide of the disclosure, the vector of the disclosure, the rAAV viral particle of the disclosure, or the delivery system of the disclosure.

In certain embodiments, the methods and/or the CRISPR-Cas system described herein lead to modification of the translation and/or transcription of one or more RNA products of the cells. For example, the modification may lead to increased transcription/translation/expression of the RNA product. In other embodiments, the modification may lead to decreased transcription/translation/expression of the RNA product.

In certain embodiments, the cell is a prokaryotic cell.

In certain embodiments, the cell is a eukaryotic cell, such as a mammalian cell, including a human cell (a primary human cell or an established human cell line). In certain embodiments, the cell is a non-human mammalian cell, such as a cell from a non-human primate (e.g., monkey), a cow/bull/cattle, sheep, goat, pig, horse, dog, cat, rodent (such as rabbit, mouse, rat, hamster, etc.). In certain embodiments, the cell is from fish (such as salmon), bird (such as poultry bird, including chick, duck, goose), reptile, shellfish (e.g., oyster, claim, lobster, shrimp), insect, worm, yeast, etc. In certain embodiments, the cell is from a plant, such as monocot or dicot. In certain embodiment, the plant is a food crop such as barley, cassava, cotton, groundnuts or peanuts, maize, millet, oil palm fruit, potatoes, pulses, rapeseed or canola, rice, rye, sorghum, soybeans, sugar cane, sugar beets, sunflower, and wheat. In certain embodiment, the plant is a cereal (barley, maize, millet, rice, rye, sorghum, and wheat). In certain embodiment, the plant is a tuber (cassava and potatoes). In certain embodiment, the plant is a sugar crop (sugar beets and sugar cane). In certain embodiment, the plant is an oil-bearing crop (soybeans, groundnuts or peanuts, rapeseed or canola, sunflower, and oil palm fruit). In certain embodiment, the plant is a fiber crop (cotton). In certain embodiment, the plant is a tree (such as a peach or a nectarine tree, an apple or pear tree, a nut tree such as almond or walnut or pistachio tree, or a citrus tree, e.g., orange, grapefruit or lemon tree), a grass, a vegetable, a fruit, or an algae. In certain embodiment, the plant is a nightshade plant; a plant of the genus Brassica; a plant of the genus Lactuca; a plant of the genus Spinacia; a plant of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.

A related aspect provides cells or progenies thereof modified by the methods of the disclosure using the CRISPR-Cas system described herein.

In certain embodiments, the cell is modified in vitro, in vivo, or ex vivo. In certain embodiments, the cell is a stem cell.

In another aspect of the disclosure, it is provided a non-human multicellular eukaryote comprising the cell or a progeny thereof of the disclosure.

In some embodiments, the non-human multicellular eukaryote is an animal (e.g., rodent or primate) model for a human genetic disorder.

### 10. Pharmaceutical Compositions and Kits

In an aspect of the disclosure, it is provided a pharmaceutical composition comprising:

(i) the CRISPR-Cas system, the gRNA, the modified Cas13 protein, the fusion protein, or the CRISPR-Cas13 system of the disclosure, the polynucleotide of the disclosure, the vector of the disclosure, the rAAV viral particle of the disclosure, the delivery system of the disclosure, or the cell or a progeny thereof of the disclosure; and

(ii) a pharmaceutically acceptable excipient.

In another aspect of the disclosure, it is provided a kit comprising

(i) the CRISPR-Cas system, the gRNA, the modified Cas13 protein, the fusion protein, or the CRISPR-Cas13 system of the disclosure, the polynucleotide of the disclosure, the vector of the disclosure, the rAAV viral particle of the disclosure, the delivery system of the disclosure, the cell or a progeny thereof of the disclosure, or the pharmaceutical composition of the disclosure; and

(ii) an instruction for using component (i).

Another aspect of the disclosure provides a pharmaceutical composition or a kit, comprising any two or more components of the subject CRISPR-Cas system described herein, such as the modified/truncated Cas13e and Cas13f proteins, derivatives, functional fragments or the various fusions or adducts thereof, guide RNA/crRNA, complexes thereof, vectors encompassing the same, or host encompassing the same.

In certain embodiments, the kit further comprises an instruction to use the components encompassed therein, and/or instructions for combining with additional components that may be available elsewhere.

In certain embodiments, the kit further comprises one or more nucleotides, such as nucleotide(s) corresponding to those useful to insert the guide RNA coding sequence into a vector and operably linking the coding sequence to one or more control elements of the vector.

In certain embodiments, the pharmaceutical composition or kit further comprises one or more buffers that may be used to dissolve any of the components, and/or to provide suitable reaction conditions for one or more of the components. Such buffers may include one or more of PBS, HEPES, Tris, MOPS, Na₂CO₃, NaHCO₃, NaB, or combinations thereof. In certain embodiments, the reaction condition includes a proper pH, such as a basic pH. In certain embodiments, the pH is between 7-10.

In certain embodiments, any one or more of the kit components may be stored in a suitable container.

### 11. Methods of Using

### Tracking and Labeling of Nucleic Acids

Cellular processes depend on a network of molecular interactions among proteins, RNAs, and DNAs. Accurate detection of protein-DNA and protein-RNA interactions is key to understanding such processes. In vitro proximity labeling techniques employ an affinity tag combined with, a reporter group, e.g., a photoactivatable group, to label polypeptides and RNAs in the vicinity of a protein or RNA of interest in vitro. After UV irradiation, the photoactivatable groups react with proteins and other molecules that are in close proximity to the tagged molecules, thereby labelling them. Labelled interacting molecules can subsequently be recovered and identified.

The targeting moiety of the subject CRISPR-Cas system can for instance be used to target probes to selected RNA sequences. These applications can also be applied in animal models for in vivo imaging of diseases or difficult-to culture cell types. The methods of tracking and labeling of nucleic acids are described, e.g., in U.S. Pat. No. 8,795,965, WO 2016205764, and WO 2017070605; each of which is incorporated herein by reference herein in its entirety.

RNA Isolation, Purification, Enrichment, and/or Depletion

The CRISPR systems (e.g., CRISPR-associated proteins) described herein can be used to isolate and/or purify the RNA. The modified Cas effector protein still retains the ability to bind to guide RNA with a DR sequence, and can be fused to an affinity tag that can be used to isolate and/or purify the RNA-CRISPR-associated protein complex. These applications are useful, e.g., for the analysis of gene expression profiles in cells.

In some embodiments, the CRISPR-Cas system (e.g., CRISPR-Cas13 system) of the disclosure can be used to target a specific noncoding RNA (ncRNA) thereby blocking its activity. In some embodiments, the CRISPR-associated proteins can be used to specifically enrich a particular RNA (including but not limited to increasing stability, etc.), or alternatively, to specifically deplete a particular RNA (e.g., particular splice variants, isoforms, etc.).

These methods are described, e.g., in U.S. Pat. No. 8,795,965, WO 2016205764, and WO 2017070605; each of which is incorporated herein by reference herein in its entirety.

### RNA-Related Applications

The CRISPR-Cas system described herein can have various RNA-related applications, e.g., modulating gene expression, degrading an RNA molecule, inhibiting RNA expression, screening RNA or RNA products, determining functions of lincRNA or non-coding RNA, inducing cell dormancy, inducing cell cycle arrest, reducing cell growth and/or cell proliferation, inducing cell anergy, inducing cell apoptosis, inducing cell necrosis, inducing cell death, and/or inducing programmed cell death. A detailed description of these applications can be found, e.g., in WO 2016/205764 A1, which is incorporated herein by reference in its entirety. In different embodiments, the methods described herein can be performed in vitro, in vivo, or ex vivo.

For example, the CRISPR-Cas system described herein can be administered to a subject having a disease or disorder to target and induce cell death in a cell in a diseased state (e.g., cancer cells or cells infected with an infectious agent). For instance, in some embodiments, the CRISPR-Cas system described herein can be used to target and induce cell death in a cancer cell, wherein the cancer cell is from a subject having a Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, or urinary bladder cancer.

### Modulating Gene Expression

The CRISPR-Cas system described herein can be used to modulate gene expression. The CRISPR-Cas system can be used, together with suitable guide RNAs, to target gene expression. The RNA targeting proteins in combination with suitable guide RNAs can also be used to control RNA activation (RNAa). RNA activation is a small RNA-guided and Argonaute (Ago)-dependent gene regulation phenomenon in which promoter-targeted short double-stranded RNAs (dsRNAs) induce target gene expression at the transcriptional/epigenetic 5 level. RNAa leads to the promotion of gene expression, so control of gene expression may be achieved that way through disruption or reduction of RNAa. In some embodiments, the methods include the use of the RNA targeting CRISPR as substitutes for e.g., interfering ribonucleic acids (such as siRNAs, shRNAs, or dsRNAs). The methods of modulating gene expression are described, e.g., in WO 2016205764, which is incorporated herein by reference in its entirety.

### RNA Base Editing

The CRISPR-Cas system described herein can be fused to a base-editing domain, such as ADAR1, ADAR2, APOBEC, or activation-induced cytidine deaminase (AID), and can be used to modify an RNA sequence (e.g., an mRNA). In some embodiments, the CRISPR-Cas system includes one or more mutations (e.g., in a catalytic domain), which renders them incapable of cleaving RNA.

In some embodiments, the CRISPR-Cas system can be used with an RNA-binding fusion polypeptide comprising a base-editing domain (e.g., ADAR1, ADAR2, APOBEC, or AID) fused to an RNA-binding domain, such as MS2 (also known as MS2 coat protein), Qbeta (also known as Qbeta coat protein), or PP7 (also known as PP7 coat protein). The amino acid sequences of the RNA-binding domains MS2, Qbeta, and PP7 are set forth in SEQ ID NOs: 82-84, respectively.

In some embodiments, the RNA binding domain can bind to a specific sequence (e.g., an aptamer sequence) or secondary structure motifs on a crRNA of the system described herein (e.g., when the crRNA is in an effector-crRNA complex), thereby recruiting the RNA binding fusion polypeptide (which has a base-editing domain) to the effector complex. For example, in some embodiments, the CRISPR system includes a CRISPR associated protein, a crRNA having an aptamer sequence (e.g., an MS2 binding loop, a QBeta binding loop, or a PP7 binding loop), and a RNA-binding fusion polypeptide having a base-editing domain fused to an RNA-binding domain that specifically binds to the aptamer sequence. In this system, the CRISPR-associated protein forms a complex with the crRNA having the aptamer sequence. Further the RNA-binding fusion polypeptide binds to the crRNA (via the aptamer sequence) thereby forming a tripartite complex that can modify a target RNA.

Methods of using CRISPR-Cas system for base editing are described, e.g., in International Publication No. WO 2017/219027, which is incorporated herein by reference in its entirety, and in particular with respect to its discussion of RNA modification.

### m6A Modification

N6-methyladenosine (m6A) is methylation that occurs in the N6-position of adenosine, which is the most prevalent internal modification on eukaryotic mRNA. Accumulating evidence suggests that m6A modulates gene expression, thereby regulating cellular processes ranging from cell self-renewal, differentiation, invasion and apoptosis. m6A is installed by m6A methyltransferases, removed by m6A demethylases and recognized by reader proteins, which regulate of RNA metabolism including translation, splicing, export, degradation and microRNA processing. Alteration of m6A levels participates in cancer pathogenesis and development via regulating expression of tumor-related genes like BRD4, MYC, SOCS2 and EGFR. (He L, Li H, Wu A, Peng Y, Shu G, Yin G. Functions of N6-methyladenosine and its role in cancer. Mol Cancer. 2019 Dec. 4; 18(1):176. doi: 10.1186/s12943-019-1109-9. PMID: 31801551; PMCID: PMC6892141.)

N6-methyladenosine is the most plentiful internal modification of mRNA and occurs in small noncoding RNAs (ncRNAs) and long noncoding RNAs (lncRNAs). The deposition of the methyl group on adenosine is conducted by a multiprotein complex in which methyltransferase-like 3 (METTL3) hosts the catalytic core, which is an S-adenosyl methionine-binding protein with methyltransferase activity. Methyltransferase-like 14 (METTL14) assists in mRNA binding. Moreover the WTAP protein (Wilms tumor 1—associated protein) is fundamental for the correct cellular methylation activity of the METTL3 and METTL14 enzymes. It is well known that m6A modification in RNAs is dynamic and reversible. In this case the demethylation potential is ascribed to the FTO (the fat mass and obesity-associated protein) and ALKBH5 (AlkB homolog 5) enzymes, both of which oxidatively remove the methyl group from adenosine. (He L, Li H, Wu A, Peng Y, Shu G, Yin G. Functions of N6-methyladenosine and its role in cancer. Mol Cancer. 2019 Dec. 4; 18(1):176. doi: 10.1186/s12943-019-1109-9. PMID: 31801551; PMCID: PMC6892141.)

Thus, in some embodiments, the heterologous functional domain comprises a m6A-associated regulation domain, such as, a m6A-associated methyltransferase domain (e.g., METTL3, METTL14, WTAP, KIAA1429, or a functional fragment thereof), a m6A-associated demethylation domain (e.g., Fat mass and obesity-associated protein (FTO), ALKBH5, or a functional fragment thereof), or a combination thereof.

For the purpose of m6A-associated epigenetic regulation, in some embodiments, a m6A-associated epigenetic regulator may be designed, comprising (1) a crRNA binding polypeptide comprising, consisting essentially of, or consisting of a crRNA binding domain of a Cas effector protein, and (2) a heterologous functional domain that may be a m6A providing moiety for providing a m6A modification to a target RNA or a m6A eliminating moiety for eliminating a m6A modification from a target RNA. The Cas effector protein may be any Cas effector protein as described herein, for example, a Cas13 effector protein or a CasPR.

In some embodiments, the m6A providing moiety is selected from METTL3, METTL14, WTAP, KIAA1429, or a functional fragment thereof, or a combination thereof.

In some embodiments, the m6A eliminating moiety is selected from FTO, ALKBH5, or a functional fragment thereof, or a combination thereof.

In some embodiments, a m6A-associated epigenetic regulating system may further designed, comprising the m6A-associated RNA regulator and a guide RNA (gRNA). The gRNA may comprise a direct repeat (DR) sequence capable of forming a complex with the crRNA binding domain and a spacer sequence capable of hybridizing to a target RNA, and guiding or recruiting the complex to the target RNA. Further, the gRNA may comprise a 5′ direct repeat (DR) sequence and a 3′ direct repeat (DR) sequence, each capable of forming a complex with the crRNA binding domain, and a spacer sequence capable of hybridizing to a target RNA, and guiding or recruiting the complex to the target RNA, wherein the spacer sequence is flanked by the 5′ and 3′ DR sequences at the 5′ end and the 3′ end of the spacer sequence, respectively, and the 5′ and 3′ DR sequences are identical or different.

The m6A-associated epigenetic regulating system may be used to provide or eliminate a m6A modification to or from a target RNA. In some embodiments, the target RNA may be a mRNA associated with a m6A-associated epigenetic characteristic.

Detection of m6A on a target RNA may be conducted by conventional methods known in the art, including high-throughput sequencing (e.g., MeRIP-seq, miCLIP-seq), colorimetry, or LC-MS (e.g., LC-MS/MS).

### Methods of Modifying

In another aspect of the disclosure, it is provided a method of modifying a target RNA, the method 5 comprising contacting the target RNA with the modified Cas13 protein, the fusion protein, or the CRISPR-Cas13 system of the disclosure, the polynucleotide of the disclosure, the vector of the disclosure, the rAAV viral particle of the disclosure, the delivery system of the disclosure, the cell or a progeny thereof of the disclosure, the pharmaceutical composition of the disclosure, or the kit of the disclosure, wherein the spacer sequence is substantially complementary to at least 15 contiguous nucleotides of the target RNA; wherein the crRNA binding polypeptide associates with the gRNA to form a complex; wherein the complex binds to the target RNA; and wherein upon binding of the complex to the target RNA, the complex modifies the target RNA (e.g., deaminates a target ribonucleotide base (e.g., A or C) in the target RNA).

In some embodiments, the target RNA is an mRNA, a tRNA, an rRNA, a non-coding RNA, a lncRNA, or a nuclear RNA.

In some embodiments, the target RNA has a mutation associated with a genetic disease or disorder or has or lacks a modification associated with epigenetics.

In some embodiments, the method of the disclosure causes one or more of: (i) in vitro or in vivo induction of cellular senescence; (ii) in vitro or in vivo cell cycle arrest; (iii) in vitro or in vivo cell growth inhibition; (iv) in vitro or in vivo induction of anergy; (v) in vitro or in vivo induction of apoptosis; and (vi) in vitro or in vivo induction of necrosis.

In some embodiments, the method is an in vitro method, an in vivo method, or an ex vivo method.

### Therapeutic Applications

In an aspect of the disclosure, it is provided a method of treating a condition or disease in a subject in need thereof, the method comprising administering to the subject the modified Cas13 protein, the fusion protein, or the CRISPR-Cas13 system of the disclosure, the polynucleotide of the disclosure, the vector of the disclosure, the rAAV viral particle of the disclosure, the delivery system of the disclosure, the cell or a progeny thereof of the disclosure, the pharmaceutical composition of the disclosure, or the kit of the disclosure, wherein the spacer sequence is substantially complementary to at least 15 contiguous nucleotides of a target RNA associated with the condition or disease; wherein the crRNA binding polypeptide associates with the gRNA to form a complex; wherein the complex binds to the target RNA; and wherein upon binding of the complex to the target RNA, the complex modifies the target RNA (e.g., deaminates a target ribonucleotide base (e.g., A or C) in the target RNA), thereby treating the condition or disease in the subject.

In some embodiments, the condition or disease is a genetic or epigenetic disease or disorder.

In some embodiments, the method is an in vitro method, an in vivo method, or an ex vivo method.

The CRISPR-Cas system described herein can have various therapeutic applications. Such applications may be based on one or more of the abilities below, both in vitro and in vivo, of the subject CRISPR-Cas system: induce cellular senescence, induce cell cycle arrest, inhibit cell growth and/or proliferation, induce apoptosis, induce necrosis, etc.

In some embodiments, the CRISPR-Cas system can be used to treat various diseases and disorders, e.g., genetic disorders (e.g., monogenetic diseases), diseases that can be treated by nuclease activity (e.g., Pcsk9 targeting, Duchenne Muscular Dystrophy (DMD), BCL11a targeting), and various cancers, etc.

The CRISPR-Cas system described herein can also be used in the treatment of various tauopathies, including, e.g., primary and secondary tauopathies, such as primary age-related tauopathy (PART)/Neurofibrillary tangle (NFT)-predominant senile dementia (with NFTs similar to those seen in Alzheimer Disease (AD), but without plaques), dementia pugilistica (chronic traumatic encephalopathy), and progressive supranuclear palsy. A useful list of tauopathies and methods of treating these diseases are described, e.g., in WO 2016205764, which is incorporated herein by reference in its entirety.

The CRISPR-Cas system described herein can also be used to target mutations disrupting the cis-acting splicing codes that can cause splicing defects and diseases. These diseases include, e.g., motor neuron degenerative disease that results from deletion of the SMN1 gene (e.g., spinal muscular atrophy), Duchenne Muscular Dystrophy (DMD), frontotemporal dementia, and Parkinsonism linked to chromosome 17 (FTDP-17), and cystic fibrosis.

The CRISPR-Cas system described herein can further be used for antiviral activity, in particular against RNA viruses. The CRISPR-Cas system can target the viral RNAs using suitable guide RNAs selected to target viral RNA sequences.

The CRISPR-Cas system described herein can also be used to treat a cancer in a subject (e.g., a human subject). For example, the CRISPR-Cas system described herein can be programmed with crRNA targeting a RNA molecule that is aberrant (e.g., comprises a point mutation or are alternatively-spliced) and found in cancer cells to induce cell death in the cancer cells (e.g., via apoptosis).

The CRISPR-Cas system described herein can also be used to treat an autoimmune disease or disorder in a subject (e.g., a human subject). For example, the CRISPR-Cas system described herein can be programmed with crRNA targeting a RNA molecule that is aberrant (e.g., comprises a point mutation or are alternatively-spliced) and found in cells responsible for causing the autoimmune disease or disorder.

Further, the CRISPR-Cas system described herein can also be used to treat an infectious disease in a subject. For example, the CRISPR-Cas system described herein can be programmed with crRNA targeting a RNA molecule expressed by an infectious agent (e.g., a bacteria, a virus, a parasite or a protozoan) in order to target and induce cell death in the infectious agent cell. The CRISPR-Cas system may also be used to treat diseases where an intracellular infectious agent infects the cells of a host subject. By programming the CRISPR-associated protein to target a RNA molecule encoded by an infectious agent gene, cells infected with the infectious agent can be targeted and cell death induced.

A detailed description of therapeutic applications of the CRISPR systems described herein can be found, e.g., in U.S. Pat. No. 8,795,965, EP 3009511, WO 2016205764, and WO 2017070605; each of which is incorporated herein by reference in its entirety.

### 12. Exemplary, Additional Embodiments

The disclosure provides the following exemplary, additional embodiments.

Embodiment 1. A targeted RNA base editor or a derivative thereof, said targeted RNA base editor comprising:

(a) a polypeptide comprising, consisting essentially of, or consisting of a crRNA binding domain of a small Clustered Regularly Interspaced Short Palindromic Repeat (CRISPR)-Cas effector enzyme (“small Cas effector enzyme”),

(b) an RNA base editor, and,

(c) an RNA guide sequence comprising a spacer sequence capable of hybridizing to a target RNA, said spacer sequence is flanked by a direct repeat (DR) sequence native to the small Cas effector enzyme at both the 5′ end and the 3′ end of the spacer sequence,

wherein said polypeptide:

(1) is linked (e.g., fused) to said RNA base editor,

(2) substantially lacks ability to process said direct repeat (DR) sequence of the RNA guide sequence, and,

(3) forms a complex with said RNA guide sequence (e.g., by binding to the DR sequence through said crRNA binding domain), and,

wherein said RNA base editor deaminates a target ribonucleotide base (e.g., A or C) in said target RNA when said RNA guide sequence hybridizes to said target RNA.

Embodiment 2. The targeted RNA base editor of Embodiment 1, wherein the small Cas effector enzyme is a Class 2, Type VI-A (Cas13a or C2c2), Type VI-B (Cas13b), Type VI-C(Cas13c), Type VI-D (Cas13d), Type VI-E (Cas13e), or Type VI-F (Cas13f) Cas effector enzyme.

Embodiment 3. The targeted RNA base editor of Embodiment 1 or 2, wherein the small Cas effector enzyme comprises an amino acid sequence of any one of SEQ ID NOs: 1-7.

Embodiment 4. The targeted RNA base editor of Embodiment 2 or 3, wherein said polypeptide substantially lacks the N-terminal HEPN domain (e.g., RxxxxH domain) and/or the C-terminal HEPN domain (e.g., RxxxxH domain).

Embodiment 5. The targeted RNA base editor of Embodiment 1, wherein the small Cas effector enzyme is a Class 2, Type VI-E (Cas13e) Cas effector enzyme (e.g., SEQ ID NO: 1), and wherein said polypeptide lacks about 180 (e.g., 170, 171, 172, 173, 174, 175, 176, 177, 178, 179, 180, 181, 182, 183, 184, 185, 186, 187, 188, 189, or 190) N-terminal residues, and lacks about 150 (e.g., 140, 141, 142, 143, 144, 145, 146, 147, 148, 149, 150, 151, 152, 153, 154, 155, 156, 157, 158, 159, or 160) C-terminal residues of said Cas13e effector enzyme (e.g., SEQ ID NO: 1).

Embodiment 6. The targeted RNA base editor of Embodiment 1, wherein the small Cas effector enzyme is a Cas6e effector enzyme, optionally, said polypeptide comprises the amino acid sequence of SEQ ID NO: 51 (EcCas6e-H20L).

Embodiment 7. The targeted RNA base editor of any one of Embodiments 1-5, wherein the DR sequence has substantially the same secondary structure as the secondary structure of any one of SEQ ID NOs: 8-14; or the targeted RNA base editor of Embodiment 6, wherein the DR sequence has substantially the same secondary structure as the secondary structure of SEQ ID NO: 47.

Embodiment 8. The targeted RNA base editor of Embodiment 7, wherein the DR sequence is encoded by any one of SEQ ID NOs: 8-14, or 47.

Embodiment 9. The targeted RNA base editor of any one of Embodiments 1-8, wherein the target RNA is encoded by a eukaryotic DNA.

Embodiment 10. The targeted RNA base editor of Embodiment 9, wherein the eukaryotic DNA is a non-human mammalian DNA, a non-human primate DNA, a human DNA, a plant DNA, an insect DNA, a bird DNA, a reptile DNA, a rodent DNA, a fish DNA, a worm/nematode DNA, a yeast DNA.

Embodiment 11. The targeted RNA base editor of any one of Embodiments 1-10, wherein the target RNA is an mRNA.

Embodiment 12. The targeted RNA base editor of any one of Embodiments 1-11, wherein the spacer sequence is between 15-60 nucleotides, between 25-50 nucleotides, about 55 nucleotides, about 50 nucleotides, about 45 nucleotides, about 40 nucleotides, about 35 nucleotides, or about 30 nucleotides.

Embodiment 13. The targeted RNA base editor of any one of Embodiments 1-12, wherein the spacer sequence is 90-100% complementary to the target RNA, or contains no more than 1, 2, 3, 4, or 5 consecutive or non-consecutive mismatches to the target RNA.

Embodiment 14. The targeted RNA base editor of any one of Embodiments 1-13, wherein the RNA base editor comprises an adenosine deaminase, such as a double-stranded RNA-specific adenosine deaminase (e.g., ADAR1 or ADAR2); apolipoprotein B mRNA editing enzyme; catalytic polypeptide-like (APOBEC); activation-induced cytidine deaminase (AID), or a functional fragment thereof.

Embodiment 15. The targeted RNA base editor of Embodiment 14, wherein the ADAR2 comprises the E488Q mutation or the E488Q/T375G double mutation, or wherein the functional fragment thereof comprises ADAR2DD optionally comprising the E488Q mutation or the E488Q/T375G double mutation.

Embodiment 16. The targeted RNA base editor of any one of Embodiments 1-15, wherein the RNA base editor is fused C-terminal to said polypeptide.

Embodiment 17. The targeted RNA base editor of Embodiment 16, comprising a GS linker linking the polypeptide and the RNA base editor.

Embodiment 18. The targeted RNA base editor of Embodiment 17, wherein the GS linker comprises GS or 2-15 repeats thereof (SEQ ID NO: 85), GSGGGGS (SEQ ID NO: 29) or 2-4 repeats thereof (SEQ ID NO: 86), GGS or 5-10 repeats thereof (SEQ ID NO: 87), GGGS (G₃S) (SEQ ID NO: 63) or 3-7 repeats thereof (SEQ ID NO: 88), GGGGS (G₄S) (SEQ ID NO: 93) or 3-5 repeats thereof (SEQ ID NO: 89), GGGGGS (G₅S) (SEQ ID NO: 94) or 3-4 repeats thereof (SEQ ID NO: 90), or a mixture thereof, or SEQ ID NO: 33; optionally, the length of the GS linker is about 15, 16, 17, 18, 19, 20, 21, 22, 23, 24, 25, 26, or 27 residues.

Embodiment 19. The targeted RNA base editor of any one of Embodiments 1-18, wherein the polypeptide and/or the RNA base editor is linked to a nuclear localization signal (NLS) sequence or a nuclear export signal (NES).

Embodiment 20. The targeted RNA base editor of Embodiment 19, wherein the polypeptide and/or the RNA base editor is linked to 2 or 3 NLS, such as SEQ ID NO: 35.

Embodiment 21. The targeted RNA base editor of Embodiment 20, comprising one each of NLS fused N- and C-terminal to the polypeptide.

Embodiment 22. The targeted RNA base editor of any one of Embodiments 1-21, wherein the RNA base editor deaminates an adenosine (A) in the target RNA to an inosine (I).

Embodiment 23. The targeted RNA base editor of Embodiment 22, wherein the spacer sequence comprises a cystine (C) mismatch opposite to the adenosine (A) in the target RNA.

Embodiment 24. The targeted RNA base editor of Embodiment 23, wherein the cystine mismatch is about 5, 6, 7, 8, 9, 10, 11, 12, 13, 14, 15, 16, 17, 18, 19, 20, 21, 22, 23, 24, 25, 26, 27, 28, 29, or 30 nucleotides (e.g., about 15-25 nucleotides) from the 5′ or 3′ DR sequence.

Embodiment 25. The targeted RNA base editor of any one of Embodiments 1-24, wherein the derivative comprises only conserved amino acid substitutions or is at least about 90%, 91%, 92%, 93%, 94%, 95%, 96%, 97%, 98%, 99%, 99.2%, 99.5%, 99.7%, or 99.8% identical to the targeted base editor; and the derivative retains substantially all functions of the targeted base editor (e.g., ability to bind to the guide RNA, ability to permit the guide RNA to hybridize with the target RNA, ability to deaminate the target ribonucleotide on the target RNA, and ability to avoid processing said direct repeat (DR) sequence of the RNA guide sequence).

Embodiment 26. The targeted RNA base editor of any one of Embodiments 1-25, further comprising, or is conjugated to, a heterologous functional domain.

Embodiment 27. The targeted RNA base editor of Embodiment 26, wherein the heterologous functional domain comprises: a nuclear localization signal (NLS), a reporter protein or a detection label (e.g., GST, HRP, CAT, GFP, HcRed, DsRed, CFP, YFP, BFP), a localization signal, a protein targeting moiety, a DNA binding domain (e.g., MBP, Lex A DBD, Gal4 DBD), an epitope tag (e.g., His, myc, V5, FLAG, HA, VSV-G, Trx, etc), a transcription activation domain (e.g., VP64 or VPR), a transcription inhibition domain (e.g., KRAB moiety or SID moiety), a nuclease (e.g., FokI), a deamination domain (e.g., ADAR1, ADAR2, APOBEC, AID, or TAD), a methylase, a demethylase, a transcription release factor, an HDAC, a polypeptide having ssRNA cleavage activity, a polypeptide having dsRNA cleavage activity, a polypeptide having ssDNA cleavage activity, a polypeptide having dsDNA cleavage activity, a DNA or RNA ligase, or any combination thereof.

Embodiment 28. The targeted RNA base editor of Embodiment 26 or 27, wherein the heterologous functional domain is fused or conjugated N-terminally, C-terminally, or internally in the targeted RNA base editor.

Embodiment 29. A polynucleotide comprising a first polynucleotide encoding the protein component of the targeted RNA base editor of any one of Embodiments 1-28, and a second polynucleotide encoding the RNA guide sequence.

Embodiment 30. The polynucleotide of Embodiment 29, wherein transcription of the protein component of the targeted RNA base editor and transcription of the RNA guide sequence are under the control of separate or independent promoters and/or enhancers.

Embodiment 31. The polynucleotide of Embodiment 30, wherein transcription of the protein component of the targeted RNA base editor is under the control of a constitutive promoter, an inducible promoter, a ubiquitous promoter, or a tissue specific promoter.

Embodiment 32. The polynucleotide of Embodiment 31, wherein the constitutive promoter is a RNA Pol II promoter, such as a CMV promoter, a CB promoter, a Cbh promoter, an EFS promoter, or a CAG promoter.

Embodiment 33. The polynucleotide of any one of Embodiments 30-32, wherein transcription of the RNA guide sequence is under the control of an RNA Pol III promoter, such as a U6 promoter.

Embodiment 34. The polynucleotide of any one of Embodiments 29-33, wherein the first polynucleotide is codon-optimized for expression in a cell, such as a eukaryotic cell, or a mammalian (e.g., human) cell.

Embodiment 35. A vector comprising the polynucleotide of any one of Embodiments 29-34.

Embodiment 36. The vector of Embodiment 35, which is a plasmid.

Embodiment 37. The vector of Embodiment 35, which is a retroviral vector, a phage vector, an adenoviral vector, a herpes simplex viral (HSV) vector, an AAV vector, or a lentiviral vector.

Embodiment 38. The vector of Embodiment 35, which is an AAV vector comprising the polynucleotide of any one of Embodiments 29-34 flanked by a 5′ ITR (such as an AAV2 5′ ITR) and a 3′ ITR (such as an AAV2 3′ ITR).

Embodiment 39. The vector of Embodiment 38, wherein the polynucleotide of any one of Embodiments 29-34 further comprise an intron and/or an exon that promotes transcription of the protein component of the targeted RNA base editor.

Embodiment 40. The vector of Embodiment 38 or 39, further comprising a coding sequence for a polyA signal sequence operably linked to the first polynucleotide encoding the protein component of the targeted RNA base editor.

Embodiment 41. The vector of any one of Embodiments 38-40, further comprising a 5′ UTR and/or a 3′ UTR coding sequence in the first polynucleotide encoding the protein component of the targeted RNA base editor.

Embodiment 42. The vector of any one of Embodiments 38-41, further comprising a WPRE sequence.

Embodiment 43. A recombinant AAV (rAAV) viral particle comprising the AAV vector of any one of Embodiments 37-42, encapsidated within a capsid of the serotype of AAV1, AAV2, AAV3A, AAV3B, AAV4, AAV5, AAV6, AAV7, AAVrh74, AAV8, AAV9, AAV10, AAV11, AAV12, or AAV13.

Embodiment 44. A delivery system comprising (1) a delivery vehicle, and (2) the targeted RNA base editor of any one of Embodiments 1-28, the polynucleotide of any one of Embodiments 29-34, the vector of any one of Embodiments 35-42, or the rAAV viral particle of Embodiment 43.

Embodiment 45. The delivery system of Embodiment 44, wherein the delivery vehicle is a nanoparticle, a liposome, an exosome, a microvesicle, or a gene-gun.

Embodiment 46. A cell or a progeny thereof, comprising the targeted RNA base editor of any one of Embodiments 1-28, the polynucleotide of any one of Embodiments 29-34, the vector of any one of Embodiments 35-42, or the rAAV viral particle of Embodiment 43.

Embodiment 47. The cell or progeny thereof of Embodiment 46, which is a eukaryotic cell (e.g., a non-human mammalian cell, a human cell, or a plant cell) or a prokaryotic cell (e.g., a bacteria cell).

Embodiment 48. A non-human multicellular eukaryote comprising the cell of Embodiment 46 or 47. Embodiment 49. The non-human multicellular eukaryote of Embodiment 48, which is an animal (e.g., rodent or primate) model for a human genetic disorder.

Embodiment 50. A method of modifying a target RNA, the method comprising contacting the target RNA with the targeted RNA base editor of any one of Embodiments 1-28, wherein the spacer sequence is complementary to at least 15 nucleotides of the target RNA; wherein the polypeptide associates with the RNA guide sequence to form a complex; wherein the complex binds to the target RNA; and wherein upon binding of the complex to the target RNA, the targeted RNA base editor deaminates a target ribonucleotide base (e.g., A or C) in said target RNA.

Embodiment 51. The method of Embodiment 50, wherein the target RNA is an mRNA, a tRNA, an rRNA, a non-coding RNA, an lncRNA, or a nuclear RNA.

Embodiment 52. The method of Embodiment 50 or 51, wherein the target RNA is within a cell.

Embodiment 53. The method of Embodiment 52, wherein the cell is a cancer cell.

Embodiment 54. The method of Embodiment 52, wherein the cell is infected with an infectious agent.

Embodiment 55. The method of Embodiment 54, wherein the infectious agent is a virus, a prion, a protozoan, a fungus, or a parasite.

Embodiment 56. The method of Embodiment 54, wherein the cell has a mutation associated with a genetic disease or disorder.

Embodiment 57. The method of any one of Embodiments 50-56, which causes one or more of: (i) in vitro or in vivo induction of cellular senescence; (ii) in vitro or in vivo cell cycle arrest; (iii) in vitro or in vivo cell growth inhibition and/or cell growth inhibition; (iv) in vitro or in vivo induction of anergy; (v) in vitro or in vivo induction of apoptosis; and (vi) in vitro or in vivo induction of necrosis.

Embodiment 58. A method of treating a condition or disease in a subject in need thereof, the method comprising administering to the subject a composition comprising the targeted RNA base editor of any one of Embodiments 1-28, the polynucleotide of any one of Embodiments 29-34, the vector of any one of Embodiments 35-42, or the rAAV viral particle of Embodiment 43, wherein the spacer sequence is complementary to at least 15 nucleotides of a target RNA associated with the condition or disease; wherein the polypeptide of the targeted RNA base editor associates with the RNA guide sequence to form a complex; wherein the complex binds to the target RNA; and wherein upon binding of the complex to the target RNA, the targeted RNA base editor deamidates a target ribonucleotide base (e.g., A or C) in said target RNA, thereby treating the condition or disease in the subject.

Embodiment 59. The method of Embodiment 58, wherein the condition or disease is a cancer or an infectious disease.

Embodiment 60. The method of Embodiment 59, wherein the cancer is Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, or urinary bladder cancer.

Embodiment 61. The method of any one of Embodiments 58-60, which is an in vitro method, an in vivo method, or an ex vivo method.

### EXAMPLES

### Example 1 RNA Single-Base Editing using shortened dCas13e.1-hADAR2_{DD} Fusion

In order to determine the minimum size of dead Cas13e.1 (dCas13e.1 or dCas13e when referred to in the Examples and drawings) that can be used in RNA single base editing, a series of five constructs expressing progressively larger C-terminal deletions (truncations) of dCas13e.1 were generated, each with 30 fewer residues from the C-terminus (i.e., 30-, 60-90-, 120-, and 150-residue deletions). The resulting constructs were used to create coding sequences for dCas13e.1 fused with high fidelity human ADAR2 deaminase domain-E488Q/T375G mutant (hADAR2_{DD}-E488Q/T375G (SEQ ID NO: 138) shown as “ADAD2DD” in FIG. 4) at the respective C-terminus. These constructs were cloned into Vysz15 (“V15”) to Vysz19 (“V19”) expression plasmids (FIG. 4). In all these constructs, each of the dCas13e.1-ADAR2DD fusion proteins was expressed under the regulation of the CMV promoter (pCMV) and enhancer (eCMV) and was immediately downstream of an intron that further enhanced protein expression. Two Nuclear Localization Sequences (NLSs) were positioned at the N- and C-termini of the dCas13e.1 portion of the fusion protein, and the hADAR2_{DD}-E488Q/T375G was fused to the C-terminal NLS through a Linker and tagged at its C-terminus with an HA tag. An EGFP coding sequence under the independent control of a EFS promoter (pEFS) was present downstream of the polyA sequence downstream of the HA tag to indicate the successful transfection and expression of the expression plasmids.

Interestingly, it was found (FIG. 6) that the progressive C-terminal deletion (truncation) steadily increased the RNA base editing activity of the fusion protein, such that the fusion protein with 150 C-terminal residue deletion (in V19) exhibited the highest RNA base editing activity. However, the 180-residue deletion from the C-terminus appeared to have abolished the RNA base editing activity, suggesting that the maximum/optimal deletion from the C-terminal end of Cas13e.1 is likely between 150-180 residues.

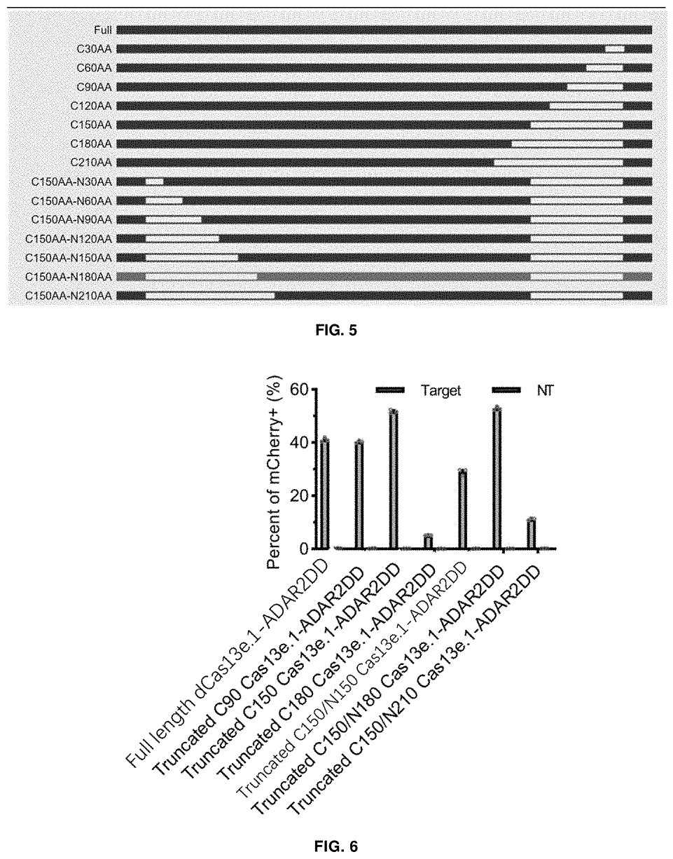

Based on this finding, a series of N-terminal deletion (truncation) mutants were generated based on the C-terminally truncated dCas13e.1 having 150 C-terminal residue deletion. Seven such N-terminal deletion (truncation) mutants were generated, with 30-, 60-, 90-, 120-, 150-, 180-, and 210-residue deletions (truncations), respectively (FIG. 5). The results in FIG. 6 show that the best RNA base editing activity was observed for the truncated dCas13e.1 mutant with 180 N-terminal residue deletion and 150 C-terminal residue deletion, i.e., a total of 330-residue deletion from the 775-residue parental Cas13e.1 protein, to generate the 445-residue optimal truncated dCas13e.1 (“minidCas13e.1”, SEQ ID NO: 32) suitable for generating a fusion protein with a heterologous function domain, such as, a deaminase domain.

### Example 2 Transcriptome-Wide Off-Target Detection of Base Editors

One key desirable attributes of a targeted RNA base editor is its ability to avoid off-target base editing at one or more unintended RNA sites and limits the base editing function to the intended target RNA sequence as much as possible.

This Example demonstrates that the subject targeted RNA base editor—a minidCas13e.1(or “miniCas13e” in the Examples and drawings)-hADAR2_{DD}-E448Q (or “ADAR2dd_E448Q” in the Examples and drawings) fusion protein, has unexpectedly low off-target RNA base editing.

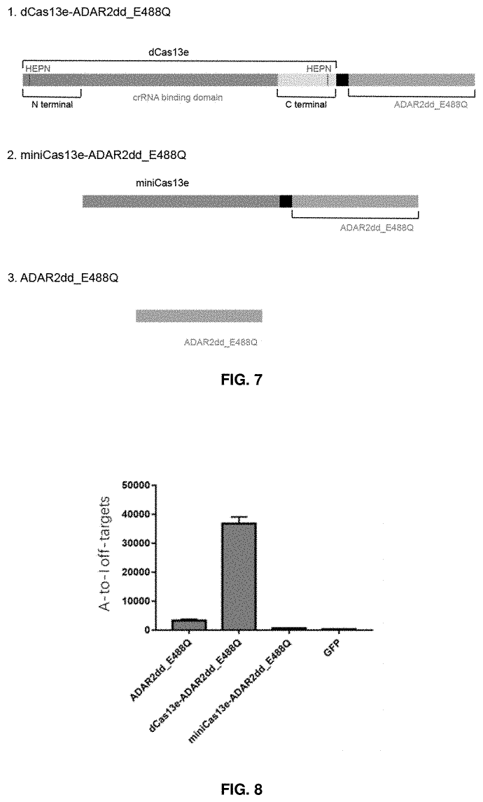

Specifically, full length dCas13e.1 (SEQ ID NO: 31) fused to the activated ADAR2 deaminase domain hADAR2_{DD}-E488Q (SEQ ID NO: 34) (dCas13e.1-hADAR2_{DD}-E448Q, SEQ ID NO: 36), and minidCas13e.1 (SEQ ID NO: 32) with 180 N-terminal residue deletion and 150 C-terminal residue deletion fused to the same activated ADAR2dd hADAR2_{DD}-E488Q (SEQ ID NO: 34) (minidCas13e.1-hADAR2_{DD}-E448Q, SEQ ID NO: 37), were constructed (FIG. 7). Each of the full length dCas13e.1 and minidCas13e.1 proteins was fused to two NLS sequences at their N- and C-termini, and the hADAR2_{DD}-E448Q domain was fused C-terminal to the full length dCas13e.1 or minidCas13e.1 moiety through a GS linker (SEQ ID NO: 33) at the N-terminus of hADAR2_{DD}-E448Q. Further, the hADAR2_{DD}-E448Q, minidCas13e.1-hADAR2_{DD}-E448Q, and dCas13e.1-hADAR2_{DD}-E448Q constructs were constructed on mammalian expression plasmids capable of expressing EGFP fluorescent protein to indicate successful transfection and expression of the expression plasmids.

Human HEK293T cells were cultured in 24-well tissue culture plates according to standard methods, before the expression plasmids encoding hADAR2_{DD}-E448Q, minidCas13e.1-hADAR2_{DD}-E448Q, or dCas13e.1-hADAR2_{DD}-E448Q, respectively (each also expressing EGFP, see above), and a control expression plasmid encoding EGFP only, were transfected into HEK293T cells separately using standard polyethylenimine (PEI) transfection. The transfected cells were then cultured at 37° C. under CO₂ for 48 hours. After 48 hours of culturing, the cultured cells were sorted by flow cytometry to obtain transfection-positive cells based on EGFP signal.

Next, through RNAseq library construction and sequencing, the whole transcriptome “A-to-I” off-target effects of the four samples were analyzed. It was surprisingly observed (FIG. 8) that the minidCas13e.1 construct dramatically reduced transcriptome-wide RNA off-target base editing by two orders of magnitude—the level associated with minidCas13e.1-hADAR2_{DD}-E448Q was only about 1% of that of dCas13e.1-hADAR2_{DD}-E448Q.

### Example 3 RT-PCR Off-target Base Editing Detection of minidCas13e.1-hADAR2_{DD}-E448Q Base Editor via Fluorescence Reporting System

This Example demonstrates that the minidCas13e.1-hADAR2_{DD}-E448Q fusion protein has less RNA off-target base editing than dCas13b-hADAR2_{DD}-E448Q by fluorescence reporting system. hADAR2_{DD}-E448Q was also termed as ADARv1 in the Examples and drawings.

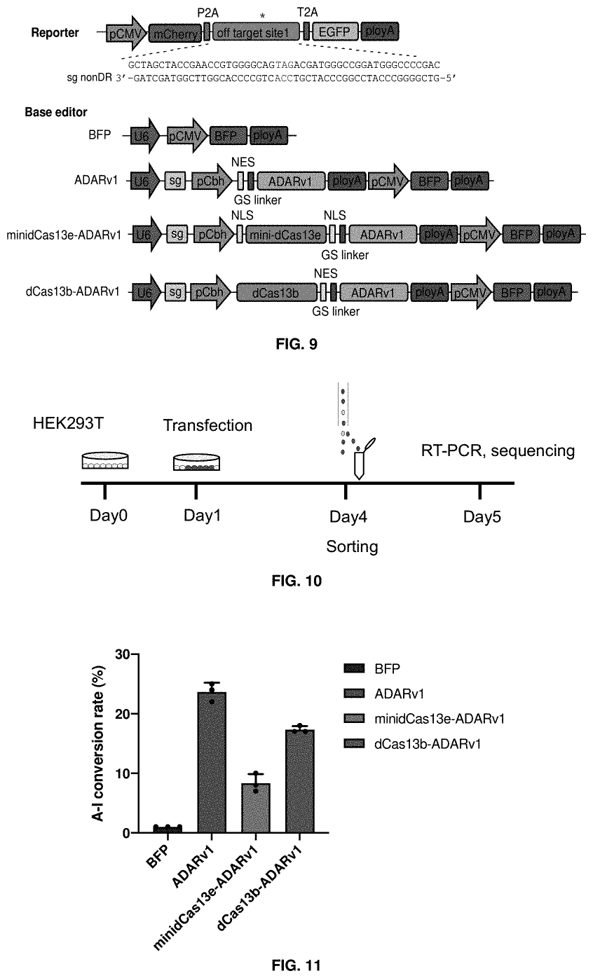

Based on the data in Example 2, a base site with higher than average off-target base editing efficiency was chosen for comparing the off-target base editing efficiency of hADAR2_{DD}-E448Q (ADARv1) when it was or was not fused to minidCas13e.1 or a dCas13b protein. Briefly, a reporter plasmid was constructed to transcribe a mCherry-P2A-off-target site 1 containing premature TAG stop codon-T2A-EGFP mRNA in FIG. 9. The sequences of P2A and T2A are set forth in SEQ ID NOs: 40 and 41, respectively. The expression of EGFP depended on the conversion of A-to-I via base editing to correct the premature TAG stop codon. Thus, the EGFP expression was used as a surrogate for base editing efficiency. The coding sequence of the off-target site is set forth in SEQ ID NO: 38. The target nucleotide “A” for A-to-I base editing is double underlined.

The expression plasmid for base editor comprised a spacer (“sg” in FIG. 9) coding sequence (SEQ ID NO: 39, targeting the off-target site set forth in SEQ ID NO: 38 and containing a “C” mismatch to enhance the base editing efficiency of A-to-I conversion) without a DR coding sequence under the regulation of a U6 promoter, a base editor coding sequence under the regulation of a Cbh promoter and a poly A sequence, and a BFP fluorescent reporter gene under the regulation of a CMV promoter and a poly A sequence. The base editor was composed of (1) hADAR2_{DD}-E448Q (SEQ ID NO: 34) downstream of a NES (SEQ ID NO: 48) and a GS linker (SEQ ID NO: 33), (2) NLS-minidCas13e.1-NLS-GS linker-hADAR2_{DD}-E488Q (SEQ ID NO: 34), or (3) dCas13b-NES-GS linker-hADAR2_{DD}-E488Q (SEQ ID NO: 42).

Human HEK293T cells were cultured in 24-well tissue culture plates according to standard methods, before the expression plasmids (expressing BFP) and the reporter plasmid (expressing mCherry) were transfected into the cells using standard polyethylenimine (PEI) transfection. The transfected cells were then cultured at 37° C. under CO₂ for 48 hours. The cultured BFP and mCherry double positive cells were sorted by flow cytometry after about 72 hours. EGFP signals as readouts for A-to-I RNA base editing were also detected using FACS.

The extent of “A-to-I” off-target editing was determined using RT-PCR. Specifically, RNA was extracted from the different transfected cell samples, and Sanger sequencing was performed after RT-PCR. The off-target base editing efficiency was analyzed according to the results of Sanger sequencing (FIG. 10).

The results (FIG. 11) show that the fusion protein of minidCas13e.1 or dCas13b protein and ADARv1 achieved significantly lower off-target RNA base editing efficiency than that of ADARv1 alone, and minidCas13e.1-hADAR2_{DD}-E448Q achieved much lower off-target base editing efficiency than dCas13b-hADAR2_{DD}-E448Q. These data demonstrates that the subject minidCas13e.1-based base editor is superior compared to similarly configured known base editors in terms of RNA off-target base editing.

Similar results were also obtained using a mutant version of small Cas effector enzyme, EcCas6e-H20L, instead of minidCas13e.1 (data not shown), which had comparably low off-target A-to-I conversion.

### Example 4 Guide RNA with Dual DR (dDR) has Higher Base Editing Efficiency than Guide RNA with Single DR (sDR) for DMD Exon 51 Based Editing

This Example demonstrates the surprising finding that using a gRNA with dual DR sequence flanking a spacer sequence can achieve higher base editing efficiency than using otherwise gRNA with a spacer sequence and a single DR sequence.

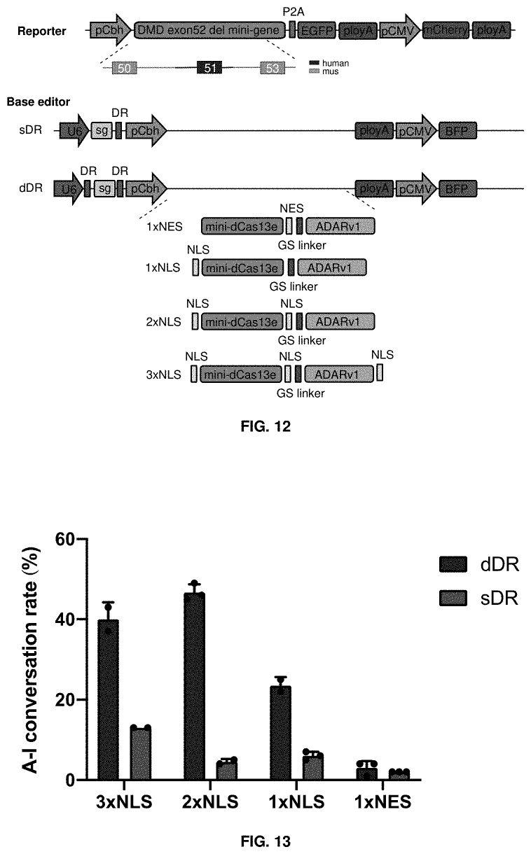

Referring to FIG. 12, a reporter plasmid was constructed with a DMD exon 51 SA (Ag>Gg) mutation being introduced into a DMD Mini gene (SEQ ID NO: 43) on the reporter plasmid. The reporter plasmid encodes an EGFP reporter, but the expression of EGFP depends on successful RNA base editing to covert an A to an I in order to eliminate a premature stop codon in the DMA exon 51 mutation. The reporter plasmid also encodes mCherry under the separate transcription control of a CMV promoter, such that the encoded mCherry acts as a positive control for plasmid transfection efficiency.

Also referring to FIG. 12, various base editor expression plasmids were constructed with combinations of different NES/NLS strategies and different DR strategies. Four NES/NLS strategies of 1xNES (SEQ ID NO: 48), 1xNLS (SEQ ID NO: 35), 2xNLS (SEQ ID NO: 35), and 3xNLS (SEQ ID NO: 35) were separately applied to the same minidCas13e.1-ADARv1 construct as mentioned above. Two DR strategies with respect to a gRNA with a single Cas13e.1 DR sequence (SEQ ID NO: 8) or dual Cas13e.1 DR sequences (SEQ ID NO: 8) and the same spacer sequence were designed to evaluate the effect of dual DR over single DR. The GS linker is set forth in SEQ ID NO: 33.

Human HEK293T cells were cultured in 24-well tissue culture plates according to standard methods, before the various expression plasmids and the reporter plasmid were transfected into the cells using standard polyethylenimine (PEI) transfection. The transfected cells were then cultured at 37° C. under CO₂ for 48 hours. EGFP signals as readout for RNA base editing efficiency were detected using FACS.

Through sanger sequencing, it was observed (FIG. 13) that with given NES/NLS, the A-to-I base editing efficiency achieved with the gRNA with dual DR configuration was higher than that with single DR configuration. On the other hand, for dual DR configuration, the strategy of 2xNLS flanking dCas13 protein achieved the highest A-to-I base editing efficiency than 1xNLS, 3xNLS, and 1xNES.

### Example 5 Guide RNA with Dual DR (dDR) has Higher Base Editing Efficiency than Guide RNA with Single DR (sDR) for DMD Exon 23X Disease Site

To demonstrate that the surprising increased base editing efficiency with dDR is not specific for a particular target site, a further target site was tested for the comparison of base editing efficiency with dDR and sDR.

Specifically, in this Example, a pathogenic site (DMD Exon23X) in DMD gene was chosen for further testing. The target DMD Exon23X (C>T) mutation created a premature stop codon TAA (from CAA), causing premature termination of DMD gene translation. The Exon23X (C>T) sequence is set forth in SEQ ID NO: 44, with the mutant T double underlined.

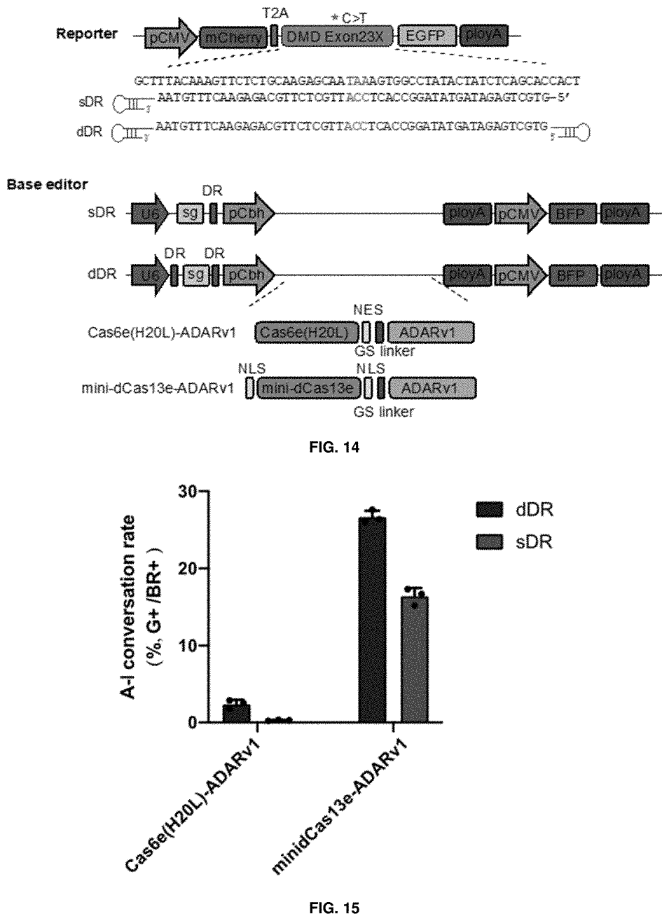

Referring to FIG. 14, in order to explore the RNA base editing efficiency more conveniently, a reporter system was designed. In this system, the expression of the reporter gene EGFP depends on the successful conversion of A-to-I (G) via RNA base editing in order to eliminate the premature stop codon TAA in the Exon23X sequence. That is, the reporter EGFP can only be expressed when the premature stop codon TAA is converted to TGG via RNA base editing.

Also referring to FIG. 14, all the base editor-encoding expression plasmids also encoded BFP as a marker for transfection and expression. The sDR expression plasmids encoded a single DR sequence linked 3′ to the spacer sequence of the guide RNA, while the dDR expression plasmids all encoded two DR sequences flanking the identical spacer sequence of the guide RNA. Two different base editors were tested. One has EcCas6e-H20L linked to ADARv1 (ADAR2_{DD_}E488Q) interposed with a NES (SEQ ID NO: 48), where EcCas6e (or “Cas6e” in the Examples and drawings) was introduced with a H20L mutation (EcCas6e-H20L, or “Cas6e(H20L)” in the Examples and drawings) that caused the EcCas6e to lose its crRNA processing endoribonuclease activity that cleaves crRNA (the ability of processing a concatemer of spacer-DR sequences to release individual spacer-DR or DR-spacer sequences as single guide RNA). The other base editor was the subject minidCas13e.1 flanked with N- and C-terminal NLS (SEQ ID NO: 35) linked to ADARv1. The GS linker is set forth in SEQ ID NO: 33. For the minidCas13e.1 base editor, the DR coding sequence is set forth in SEQ ID NO: 8. For the EcCas6e-H20L base editor, the DR coding sequence is set forth in SEQ ID NO: 47.

As in Example 4, the reporter plasmid and the expression plasmids were transfected into HEK293 cell lines, and the percentage of EGFP (“G+”) & BFP⁺/mCherry⁺ (“BR+”) was analyzed with flow cytometry 48 hours post transfection. A higher ratio represented more successful base editing.

The results (FIG. 15) show that for the different base editors based on EcCas6e and Cas13e.1, respectively, and the target site different from Example 4, a higher A-to-I base editing efficiency was still achieved for the dual DR (dDR) gRNA construct as compared to the single DR (sDR) gRNA construct with otherwise identical configuration.

### Example 6 Guide RNA with Dual DR (dDR) has Higher Base Editing Efficiency than Guide RNA with Single DR (sDR) for DMD Exon 54X Disease Site

As in Example 5, this Example demonstrates that the dDR constructs have higher editing efficiency compared to the corresponding sDR constructs, based on data obtained in another DMD pathogenic site (DMD Exon54X).

As in Example 5, this DMD pathogenic site contains a G>A mutation that created a premature stop codon TAG, and the expression of the reporter EGFP depends on successful RNA base editing to convert the TAG stop codon to TGG. The DMD Exon54X (G>A) target sequence is set forth in SEQ ID NO: 49, with the mutant T double underlined.

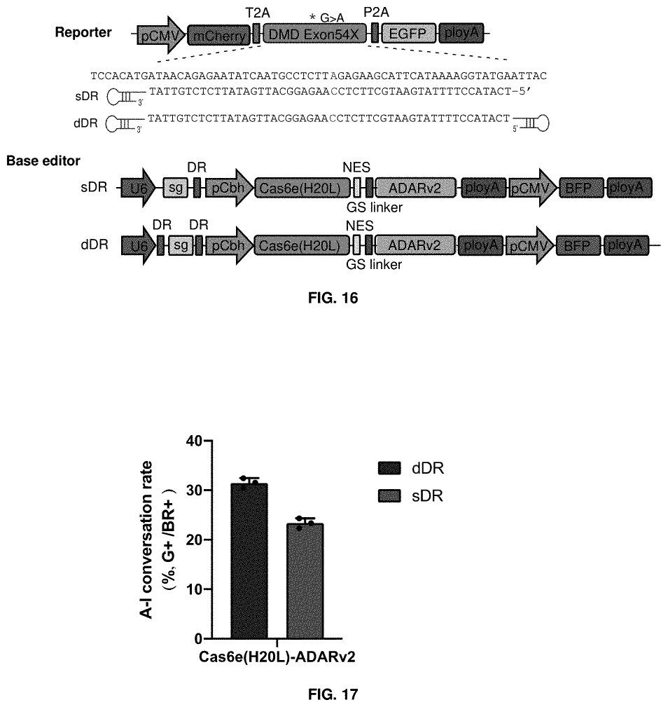

Referring to FIG. 16, as in Example 5, a reporter plasmid encompassing the DMD Exon 54X (G>A) target sequence was designed. Meanwhile, the sDR/dDR gRNA-EcCas6e-H20L-ADARv2 constructs as the sDR/dDR gRNA—EcCas6e-H20L-ADARv1 in Example 5 except for ADARv1 replaced with ADARv2 were used as the base editors in this Example. The reporter plasmid was co-transferred into HEK293T cells with the dDR or sDR base editor (EcCas6e-H20L-ADARv2) expression plasmid. After 48 hours, the ratio of EGFP/(BFP⁺ & mCherry⁺) was analyzed with flow cytometry.

It was observed (FIG. 17) that a higher A-to-I base editing efficiency was also achieved with the dual DR (dDR) construct than the corresponding single DR (sDR) construct for yet another target site and yet another base editor based on a different deaminase domain.

### Example 7 Guide RNA with Dual DR (dDR) has Higher Base Editing Efficiency than Guide RNA with Single DR (sDR) for RPE65 Q64X Disease Site

This Example further demonstrates the surprising dDR advantage over sDR as in Examples 4-6, using yet another disease site—the Rpe65 Q64X disease site mutation (SEQ ID NO: 50). The Rpe65 Q64X disease site mutation leads to abnormal alternative splicing, and the proportion of full-length mRNA decreases as a result. Therefore, this disease model provides a different context of pre-RNA base editing to enhance translation, as opposed to mRNA base editing to alleviate premature termination of translation.

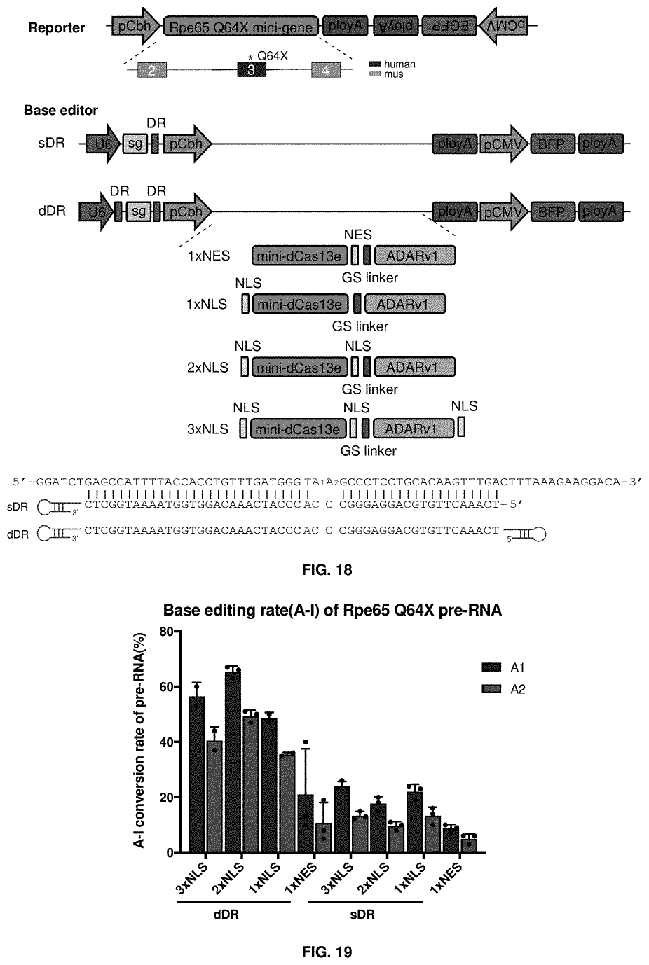

Further, the impact of the different nuclear entry sequences in minidCas13e.1-ADARv1 system was verified again, as in Example 4 (FIG. 18).

Referring to FIG. 18, as in the previous Examples, sDR and dDR gRNA constructs (one or two copies of the DR coding sequence of SEQ ID NO: 8) were constructed for each base editor tested. The base editors differ in that they have 1xNES, 1xNLS, 2xNLS, or 3xNLS, but are otherwise similar with the subject minidCas13e.1 moiety and the ADARv1 moiety. The spacer sequence of sDR and dDR gRNA was designed to correct TA(A₁)A(A₂) to TGG.

The reporter plasmid was transfected into HEK293 cell line together with the different base editor expression plasmid, respectively. After 72 hours of culturing, the cells were sorted by flow cytometry to obtain transfection-positive cells (BFP and EGFP double positive). RNA was extracted, Sanger sequencing or gel electrophoresis was performed after RT-PCR. The A-to-I base editing efficiency of the different base editing systems was analyzed based on Sanger sequencing.

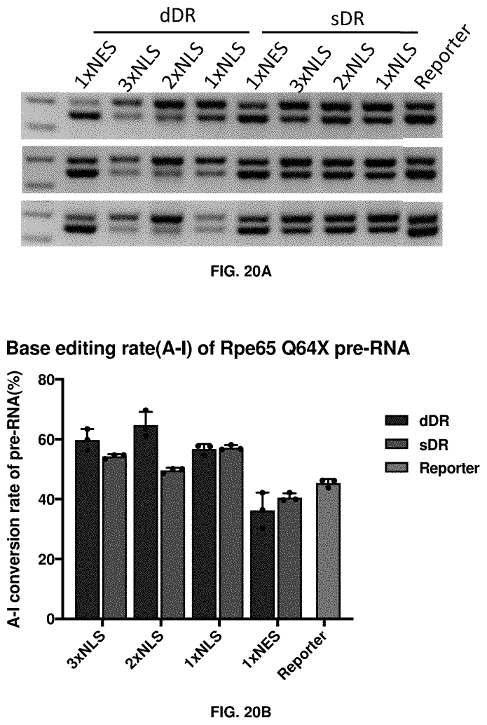

The results (FIG. 19) show that, regardless of the different nuclear entry sequences, all the double DR (dDR) gRNA constructs achieved higher A-to-I base editing efficiency than the corresponding single DR (sDR) gRNA constructs for both A1 site and A2 site, once again confirming the superior base editing efficiency of dDR-gRNA based base editing systems.

On the other hand, based on the result of the gel images of RT-PCR gel electrophoresis (FIGS. 20A and 20B), it was roughly found that the percentage of full-length mRNA (correctly processed mRNA) achieved by the double DR (dDR) gRNA base editing system was higher than or comparable to that by the single DR (sDR) gRNA base editing system.

Overall, the best nuclear entry sequence combination seemed to be 2xNLS and 3xNLS, as also found in Example 4 (FIG. 13).

### Example 8 Mutation in the H₂O Locus Renders EcCas6e Losing DR-Processing Ability

This Example demonstrates that certain mutations in the small Cas effector enzyme of the invention, such as EcCas6e, cause them to lose DR-processing ability, yet retains binding to DR sequences and guide RNA sequences.

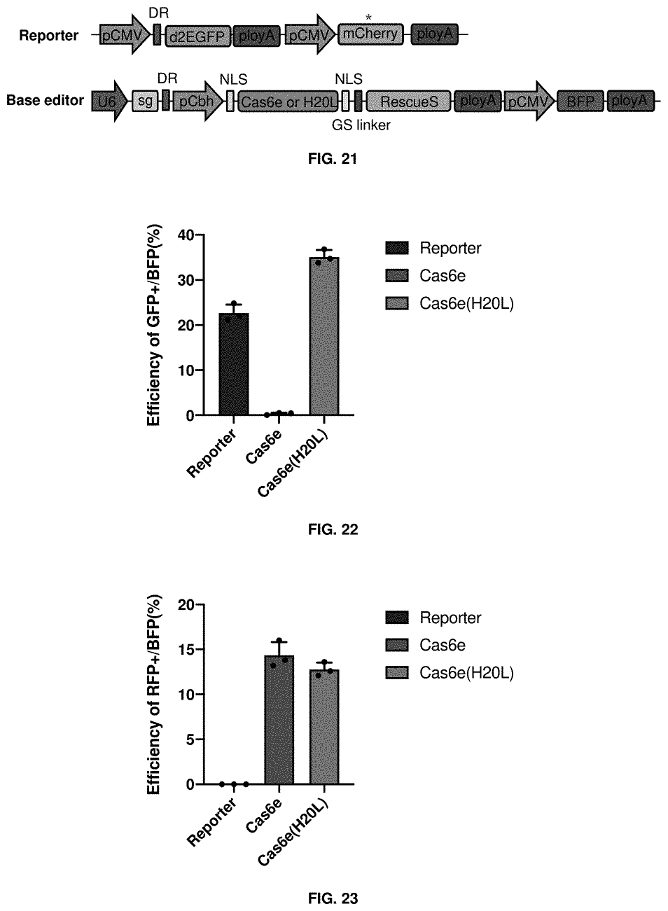

Referring to FIG. 21, for the reporter construct, EcCas6e DR coding sequence (SEQ ID NO: 47) was inserted in front of a d2EGFP (SEQ ID NO: 52) coding sequence, so that the positive rate of EGFP expression was used to represent the loss of DR cutting/processing function of EcCas6e mutant. The higher the positive rate of EGFP expression is, the more serious the loss of DR processing function by the EcCas6e mutation is. A premature stop codon mutation was made in mCherry (SEQ ID NO: 53) coding sequence, so that the mCherry positive rate was used to reflect base editing efficiency. The base editor expression construct was similarly constructed as the previous Examples, where the Cas moiety was either EcCas6e (SEQ ID NO: 55) or EcCas6e-H20L (SEQ ID NO: 51), the deaminase domain was RescueS (SEQ ID NO: 56), and the gRNA was single DR configuration with EcCas6e DR coding sequence (SEQ ID NO; 47) and a spacer coding sequence (SEQ ID NO: 54) targeting the premature stop codon (target site) in the mCherry coding sequence.

The reporter plasmid and the base editor expression plasmid were transferred to HEK293T cells, and the positive rates of EGFP or mCherry were analyzed by flow cytometry after 72 hours to indicate the DR-processing ability of EcCas6e or EcCas6e-H20L and the base editing efficiency of the two base editors, respectively.

The results (FIG. 22) show that EcCas6e (“Cas6e”) protein exhibited a good DR cutting/processing function (see that the positive rate of EGFP is almost 0), whereas the EcCas6e-H20L mutant (“Cas6e(H20L)”) almost completely lost the DR cutting/processing function, yet still exhibited a comparably high base editing efficiency at the mCherry target site (FIG. 23).

### Example 9: Evaluation of the Loss of DR Sequence-Processing Ability of minidCas13e.1 Protein

The previous data has demonstrated that for the RNA base editing mediated by an RNA base editor comprising either minidCas13e.1 (SEQ ID NO: 32) or EcCas6e-H20L (SEQ ID NO: 51), a higher RNA base editing efficiency was achieved for the gRNA construct with dual DR sequences compared to the gRNA construct with a single DR sequence. It has also been demonstrated that EcCas6e-H20L mutant has lost its ability to process the DR sequence of a gRNA. Therefore, it is further investigated in this Example whether minidCas13e.1 also lose its DR sequence-processing ability.

Designs and Constructions:

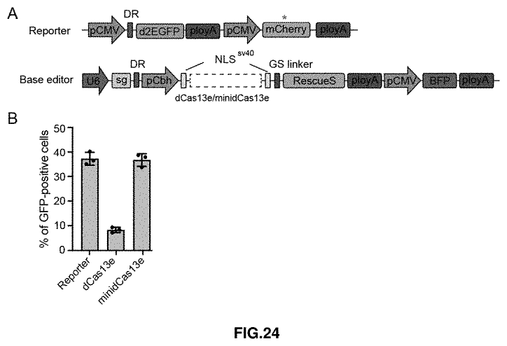

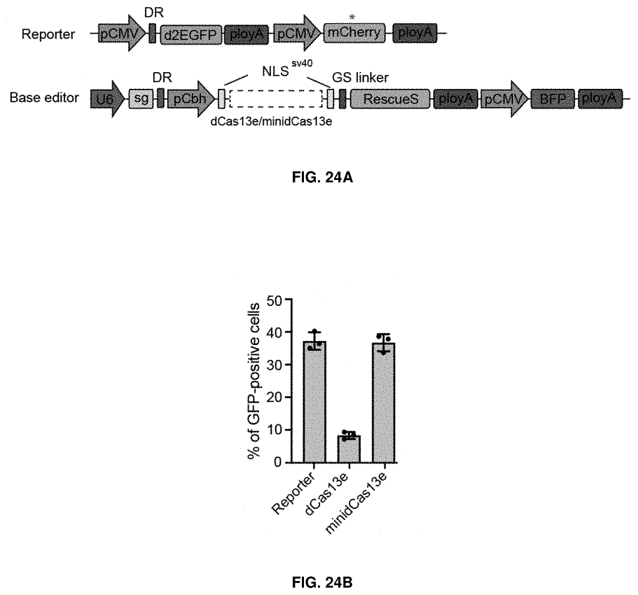

A reporter plasmid and an expression plasmid were constructed for the fluorescent detection of DR sequence-processing ability of minidCas13e.1, as shown in FIG. 24A.

The reporter plasmid comprised a d2EGFP fluorescent reporter gene under the regulation of a CMV promoter and a polyA sequence and a premature stop codon-containing mCherry fluorescent reporter gene under the regulation of a CMV promoter and a poly A sequence. A Cas13e.1 DR coding sequence (SEQ ID NO: 8) was inserted between the CMV promoter and the d2EGFP fluorescent reporter gene.

The expression plasmid for base editor comprised a gRNA coding sequence in 5′-spacer-DR-3′ configuration comprising a Cas13e.1 DR coding sequence (SEQ ID NO: 8) under the regulation of a U6 promoter, a base editor coding sequence under the regulation of a Cbh promoter and a poly A sequence, and a BFP fluorescent reporter gene under the regulation of a CMV promoter and a poly A sequence. The base editor was composed of either full length dCas13e.1 (SEQ ID NO: 31) or minidCas13e.1 (SEQ ID NO: 32) protein flanked by a SV40 NLS (SEQ ID NO: 35) at both N- and C-termini of the dead Cas protein linked to RescueS deaminase domain (human ADAR2_{DD}-E488QN351G/S486A/T375A/S370C/P462A/N597I/L332I/I398V/K3501/M383L/D619G/S582T/V440I/S495 N/K418E/S661T mutant, SEQ ID NO: 56) via a GS linker (SEQ ID NO: 33). The coding sequence (SEQ ID 5 NO: 164) of the spacer sequence comprised in the gRNA was designed to target the premature stop codon on the transcribed mCherry mRNA. The blue fluorescence from BFP would indicate successful transfection and expression of the expression plasmid in host cells.

In the case that the native DR sequence-processing ability of minidCas13e.1 remained, the Cas13e.1 DR transcript section of the Cas13e.1 DR-d2EGFP transcript transcribed from the reporter plasmid would be cleaved, leading to instability and degradation of the latter d2EGFP transcript section and hence none or little green fluorescence signal. In the case that the native DR sequence-processing ability of minidCas13e.1 was reduced or eliminated, d2EGFP would be correctly translated and emit green fluorescence to indicate successful reduction or elimination of the DR sequence-processing ability of minidCas13e.1.

Transfection and Detection

HEK293T cells were cultured in 24-well tissue culture plates according to standard methods for 12 hours, before the reporter and expression plasmids were co-transfected into the cells using standard polyethylenimine (PEI) transfection. The transfected cells were then cultured at 37° C. under CO₂ for 72 hrs. Then the cultured cells were analyzed by flow cytometry. As a negative control, only the reporter plasmid was transfected to the cells. The DR sequence-processing activity was inversely correlated to the percentage proportion of EGFP positive cells in BFP positive cells. The higher the % EGFP/BFP is, the lower the DR sequence-processing ability would be.

Results:

The flow cytometry results (Table 1, FIG. 24B) show that the full length dCas13e.1 protein retained a significant DR sequence-processing ability, because the percentage proportion of EGFP positive cells in BFP positive cells (8.3%) is significantly lower than the negative control with no DR sequence processing (37.2%), whereas minidCas13e.1 nearly lost its DR sequence-processing ability completely and shows a percentage proportion of EGFP positive cells in BFP positive cells (36.7%) comparable to the negative control with no DR sequence processing (37.2%). Thus, the N180+C150 truncation of minidCas13e.1 not only eliminated the RNA cleavage activity but also the DR sequence-processing ability of its parental Cas13e.1 effector protein.

TABLE 1

| | Averaged DR sequence-processing |
|---|---|
| | ability (% of EGFP positive cells in |
| Base editor | BFP positive cells) (n = 3) |
| Negative control | 37.2 |
| dCas13e.1-RescueS | 8.3 |
| minidCas13e.1-RescueS | 36.7 |

The results above again provide a reasonable explanation as to why the gRNA configuration with dual DR sequence is more efficient in base editing than that with a single DR sequence when used in combination with the base editor comprising minidCas13e.1.

### Example 10: Evaluation of the Loss of DR Sequence-Processing Ability of ddCas13b Protein and the Base Editing Efficiency of ddCas13b-Based Base Editor

Following the same procedure to evaluate the loss of DR sequence-processing ability of minidCas13e.1 and EcCas6e-H20L protein and the base editing efficiency of minidCas13e.1- and EcCas6e-H20L-based base editors, another Class 2, Type VI effector protein, PspCas13b, was selected for the double evaluation of DR sequence-processing ability and base editing efficiency to expand the applicability of the dual DR gRNA strategy.

Designs and Constructions:

Mutations D369A+K370A+R380A were introduced into dPspCas13b (SEQ ID NO: 174, from REPAIRv1) to generate ddPspCas13b (SEQ ID NO: 176) for the double evaluation.

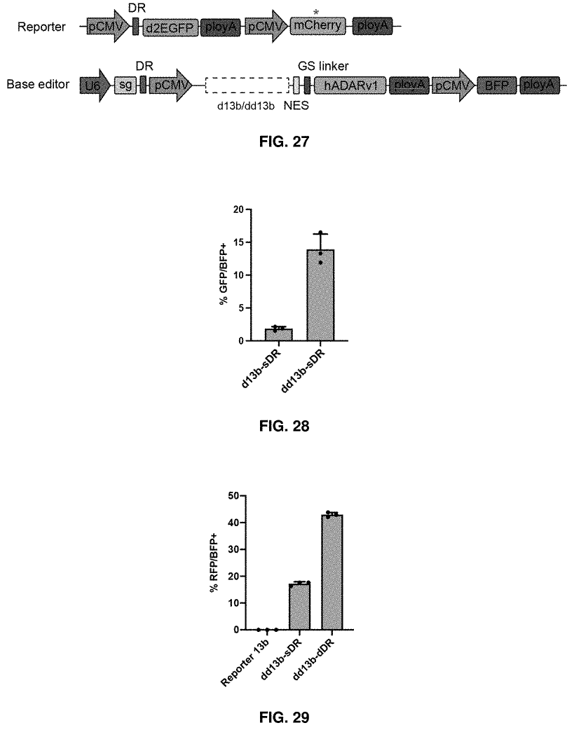

A reporter plasmid and an expression plasmid were constructed for the fluorescent detection of DR sequence-processing ability and base editing efficiency, as shown in FIG. 27.

The reporter plasmid comprised a d2EGFP fluorescent reporter gene under the regulation of a CMV promoter and a polyA sequence and a premature stop codon-containing mCherry fluorescent reporter gene under the regulation of a CMV promoter and a poly A sequence. A PspCas13b DR coding sequence (SEQ ID NO: 173) was inserted between the CMV promoter and the d2EGFP fluorescent reporter gene. The premature stop codon-containing mCherry fluorescent reporter gene contained a W148* premature stop codon TAG mutated from TGG codon (W) at position W148 in its mCherry coding sequence that led to premature termination to prevent the expression of mCherry protein and hence the emission of red fluorescence.

The expression plasmid for base editor comprised a gRNA coding sequence in 5′-spacer-DR-3′ configuration only for the evaluation of DR sequence-processing ability and in both 5′-spacer-DR-3′ configuration and 5′-DR-spacer-DR-3′ configuration (not shown) for the evaluation of base editing efficiency, with the gRNA comprising a PspCas13b DR coding sequence (SEQ ID NO: 173) under the regulation of a U6 promoter, a base editor coding sequence under the regulation of a CMV promoter and a poly A sequence, and a BFP fluorescent reporter gene under the regulation of a CMV promoter and a poly A sequence. The base editor was composed of either dPspCas13b (“d13b”, SEQ ID NO: 174) or ddCas13b (“dd13b”, SEQ ID NO: 176) protein flanked by a NES (SEQ ID NO: 48) at the C-termini of the dead Cas protein linked to ADARv1 deaminase domain (SEQ ID NO: 34) via a GS linker (SEQ ID NO: 33). A short linker of GSLQ was interposed between the Cas protein and the NES. The coding sequence (SEQ ID NO: 166) of the spacer sequence (targeting spacer sequence) comprised in the gRNA was designed to target the W148* premature stop codon on the transcribed mCherry mRNA while containing C corresponding to mismatch G against the target A of the premature stop codon to be edited at W148. The blue fluorescence from BFP would indicate successful transfection and expression of the expression plasmid in host cells.

In the case that the native DR sequence-processing ability of ddPspCas13b remained, the PspCas13b DR transcript section of the PspCas13b DR-d2EGFP transcript transcribed from the reporter plasmid would be cleaved, leading to instability and degradation of the latter d2EGFP transcript section and hence none or little green fluorescence signal. In the case that the native DR sequence-processing ability of ddPspCas13b was reduced or eliminated, d2EGFP would be correctly translated and emit green fluorescence to indicate successful reduction or elimination of the DR sequence-processing ability of ddPspCas13b.

In the case that the W148* premature stop codon on the transcribed mCherry mRNA was successfully edited by the A-to-I base editor, mCherry protein would be correctly translated and emit red fluorescence to indicate the successful on-target A-to-I base editing by the A-to-I base editor.

Transfection and Detection

HEK293T cells were cultured in 24-well tissue culture plates according to standard methods for 12 hours, before the reporter and expression plasmids were co-transfected into the cells using standard polyethylenimine (PEI) transfection. The transfected cells were then cultured at 37° C. under CO₂ for 72 hrs. Then the cultured cells were analyzed by flow cytometry. As a negative control, only the reporter plasmid was transfected to the cells.

The DR sequence-processing activity was inversely correlated to the percentage proportion of EGFP positive cells in BFP positive cells. The higher the % EGFP⁺/BFP⁺ is, the lower the DR sequence-processing ability would be.

The RNA base editing efficiency of each base editor was calculated as the ratio of mCherry positive cells (“R+”, indicating positive base editing at the indicated position) to BFP positive cells (“BFP+”, indicating successful co-transfection and co-expression).

Results:

The flow cytometry results regarding the DR sequence-processing ability (Table 2, FIG. 28) show that the dPspCas13b protein (1.85%) retained a significant DR sequence-processing ability as compared to ddPspCas13b showing a much higher percentage proportion of EGFP positive cells in BFP positive cells (13.90%). Thus, the mutations D369A+K370A+R380A introduced into dPspCas13b eliminated the DR sequence-processing ability of its parental dPspCas13b protein.

The flow cytometry results regarding the A-to-I base editing efficiency (Table 2, FIG. 29) show that similar to the base editors based on minidCas13e.1 and EcCas6e-H20L, the ddPspCas13b-based base editor also achieved a significantly higher A-to-I base editing efficiency with dual DR gRNA configuration (42.93%) than single DR gRNA configuration (17.17%).

TABLE 2

| | Averaged DR sequence- | Averaged A-to-I |
|---|---|---|
| | processing ability | conversion rate |
| | (% EGFP+/BFP+) | (% R+/BFP+) |
| Base editor | (n = 3) | (n = 3) |
| Negative control | — | 0.00 |
| dPspCas13b-ADARv1-sDR | 1.85 | — |
| ddPspCas13b-ADARv1-sDR | 13.90 | 17.17 |
| ddPspCas13b-ADARv1-dDR | — | 42.93 |

Discussion:

The results above again provide a reasonable explanation as to why the gRNA configuration with dual DR sequence is more efficient in base editing than that with a single DR sequence when used in combination with the base editor comprising ddPspCas13b.

Together with the demonstration of more efficient gRNA configuration with dual DR sequence in base editing than that with a single DR sequence when used in combination with the base editor comprising EcCas6e-H20L or minidCas13e.1, of which the DR sequence-processing ability is lost either, it is believed that it is the loss of DR sequence-processing ability of Cas effector proteins that contributes to the maintenance of dual DR gRNA configuration and possibly higher recruitment of dead Cas proteins by the dual DR, leading to higher base editing efficiency when the dead Cas proteins are modified for base editing. It is therefore suggested that by removing the DR sequence-processing ability of Cas effector proteins, the highly efficient guide RNA configuration with dual DR sequence can be applied with such Cas proteins substantially lacking ability to process DR sequence of guide RNAs for various purpose of e.g., base editing, transcription regulation, epigenetic modification.

### Example 11: Identification of Additional, Truncated Cas13 Proteins

A dead version of Cas13e.1, N180+C150 truncation, was constructed by truncations at both N- and C-termini and shown to have the best RNA base editing efficiency and also mini molecular size when combined with a deaminase domain compared with other truncation patterns, which makes it a suitable base for building various RNA tools for the purpose of e.g., base editing, transcription regulation, epigenetic modification.

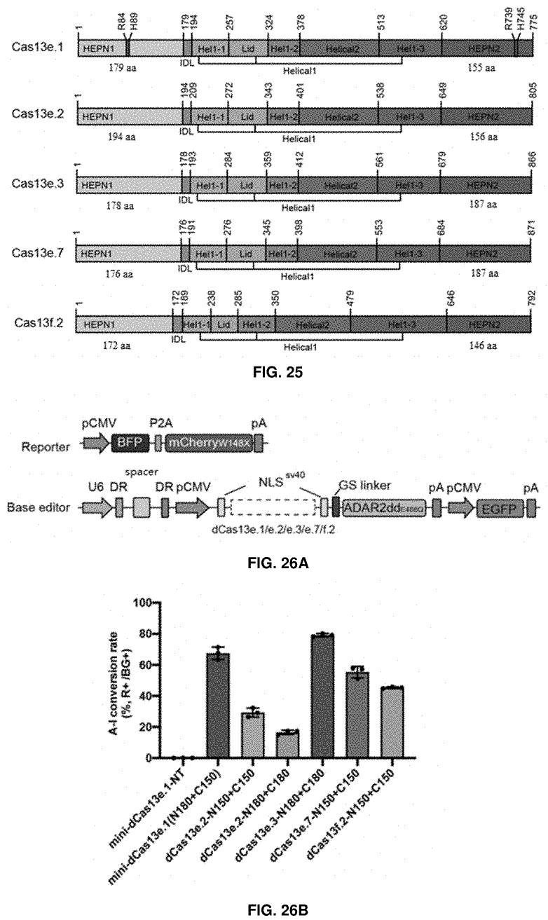

In this Example, some more Cas13 effector proteins (Cas13e.2, Cas13e.3, Cas13e.7, and Cas13f2; FIG. 25) were truncated at the N- and C-termini. Two types of truncations, N150+C150 and N180+C180, were applied herein for primary investigation, representing the deletion of 150 amino acids from both N- and C-termini and the deletion of 180 amino acids from both N- and C-termini, respectively.

It is believed that considering the position of the critical catalytic sites, RxxxxH motifs, predicted on reference Cas13e.1 and the prediction of the functional domain structure of those Cas13 effector proteins by the applicant, the designed truncation should also be sufficient to delete the critical catalytic sites of those Cas13 effector proteins, leading to dead Cas13 proteins as RNA targeting domains suitable for association with various heterologous functional domains for the purpose of e.g., base editing, transcription regulation, epigenetic modification.

To evaluate the applicability of such truncated Cas13 proteins as RNA targeting domains, RNA base editing efficiency of a A-to-I base editor formed by fusing each of the truncated Cas13 proteins to an ADAR deaminase domain to form a fusion protein was detected.

Designs and Constructions:

A reporter plasmid and an expression plasmid were constructed for the fluorescent detection of RNA base editing efficiency as shown in FIG. 26A.

The reporter plasmid comprised a BFP-P2A-mCherry-W148X dual fluorescent reporter gene under the regulation of a CMV promoter and a poly A sequence (SEQ ID NO: 165). The blue fluorescence from BFP would indicate successful transfection and expression of the reporter plasmid in host cells. The dual fluorescent reporter gene contained a W148* premature stop codon TAG mutated from TGG codon (W) at position W148 in its mCherry coding sequence that led to premature termination to prevent the expression of mCherry protein and hence the emission of red fluorescence.

The expression plasmid for base editor comprised a gRNA coding sequence in 5′-DR-spacer-DR-3′ configuration under the regulation of a U6 promoter, a base editor coding sequence under the regulation of a CMV promoter and a poly A sequence, and a EGFP fluorescent reporter gene under the regulation of a CMV promoter and a poly A sequence. The base editor was composed of a truncated Cas13 (dead Cas13) protein flanked by two SV40 NLS (SEQ ID NO: 35) linked to human ADAR2_{DD}-E488Q via a GS linker (SEQ ID NO: 33). The truncated Cas13 tested included dCas13e.2-N150+C150 (SEQ ID NO: 168), dCas13e.2-N180+C180 (SEQ ID NO: 169), dCas13e.3-N180+C180 (SEQ ID NO: 170), dCas13e.7-N150+C150 (SEQ ID NO: 171), dCas13f.2-N150+C150 (SEQ ID NO: 172), and as a positive control, minidCas13e.1-N180+C150 (SEQ ID NO: 32) in Example 1. The coding sequence (SEQ ID NO: 166) of the spacer sequence (targeting spacer sequence) comprised in the gRNA was designed to target the W148* premature stop codon on the transcribed mCherry mRNA while containing C corresponding to mismatch G against the target A of the premature stop codon to be edited at W148. The green fluorescence from EGFP would indicate successful transfection and expression of the expression plasmid in host cells. As a negative control, a coding sequence (SEQ ID NO: 167) of a non-targeting spacer sequence (“NT”) was used in place of the coding sequence (SEQ ID NO: 166) of the targeting spacer sequence.

In the case that the W148* premature stop codon on the transcribed mCherry mRNA was successfully edited by the A-to-I base editor, mCherry protein would be correctly translated and emit red fluorescence to indicate the successful on-target A-to-I base editing by the A-to-I base editor.

Transfection and Detection:

HEK293T cells were cultured in 24-well tissue culture plates according to standard methods for 12 hours, before the reporter and expression plasmids were co-transfected into the cells using standard polyethylenimine (PEI) transfection. The transfected cells were then cultured at 37° C. under CO₂ for 48 hrs. Then the cultured cells were analyzed by flow cytometry. The RNA base editing efficiency of each base editor was calculated as the ratio of mCherry positive cells (“R+”, indicating positive base editing at the indicated position) to BFP/EGFP dual-positive cells (“BG-k”, indicating successful co-transfection and co-expression).

Results:

The flow cytometry results (Table 3, FIG. 26B) show that all the five subject base editors (hADAR2_{DD}-E488Q combined with dCas13e.2-N150+C150, dCas13e.2-N180+C180, dCas13e.3-N180+C180, dCas13e.7-N150+C150, or dCas13f.2-N150+C150) achieved significant A-to-I conversion, and the base editor comprising dCas13e.3-N180+C180 even achieved a higher A-to-I conversion rate than the positive control.

TABLE 3
RNA base editing efficiency of each subject base editor compared with
the base editor comprising minidCas13e.l

| | | | Averaged |
|---|---|---|---|
| | Cas | Truncated | A-to-I |
| | molecular | Cas | conversion |
| | size | molecular | rate |
| A-to-I base editor + spacer | (amino | size | (% R+/BG+) |
| sequence | acids) | (amino acids) | (n = 3) |
| minidCas13e.1-N180 + C150 | 775 | 445 | 0.07 |
| (dCas13e.2-v2) + hADAR2_{DD}- | | | |
| E488Q + non-targeting spacer | | | |
| sequence (negative control) | | | |
| minidCas13e.1-N180 + C150 | 775 | 445 | 67.43 |
| (dCas13e.2-v2) + hADAR2_{DD}- | | | |
| E488Q + targeting spacer | | | |
| sequence (positive control) | | | |
| dCas13e.2-N150 + C150 | 805 | 505 | 29.30 |
| (dCas13e.2-v1) + hADAR2_{DD}- | | | |
| E488Q + targeting spacer | | | |
| sequence | | | |
| dCas13e.2-N180 + C180 | 805 | 445 | 16.57 |
| (dCas13e.2-v2) + hADAR2_{DD}- | | | |
| E488Q + targeting spacer | | | |
| sequence | | | |
| dCas13e.3-N180 + C180 | 866 | 506 | 79.17 |
| (dCas13e.3-v1) + hADAR2_{DD}- | | | |
| E488Q + targeting spacer | | | |
| sequence | | | |
| dCas13e.7-N150 + C150 | 871 | 571 | 55.33 |
| (dCas13e.7-v1) + hADAR2_{DD}- | | | |
| E488Q + targeting spacer | | | |
| sequence | | | |
| dCas13f.2-N150 + C150 | 792 | 492 | 45.33 |
| (dCas13f.2-v1) + hADAR2_{DD}- | | | |
| E488Q + targeting spacer | | | |
| sequence | | | |

Discussion:

It is thus concluded from the above that smaller RNA targeting domains suitable for association with various heterologous functional domains for the purpose of e.g., base editing, transcription regulation, epigenetic modification can be constructed by truncating the N- and C-termini of parental Cas13 proteins to generate dead Cas proteins.

TABLE 4
Analysis of the effect of removing HEPN1 and HEPN2 on
A-to-I base editing efficiency

| | | | | | Averaged | |
|---|---|---|---|---|---|---|
| | | | | | A-to-I | A-to-I |
| | | N-terminal | | C-terminal | conversion | conversion |
| dead Cas | HEPN1 | truncation | HEPN2 | truncation | rate (%) | rate (%) |
| protein | (aa) | (aa) | (aa) | (aa) | (n = 3) | in FIG. 6 |
| dCas13e.1-v1 | 179 | 150 | 155 | 150 | | ~30* |
| dCas13e.1-v2 | 179 | 180 | 155 | 150 | 67.43 | ~53* |
| dCas13e.1-v3 | 179 | 210 | 155 | 150 | | ~10* |
| dCas13e.2-v1 | 194 | 150 | 156 | 150 | 29.30 | |
| dCas13e.2-v2 | 194 | 180 | 156 | 180 | 16.57 | |
| dCas13e.3-v1 | 178 | 180 | 187 | 180 | 79.17 | |
| dCas13e.7-v1 | 176 | 150 | 187 | 150 | 55.33 | |
| dCas13f.2-v1 | 172 | 150 | 146 | 150 | 45.33 | |

*where each of the truncated Casl3e.1 proteins was fused to hADAR2pD-E488Q/T375G instead of hADAR2DD-E488Q.

To make further analysis, the N- and C-terminal truncations and HEPN domains and the associated A-to-I base editing efficiency from Table 3 and from FIG. 6 are listed in the same Table 4 above. By comparing the lengths of the N- and C-terminal truncations and the lengths of HEPN1 and HEPN2 domains, it can be determined whether the HEPN1 and/or HEPN2 domains are removed completely or almost completely, or not completely but retain a substantial portion. For example, dCas13e.1-v1 contains a substantial portion of HEPN1 (retain a substantial portion) and nearly no HEPN2 (removed almost completely); dCas13e.1-v2 contains no HEPN1 (removed almost completely) and nearly no HEPN2 (removed almost completely).

It is observed that when both HEPN1 and HEPN2 domains are removed completely or almost completely, dCas13e.1-v2 (N180+C150) and dCas13e.3-v1(N180+C180) achieved quite high base editing efficiency of 67.43 and 79.17, respectively. For dCas13e.1-v2, HEPN1 in a length of 179 aa is removed completely by 180 aa N-terminal truncation, and HEPN2 in a length of 155 aa is removed almost completely by 150 aa C-terminal truncation. For dCas13e.3-v1, HEPN1 in a length of 178 aa is removed completely by 180 aa N-terminal truncation, and HEPN2 in a length of 187 aa is removed almost completely by 180 aa C-terminal truncation.

In contrast, when either HEPN1 or HEPN2 domain is not removed completely or almost completely but retain a substantial portion, the base editing efficiency of each of dCas13e.1-v1, dCas13e.2-v1, dCas13e.7-v1, and dCas13f.2-v1 was relatively low as compared to dCas13e.1-v2 and dCas13e.3-v1 with complete or almost complete removal of both HEPN1 and HEPN2 domains. Each of dCas13e.1-v1, dCas13e.2-v1, dCas13e.7-v1, and dCas13f.2-v1 retains a substantial portion of HEPN1, and dCas13e.7-v1 also retains a substantial portion of HEPN2.

Interesting, it is also noted that the excessive removal of HEPN1 and/or HEPN2 domain may also disadvantageously affect the base editing efficiency. For example, the 210 aa N-terminal truncation of dCas13e.1-v3 not only remove the whole 179 aa HEPN1 domain but also the whole IDL domain and a substantial portion of the Hel1-1 domain, leading to a quite low base editing efficiency of about 10%, and the 180 aa C-terminal truncation of dCas13e.2-v2 not only remove the whole 156 aa HEPN2 domain but also a substantial portion of the Hel1-3 domain, leading to a quite low base editing efficiency of 16.57%.

It is therefore suggested that the substantially complete removal of both HEPN1 and HEPN2 domain, preferably no more and no less, of Cas13 effector proteins would result in high applicability of mini dead Cas13 proteins as desired RNA targeting domains.

### Example 12: m6A-Associated Epigenetic Up-Regulation of Endogenous Target RNA

For the purpose of m6A-associated epigenetic up-regulation, a m6A-associated epigenetic up-regulating system is designed and tested in this Example.

An expression plasmid for m6A-associated epigenetic up-regulating system is designed to provide a m6A modification to a m6A associated endogenous target RNA in HEK293T cells, comprising a gRNA coding sequence in 5 ‘-DR-spacer-DR-3’ configuration under the regulation of a U6 promoter, a m6A-associated epigenetic regulator coding sequence under the regulation of a Cbh promoter and a poly A sequence, and a BFP fluorescent reporter gene under the regulation of a CMV promoter and a poly A sequence. The m6A-associated epigenetic regulator is composed of minidCas13e.1-N180+C150 (SEQ ID NO: 32) flanked by two SV40 NLS (SEQ ID NO: 35) linked to a m6A providing moiety, human METTL3 (Accession No.: Q86U44), via a GS linker (SEQ ID NO: 33). The spacer sequence (targeting spacer sequence) comprised in the gRNA is designed to target the m6A-associated target RNA. The blue fluorescence from BFP would indicate successful transfection and expression of the expression plasmid in HEK293T cells. As a negative control, a non-targeting spacer sequence (“NT”) is used in place of the targeting spacer sequence.

HEK293T cells are cultured in 24-well tissue culture plates according to standard methods for 12 hours, before the expression plasmid is co-transfected into the cells using standard polyethylenimine (PEI) transfection. The transfected cells are then cultured at 37° C. under CO₂ for 48 hrs. Then the cultured cells are analyzed by flow cytometry. RNA is extracted from the cultured cells, and the introduction of m6A modification onto the target RNA is confirmed by sequencing the extracted RNA with miCLIP-seq technology.

### Example 13: m6A-Associated Epigenetic Down-Regulation of Endogenous Target RNA

For the purpose of m6A-associated epigenetic down-regulation, a m6A-associated epigenetic down-regulating system is designed and tested in this Example.

An expression plasmid for m6A-associated epigenetic down-regulating system is designed to eliminate a m6A modification from a m6A associated endogenous target RNA in HEK293T cells, comprising a gRNA coding sequence in 5′-DR-spacer-DR-3′ configuration under the regulation of a U6 promoter, a m6A-associated epigenetic regulator coding sequence under the regulation of a Cbh promoter and a poly A sequence, and a BFP fluorescent reporter gene under the regulation of a CMV promoter and a poly A sequence. The m6A-associated epigenetic regulator is composed of minidCas13e.1-N180+C150 (SEQ ID NO: 32) flanked by two SV40 NLS (SEQ ID NO: 35) linked to a m6A eliminating moiety, human FTO (Accession No.: Q9C0B1), via a GS linker (SEQ ID NO: 33). The spacer sequence (targeting spacer sequence) comprised in the gRNA is designed to target the m6A-associated target RNA. The blue fluorescence from BFP would indicate successful transfection and expression of the expression plasmid in HEK293T cells. As a negative control, a non-targeting spacer sequence (“NT”) is used in place of the targeting spacer sequence.

HEK293T cells are cultured in 24-well tissue culture plates according to standard methods for 12 hours, before the expression plasmid is co-transfected into the cells using standard polyethylenimine (PEI) transfection. The transfected cells are then cultured at 37° C. under CO₂ for 48 hrs. Then the cultured cells are analyzed by flow cytometry. RNA is extracted from the cultured cells, and the elimination of m6A modification from the target RNA is confirmed by sequencing the extracted RNA with miCLIP-seq technology.

Various modifications and variations of the described systems, elements, compositions, kits, methods, and uses of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

SEQUENCES
Cas protein sequences:

| | |
|---|---|
| Cas13e.1 | MAQVSKQTSKKRELSIDEYQGARKWCFTIAFNKALVNRDKNDGLFVESLLRHEKYSKHDWYDEDTRALIKC |
| amino acid | STOAANAKAEALRNYFSHYRHSPGCLTFTAEDELRTIMERAYERAIFECRRRETEVIIEFPSLFEGDRITT |
| sequence | AGVVFFVSFFVERRVLDRLYGAVSGLKKNEGQYKLTRKALSMYCLKDSRFTKAWDKRVLLFRDILAQLGRI |
| (SEQ ID | PAEAYEYYHGEQGDKKRANDNEGTNPKRHKDKFIEFALHYLEAQHSEICFGRRHIVREEAGAGDEHKKHRT |
| NO: 1) | KGKVVVDFSKKDEDQSYYISKNNVIVRIDKNAGPRSYRMGLNELKYLVLLSLQGKGDDAIAKLYRYRQHVE |
| | NILDVVKVTDKDNHVFLPRFVLEQHGIGRKAFKQRIDGRVKHVRGVWEKKKAATNEMTLHEKARDILQYVN |
| | ENCTRSFNPGEYNRLLVCLVGKDVENFQAGLKRLQLAERIDGRVYSIFAQTSTINEMHQVVCDQILNRLCR |
| | IGDQKLYDYVGLGKKDEIDYKQKVAWFKEHISIRRGFLRKKFWYDSKKGFAKLVEEHLESGGGQRDVGLDK |
| | KYYHIDAIGRFEGANPALYETLARDRLCLMMAQYFLGSVRKELGNKIVWSNDSIELPVEGSVGNEKSIVFS |
| | VSDYGKLYVLDDAEFLGRICEYFMPHEKGKIRYHTVYEKGFRAYNDLQKKCVEAVLAFEEKVVKAKKMSEK |
| | EGAHYIDFREILAQTMCKEAEKTAVNKVRRAFFHHHLKFVIDEFGLFSDVMKKYGIEKEWKFPVK |
| Cas13e.2 | MKVENIKEKSKKAMYLINHYEGPKKWCFAIVLNRACDNYEDNPHLFSKSLLEFEKTSRKDWFDEETRELVE |
| amino acid | QADTEIQPNPNLKPNTTANRKLKDIRNYFSHHYHKNECLYFKNDDPIRCIMEAAYEKSKIYIKGKQIEQSD |
| sequence | IPLPELFESSGWITPAGILLLASFFVERGILHRLMGNIGGFKDNRGEYGLTHDIFTTYCLKGSYSIRAQDH |
| (SEQ ID | DAVMFRDILGYLSRVPTESFQRIKQPQIRKEGQLSERKTDKFITFALNYLEDYGLKDLEGCKACFARSKIV |
| NO: 2) | REQENVESINDKEYKPHENKKKVEIHFDQSKEDRFYINRNNVILKIQKKDGHSNIVRMGVYELKYLVLMSL |
| | VGKAKEAVEKIDNYIQDLRDQLPYIEGKNKEEIKEYVRFFPRFIRSHLGLLQINDEEKIKARLDYVKTKWL |
| | DKKEKSKELELHKKGRDILRYINERCDRELNRNVYNRILELLVSKDLTGFYRELEELKRTRRIDKNIVQNL |
| | SGQKTINALHEKVCDLVLKEIESLDTENLRKYLGLIPKEEKEVTFKEKVDRILKQPVIYKGFLRYQFFKDD |
| | KKSFVLLVEDALKEKGGGCDVPLGKEYYKIVSLDKYDKENKTLCETLAMDRLCLMMARQYYLSLNAKLAQE |
| | AQQIEWKKEDSIELIIFTLKNPDQSKQSFSIRFSVRDFTKLYVTDDPEFLARLCSYFFPVEKEIEYHKLYS |
| | EGINKYTNLQKEGIEAILELEKKLIERNRIQSAKNYLSFNEIMNKSGYNKDEQDDLKKVRNSLLHYKLIFE |
| | KEHLKKFYEVMRGEGIEKKWSLIV |
| Cas13f.1 | MNGIELKKEEAAFYFNQAELNLKAIEDNIFDKERRKTLLNNPQILAKMENFIFNFRDVTKNAKGEIDCLLL |
| amino acid | KLRELRNFYSHYVHKRDVRELSKGEKPILEKYYQFAIESTGSENVKLEIIENDAWLADAGVLFFLCIFLKK |
| sequence | SQANKLISGISGFKRNDDTGQPRRNLFTYFSIREGYKVVPEMQKHFLLFSLVNHLSNQDDYIEKAHQPYDI |
| (SEQID | GEGLFFHRIASTFLNISGILRNMKFYTYQSKRLVEQRGELKREKDIFAWEEPFQGNSYFEINGHKGVIGED |
| NO: 3) | ELKELCYAFLIGNQDANKVEGRITQFLEKFRNANSVQQVKDDEMLKPEYFPANYFAESGVGRIKDRVLNRL |
| | NKAIKSNKAKKGEIIAYDKMREVMAFINNSLPVDEKLKPKDYKRYLGMVRFWDREKDNIKREFETKEWSKY |
| | LPSNFWTAKNLERVYGLAREKNAELFNKLKADVEKMDERELEKYQKINDAKDLANLRRLASDFGVKWEEKD |
| | WDEYSGQIKKQITDSQKLTIMKQRITAGLKKKHGIENLNLRITIDINKSRKAVLNRIAIPRGFVKRHILGW |
| | QESEKVSKKIREAECEILLSKEYEELSKQFFQSKDYDKMTRINGLYEKNKLIALMAVYLMGQLRILFKEHT |
| | KLDDITKTTVDFKISDKVTVKIPFSNYPSLVYTMSSKYVDNIGNYGFSNKDKDKPILGKIDVIEKQRMEFI |
| | KEVLGFEKYLFDDKIIDKSKFADTATHISFAEIVEELVEKGWDKDRLTKLKDARNKALHGEILTGTSFDET |
| | KSLINELKK |
| Cas13f.2 | MSPDFIKLEKQEAAFYFNQTELNLKAIESNILDKQQRMILLNNPRILAKVGNFIFNFRDVTKNAKGEIDCL |
| amino acid | LFKLEELRNFYSHYVHTDNVKELSNGEKPLLERYYQIAIQATRSEDVKFELFETRNENKITDAGVLFFLCM |
| sequence | FLKKSQANKLISGISGFKRNDPTGQPRRNLFTYFSAREGYKALPDMQKHFLLFTLVNYLSNQDEYISELKQ |
| (SEQ ID | YGEIGQGAFFNRIASTFLNISGISGNTKFYSYQSKRIKEQRGELNSEKDSFEWIEPFQGNSYFEINGHKGV |
| NO: 4) | IGEDELKELCYALLVAKQDINAVEGKIMQFLKKFRNTGNLQQVKDDEMLEIEYFPASYFNESKKEDIKKEI |
| | LGRLDKKIRSCSAKAEKAYDKMKEVMEFINNSLPAEEKLKRKDYRRYLKMVRFWSREKGNIEREFRTKEWS |
| | KYFSSDFWRKNNLEDVYKLATQKNAELFKNLKAAAEKMGETEFEKYQQINDVKDLASLRRLTQDFGLKWEE |
| | KDWEEYSEQIKKQITDRQKLTIMKQRVTAELKKKHGIENLNLRITIDSNKSRKAVLNRIAIPRGFVKKHIL |
| | GWQGSEKISKNIREAECKILLSKKYEELSRQFFEAGNFDKLTQINGLYEKNKLTAFMSVYLMGRLNIQLNK |
| | HTELGNLKKTEVDFKISDKVTEKIPFSQYPSLVYAMSRKYVDNVDKYKFSHQDKKKPFLGKIDSIEKERIE |
| | FIKEVLDFEEYLFKNKVIDKSKFSDTATHISFKEICDEMGKKGCNRNKLTELNNARNAALHGEIPSETSFR |
| | EAKPLINELKK |
| Cas13f.3 | MSPDFIKLEKQEAAFYFNQTELNLKAIESNIFDKQQRVILLNNPQILAKVGDFIFNFRDVTKNAKGEIDCL |
| amino acid | LLKLRELRNFYSHYVYTDDVKILSNGERPLLEKYYQFAIEATGSENVKLEIIESNNRLTEAGVLFFLCMFL |
| sequence | KKSQANKLISGISGFKRNDPTGQPRRNLFTYFSVREGYKVVPDMQKHFLLFVLVNHLSGQDDYIEKAQKPY |
| (SEQ ID | DIGEGLFFHRIASTFLNISGILRNMEFYIYQSKRLKEQQGELKREKDIFPWIEPFQGNSYFEINGNKGIIG |
| NO: 5) | EDELKELCYALLVAGKDVRAVEGKITQFLEKFKNADNAQQVEKDEMLDRNNFPANYFAESNIGSIKEKILN |
| | RLGKTDDSYNKTGTKIKPYDMMKEVMEFINNSLPADEKLKRKDYRRYLKMVRIWDSEKDNIKREFESKEWS |
| | KYFSSDFWMAKNLERVYGLAREKNAELFNKLKAVVEKMDEREFEKYRLINSAEDLASLRRLAKDFGLKWEE |
| | KDWQEYSGQIKKQISDRQKLTIMKQRITAELKKKHGIENLNLRITIDSNKSRKAVLNRIAVPRGFVKEHIL |
| | GWQGSEKVSKKTREAKCKILLSKEYEELSKQFFQTRNYDKMTQVNGLYEKNKLLAFMVVYLMERLNILLNK |
| | PTELNELEKAEVDFKISDKVMAKIPFSQYPSLVYAMSSKYADSVGSYKFENDEKNKPFLGKIDTIEKQRME |
| | FIKEVLGFEEYLFEKKIIDKSEFADTATHISFDEICNELIKKGWDKDKLTKLKDARNAALHGEIPAETSFR |
| | EAKPLINGLKK |
| Cas13f.4 | MNIIKLKKEEAAFYFNQTILNLSGLDEIIEKQIPHIISNKENAKKVIDKIFNNRLLLKSVENYIYNFKDVA |
| amino acid | KNARTEIEAILLKLVELRNFYSHYVHNDTVKILSNGEKPILEKYYQIAIEATGSKNVKLVIIENNNCLTDS |
| sequence | GVLFLLCMFLKKSQANKLISSVSGFKRNDKEGQPRRNLFTYYSVREGYKVVPDMQKHFLLFALVNHLSEQD |
| (SEQ ID | DHIEKQQQSDELGKGLFFHRIASTFLNESGIFNKMQFYTYQSNRLKEKRGELKHEKDTFTWIEPFQGNSYF |
| NO: 6) | TLNGHKGVISEDQLKELCYTILIEKQNVDSLEGKIIQFLKKFQNVSSKQQVDEDELLKREYFPANYFGRAG |
| | TGTLKEKILNRLDKRMDPTSKVTDKAYDKMIEVMEFINMCLPSDEKLRQKDYRRYLKMVRFWNKEKHNIKR |
| | EFDSKKWTRFLPTELWNKRNLEEAYQLARKENKKKLEDMRNQVRSLKENDLEKYQQINYVNDLENLRLLSQ |
| | ELGVKWQEKDWVEYSGQIKKQISDNQKLTIMKQRITAELKKMHGIENLNLRISIDTNKSRQTVMNRIALPK |
| | GFVKNHIQQNSSEKISKRIREDYCKIELSGKYEELSRQFFDKKNFDKMTLINGLCEKNKLIAFMVIYLLER |
| | LGFELKEKTKLGELKQTRMTYKISDKVKEDIPLSYYPKLVYAMNRKYVDNIDSYAFAAYESKKAILDKVDI |
| | IEKQRMEFIKQVLCFEEYIFENRIIEKSKFNDEETHISFTQIHDELIKKGRDTEKLSKLKHARNKALHGEI |
| | PDGTSFEKAKLLINEIKK |
| Cas13f.5 | MNAIELKKEEAAFYFNQARLNISGLDEIIEKQLPHIGSNRENAKKTVDMILDNPEVLKKMENYVFNSRDIA |
| amino acid | KNARGELEALLLKLVELRNFYSHYVHKDDVKTLSYGEKPLLDKYYEIAIEATGSKDVRLEIIDDKNKLTDA |
| sequence | GVLFLLCMFLKKSEANKLISSIRGFKRNDKEGQPRRNLFTYYSVREGYKVVPDMQKHFLLFTLVNHLSNQD |
| (SEQ ID | EYISNLRPNQEIGQGGFFHRIASKFLSDSGILHSMKFYTYRSKRLTEQRGELKPKKDHFTWIEPFQGNSYF |
| NO: 7) | SVQGQKGVIGEEQLKELCYVLLVAREDFRAVEGKVTQFLKKFQNANNVQQVEKDEVLEKEYFPANYFENRD |
| | VGRVKDKILNRLKKITESYKAKGREVKAYDKMKEVMEFINNCLPTDENLKLKDYRRYLKMVRFWGREKENI |
| | KREFDSKKWERFLPRELWQKRNLEDAYQLAKEKNTELFNKLKTTVERMNELEFEKYQQINDAKDLANLRQL |
| | ARDFGVKWEEKDWQEYSGQIKKQITDRQKLTIMKQRITAALKKKQGIENLNLRITTDTNKSRKVVLNRIAL |
| | PKGFVRKHILKTDIKISKQIRQSQCPIILSNNYMKLAKEFFEERNFDKMTQINGLFEKNVLIAFMIVYLME |
| | QLNLRLGKNTELSNLKKTEVNFTITDKVTEKVQISQYPSLVFAINREYVDGISGYKLPPKKPKEPPYTFFE |
| | KIDAIEKERMEFIKQVLGFEEHLFEKNVIDKTRFTDTATHISFNEICDELIKKGWDENKIIKLKDARNAAL |
| | HGKIPEDTSFDEAKVLINELKK |

DR coding sequences:

| | |
|---|---|
| Cas13e.1 DR coding sequence | GCTGGAGCAGCCCCCGATTTGTGGGGTGATTACAGC |
| (SEQ ID NO: 8) | |
| Cas13e.2 DR coding sequence | GCTGAAGAAGCCTCCGATTTGAGAGGTGATTACAGC |
| (SEQ ID NO: 9) | |
| Cas13f.1 DR coding sequence | GCTGTGATAGACCTCGATTTGTGGGGTAGTAACAGC |
| (SEQ ID NO: 10) | |
| Cas13f.2 DR coding sequence | GCTGTGATAGACCTCGATTTGTGGGGTAGTAACAGC |
| (SEQ ID NO: 11) | |
| Cas13f.3 DR coding sequence | GCTGTGATAGACCTCGATTTGTGGGGTAGTAACAGC |
| (SEQ ID NO: 12) | |
| Cas13f.4 DR coding sequence | GCTGTGATGGGCCTCAATTTGTGGGGAAGTAACAGC |
| (SEQ ID NO: 13) | |
| Cas13f.5 DR coding sequence | GCTGTGATAGGCCTCGATTTGTGGGGTAGTAACAGC |
| (SEQ ID NO: 14) | |

Cas protein coding sequences:
ATGGCGCAAGTGTCAAAGCAGACTTCGAAAAAGAGAGAGTTGTCTATCGATGAATATCAAGGTGCTCGGAAATGGTGTTTTACGAT
TGCCTTCAACAAGGCTCTTGTGAATCGAGATAAGAACGACGGGCTTTTTGTCGAGTCGCTGTTACGCCATGAAAAGTATTCAAAGC
ACGACTGGTACGATGAGGATACACGCGCTTTGATCAAGTGTAGCACACAAGCGGCCAATGCGAAGGCCGAGGCGTTAAGAAACTAT
TTCTCCCACTATCGACATTCGCCCGGGTGTCTGACATTTACAGCAGAAGATGAGTTGCGGACAATCATGGAAAGGGCGTATGAGCG
GGCGATCTTTGAATGCAGGAGACGCGAAACTGAAGTGATCATCGAGTTTCCCAGCCTGTTCGAAGGCGACCGGATCACTACGGCGG
GGGTTGTGTTTTTCGTTTCGTTCTTTGTTGAACGGCGGGTGCTGGATCGTTTGTACGGTGCGGTAAGTGGGCTTAAGAAAAACGAA
GGACAGTACAAGCTGACTCGGAAGGCGCTTTCGATGTATTGCCTGAAAGACAGTCGTTTCACGAAGGCGTGGGACAAACGCGTGCT
GCTTTTCAGGGATATACTCGCGCAGCTTGGACGCATCCCTGCGGAGGCGTATGAATACTACCACGGAGAGCAGGGCGACAAGAAAA
GAGCAAACGACAATGAGGGGACGAATCCGAAACGCCATAAAGACAAGTTCATCGAGTTTGCACTGCATTATCTGGAGGCGCAACAC
AGTGAGATATGCTTCGGGCGGCGACACATTGTCAGGGAGGAGGCCGGGGCAGGCGACGAACACAAAAAGCACAGGACCAAAGGCAA
GGTAGTTGTCGACTTTTCAAAAAAAGACGAAGATCAGTCATACTATATCAGTAAGAACAATGTTATCGTCAGGATTGATAAGAATG
CCGGGCCTCGGAGTTATCGCATGGGGCTTAACGAATTGAAATACCTTGTATTGCTTAGCCTTCAGGGAAAGGGCGACGATGCGATT
GCAAAACTGTACAGGTATCGGCAGCATGTGGAGAACATTCTGGATGTAGTGAAGGTCACAGATAAGGATAATCACGTCTTCCTGCC
GCGATTTGTGCTGGAGCAACATGGGATTGGCAGGAAAGCTTTTAAGCAAAGAATAGACGGCAGAGTAAAGCATGTTCGAGGGGTGT
GGGAAAAGAAGAAGGCGGCGACCAACGAGATGACACTTCACGAGAAGGCGCGGGACATTCTTCAATACGTAAATGAAAATTGCACG
AGGTCTTTCAATCCCGGCGAGTACAACCGGCTGCTGGTGTGTCTGGTTGGCAAGGATGTTGAGAATTTTCAGGCGGGACTGAAACG
CCTGCAACTGGCCGAGCGAATCGACGGGCGGGTATATTCAATTTTTGCGCAGACCTCCACAATAAACGAGATGCATCAGGTGGTGT
GTGATCAGATTCTCAACAGACTTTGCCGAATCGGCGATCAGAAGCTCTACGATTATGTGGGGCTTGGGAAGAAGGATGAAATAGAT
TACAAGCAGAAGGTTGCATGGTTCAAGGAGCATATTTCTATCCGCAGGGGTTTCTTGCGCAAGAAGTTCTGGTATGACAGCAAGAA
GGGATTCGCGAAGCTTGTGGAAGAGCATTTGGAAAGCGGCGGCGGACAGAGGGACGTTGGGCTGGATAAAAAGTATTATCATATTG
ATGCGATTGGGCGATTCGAGGGTGCTAATCCAGCCTTGTATGAAACGCTGGCGCGAGACCGTTTGTGTCTGATGATGGCGCAATAC
TTCCTGGGGAGTGTACGCAAGGAATTGGGTAATAAAATTGTGTGGTCGAATGATAGCATCGAGTTGCCCGTGGAGGGCTCAGTGGG
TAACGAAAAAAGCATCGTCTTCTCAGTGAGTGATTACGGCAAGTTATATGTGTTGGATGACGCTGAGTTTCTTGGGCGGATATGTG
AGTACTTTATGCCGCACGAAAAAGGGAAGATACGGTATCATACAGTTTACGAAAAAGGGTTTAGGGCATATAATGATCTGCAGAAG
AAATGTGTCGAGGCGGTGCTGGCGTTTGAAGAGAAGGTTGTCAAAGCCAAAAAGATGAGCGAGAAGGAAGGGGCGCATTATATTGA
TTTTCGTGAGATACTGGCACAAACAATGTGTAAAGAGGCGGAGAAGACCGCCGTGAATAAGGTGCGTAGAGCGTTTTTCCATCATC
ATTTAAAGTTTGTGATAGATGAATTTGGGTTGTTTAGTGATGTTATGAAGAAATATGGAATTGAAAAGGAGTGGAAGTTTCCTGTT
AAATGA (SEQ ID NO: 15)
ATGAAGGTTGAAAATATTAAAGAAAAAAGCAAAAAAGCAATGTATTTAATCAACCATTATGAGGGACCCAAAAAATGGTGTTTTGC
AATAGTTCTGAATAGGGCATGTGATAATTACGAGGACAATCCACACTTGTTTTCCAAATCACTTTTGGAATTTGAAAAAACAAGTC
GAAAAGATTGGTTTGACGAAGAAACACGAGAGCTTGTTGAGCAAGCAGATACAGAAATACAGCCAAATCCTAACCTGAAACCTAAT
ACAACAGCTAACCGAAAACTCAAAGATATAAGAAACTATTTTTCGCATCATTATCACAAGAACGAATGCCTGTATTTTAAGAACGA
TGATCCCATACGCTGCATTATGGAAGCGGCGTATGAAAAATCTAAAATTTATATCAAAGGAAAGCAGATTGAGCAAAGCGATATAC
CATTGCCCGAATTGTTTGAAAGCAGCGGTTGGATTACACCGGCGGGGATTTTGTTACTGGCATCCTTTTTTGTTGAACGAGGGATT
CTACATCGCTTGATGGGAAATATCGGAGGATTTAAAGATAATCGAGGCGAATACGGTCTTACACACGATATTTTTACCACCTATTG
TCTTAAGGGTAGTTATTCAATTCGGGCGCAGGATCATGATGCGGTAATGTTCAGAGATATTCTCGGCTATCTGTCACGAGTTCCCA
CTGAGTCATTTCAGCGTATCAAGCAACCTCAAATACGAAAAGAAGGCCAATTAAGTGAAAGAAAGACGGACAAATTTATAACATTT
GCACTAAATTATCTTGAGGATTATGGGCTGAAAGATTTGGAAGGCTGCAAAGCCTGTTTTGCCAGAAGTAAAATTGTAAGGGAACA
AGAAAATGTTGAAAGCATAAATGATAAGGAATACAAACCTCACGAGAACAAAAAGAAAGTTGAAATTCACTTCGATCAGAGCAAAG
AAGACCGATTTTATATTAATCGCAATAACGTTATTTTGAAGATTCAGAAGAAAGATGGACATTCCAACATAGTTAGGATGGGAGTA
TATGAACTTAAATATCTCGTTCTTATGAGTTTAGTGGGAAAAGCAAAAGAAGCAGTTGAAAAAATTGACAACTATATCCAGGATTT
GCGAGACCAGTTGCCTTACATAGAGGGGAAAAATAAGGAAGAGATTAAAGAATACGTCAGGTTCTTTCCACGATTTATACGTTCTC
ACCTCGGTTTACTACAGATTAACGATGAAGAAAAGATAAAAGCTCGATTAGATTATGTTAAGACCAAGTGGTTAGATAAAAAGGAA
AAATCGAAAGAGCTTGAACTTCATAAAAAAGGACGGGACATCCTCAGGTATATCAACGAGCGATGTGATAGAGAGCTTAACAGGAA
TGTATATAACCGTATTTTAGAGCTCCTGGTCAGCAAAGACCTCACTGGTTTTTATCGTGAGCTTGAAGAACTAAAAAGAACAAGGC
GGATAGATAAAAATATTGTCCAGAATCTTTCTGGGCAAAAAACCATTAATGCACTGCATGAAAAGGTCTGTGATCTGGTGCTGAAG
GAAATCGAAAGTCTCGATACAGAAAATCTCAGGAAATATCTTGGATTGATACCCAAAGAAGAAAAAGAGGTCACTTTCAAAGAAAA
GGTCGATAGGATTTTGAAACAGCCAGTTATTTACAAAGGGTTTCTGAGATACCAATTCTTCAAAGATGACAAAAAGAGTTTTGTCT
TACTTGTTGAAGACGCATTGAAGGAAAAAGGAGGAGGTTGTGATGTTCCTCTTGGGAAAGAGTATTATAAAATCGTGTCACTTGAT
AAGTATGATAAAGAAAATAAAACCCTGTGTGAAACTCTGGCGATGGATAGGCTTTGCCTTATGATGGCAAGACAATATTATCTCAG
TCTGAATGCAAAACTTGCACAGGAAGCTCAGCAAATCGAATGGAAGAAAGAAGATAGTATAGAATTGATTATTTTCACCTTAAAAA
ATCCCGATCAATCAAAGCAGAGTTTTTCTATACGGTTTTCGGTCAGAGATTTTACGAAGTTGTATGTAACGGATGATCCTGAATTT
CTGGCCCGGCTTTGTTCCTACTTTTTCCCAGTTGAAAAAGAGATTGAATATCACAAGCTCTATTCAGAAGGGATAAATAAATACAC
AAACCTGCAAAAAGAGGGAATCGAAGCAATACTCGAGCTTGAAAAAAAGCTTATTGAACGAAATCGGATTCAATCTGCAAAAAATT
ATCTCTCATTTAATGAGATAATGAATAAAAGCGGTTATAATAAAGATGAGCAGGATGATCTAAAGAAGGTGCGAAATTCTCTTTTG
CATTATAAGCTTATCTTTGAGAAAGAACATCTCAAGAAGTTCTATGAGGTTATGAGAGGAGAAGGGATAGAGAAAAAGTGGTCTTT
AATAGTATGA (SEQ ID NO: 16)
ATGAATGGCATTGAATTAAAAAAAGAAGAAGCAGCATTTTATTTTAATCAGGCAGAGCTTAATTTAAAAGCCATAGAAGACAATAT
TTTTGATAAAGAAAGACGAAAGACTCTGCTTAATAATCCACAGATACTTGCCAAAATGGAAAATTTCATTTTCAATTTCAGAGATG
TAACAAAAAATGCAAAAGGGGAAATTGACTGCTTGCTGTTGAAACTAAGAGAGCTGAGAAACTTTTACTCGCATTATGTCCACAAA
CGAGATGTAAGAGAATTAAGCAAGGGCGAGAAACCTATACTTGAAAAGTATTACCAATTTGCGATTGAATCAACCGGAAGTGAAAA
TGTTAAACTTGAGATAATAGAAAACGACGCGTGGCTTGCAGATGCCGGTGTGTTGTTTTTCTTATGTATTTTTTTGAAGAAATCTC
AGGCAAATAAGCTTATAAGCGGTATCAGCGGTTTTAAAAGAAACGATGATACCGGTCAGCCGAGAAGGAATTTATTTACCTATTTC
AGTATAAGGGAGGGATACAAGGTTGTTCCGGAAATGCAGAAACATTTCCTTTTGTTTTCTCTTGTTAATCATCTCTCTAATCAAGA
TGATTATATTGAAAAAGCGCATCAGCCATACGATATAGGCGAGGGTTTATTTTTTCATCGAATAGCTTCTACATTTCTTAATATAA
GTGGGATTTTAAGAAATATGAAATTCTATACCTATCAGAGTAAAAGGTTAGTAGAGCAGCGGGGAGAACTCAAACGAGAAAAGGAT
ATTTTTGCGTGGGAAGAACCGTTTCAAGGAAATAGTTATTTTGAAATAAATGGTCATAAAGGAGTAATCGGTGAAGATGAATTGAA
GGAACTATGTTATGCATTTCTGATTGGCAATCAAGATGCTAATAAAGTGGAAGGCAGGATTACACAATTTCTAGAAAAGTTTAGAA
ATGCGAACAGTGTGCAACAAGTTAAAGATGATGAAATGCTAAAACCAGAGTATTTTCCTGCAAATTATTTTGCTGAATCAGGCGTC
GGAAGAATAAAGGATAGAGTGCTTAATCGTTTGAATAAAGCGATTAAAAGCAATAAGGCCAAGAAAGGAGAGATTATAGCATACGA
TAAGATGAGAGAGGTTATGGCGTTCATAAATAATTCTCTGCCGGTAGATGAAAAATTGAAACCAAAAGATTACAAACGATATCTGG
GAATGGTTCGTTTCTGGGACAGGGAAAAAGATAACATAAAGCGGGAGTTCGAGACAAAAGAATGGTCTAAATATCTTCCATCTAAT
TTCTGGACGGCAAAAAACCTTGAAAGGGTCTATGGTCTGGCAAGAGAGAAAAACGCAGAATTATTCAATAAACTAAAAGCGGATGT
AGAAAAAATGGACGAACGGGAACTTGAGAAGTATCAGAAGATAAATGATGCAAAGGATTTGGCAAATTTACGCCGGCTTGCAAGCG
ACTTTGGTGTGAAGTGGGAAGAAAAAGACTGGGATGAGTATTCAGGACAGATAAAAAAACAAATTACAGACAGCCAGAAACTAACA
ATAATGAAGCAGCGGATAACCGCAGGACTAAAGAAAAAGCACGGCATAGAAAATCTTAACCTGAGAATAACTATCGACATCAATAA
AAGCAGAAAGGCAGTTTTGAACAGAATTGCGATTCCGAGGGGTTTTGTAAAAAGGCATATTTTAGGATGGCAAGAGTCTGAGAAGG
TATCGAAAAAGATAAGAGAGGCAGAATGCGAAATTCTGCTGTCGAAAGAATACGAAGAACTATCGAAACAATTTTTCCAAAGCAAA
GATTATGACAAAATGACACGGATAAATGGCCTTTATGAAAAAAACAAACTTATAGCCCTGATGGCAGTTTATCTAATGGGGCAATT
GAGAATCCTGTTTAAAGAACACACAAAACTTGACGATATTACGAAAACAACTGTGGATTTCAAAATATCTGATAAGGTGACGGTAA
AAATCCCCTTTTCAAATTATCCTTCGCTCGTTTATACAATGTCCAGTAAGTATGTTGATAATATAGGGAATTATGGATTTTCCAAC
AAAGATAAAGACAAGCCGATTTTAGGTAAGATTGATGTAATAGAAAAACAGCGAATGGAATTTATAAAAGAGGTTCTTGGTTTTGA
AAAATATCTTTTTGATGATAAAATAATAGATAAAAGCAAATTTGCTGATACAGCGACTCATATAAGTTTTGCAGAAATAGTTGAGG
AGCTTGTTGAAAAAGGATGGGACAAAGACAGACTGACAAAACTTAAAGATGCAAGAAATAAAGCCCTGCATGGTGAAATACTGACG
GGAACCAGCTTTGATGAAACAAAATCATTGATAAACGAATTAAAAAAATGA (SEQ ID NO: 17)
ATGTCCCCAGATTTCATCAAATTAGAAAAACAGGAAGCAGCTTTTTACTTTAATCAGACAGAGCTTAATTTAAAAGCCATAGAAAG
CAATATTTTAGACAAACAACAGCGAATGATTCTGCTTAATAATCCACGGATACTTGCCAAAGTAGGAAATTTCATTTTCAATTTCA
GAGATGTAACAAAAAATGCAAAAGGAGAAATAGACTGTCTGCTATTTAAACTGGAAGAGCTAAGAAACTTTTACTCGCATTATGTT
CATACCGACAATGTAAAGGAATTGAGTAACGGAGAAAAACCCCTACTGGAAAGATATTATCAAATCGCTATTCAGGCAACCAGGAG
TGAGGATGTTAAGTTCGAATTGTTTGAAACAAGAAACGAGAATAAGATTACGGATGCCGGTGTATTGTTTTTCTTATGTATGTTTT
TAAAAAAATCACAGGCAAACAAGCTTATAAGCGGTATCAGCGGCTTCAAAAGAAATGATCCAACAGGCCAGCCGAGAAGAAACTTA
TTTACCTATTTCAGTGCAAGAGAAGGATATAAGGCTTTGCCTGATATGCAGAAACATTTTCTTCTTTTTACTCTGGTTAATTATTT
GTCGAATCAGGATGAGTATATCAGCGAGCTTAAACAATATGGAGAGATTGGTCAAGGAGCCTTTTTTAATCGAATAGCTTCAACAT
TTTTGAATATCAGCGGGATTTCAGGAAATACGAAATTCTATTCGTATCAAAGTAAAAGGATAAAAGAGCAGCGAGGCGAACTCAAT
AGCGAAAAGGACAGCTTTGAATGGATAGAGCCTTTCCAAGGAAACAGCTATTTTGAAATAAATGGGCATAAAGGAGTAATCGGCGA
AGACGAATTAAAAGAACTTTGTTATGCATTGTTGGTTGCCAAGCAAGATATTAATGCCGTTGAAGGCAAAATTATGCAATTCCTGA
AAAAGTTTAGAAATACTGGCAATTTGCAGCAAGTTAAAGATGATGAAATGCTGGAAATAGAATATTTTCCCGCAAGTTATTTTAAT
GAATCAAAAAAAGAGGACATAAAGAAAGAGATTCTTGGCCGGCTGGATAAAAAGATTCGCTCCTGCTCTGCAAAGGCAGAAAAAGC
CTATGATAAGATGAAAGAGGTGATGGAGTTTATAAATAATTCTCTGCCGGCAGAGGAAAAATTGAAACGCAAAGATTATAGAAGAT
ATCTAAAGATGGTTCGTTTCTGGAGCAGAGAAAAAGGCAATATAGAGCGGGAATTTAGAACAAAGGAATGGTCAAAATATTTTTCA
TCTGATTTTTGGCGGAAGAACAATCTTGAAGATGTGTACAAACTGGCAACACAAAAAAACGCTGAACTGTTCAAAAATCTAAAAGC
GGCAGCAGAGAAAATGGGTGAAACGGAATTTGAAAAGTATCAGCAGATAAACGATGTAAAGGATTTGGCAAGTTTAAGGCGGCTTA
CGCAAGATTTTGGTTTGAAGTGGGAAGAAAAGGACTGGGAGGAGTATTCCGAGCAGATAAAAAAACAAATTACGGACAGGCAGAAA
CTGACAATAATGAAACAAAGGGTTACGGCTGAACTAAAGAAAAAGCACGGCATAGAAAATCTTAATCTGAGAATAACCATCGACAG
CAATAAAAGCAGAAAGGCGGTTTTGAACAGAATAGCAATTCCAAGAGGATTTGTAAAAAAACATATTTTAGGCTGGCAGGGATCTG
AGAAGATATCGAAAAATATAAGGGAAGCAGAATGCAAAATTCTGCTATCGAAAAAATATGAAGAGTTATCAAGGCAGTTTTTTGAA
GCCGGTAATTTCGATAAGCTGACGCAGATAAATGGTCTTTATGAAAAGAATAAACTTACAGCTTTTATGTCAGTATATTTGATGGG
TCGGTTGAATATTCAGCTTAATAAGCACACAGAACTTGGAAATCTTAAAAAAACAGAGGTGGATTTTAAGATATCTGATAAGGTGA
CTGAAAAAATACCGTTTTCTCAGTATCCTTCGCTTGTCTATGCGATGTCTCGCAAATATGTTGACAATGTGGATAAATATAAATTT
TCTCATCAAGATAAAAAGAAGCCATTTTTAGGTAAAATTGATTCAATTGAAAAAGAACGTATTGAATTCATAAAAGAGGTTCTCGA
TTTTGAAGAGTATCTTTTTAAAAATAAGGTAATAGATAAAAGCAAATTTTCCGATACAGCGACTCATATTAGCTTTAAGGAAATAT
GTGATGAAATGGGTAAAAAAGGATGTAACCGAAACAAACTAACCGAACTTAACAACGCAAGGAACGCAGCCCTGCATGGTGAAATA
CCGTCGGAGACCTCTTTTCGTGAAGCAAAACCGTTGATAAATGAATTGAAAAAATGA (SEQ ID NO: 18)
ATGTCCCCAGATTTCATCAAATTAGAAAAACAAGAAGCAGCTTTTTACTTTAATCAGACAGAGCTTAATTTAAAAGCCATAGAAAG
CAATATTTTCGACAAACAACAGCGAGTGATTCTGCTTAATAATCCACAGATACTTGCCAAAGTAGGAGATTTTATTTTCAATTTCA
GAGATGTAACAAAAAACGCAAAAGGAGAAATAGACTGTTTGCTATTGAAACTAAGAGAGCTGAGAAACTTTTACTCACACTATGTC
TATACCGATGACGTGAAGATATTGAGTAACGGCGAAAGACCTCTGCTGGAAAAATATTATCAATTTGCGATTGAAGCAACCGGAAG
TGAAAATGTTAAACTTGAAATAATAGAAAGCAACAACCGACTTACGGAAGCGGGCGTGCTGTTTTTCTTGTGTATGTTTTTGAAAA
AGTCTCAGGCAAATAAGCTTATAAGCGGTATCAGCGGTTTTAAAAGAAATGACCCGACAGGTCAGCCGAGAAGGAATTTATTTACC
TACTTCAGTGTAAGGGAGGGATACAAGGTTGTGCCGGATATGCAGAAACATTTTCTTTTGTTTGTTCTTGTCAATCATCTCTCTGG
TCAGGATGATTATATTGAAAAGGCGCAAAAGCCATACGATATAGGCGAGGGTTTATTTTTTCATCGAATAGCTTCTACATTTCTTA
ATATCAGTGGGATTTTAAGAAATATGGAATTCTATATTTACCAGAGCAAAAGACTAAAGGAGCAGCAAGGAGAGCTCAAACGTGAA
AAGGATATTTTTCCATGGATAGAGCCTTTCCAGGGAAATAGTTATTTTGAAATAAATGGTAATAAAGGAATAATCGGCGAAGATGA
ATTGAAAGAGCTTTGTTATGCGTTGCTGGTTGCAGGAAAAGATGTCAGAGCCGTCGAAGGTAAAATAACACAATTTTTGGAAAAGT
TTAAAAATGCGGACAATGCTCAGCAAGTTGAAAAAGATGAAATGCTGGACAGAAACAATTTTCCCGCCAATTATTTCGCCGAATCG
AACATCGGCAGCATAAAGGAAAAAATACTTAATCGTTTGGGAAAAACTGATGATAGTTATAATAAGACGGGGACAAAGATTAAACC
ATACGACATGATGAAAGAGGTAATGGAGTTTATAAATAATTCTCTTCCGGCAGATGAAAAATTGAAACGCAAAGATTACAGAAGAT
ATCTAAAGATGGTTCGTATCTGGGACAGTGAGAAAGATAATATAAAGCGGGAGTTTGAAAGCAAAGAATGGTCAAAATATTTTTCA
TCTGATTTCTGGATGGCAAAAAATCTTGAAAGGGTCTATGGGTTGGCAAGAGAGAAAAACGCCGAATTATTCAATAAGCTAAAAGC
GGTTGTGGAGAAAATGGACGAGCGGGAATTTGAGAAGTATCGGCTGATAAATAGCGCAGAGGATTTGGCAAGTTTAAGACGGCTTG
CGAAAGATTTTGGCCTGAAGTGGGAAGAAAAGGACTGGCAAGAGTATTCTGGGCAGATAAAAAAACAAATTTCTGACAGGCAGAAA
CTGACAATAATGAAACAAAGGATTACGGCTGAACTAAAGAAAAAGCACGGCATAGAAAATCTCAATCTTAGAATAACCATCGACAG
CAATAAAAGCAGAAAGGCAGTTTTGAACAGAATCGCAGTTCCAAGAGGTTTTGTGAAAGAGCATATTTTAGGATGGCAGGGGTCTG
AGAAGGTATCGAAAAAGACAAGAGAAGCAAAGTGCAAAATTCTGCTCTCGAAAGAATATGAAGAATTATCAAAGCAATTTTTCCAA
ACCAGAAATTACGACAAGATGACGCAGGTAAACGGTCTTTACGAAAAGAATAAACTCTTAGCATTTATGGTCGTTTATCTTATGGA
GCGGTTGAATATCCTGCTTAATAAGCCCACAGAACTTAATGAACTTGAAAAAGCAGAGGTGGATTTCAAGATATCTGATAAGGTGA
TGGCCAAAATCCCGTTTTCACAGTATCCTTCGCTTGTGTACGCGATGTCCAGCAAATATGCTGATAGTGTAGGCAGTTATAAATTT
GAGAATGATGAAAAAAACAAGCCGTTTTTAGGCAAGATCGATACAATAGAAAAACAACGAATGGAGTTTATAAAAGAAGTCCTTGG
TTTTGAAGAGTATCTTTTTGAAAAGAAGATAATAGATAAAAGCGAATTTGCCGACACAGCGACTCATATAAGTTTTGATGAAATAT
GTAATGAGCTTATTAAAAAAGGATGGGATAAAGACAAACTAACCAAACTTAAAGATGCCAGGAACGCGGCCCTGCATGGCGAAATA
CCGGCGGAGACCTCTTTTCGTGAAGCAAAACCGTTGATAAATGGATTGAAAAAATGA (SEQ ID NO: 19)
ATGAACATCATTAAATTAAAAAAAGAAGAAGCTGCGTTTTATTTTAATCAGACGATCCTCAATCTTTCAGGGCTTGATGAAATTAT
TGAAAAACAAATTCCGCACATAATCAGCAACAAGGAAAATGCAAAGAAAGTGATTGATAAGATTTTCAATAACCGCTTATTATTAA
AAAGTGTGGAGAATTATATCTACAACTTTAAAGATGTGGCTAAAAACGCAAGAACTGAAATTGAGGCTATATTGTTGAAATTAGTA
GAGCTACGTAATTTTTACTCACATTACGTTCATAATGATACCGTCAAGATACTAAGTAACGGTGAAAAACCTATACTGGAAAAATA
TTATCAAATTGCTATAGAAGCAACCGGAAGTAAAAATGTTAAACTTGTAATCATAGAAAACAACAACTGTCTCACGGATTCTGGCG
TGCTGTTTTTGCTGTGTATGTTCTTAAAAAAATCACAGGCAAACAAGCTTATAAGTTCCGTTAGTGGTTTTAAAAGGAATGATAAA
GAAGGACAACCGAGAAGAAATCTATTCACTTATTATAGTGTGAGGGAGGGATATAAGGTTGTGCCTGATATGCAGAAGCATTTCCT
TCTATTCGCTCTGGTCAATCATCTATCTGAGCAGGATGATCATATTGAGAAGCAGCAGCAGTCAGACGAGCTCGGTAAGGGTTTGT
TTTTCCATCGTATAGCTTCGACTTTTTTAAACGAGAGCGGCATCTTCAATAAAATGCAATTTTATACATATCAGAGCAACAGGCTA
AAAGAGAAAAGAGGAGAACTCAAACACGAAAAGGATACCTTTACATGGATAGAGCCTTTTCAAGGCAATAGTTATTTTACGTTAAA
TGGACATAAGGGAGTGATTAGTGAAGATCAATTGAAGGAGCTTTGTTACACAATTTTAATTGAGAAGCAAAACGTTGATTCCTTGG
AAGGTAAAATTATACAATTTCTCAAAAAATTTCAGAATGTCAGCAGCAAGCAGCAAGTTGACGAAGATGAATTGCTTAAAAGAGAA
TATTTCCCTGCAAATTACTTTGGCCGGGCAGGAACAGGGACCCTAAAAGAAAAGATTCTAAACCGGCTTGATAAGAGGATGGATCC
TACATCTAAAGTGACGGATAAAGCTTATGACAAAATGATTGAAGTGATGGAATTTATCAATATGTGCCTTCCGTCTGATGAGAAGT
TGAGGCAAAAGGATTATAGACGATACTTAAAGATGGTTCGTTTCTGGAATAAGGAAAAGCATAACATTAAGCGCGAGTTTGACAGT
AAAAAATGGACGAGGTTTTTGCCGACGGAATTGTGGAATAAAAGAAATCTAGAAGAAGCCTATCAATTAGCACGGAAAGAGAACAA
AAAGAAACTTGAAGATATGAGAAATCAAGTACGAAGCCTTAAAGAAAATGACCTTGAAAAATATCAGCAGATTAATTACGTTAATG
ACCTGGAGAATTTAAGGCTTCTGTCACAGGAGTTAGGTGTGAAATGGCAGGAAAAGGACTGGGTTGAATATTCCGGGCAGATAAAG
AAGCAGATATCAGACAATCAGAAACTTACAATCATGAAACAAAGGATTACCGCTGAACTAAAGAAAATGCACGGCATCGAGAATCT
TAATCTTAGAATAAGCATTGACACGAATAAAAGCAGGCAGACGGTTATGAACAGGATAGCTTTGCCCAAAGGTTTTGTGAAGAATC
ATATCCAGCAAAATTCGTCTGAGAAAATATCGAAAAGAATAAGAGAGGATTATTGTAAAATTGAGCTATCGGGAAAATATGAAGAA
CTTTCAAGGCAATTTTTTGATAAAAAGAATTTCGATAAGATGACACTGATAAACGGCCTTTGTGAAAAGAACAAACTTATCGCATT
TATGGTTATCTATCTTTTGGAGCGGCTTGGATTTGAATTAAAGGAGAAAACAAAATTAGGCGAGCTTAAACAAACAAGGATGACAT
ATAAAATATCCGATAAGGTAAAAGAAGATATCCCGCTTTCCTATTACCCCAAGCTTGTGTATGCAATGAACCGAAAATATGTTGAC
AATATCGATAGTTATGCATTTGCGGCTTACGAATCCAAAAAAGCTATTTTGGATAAAGTGGATATCATAGAAAAGCAACGTATGGA
ATTTATCAAACAAGTTCTCTGTTTTGAGGAATATATTTTCGAAAATAGGATTATCGAAAAAAGCAAATTTAATGACGAGGAGACTC
ATATAAGTTTTACACAAATACATGATGAGCTTATTAAAAAAGGACGGGACACAGAAAAACTCTCTAAACTCAAACATGCAAGGAAT
AAAGCCTTGCACGGCGAGATTCCTGATGGGACTTCTTTTGAAAAAGCAAAGCTATTGATAAATGAAATCAAAAAATGA (SEQ ID
NO: 20)
ATGAATGCTATCGAACTAAAAAAAGAGGAAGCAGCATTTTATTTTAATCAGGCAAGACTCAACATTTCAGGACTTGATGAAATTAT
TGAAAAGCAGTTACCACATATAGGTAGTAACAGGGAGAATGCGAAAAAAACTGTTGATATGATTTTGGATAATCCCGAAGTCTTGA
AGAAGATGGAAAATTATGTCTTTAACTCACGAGATATAGCAAAGAACGCAAGAGGTGAACTTGAAGCATTGTTGTTGAAATTAGTA
GAACTGCGTAATTTTTATTCACATTATGTTCATAAAGATGATGTTAAGACATTGAGTTACGGAGAAAAACCTTTACTGGATAAATA
TTATGAAATTGCGATTGAAGCGACCGGAAGTAAAGATGTCAGACTTGAGATAATAGATGATAAAAATAAGCTTACAGATGCCGGTG
TGCTTTTTTTATTGTGTATGTTTTTGAAAAAATCAGAGGCAAACAAACTTATCAGTTCAATCAGGGGCTTTAAAAGAAACGATAAA
GAAGGCCAGCCGAGAAGAAATCTATTCACTTACTACAGTGTCAGAGAGGGATATAAGGTTGTGCCTGATATGCAGAAACATTTTCT
TTTATTCACACTGGTTAACCATTTGTCAAATCAGGATGAATACATCAGTAATCTTAGGCCGAATCAAGAAATCGGCCAAGGGGGAT
TTTTCCATAGAATAGCATCAAAATTTTTGAGCGATAGCGGGATTTTACATAGTATGAAATTCTACACCTACCGGAGTAAAAGACTA
ACAGAACAACGGGGGGAGCTTAAGCCGAAAAAAGATCATTTTACATGGATAGAGCCTTTTCAGGGAAACAGTTATTTTTCAGTGCA
GGGCCAAAAAGGAGTAATTGGTGAAGAGCAATTAAAGGAGCTTTGTTATGTATTGCTGGTTGCCAGAGAAGATTTTAGGGCCGTTG
AGGGCAAAGTTACACAATTTCTGAAAAAGTTTCAGAATGCTAATAACGTACAGCAAGTTGAAAAAGATGAAGTGCTGGAAAAAGAA
TATTTTCCTGCAAATTATTTTGAAAATCGAGACGTAGGCAGAGTAAAGGATAAGATACTTAATCGTTTGAAAAAAATCACTGAAAG
CTATAAAGCTAAAGGGAGGGAGGTTAAAGCCTATGACAAGATGAAAGAGGTAATGGAGTTTATAAATAATTGCCTGCCAACAGATG
AAAATTTGAAACTCAAAGATTACAGAAGATATCTGAAAATGGTTCGTTTCTGGGGCAGGGAAAAGGAAAATATAAAGCGGGAATTT
GACAGTAAAAAATGGGAGAGGTTTTTGCCAAGAGAACTCTGGCAGAAAAGAAACCTCGAAGATGCGTATCAACTGGCAAAAGAGAA
AAACACCGAGTTATTCAATAAATTGAAAACAACTGTTGAGAGAATGAACGAACTGGAATTCGAAAAGTATCAGCAGATAAACGACG
CAAAAGATTTGGCAAATTTAAGGCAACTGGCGCGGGACTTCGGCGTGAAGTGGGAAGAAAAGGACTGGCAAGAGTATTCGGGGCAG
ATAAAAAAACAAATTACAGACAGGCAAAAACTTACAATAATGAAACAAAGGATTACTGCTGCATTGAAGAAAAAGCAAGGCATAGA
AAATCTTAATCTTAGGATAACAACCGACACCAATAAAAGCAGAAAGGTGGTATTGAACAGAATAGCGCTACCTAAAGGTTTTGTAA
GGAAGCATATCTTAAAAACAGATATAAAGATATCAAAGCAAATAAGGCAATCACAATGTCCTATTATACTGTCAAACAATTATATG
AAGCTGGCAAAGGAATTCTTTGAGGAGAGAAATTTTGATAAGATGACGCAGATAAACGGGCTATTTGAGAAAAATGTACTTATAGC
GTTTATGATAGTTTATCTGATGGAACAACTGAATCTTCGACTTGGTAAGAATACGGAACTTAGCAATCTTAAAAAAACGGAGGTTA
ATTTTACGATAACCGACAAGGTAACGGAAAAAGTCCAGATTTCGCAGTATCCATCGCTTGTTTTCGCCATAAACAGAGAATATGTT
GATGGAATCAGCGGTTATAAGTTACCGCCCAAAAAACCGAAAGAGCCTCCGTATACTTTCTTCGAGAAAATAGACGCAATAGAAAA
AGAACGAATGGAATTCATAAAACAGGTCCTCGGTTTCGAAGAACATCTTTTTGAGAAGAATGTAATAGACAAAACTCGCTTTACTG
ATACTGCGACTCATATAAGTTTTAATGAAATATGTGATGAGCTTATAAAAAAAGGATGGGACGAAAACAAAATAATAAAACTTAAA
GATGCGAGGAATGCAGCATTGCATGGTAAGATACCGGAGGATACGTCTTTTGATGAAGCGAAAGTACTGATAAATGAATTAAAAAA
ATGA (SEQ ID NO: 21)
Codon-optimized Cas protein coding sequences:
ATGGCCCAGGTGAGCAAGCAGACCTCCAAGAAGAGGGAGCTGAGCATCGACGAGTACCAGGGCGCCCGGAAGTGGTGCTTCACCAT
TGCCTTCAACAAGGCCCTGGTGAACCGGGACAAGAACGACGGCCTGTTCGTGGAAAGCCTGCTGAGACACGAGAAGTACAGCAAGC
ACGACTGGTACGACGAAGATACCCGGGCCCTGATCAAGTGCAGCACCCAGGCCGCCAACGCCAAGGCTGAAGCCCTGCGGAACTAC
TTCAGTCACTACCGGCATAGCCCTGGCTGCCTGACCTTCACCGCCGAGGACGAACTGCGGACCATCATGGAGAGAGCCTATGAGCG
GGCCATCTTCGAGTGCAGAAGAAGAGAGACAGAGGTGATCATCGAGTTTCCCAGCCTGTTCGAGGGCGACCGGATCACCACCGCCG
GCGTGGTGTTTTTCGTGAGCTTTTTCGTGGAAAGAAGAGTGCTGGATCGGCTGTATGGAGCCGTGTCCGGCCTGAAGAAGAATGAG
GGACAGTACAAGCTGACCCGGAAGGCCCTGAGCATGTACTGCCTGAAGGACAGCAGATTCACCAAGGCCTGGGATAAGCGGGTGCT
GCTGTTCAGAGACATCCTGGCCCAGCTGGGAAGAATCCCCGCCGAGGCCTACGAGTACTACCACGGCGAGCAGGGTGATAAGAAGA
GAGCTAACGACAATGAGGGCACAAATCCCAAGCGGCACAAGGACAAGTTCATCGAATTTGCACTGCACTACCTGGAAGCCCAGCAC
AGCGAGATCTGCTTCGGCAGACGCCACATCGTGCGGGAAGAGGCCGGCGCCGGCGATGAGCACAAGAAGCACCGGACCAAGGGAAA
GGTGGTGGTGGACTTCAGCAAGAAGGACGAGGACCAGAGCTACTATATCTCCAAGAACAACGTGATCGTGCGGATCGACAAGAACG
CCGGCCCTAGAAGCTACCGGATGGGCCTGAACGAGCTGAAGTACCTCGTGCTGCTGAGCCTGCAGGGGAAGGGCGACGATGCCATC
GCCAAGCTGTACAGATACAGACAGCACGTGGAGAACATCCTGGATGTGGTGAAGGTGACCGATAAGGATAACCACGTGTTCCTGCC
CCGCTTCGTGCTGGAGCAGCACGGCATCGGCAGAAAGGCCTTCAAGCAGCGGATCGATGGACGGGTGAAGCACGTGCGGGGCGTGT
GGGAGAAGAAGAAGGCCGCCACCAATGAAATGACCCTGCACGAGAAGGCCAGAGACATCCTGCAGTACGTGAACGAAAACTGCACC
CGGTCCTTCAACCCTGGCGAATACAACAGACTGCTGGTGTGCCTGGTGGGCAAGGACGTGGAGAACTTTCAGGCCGGCCTGAAGCG
GCTGCAGCTGGCCGAAAGGATCGATGGCCGGGTGTACTCCATCTTCGCCCAGACCAGCACCATCAATGAGATGCACCAGGTGGTGT
GCGACCAGATCCTGAACCGGCTGTGCAGAATCGGCGACCAGAAGCTGTACGATTACGTGGGACTGGGCAAGAAGGACGAAATCGAC
TACAAGCAGAAGGTGGCCTGGTTCAAGGAGCACATCAGCATCCGGAGAGGATTCCTGAGAAAGAAGTTCTGGTACGATAGCAAGAA
GGGATTCGCAAAGCTGGTGGAGGAACACCTGGAGTCCGGCGGCGGCCAGCGCGACGTGGGCCTGGACAAGAAGTACTACCACATCG
ACGCCATCGGCAGATTCGAGGGCGCCAACCCCGCCCTGTACGAGACCCTGGCCAGAGATCGGCTGTGCCTCATGATGGCCCAGTAC
TTCCTGGGCAGCGTGAGAAAGGAACTGGGCAACAAGATTGTGTGGAGCAACGACAGCATCGAACTGCCTGTGGAAGGCTCTGTGGG
AAATGAGAAGAGCATCGTGTTCTCCGTGTCTGACTACGGCAAGCTGTACGTGCTGGACGATGCCGAATTCCTGGGCCGGATCTGCG
AATACTTCATGCCCCACGAAAAGGGCAAGATCCGGTACCACACAGTGTACGAAAAGGGCTTTAGAGCATACAACGACCTGCAGAAG
AAGTGCGTGGAGGCCGTGCTGGCTTTCGAAGAGAAGGTGGTGAAGGCCAAGAAGATGAGCGAGAAGGAAGGCGCCCACTACATCGA
CTTCCGGGAGATCCTGGCCCAGACCATGTGCAAGGAGGCCGAGAAGACCGCAGTGAACAAGGTGAGACGCGCCTTCTTCCACCACC
ACCTGAAGTTCGTGATTGACGAGTTCGGCCTGTTCAGCGACGTGATGAAGAAGTACGGCATCGAGAAGGAATGGAAGTTCCCTGTC
AAGTAA (SEQ ID NO: 22)
ATGAAGGTGGAGAACATCAAGGAAAAGTCCAAGAAGGCTATGTATCTGATCAACCACTATGAAGGCCCTAAGAAGTGGTGCTTCGC
CATCGTGCTGAATAGGGCCTGCGACAACTATGAGGATAACCCCCACCTGTTCAGCAAGAGCCTGCTGGAATTTGAAAAGACCAGCA
GAAAGGACTGGTTCGACGAGGAGACCAGGGAACTGGTGGAGCAGGCCGACACCGAGATCCAGCCCAACCCCAACCTGAAGCCTAAC
ACCACCGCCAACAGAAAGCTGAAGGACATCCGGAACTACTTCAGCCACCACTACCACAAGAATGAGTGCCTGTACTTCAAGAACGA
CGACCCTATCCGGTGCATCATGGAGGCAGCCTACGAGAAGTCCAAGATCTACATCAAGGGCAAGCAGATTGAGCAGTCCGACATCC
CCCTCCCTGAGCTGTTTGAGTCTAGCGGCTGGATCACCCCAGCCGGCATCCTGCTGCTGGCCAGCTTCTTTGTGGAGAGAGGCATT
CTGCACAGACTGATGGGCAACATCGGCGGCTTCAAGGACAACCGGGGCGAATACGGACTGACCCACGATATCTTCACCACCTACTG
CCTGAAGGGCAGCTACTCCATCAGAGCCCAGGACCACGACGCCGTGATGTTCAGAGACATCCTGGGCTACCTGAGCAGAGTGCCGA
CCGAGAGCTTTCAGCGCATCAAGCAGCCACAGATCAGAAAGGAGGGGCAGCTGAGCGAGCGGAAGACAGACAAGTTTATCACCTTC
GCCCTGAACTACCTGGAAGATTATGGACTGAAGGATCTGGAAGGCTGCAAGGCCTGCTTCGCCCGGAGCAAGATCGTGAGAGAGCA
GGAGAACGTGGAAAGCATCAATGACAAGGAGTACAAGCCTCACGAAAACAAGAAGAAGGTGGAAATCCACTTCGATCAGTCTAAGG
AAGACCGGTTCTACATCAACCGGAACAACGTGATCCTGAAGATCCAGAAGAAGGACGGCCACAGCAACATCGTGAGAATGGGCGTG
TACGAGCTGAAGTATCTGGTGCTGATGTCCCTGGTGGGCAAGGCCAAGGAAGCCGTGGAGAAGATCGACAACTACATCCAGGATCT
GAGAGACCAGCTGCCCTACATCGAGGGCAAGAACAAGGAAGAAATCAAGGAGTACGTGAGATTCTTCCCCAGATTCATCAGATCCC
ACCTGGGCCTGCTGCAGATTAACGATGAGGAGAAGATCAAGGCCCGGCTGGACTATGTGAAGACAAAGTGGCTGGACAAGAAGGAG
AAGTCCAAGGAGCTGGAGCTGCACAAGAAGGGCCGGGATATCCTGCGGTACATCAACGAGCGGTGCGACCGGGAGCTGAACCGGAA
CGTGTACAACCGGATCCTGGAGCTGCTGGTGAGCAAGGACCTGACCGGCTTCTACCGGGAGCTGGAGGAGCTGAAGCGGACCAGAC
GGATCGATAAGAACATTGTGCAGAACCTGTCCGGCCAGAAGACCATCAACGCCCTGCACGAAAAGGTGTGCGATCTCGTGCTGAAG
GAGATCGAGAGCCTGGACACCGAGAACCTGCGGAAGTACCTGGGCCTGATCCCCAAGGAGGAGAAGGAAGTGACCTTTAAGGAGAA
GGTGGACAGGATCCTGAAGCAGCCGGTGATCTACAAGGGCTTCCTGCGGTACCAGTTCTTCAAGGACGACAAGAAGAGCTTCGTGC
TGCTGGTGGAAGACGCCCTGAAGGAGAAGGGAGGCGGCTGCGACGTGCCCCTGGGCAAGGAGTACTACAAGATCGTGTCCCTGGAC
AAGTATGACAAGGAAAATAAGACCCTGTGCGAGACCCTGGCAATGGATAGACTGTGCCTGATGATGGCCCGGCAGTATTACCTGAG
CCTGAACGCCAAGCTGGCCCAGGAGGCCCAGCAGATCGAATGGAAGAAGGAGGATAGCATTGAGCTGATCATCTTCACACTGAAGA
ATCCTGACCAGTCCAAGCAGAGCTTCTCCATCCGGTTCAGCGTGCGGGACTTCACCAAGCTGTACGTGACCGACGACCCCGAATTC
CTGGCCCGGCTGTGCAGCTACTTCTTCCCCGTGGAGAAGGAGATCGAATACCACAAGCTGTACTCTGAAGGCATTAACAAGTACAC
CAACCTGCAGAAGGAGGGGATCGAAGCCATCCTGGAGCTGGAGAAGAAGCTGATCGAAAGAAACCGGATCCAGTCCGCCAAGAACT
ACCTGAGCTTTAACGAAATCATGAACAAGAGCGGCTACAACAAGGATGAGCAGGATGACCTGAAGAAGGTGAGGAACTCCCTGCTG
CACTACAAGCTGATCTTCGAAAAGGAGCACCTGAAGAAGTTCTATGAAGTGATGCGGGGCGAGGGAATCGAGAAGAAGTGGTCCCT
GATCGTGTAA (SEQ ID NO: 23)
ATGAATGGCATCGAGCTGAAGAAGGAAGAAGCCGCCTTCTACTTCAATCAGGCCGAGCTGAACCTGAAGGCCATTGAGGACAACAT
CTTCGACAAGGAGAGACGGAAGACACTGCTGAACAACCCCCAGATCCTGGCCAAGATGGAGAACTTTATCTTCAATTTCCGGGACG
TGACCAAGAACGCCAAGGGCGAAATCGACTGCCTGCTGCTGAAGCTGAGAGAGCTGCGGAACTTTTACAGCCACTACGTGCACAAG
CGGGACGTCAGAGAACTGAGCAAGGGCGAGAAGCCGATCCTGGAGAAGTACTACCAGTTCGCCATCGAATCCACCGGCTCTGAGAA
CGTGAAGCTCGAAATCATCGAAAACGACGCCTGGCTGGCCGACGCCGGCGTGCTGTTCTTCCTGTGCATCTTCCTGAAGAAGAGCC
AGGCAAACAAGCTGATCAGCGGCATCAGCGGCTTCAAGAGAAACGACGACACCGGCCAGCCTCGGAGAAACCTGTTCACCTACTTC
TCCATCCGGGAGGGCTACAAGGTGGTGCCCGAAATGCAGAAGCACTTCCTGCTGTTCTCCCTGGTGAACCACCTGAGCAACCAGGA
CGATTATATCGAAAAGGCCCACCAGCCCTACGACATCGGCGAGGGCCTCTTCTTCCACCGGATTGCCAGCACCTTCCTGAACATCT
CCGGAATCCTGAGAAACATGAAGTTCTACACCTATCAGAGCAAGAGACTGGTGGAGCAGAGAGGCGAGCTGAAGCGGGAAAAGGAC
ATCTTCGCCTGGGAAGAACCGTTTCAGGGCAATTCCTACTTTGAGATCAACGGCCACAAGGGCGTGATTGGCGAAGACGAGCTGAA
GGAGCTGTGCTACGCCTTCCTGATCGGCAACCAGGACGCCAACAAGGTGGAGGGCCGGATCACCCAGTTCCTGGAGAAGTTCAGAA
ACGCCAACAGCGTGCAGCAGGTGAAGGACGACGAGATGCTGAAGCCTGAATATTTCCCCGCCAACTACTTTGCCGAGAGCGGCGTG
GGCCGGATCAAGGACCGGGTGCTGAACAGACTGAACAAGGCCATCAAGAGCAACAAGGCCAAGAAGGGCGAGATCATCGCCTATGA
CAAGATGAGAGAAGTGATGGCTTTCATCAATAACTCTCTGCCCGTGGACGAGAAGCTGAAGCCCAAGGATTACAAGAGATACCTGG
GCATGGTGAGATTCTGGGATAGAGAAAAGGACAATATCAAGCGCGAGTTCGAAACGAAGGAGTGGAGCAAGTATCTGCCCTCCAAC
TTCTGGACCGCCAAGAACCTGGAGAGAGTGTACGGACTGGCCCGGGAAAAGAACGCAGAGCTGTTTAACAAGCTGAAGGCCGACGT
GGAGAAGATGGACGAAAGAGAGCTGGAAAAGTATCAGAAGATCAACGACGCCAAGGATCTGGCCAACCTGCGGCGGCTGGCCAGCG
ACTTCGGAGTGAAGTGGGAGGAGAAGGATTGGGACGAGTACTCCGGCCAGATCAAGAAGCAGATCACAGATTCCCAGAAGCTGACC
ATCATGAAGCAGAGAATCACAGCCGGCCTGAAGAAGAAGCACGGCATCGAAAACCTGAACCTGAGGATCACCATCGACATCAACAA
GTCCAGAAAGGCCGTGCTGAATCGGATCGCCATCCCCAGAGGATTTGTGAAGCGGCACATCCTGGGCTGGCAGGAATCCGAGAAGG
TGAGCAAGAAGATCAGAGAAGCCGAATGCGAGATTCTGCTGAGCAAGGAGTACGAGGAGCTGAGCAAGCAGTTCTTTCAGAGCAAG
GACTACGACAAGATGACCCGCATCAACGGCCTGTACGAGAAGAATAAGCTGATCGCCCTGATGGCCGTGTATCTGATGGGGCAGCT
GAGAATCCTGTTCAAGGAGCACACCAAGCTGGACGACATCACCAAGACCACCGTGGATTTCAAGATCAGCGACAAGGTGACCGTGA
AGATCCCCTTCTCCAACTATCCCTCCCTGGTGTACACCATGAGCAGCAAGTACGTGGACAATATCGGCAACTACGGCTTCAGCAAC
AAGGACAAGGATAAGCCCATTCTGGGCAAGATCGACGTGATCGAGAAGCAGCGGATGGAGTTTATCAAGGAGGTGCTGGGATTCGA
GAAGTACCTGTTTGACGATAAGATCATCGACAAGAGCAAGTTCGCCGACACCGCCACCCACATCAGCTTTGCCGAAATCGTGGAAG
AACTGGTGGAGAAGGGCTGGGACAAGGACCGGCTGACGAAGCTGAAGGATGCCCGGAACAAGGCCCTGCACGGCGAGATCCTGACC
GGCACCAGCTTCGACGAGACAAAGTCCCTGATCAACGAGCTGAAGAAGTAA (SEQ ID NO: 24)
ATGAGCCCTGATTTCATCAAGCTGGAGAAGCAGGAAGCAGCCTTCTACTTTAACCAGACCGAGCTGAACCTGAAGGCCATCGAATC
CAATATCCTGGATAAGCAGCAGAGAATGATCCTGCTGAACAACCCCAGAATCCTGGCCAAGGTGGGCAACTTCATCTTCAATTTCC
GGGACGTGACCAAGAACGCAAAGGGCGAAATCGACTGCCTGCTGTTCAAGCTGGAGGAACTGCGGAACTTCTACAGCCACTACGTG
CACACCGATAACGTGAAGGAACTGTCCAACGGAGAGAAGCCTCTGCTGGAGCGGTACTACCAGATCGCCATCCAGGCCACAAGAAG
CGAGGACGTGAAGTTCGAGCTGTTCGAGACCAGGAACGAGAACAAGATCACCGACGCAGGCGTGCTGTTCTTCCTGTGCATGTTCC
TGAAGAAGAGCCAGGCTAATAAGCTGATTTCCGGCATCAGCGGCTTCAAGCGGAACGACCCCACCGGCCAGCCCAGACGGAACCTC
TTTACCTACTTCTCTGCCCGGGAGGGCTACAAGGCCCTGCCTGACATGCAGAAGCACTTCCTGCTGTTCACCCTGGTGAACTACCT
GAGCAACCAGGACGAGTACATCTCCGAGCTGAAGCAGTACGGAGAGATCGGACAGGGAGCCTTCTTCAACAGAATCGCCAGCACCT
TCCTGAACATCAGCGGCATCAGCGGCAACACCAAGTTCTACAGCTACCAGAGCAAGAGAATCAAGGAGCAGCGGGGCGAACTGAAC
AGCGAAAAGGACAGCTTCGAGTGGATCGAGCCCTTTCAGGGCAACTCTTATTTTGAGATCAACGGCCACAAGGGCGTGATCGGCGA
AGACGAGCTGAAGGAGCTGTGCTACGCCCTGCTGGTGGCCAAGCAGGACATCAATGCCGTGGAGGGAAAGATCATGCAGTTCCTGA
AGAAGTTCAGGAACACCGGCAACCTGCAGCAGGTGAAGGACGACGAGATGCTGGAAATCGAGTACTTTCCCGCCAGCTACTTCAAC
GAGAGCAAGAAGGAGGACATCAAGAAGGAGATCCTGGGCAGACTGGACAAGAAGATCCGGTCCTGCAGCGCCAAGGCCGAGAAGGC
CTACGACAAGATGAAGGAGGTGATGGAGTTTATCAATAACAGCCTGCCCGCCGAGGAGAAGCTGAAGAGGAAGGACTACCGCAGAT
ACCTGAAGATGGTGAGATTCTGGTCCAGAGAAAAGGGCAACATCGAGAGAGAGTTCAGAACCAAGGAGTGGTCCAAGTACTTCAGC
AGCGACTTCTGGAGAAAGAACAATCTGGAGGATGTGTACAAGCTGGCCACCCAGAAGAACGCCGAGCTGTTCAAGAATCTGAAGGC
CGCCGCCGAGAAGATGGGCGAAACAGAATTCGAAAAGTACCAGCAGATCAACGATGTGAAGGACCTGGCCAGCCTGAGACGGCTGA
CCCAGGATTTCGGCCTGAAGTGGGAGGAGAAGGATTGGGAGGAGTACAGCGAACAGATCAAGAAGCAGATCACCGACCGGCAGAAG
CTGACAATCATGAAGCAGCGGGTGACCGCCGAGCTGAAGAAGAAGCACGGCATCGAGAATCTGAACCTCAGAATTACCATCGATTC
CAACAAGAGCAGAAAGGCCGTGCTGAACAGAATCGCCATTCCCCGGGGCTTCGTGAAGAAGCACATTCTGGGCTGGCAGGGCAGCG
AAAAGATCAGCAAGAATATCCGGGAGGCCGAGTGCAAGATCCTGCTGTCCAAGAAGTATGAGGAGCTGTCTCGGCAGTTCTTTGAG
GCTGGCAACTTCGACAAGCTGACCCAGATCAACGGCCTGTACGAAAAGAATAAGCTGACCGCCTTCATGTCCGTCTACCTGATGGG
CAGACTGAACATCCAGCTGAACAAGCACACGGAGCTGGGAAATCTGAAGAAGACCGAGGTGGACTTCAAGATTTCCGACAAGGTGA
CAGAAAAGATCCCCTTCTCCCAGTACCCTAGCCTGGTGTACGCTATGAGCCGGAAGTACGTGGACAACGTGGACAAGTACAAGTTC
AGCCACCAGGACAAGAAGAAGCCCTTCCTGGGCAAGATCGACAGCATCGAAAAGGAGAGAATCGAATTCATCAAGGAGGTGCTGGA
CTTCGAAGAGTACCTGTTTAAGAACAAGGTGATCGACAAGAGCAAGTTCAGCGATACCGCCACCCATATCTCTTTCAAGGAAATCT
GCGACGAGATGGGCAAGAAGGGCTGCAACCGCAACAAGCTGACCGAGCTGAATAACGCTAGAAACGCCGCACTGCACGGAGAAATC
CCCAGCGAGACCAGCTTCCGGGAGGCCAAGCCCCTGATCAACGAACTGAAGAAGTAA (SEQ ID NO: 25)
ATGAGCCCTGACTTCATCAAGCTGGAAAAGCAGGAAGCCGCCTTCTACTTTAATCAGACCGAGCTGAACCTGAAGGCCATCGAGAG
CAACATCTTCGACAAGCAGCAGCGGGTGATCCTGCTGAATAACCCCCAGATCCTGGCCAAGGTGGGCGACTTCATCTTCAACTTCC
GGGACGTGACCAAGAACGCCAAGGGAGAAATCGACTGCCTGCTGCTGAAGCTGCGGGAGCTGAGAAACTTCTACAGCCACTATGTG
TACACCGACGACGTGAAGATCCTGAGCAACGGCGAGAGGCCCCTGCTGGAGAAGTACTACCAGTTTGCCATCGAGGCCACCGGATC
TGAGAATGTGAAGCTGGAGATCATCGAGAGCAACAACCGGCTGACCGAAGCGGGCGTGCTGTTCTTCCTGTGCATGTTCCTGAAGA
AGAGCCAGGCCAACAAGCTGATTTCCGGCATCTCCGGATTCAAGCGCAACGACCCTACCGGACAGCCTCGGCGGAACCTGTTCACC
TACTTTAGCGTGCGGGAGGGCTACAAGGTGGTGCCCGACATGCAGAAGCACTTCCTGCTGTTCGTGCTGGTGAACCACCTGTCCGG
CCAGGATGACTATATTGAGAAGGCCCAGAAGCCCTACGACATCGGCGAAGGCCTGTTCTTCCACAGAATCGCCAGCACCTTTCTCA
ACATCAGCGGCATCCTGAGAAACATGGAATTCTACATCTACCAGAGCAAGCGGCTGAAGGAGCAGCAGGGAGAGCTGAAGAGAGAG
AAGGACATCTTCCCTTGGATCGAGCCTTTCCAGGGCAACAGCTACTTTGAGATCAACGGAAACAAGGGCATCATCGGCGAGGACGA
ACTGAAGGAACTGTGCTACGCCCTGCTGGTGGCCGGCAAGGACGTGAGAGCCGTGGAAGGAAAGATCACCCAGTTCCTGGAGAAGT
TCAAGAACGCCGATAACGCCCAGCAGGTGGAGAAGGATGAAATGCTGGACCGGAACAACTTCCCTGCCAATTACTTTGCCGAAAGC
AACATCGGCAGCATCAAGGAAAAGATCCTGAATAGACTGGGCAAGACCGACGACTCCTACAACAAGACCGGCACCAAGATCAAGCC
CTACGACATGATGAAGGAGGTGATGGAGTTCATCAATAATTCTCTGCCCGCCGATGAGAAGCTGAAGCGGAAGGACTACCGGAGAT
ACCTGAAGATGGTCCGGATCTGGGACAGCGAAAAGGACAATATCAAGCGGGAGTTTGAGAGCAAGGAATGGAGCAAGTATTTCAGC
AGCGACTTCTGGATGGCCAAGAACCTGGAAAGAGTGTACGGCCTGGCCAGGGAAAAGAACGCCGAGCTGTTTAACAAGCTGAAGGC
CGTGGTGGAGAAGATGGACGAGCGGGAGTTCGAAAAGTACCGGCTGATCAACAGCGCCGAAGACCTGGCCAGCCTGCGGAGACTGG
CCAAGGACTTCGGCCTGAAGTGGGAGGAGAAGGACTGGCAGGAGTATTCTGGCCAGATCAAGAAGCAGATCTCCGACAGACAGAAG
CTGACAATTATGAAGCAGCGGATCACAGCCGAACTGAAGAAGAAGCACGGAATCGAGAACCTGAATCTGCGGATCACCATCGACAG
CAACAAGTCCAGAAAGGCCGTGCTGAACCGGATCGCCGTGCCCCGGGGCTTCGTGAAGGAACACATCCTGGGCTGGCAAGGCTCTG
AAAAGGTGAGCAAGAAGACCAGAGAAGCCAAGTGCAAGATCCTGCTGAGCAAGGAGTACGAGGAACTGAGCAAGCAGTTCTTTCAG
ACACGGAATTACGACAAGATGACCCAGGTGAACGGCCTGTACGAGAAGAACAAGCTGCTGGCCTTCATGGTGGTGTACCTGATGGA
GAGACTGAACATCCTGCTGAACAAGCCCACAGAGCTGAACGAACTGGAAAAGGCCGAAGTGGACTTCAAGATCTCCGACAAGGTGA
TGGCCAAGATCCCTTTCTCTCAGTACCCCAGCCTGGTGTATGCAATGAGCTCCAAGTACGCCGACAGCGTGGGCTCTTACAAGTTC
GAAAACGACGAGAAGAACAAGCCCTTTCTGGGCAAGATCGACACAATCGAGAAGCAGAGAATGGAGTTCATCAAGGAGGTGCTGGG
CTTCGAGGAATACCTGTTCGAGAAGAAGATCATCGATAAGAGCGAATTCGCCGACACCGCCACCCACATCAGCTTCGACGAGATCT
GCAACGAGCTGATCAAGAAGGGCTGGGACAAGGACAAGCTGACCAAGCTGAAGGACGCCCGGAACGCCGCCCTGCACGGCGAGATC
CCCGCCGAGACCAGCTTCCGGGAGGCCAAGCCCCTGATTAACGGCCTGAAGAAGTAA (SEQ ID NO: 26)
ATGAACATCATCAAGCTGAAGAAGGAGGAAGCCGCCTTTTACTTTAACCAGACAATCCTGAATCTGAGCGGCCTGGACGAGATCAT
CGAGAAGCAGATCCCCCACATCATCTCCAATAAGGAAAACGCCAAGAAGGTGATTGATAAGATCTTCAATAACAGACTGCTGCTGA
AGAGCGTGGAAAACTATATCTACAACTTCAAGGACGTGGCCAAGAACGCCCGGACCGAAATCGAAGCCATCCTGCTGAAGCTGGTG
GAGCTGAGAAACTTCTACTCCCACTACGTGCACAACGACACCGTGAAGATCCTGTCCAATGGCGAGAAGCCCATCCTGGAAAAGTA
CTACCAGATCGCCATCGAAGCCACCGGCTCTAAGAACGTGAAGCTGGTCATTATCGAAAACAACAACTGCCTGACCGACTCCGGCG
TGCTGTTCCTGCTGTGCATGTTCCTGAAGAAGAGCCAGGCCAACAAGCTGATTAGCAGCGTGAGCGGCTTTAAGCGGAACGACAAG
GAAGGCCAGCCCAGAAGGAACCTCTTTACTTACTATAGCGTGAGGGAAGGCTACAAGGTGGTGCCAGACATGCAGAAGCACTTCCT
GCTGTTCGCCCTGGTCAACCACCTGTCCGAGCAGGACGACCACATCGAGAAGCAGCAGCAGAGCGACGAGCTGGGCAAGGGCCTGT
TCTTCCACAGAATCGCCAGCACATTCCTGAATGAAAGCGGCATCTTCAACAAGATGCAGTTTTACACCTACCAGAGCAATCGGCTG
AAGGAGAAGCGGGGCGAGCTGAAGCACGAGAAGGACACCTTCACCTGGATCGAGCCTTTCCAGGGAAACAGCTACTTCACCCTGAA
CGGGCACAAGGGCGTGATCAGCGAGGATCAGCTGAAGGAACTGTGCTACACAATCCTGATCGAGAAGCAGAACGTGGACAGCCTGG
AGGGCAAGATCATTCAGTTCCTGAAGAAGTTTCAGAACGTGTCTAGCAAGCAGCAGGTGGATGAGGACGAGCTGCTGAAGCGGGAA
TACTTCCCCGCCAACTACTTCGGCCGGGCCGGCACCGGCACCCTGAAGGAGAAGATCCTGAACCGGCTGGACAAGCGGATGGACCC
CACCAGCAAGGTGACCGACAAGGCCTATGACAAGATGATCGAGGTGATGGAGTTCATCAACATGTGCCTGCCCAGCGACGAGAAGC
TGCGGCAGAAGGATTACCGGAGATATCTGAAGATGGTCAGATTCTGGAACAAGGAGAAGCACAACATCAAGAGAGAATTCGACAGC
AAGAAGTGGACCAGATTCCTGCCCACCGAGCTGTGGAATAAGCGGAACCTGGAGGAAGCCTACCAGCTGGCCCGGAAGGAGAACAA
GAAGAAGCTGGAGGACATGAGGAATCAGGTGAGGAGCCTGAAGGAGAACGACCTGGAGAAGTACCAGCAGATCAACTATGTGAACG
ACCTGGAAAACCTGCGGCTGCTGTCCCAAGAGCTGGGCGTGAAGTGGCAGGAGAAGGACTGGGTGGAATACAGCGGCCAGATCAAG
AAGCAGATCAGCGATAACCAGAAGCTGACAATCATGAAGCAGAGAATCACCGCCGAGCTGAAGAAGATGCACGGCATCGAGAACCT
GAACCTGAGAATCAGCATCGACACCAACAAGTCCCGGCAGACTGTGATGAACAGAATTGCCCTGCCCAAGGGCTTCGTGAAGAACC
ACATTCAGCAGAACAGCAGCGAGAAGATCAGCAAGAGAATCAGAGAGGACTACTGCAAGATCGAGCTGTCCGGCAAGTACGAAGAG
CTGAGCAGACAGTTTTTCGACAAGAAGAACTTTGACAAGATGACCCTGATCAACGGACTGTGCGAGAAGAATAAGCTCATCGCCTT
CATGGTGATTTACCTGCTGGAGCGGCTGGGCTTCGAGCTGAAGGAGAAGACCAAGCTGGGCGAGCTGAAGCAGACCCGGATGACAT
ATAAGATCAGCGACAAGGTGAAGGAGGACATCCCCCTCTCCTACTACCCCAAGCTGGTGTACGCCATGAATCGGAAGTATGTGGAC
AACATCGATAGCTACGCCTTCGCCGCCTACGAGTCTAAGAAGGCCATCCTGGACAAGGTGGACATCATTGAGAAGCAGAGAATGGA
ATTCATCAAGCAGGTGCTGTGCTTCGAGGAATACATCTTCGAGAACAGAATCATCGAGAAGAGCAAGTTCAACGATGAGGAGACCC
ACATCAGCTTCACCCAGATCCACGACGAACTGATCAAGAAGGGCAGAGATACCGAAAAGCTGAGCAAGCTGAAGCACGCCAGAAAC
AAGGCCCTGCACGGCGAGATCCCCGACGGGACCAGCTTTGAGAAGGCCAAGCTGCTGATCAACGAAATCAAGAAGTAA (SEQ ID
NO: 27)
ATGAACGCCATCGAGCTGAAGAAGGAAGAGGCCGCCTTCTACTTCAACCAGGCCAGACTGAACATCTCTGGCCTGGACGAAATCAT
CGAGAAGCAACTGCCACACATCGGCTCTAACAGAGAGAACGCCAAGAAGACTGTGGACATGATCCTGGATAACCCCGAGGTGCTGA
AGAAGATGGAAAACTACGTGTTCAACTCCCGCGATATTGCCAAGAATGCCCGGGGCGAGCTGGAGGCCCTGCTGCTGAAGCTGGTC
GAGCTGAGAAACTTCTATAGCCACTACGTGCACAAGGACGACGTCAAGACACTGAGCTACGGTGAGAAGCCTCTGCTGGATAAGTA
CTACGAGATCGCCATCGAAGCCACCGGATCCAAGGACGTGCGGCTGGAGATCATTGACGACAAGAATAAGCTGACCGACGCCGGAG
TGCTGTTCCTGCTGTGCATGTTCCTGAAGAAGAGCGAGGCTAACAAGCTGATTTCCAGCATCCGGGGCTTCAAGAGGAACGACAAG
GAGGGCCAGCCTAGAAGAAACCTGTTCACCTACTACAGCGTGAGAGAGGGCTATAAGGTGGTGCCCGACATGCAGAAGCACTTTCT
GCTGTTCACCCTGGTGAACCACCTGTCCAATCAGGACGAGTACATCTCCAACCTGCGCCCAAACCAGGAAATCGGCCAGGGCGGAT
TTTTCCACCGGATCGCCAGCAAGTTCCTGAGCGACAGCGGAATCCTGCACAGCATGAAGTTCTACACATACAGATCCAAGCGGCTG
ACCGAGCAGCGGGGAGAGCTGAAGCCCAAGAAGGACCACTTTACATGGATCGAGCCTTTCCAGGGCAATTCCTACTTCAGCGTGCA
GGGCCAGAAGGGCGTGATCGGAGAGGAGCAGCTCAAGGAGCTGTGCTACGTGCTGCTGGTGGCCCGGGAGGACTTCAGAGCCGTGG
AGGGCAAGGTGACCCAGTTCCTGAAGAAGTTCCAGAATGCCAATAACGTGCAGCAGGTGGAGAAGGACGAGGTGCTGGAAAAGGAG
TACTTCCCCGCCAACTACTTTGAGAACCGGGACGTGGGAAGAGTCAAGGACAAGATCCTGAACAGACTGAAGAAGATCACCGAGAG
TTATAAGGCCAAGGGTAGAGAGGTGAAGGCCTACGACAAGATGAAGGAAGTGATGGAGTTCATCAACAACTGCCTGCCCACCGATG
AAAACCTGAAGCTGAAGGACTACCGGCGGTACCTGAAGATGGTGAGATTCTGGGGCAGAGAGAAGGAAAACATCAAGCGGGAGTTC
GACTCCAAGAAGTGGGAGCGCTTTCTCCCCCGGGAGCTGTGGCAGAAGAGAAACCTGGAGGACGCCTACCAGCTCGCCAAGGAGAA
GAACACAGAGCTGTTCAACAAGCTGAAGACCACCGTGGAGAGAATGAACGAACTGGAGTTCGAGAAGTACCAGCAGATCAATGACG
CCAAGGACCTGGCCAACCTGAGACAGCTGGCCAGAGACTTTGGAGTGAAGTGGGAGGAAAAGGACTGGCAGGAATACTCTGGACAG
ATCAAGAAGCAGATCACCGACCGGCAGAAGCTGACCATCATGAAGCAGCGGATCACCGCCGCCCTGAAGAAGAAGCAGGGAATCGA
AAACCTGAACCTGAGAATCACAACAGATACGAATAAGAGCAGGAAGGTGGTGCTGAACCGGATCGCACTGCCCAAGGGATTCGTCA
GAAAGCACATCCTGAAGACCGACATCAAGATCAGCAAGCAGATCCGGCAGAGCCAGTGCCCTATCATCCTGTCTAACAACTACATG
AAGCTGGCCAAGGAGTTCTTTGAAGAGCGGAACTTCGATAAGATGACCCAGATCAATGGCCTGTTCGAGAAGAACGTGCTGATCGC
CTTCATGATCGTGTACCTGATGGAGCAGCTGAACCTGAGACTGGGCAAGAACACCGAGCTGTCCAACCTGAAGAAGACCGAGGTGA
ACTTTACCATCACCGACAAGGTGACCGAGAAGGTGCAAATCTCCCAGTACCCCAGCCTGGTGTTCGCCATTAACCGGGAGTACGTG
GACGGCATCAGCGGCTACAAGCTGCCCCCCAAGAAGCCCAAGGAACCTCCCTACACCTTCTTCGAAAAGATCGACGCCATCGAAAA
GGAGCGGATGGAATTCATCAAGCAGGTGCTGGGCTTCGAGGAGCACCTCTTCGAAAAGAACGTGATCGACAAGACCCGGTTTACCG
ACACCGCCACCCACATCAGCTTCAATGAGATCTGCGATGAGCTGATCAAGAAGGGCTGGGACGAAAACAAGATCATCAAGCTGAAG
GATGCACGGAACGCTGCCCTGCACGGCAAGATCCCTGAAGATACCTCCTTTGACGAAGCCAAGGTGCTGATCAACGAACTGAAGAA
GTAA (SEQ ID NO: 28)
GGGS (G₃S) (SEQ ID NO: 63)

| | |
|---|---|
| Linker 1 | GS |
| Linker 2 | GSGGGGS (SEQ ID NO: 29) |
| Linker 3 | GGGGSGGGGSGGGGS (G₄S)₃ (SEQ ID NO: 30) |
| Linker 4 | GGGSGGGSGGGS (G₃S)₃ (SEQ ID NO: 91) |
| Linker 5 | (GGS)ₙ wherein n is 1, 2, 3, 4, 5, 6, 7, 8, 9, or 10 (SEQ ID NO: 95) |
| Linker 6 | GGGGGSGGGGSGGGGSGGGGS (SEQ ID NO: 33) |
| hADAR1_{DD}- | SLGTGNRCVKGDSLSLKGETVNDCHAEIISRRGFIRFLYSELMKYNSQTAKDSIFEPAKGGEKLQIKKTVS |
| WT | FHLYISTAPCGDGALFDKSCSDRAMESTESRHYPVFENPKQGKLRTKVENGEGTIPVESSDIVPTWDGIRL |
| | GERLRTMSCSDKILRWNVLGLQGALLTHFLQPIYLKSVTLGYLFSQGHLTRAICCRVTRDGSAFEDGLRHP |
| | FIVNHPKVGRVSIYDSKRQSGKTKETSVNWCLADGYDLEILDGTRGTVDGPRNELSRVSKKNIFLLFKKLC |
| | SFRYRRDLLRLSYGEAKKAARDYETAKNYFKKGLKDMGYGNWISKPQEEKNF (SEQ ID NO: 57) |
| hADAR1_{DD}- | SLGTGNRCVKGDSLSLKGETVNDCHAEIISRRGFIRFLYSELMKYNSQTAKDSIFEPAKGGEKLQIKKTVS |
| E1008Q | FHLYISTAPCGDGALFDKSCSDRAMESTESRHYPVFENPKQGKLRTKVENGQGTIPVESSDIVPTWDGIRL |
| | GERLRTMSCSDKILRWNVLGLQGALLTHFLQPIYLKSVTLGYLFSQGHLTRAICCRVTRDGSAFEDGLRHP |
| | FIVNHPKVGRVSIYDSKRQSGKTKETSVNWCLADGYDLEILDGTRGTVDGPRNELSRVSKKNIFLLFKKLC |
| | SFRYRRDLLRLSYGEAKKAARDYETAKNYFKKGLKDMGYGNWISKPQEEKNF (SEQ ID NO: 58) |
| hADAR2_{DD}- | QLHLPQVLADAVSRLVLGKFGDLTDNFSSPHARRKVLAGVVMTTGTDVKDAKVISVSTGTKCINGEYMSDR |
| WT | GLALNDCHAEIISRRSLLRFLYTQLELYLNNKDDQKRSIFQKSERGGFRLKENVQFHLYISTSPCGDARIF |
| | SPHEPILEEPADRHPNRKARGQLRTKIESGEGTIPVRSNASIQTWDGVLQGERLLTMSCSDKIARWNVVGI |
| | QGSLLSIFVEPIYFSSIILGSLYHGDHLSRAMYQRISNIEDLPPLYTLNKPLLSGISNAEARQPGKAPNFS |
| | VNWTVGDSAIEVINATTGKDELGRASRLCKHALYCRWMRVHGKVPSHLLRSKITKPNVYHESKLAAKEYQA |
| | AKARLFTAFIKAGLGAWVEKPTEQDQFSLT (SEQ ID NO: 59) |
| hADAR2_{DD}- | QLHLPQVLADAVSRLVLGKFGDLTDNFSSPHARRKVLAGVVMTTGTDVKDAKVISVSTGTKCINGEYMSDR |
| E488Q | GLALNDCHAEIISRRSLLRFLYTQLELYLNNKDDQKRSIFQKSERGGFRLKENVQFHLYISTSPCGDARIF |
| | SPHEPILEEPADRHPNRKARGQLRTKIESGQGTIPVRSNASIQTWDGVLQGERLLTMSCSDKIARWNVVGI |
| | QGSLLSIFVEPIYFSSIILGSLYHGDHLSRAMYQRISNIEDLPPLYTLNKPLLSGISNAEARQPGKAPNFS |
| | VNWTVGDSAIEVINATTGKDELGRASRLCKHALYCRWMRVHGKVPSHLLRSKITKPNVYHESKLAAKEYQA |
| | AKARLFTAFIKAGLGAWVEKPTEQDQFSLT (SEQ ID NO: 34) |
| AID- | MDSLLMNRRKFLYQFKNVRWAKGRRETYLCYVVKRRDSATSFSLDFGYLRNKNGCHVELLFLRYISDWDLD |
| APOBEC1 | PGRCYRVTWFTSWSPCYDCARHVADFLRGNPNLSLRIFTARLYFCEDRKAEPEGLRRLHRAGVQIAIMTFK |
| | DYFYCWNTFVENHERTFKAWEGLHENSVRLSRQLRRILLPLYEVDDLRDAFRTLGL (SEQ ID NO: 60) |
| Lamprey_A | MTDAEYVRIHEKLDIYTFKKQFFNNKKSVSHRCYVLFELKRRGERRACFWGYAVNKPQSGTERGIHAEIFS |
| ID- | IRKVEEYLRDNPGQFTINWYSSWSPCADCAEKILEWYNQELRGNGHTLKIWACKLYYEKNARNQIGLWNLR |
| APOBEC1 | DNGVGLNVMVSEHYQCCRKIFIQSSHNQLNENRWLEKTLKRAEKRRSELSIMIQVKILHTTKSPAV (SEQ |
| | ID NO: 61) |
| APOBEC1_B | MSSETGPVAVDPTLRRRIEPHEFEVFFDPRELRKSTCLLYEINWGGRHSIWRHTSQNTNKHVEVNFIEKFT |
| E1 | TERYFCPNTRCSITWFLSWSPCGECSRAITEFLSRYPHVTLFIYIARLYHHADPRNRQGLRDLISSGVTIQ |
| | IMTEQESGYCWRNFVNYSPSNEAHWPRYPHLWVRLYVLELYCIILGLPPCLNILRRKQPQLTFFTIALQSC |
| | HYQRLPPHILWATGLK (SEQ ID NO: 62) |

>dCas13e.1-R84A, H89A, R739A, R740A, H744A, H745A without starting M
AQVSKQTSKKRELSIDEYQGARKWCFTIAFNKALVNRDKNDGLFVESLLRHEKYSKHDWYDEDTRALIKCSTQAANAKAEALANYF
SAYRHSPGCLTFTAEDELRTIMERAYERAIFECRRRETEVIIEFPSLFEGDRITTAGVVFFVSFFVERRVLDRLYGAVSGLKKNEG
QYKLTRKALSMYCLKDSRETKAWDKRVLLFRDILAQLGRIPAEAYEYYHGEQGDKKRANDNEGTNPKRHKDKFIEFALHYLEAQHS
EICFGRRHIVREEAGAGDEHKKHRTKGKVVVDFSKKDEDQSYYISKNNVIVRIDKNAGPRSYRMGLNELKYLVLLSLQGKGDDAIA
KLYRYRQHVENILDVVKVTDKDNHVFLPRFVLEQHGIGRKAFKQRIDGRVKHVRGVWEKKKAATNEMTLHEKARDILQYVNENCTR
SFNPGEYNRLLVCLVGKDVENFQAGLKRLQLAERIDGRVYSIFAQTSTINEMHQVVCDQILNRLCRIGDQKLYDYVGLGKKDEIDY
KQKVAWFKEHISIRRGFLRKKFWYDSKKGFAKLVEEHLESGGGQRDVGLDKKYYHIDAIGRFEGANPALYETLARDRLCLMMAQYF
LGSVRKELGNKIVWSNDSIELPVEGSVGNEKSIVFSVSDYGKLYVLDDAEFLGRICEYFMPHEKGKIRYHTVYEKGFRAYNDLQKK
CVEAVLAFEEKVVKAKKMSEKEGAHYIDFREILAQTMCKEAEKTAVNKVAAAFFAAHLKFVIDEFGLFSDVMKKYGIEKEWKFPVK
(SEQ ID NO: 31)
>minidCas13e.1-N180 + C150 (with N-terminal 180 aa truncation and C-terminal 150-residue
truncation)
LSMYCLKDSRFTKAWDKRVLLFRDILAQLGRIPAEAYEYYHGEQGDKKRANDNEGTNPKRHKDKFIEFALHYLEAQHSEICFGRRH
IVREEAGAGDEHKKHRTKGKVVVDFSKKDEDQSYYISKNNVIVRIDKNAGPRSYRMGLNELKYLVLLSLQGKGDDAIAKLYRYRQH
VENILDVVKVTDKDNHVFLPRFVLEQHGIGRKAFKQRIDGRVKHVRGVWEKKKAATNEMTLHEKARDILQYVNENCTRSFNPGEYN
RLLVCLVGKDVENFQAGLKRLQLAERIDGRVYSIFAQTSTINEMHQWCDQILNRLCRIGDQKLYDYVGLGKKDEIDYKQKVAWFK
EHISIRRGFLRKKFWYDSKKGFAKLVEEHLESGGGQRDVGLDKKYYHIDAIGRFEGANPALYETLARDRLCLMMAQYFLGSVRKEL
GNKIVWSNDSIELP (SEQ ID NO: 32)
>Linker
GGGGGSGGGGSGGGGSGGGGS (SEQ ID NO: 33)
>hADAR2_{DD}-E488Q (double underlined)
QLHLPQVLADAVSRLVLGKFGDLTDNFSSPHARRKVLAGVVMTTGTDVKDAKVISVSTGTKCINGEYMSDRGLALNDCHAEIISRR
SLLRFLYTQLELYLNNKDDQKRSIFQKSERGGFRLKENVQFHLYISTSPCGDARIFSPHEPILEEPADRHPNRKARGQLRTKIESG
QGTIPVRSNASIQTWDGVLQGERLLTMSCSDKIARWNVVGIQGSLLSIFVEPIYFSSIILGSLYHGDHLSRAMYQRISNIEDLPPL
YTLNKPLLSGISNAEARQPGKAPNFSVNWTVGDSAIEVINATTGKDELGRASRLCKHALYCRWMRVHGKVPSHLLRSKITKPNVYH
ESKLAAKEYQAAKARLFTAFIKAGLGAWVEKPTEQDQFSLT (SEQ ID NO: 34)
>SV40 NLS
PKKKRKV (SEQ ID NO: 35)
>NLS-full length dCas13e.1 without starting M-NLS-GS Linker-hADAR2_{DD}-E488Q
PKKKRKVAQVSKQTSKKRELSIDEYQGARKWCFTIAFNKALVNRDKNDGLFVESLLRHEKYSKHDWYDEDTRALIKCSTQAANAKA
EALANYFSAYRHSPGCLTFTAEDELRTIMERAYERAIFECRRRETEVIIEFPSLFEGDRITTAGVVFFVSFFVERRVLDRLYGAVS
GLKKNEGQYKLTRKALSMYCLKDSRFTKAWDKRVLLFRDILAQLGRIPAEAYEYYHGEQGDKKRANDNEGTNPKRHKDKFIEFALH
YLEAQHSEICFGRRHIVREEAGAGDEHKKHRTKGKVVVDFSKKDEDQSYYISKNNVIVRIDKNAGPRSYRMGLNELKYLVLLSLQG
KGDDAIAKLYRYRQHVENILDVVKVTDKDNHVFLPRFVLEQHGIGRKAFKQRIDGRVKHVRGVWEKKKAATNEMTLHEKARDILQY
VNENCTRSFNPGEYNRLLVCLVGKDVENFQAGLKRLQLAERIDGRVYSIFAQTSTINEMHQVVCDQILNRLCRIGDQKLYDYVGLG
KKDEIDYKQKVAWFKEHISIRRGFLRKKFWYDSKKGFAKLVEEHLESGGGQRDVGLDKKYYHIDAIGRFEGANPALYETLARDRLC
LMMAQYFLGSVRKELGNKIVWSNDSIELPVEGSVGNEKSIVFSVSDYGKLYVLDDAEFLGRICEYFMPHEKGKIRYHTVYEKGFRA
YNDLQKKCVEAVLAFEEKVVKAKKMSEKEGAHYIDFREILAQTMCKEAEKTAVNKVAAAFFAAHLKFVIDEFGLFSDVMKKYGIEK
EWKFPVKPKKKRKVGGGGGSGGGGSGGGGSGGGGSQLHLPQVLADAVSRLVLGKFGDLTDNFSSPHARRKVLAGVVMTTGTDVKDA
KVISVSTGTKCINGEYMSDRGLALNDCHAEIISRRSLLRFLYTQLELYLNNKDDQKRSIFQKSERGGFRLKENVQFHLYISTSPCG
DARIFSPHEPILEEPADRHPNRKARGQLRTKIESGQGTIPVRSNASIQTWDGVLQGERLLTMSCSDKIARWNWGIQGSLLSIFVE
PIYFSSIILGSLYHGDHLSRAMYQRISNIEDLPPLYTLNKPLLSGISNAEARQPGKAPNFSVNWTVGDSAIEVINATTGKDELGRA
SRLCKHALYCRWMRVHGKVPSHLLRSKITKPNVYHESKLAAKEYQAAKARLFTAFIKAGLGAWVEKPTEQDQFSLT (SEQ ID
NO: 36)
>M-NLS-minidCas13e.1-NLS-GS Linker-hADAR2_{DD}-E488Q
MPKKKRKVLSMYCLKDSRFTKAWDKRVLLFRDILAQLGRIPAEAYEYYHGEQGDKKRANDNEGTNPKRHKDKFIEFALHYLEAQHS
EICFGRRHIVREEAGAGDEHKKHRTKGKVVVDFSKKDEDQSYYISKNNVIVRIDKNAGPRSYRMGLNELKYLVLLSLQGKGDDAIA
KLYRYRQHVENILDVVKVTDKDNHVFLPRFVLEQHGIGRKAFKQRIDGRVKHVRGVWEKKKAATNEMTLHEKARDILQYVNENCTR
SFNPGEYNRLLVCLVGKDVENFQAGLKRLQLAERIDGRVYSIFAQTSTINEMHQVVCDQILNRLCRIGDQKLYDYVGLGKKDEIDY
KQKVAWFKEHISIRRGFLRKKFWYDSKKGFAKLVEEHLESGGGQRDVGLDKKYYHIDAIGRFEGANPALYETLARDRLCLMMAQYF
LGSVRKELGNKIVWSNDSIELPPKKKRKVGGGGGSGGGGSGGGGSGGGGSQLHLPQVLADAVSRLVLGKFGDLTDNFSSPHARRKV
LAGVVMTTGTDVKDAKVISVSTGTKCINGEYMSDRGLALNDCHAEIISRRSLLRFLYTQLELYLNNKDDQKRSIFQKSERGGFRLK
ENVQFHLYISTSPCGDARIFSPHEPILEEPADRHPNRKARGQLRTKIESGQGTIPVRSNASIQTWDGVLQGERLLTMSCSDKIARW
NVVGIQGSLLSIFVEPIYFSSIILGSLYHGDHLSRAMYQRISNIEDLPPLYTLNKPLLSGISNAEARQPGKAPNFSVNWTVGDSAI
EVINATTGKDELGRASRLCKHALYCRWMRVHGKVPSHLLRSKITKPNVYHESKLAAKEYQAAKARLFTAFIKAGLGAWVEKPTEQD
QFSLT (SEQ ID NO: 37)
>Coding sequence of off-target site 1
GCTACCGAACCGTGGGGCAGTAGACGATGGGCCGGATGGGCCCCGAC (SEQ ID NO: 38)
>Spacer sequence for off-target site 1:
GTCGGGGCCCATCCGGCCCATCGTC£ACTGCCCCACGGTTCGGTAGCTAG (SEQ ID NO: 39)
>T2A (SEQ ID NO: 40)
EGRGSLLTCGDVEENPGP
>P2A (SEQ ID NO: 41)
ATNFSLLKQAGDVEENPGP
>dCas13b-NES-Linker-hADAR2_{DD}-E488Q (ADARv1)
MNIPALVENQKKYFGTYSVMAMLNAQTVLDHIQKVADIEGEQNENNENLWFHPVMSHLYNAKNGYDKQPEKTMFIIERLQSYFPFL
KIMAENQREYSNGKYKQNRVEVNSNDIFEVLKRAFGVLKMYRDLTNAYKTYEEKLNDGCEFLTSTEQPLSGMINNYYTVALRNMNE
RYGYKTEDLAFIQDKRFKFVKDAYGKKKSQVNTGFFLSLQDYNGDTQKKLHLSGVGIALLICLFLDKQYINIFLSRLPIFSSYNAQ
SEERRIIIRSFGINSIKLPKDRIHSEKSNKSVAMDMLNEVKRCPDELFTTLSAEKQSRFRIISDDHNEVLMKRSSDRFVPLLLQYI
DYGKLFDHIRFHVNMGKLRYLLKADKTCIDGQTRVRVIEQPLNGFGRLEEAETMRKQENGTFGNSGIRIRDFENMKRDDANPANYP
YIVDTYTHYILENNKVEMFINDKEDSAPLLPVIEDDRYVVKTIPSCRMSTLEIPAMAFHMFLFGSKKTEKLIVDVHNRYKRLFQAM
QKEEVTAENIASFGIAESDLPQKILDLISGNAHGKDVDAFIRLTVDDMLTDTERRIKRFKDDRKSIRSADNKMGKRGFKQISTGKL
ADFLAKDIVLFQPSVNDGENKITGLNYRIMQSAIAVYDSGDDYEAKQQFKLMFEKARLIGKGTTEPHPFLYKVFARSIPANAVEFY
ERYLIERKFYLTGLSNEIKKGNRVDVPFIRRDQNKWKTPAMKTLGRIYSEDLPVELPRQMFDNEIKSHLKSLPQMEGIDFNNANVT
YLIAEYMKRVLDDDFQTFYQWNRNYRYMDMLKGEYDRKGSLQHCFTSVEEREGLWKERASRTERYRKQASNKIRSNRQMRNASSEE
IETILDKRLSNSRNEYQKSEKVIRRYRVQDALLFLLAKKTLTELADFDGERFKLKEIMPDAEKGILSEIMPMSFTFEKGGKKYTIT
SEGMKLKNYGDFFVLASDKRIGNLLELVGSDIVSKEDGSLQLPPLERLTLGGGGGSGGGGSGGGGSGGGGSQLHLPQVLADAVSRL
VLGKFGDLTDNFSSPHARRKVLAGVVMTTGTDVKDAKVISVSTGTKCINGEYMSDRGLALNDCHAEIISRRSLLRFLYTQLELYLN
NKDDQKRSIFQKSERGGFRLKENVQFHLYISTSPCGDARIFSPHEPILEEPADRHPNRKARGQLRTKIESGQGTIPVRSNASIQTW
DGVLQGERLLTMSCSDKIARWNVVGIQGSLLSIFVEPIYFSSIILGSLYHGDHLSRAMYQRISNIEDLPPLYTLNKPLLSGISNAE
ARQPGKAPNFSVNWTVGDSAIEVINATTGKDELGRASRLCKHALYCRWMRVHGKVPSHLLRSKITKPNVYHESKLAAKEYQAAKAR
LFTAFIKAGLGAWVEKPTEQDQFSLT* (SEQ ID NO: 42)
>DMD exon52 del mini-gene
ATGaggaagttagaagatctgaggtctgagtgggaggctgtaaaccatttacttcgggagctgaggacaaagcagcctgaccgtgc
ccctggactgagcactactggagcctgtaagtacattttgtcccaggtgttctcttttgctttagctgtttgttcaaaagcgtggc
tgggaggttatctctgagtgaaaggaaaattggtttctgattctgcataaaggagtaaacatttatgttatttattatgcaaaagg
tctttgagtgtgttccttggatactgagagcattacaagctgtgttttctgaagattcaactgcaaggaaaagagggcagacgagg
agaagccataatggtttaacctgatactttatcctgttccttagcattaaaactggatggctttgtggaggcacgttgtatctcta
ggagaaggtctctcctttgaatttctgcttcactggaagcagagtaatcgtggcagcagttttaaattaaaattaaaggtggttct
tgataaggaaacccaacagaccaccacattactagcatctaaacacagatatcaaggtgaaagttcagtgacactggagactttaa
aaacatgtgcgatttccctagggtccagcttcaaattaatttacttcctattcaagggaattttaaatcagaaagaagatcttatc
ccatcttgttttgcctttgttttttcttgaataaaaaaaaaataagtaaaatttatttccctggcaaggtctgaaaacttttgttt
tctttaccacttccacaatgtatatgattgttactgagaaggcttatttaacttaagttacttgtccaggcatgagaatgagcaaa
atcgttttttaaaaaattgttaaatgtatattaatgaaaaggttgaatcttttcattttctaccatgtattgctaaacaaagtatc
cacattgttagaaaaagatatataatgtcatgaataagagtttggctcaaattgttactcttcaattaaatttgacttattgttat
tgaaattggctctttagcttgtgtttctaatttttctttttcttcttttttcctttttgcaaaaacccaaaatattttagctccta
ctcagactgttactctggtgacacaacctgtggttactaaggaaactgccatctccaaactagaaatgccatcttccttgatgttg
gaggtacctgctctggcagatttcaaccgggcttggacagaacttaccgactggctttctctgcttgatcaagttataaaatcaca
gagggtgatggtgggtgaccttgaggatatcaacgagatgatcatcaagcagaaggtatgagaaaaaatgataaaagttggcagaa
gtttttctttaaaatgaagattttccaccaatcactttactctcctagaccatttcccaccagttcttaggcaactgtttctctct
cagcaaacacattactctcactattcagcctaagtataatcaaggatataaattaatgcaaataacaaaagtagccatacattaaa
aaggaaatatacaaaaaaaaaaaaaaaaaaaaAgcagaaaccttacaagaatagttgtctcagttaaatttactaaacaacctggt
attttaaaaatctattttataccaaataagtcactcaactgagctatttacatttaaactgtttgttttggcactacgcagcccaa
catattgcagaatcaaatataatagtctgggaattgattattatccactcttctaagttgtctgtgccaatttgccttctccaatg
ataaggataattgaaagagagctataacttaaaaagagaagagcacaaccgttggagtcagtcctcaggtgttttccaccatttct
ttgagacaatgtccctctttgaaacctcactaagtaagagagactagatacccaatgagctcatctgggcctgtctctgcttccca
tcttgactctggttttggaacatccaaacacagatccttatgcccacaagtaagtgctgaggtaatagagccaagccctctagtac
ctttctaataaataattgttatttagtgtcagagtctaaagttgaatttatatttctaaacatggcaccaatattgtagtttattt
caatgcaagtaatttaatagaaagtcaaatttgtcacctgaagaaatgattttgttaattattttacctatatcactcatagcacc
ttggatatatttaatgagaaatatacatgtgcaatgacgtttagattctaaatttccactgtcttctcttgagtaataattactgt
tctttattcttatttttattccagttgaaagaattcagattcagtgggatgaggttcaagaacagctgcagaacaggagacaacag
ttgaatgaattaaaggattcaacacaatggctggaagctaaggaagaagccgaacaggtcataggacaggtcagaggcaagcttga
ctcatggaaagaaggtcctcacacagtagatgcaatccaaaagaagatcacagaaaccaag (SEQ ID NO: 43)
>DMD Exon23X (C > T) target sequence
gctttacaaagttctctgcaagagcaaTaaagtggcctatactatctcagcaccactgtg (SEQ ID NO: 44)
>EcCas6e-H20L
YLSKVIIARAWSRDLYQLLQGLWHLFPNRPDAARDFLFHVEKRNTPEGCHVLLQSAQMPVSTAVATVIKTKQVEFQLQVGVPLYFR
LRANPIKTILDNQKRLDSKGNIKRCRVPLIKEAEQIAWLQRKLGNAARVEDVHPISERPQYFSGDGKSGKIQTVCFEGVLTINDAP
ALIDLVQQGIGPAKSMGCGLLSLAPL (SEQ ID NO: 51)
>EcCas6e-H20L-NES-Linker-hADAR2_{DD}-E488Q (ADARv1)
MYLSKVIIARAWSRDLYQLLQGLWHLFPNRPDAARDFLFHVEKRNTPEGCHVLLQSAQMPVSTAVATVIKTKQVEFQLQVGVPLYF
RLRANPIKTILDNQKRLDSKGNIKRCRVPLIKEAEQIAWLQRKLGNAARVEDVHPISERPQYFSGDGKSGKIQTVCFEGVLTINDA
PALIDLVQQGIGPAKSMGCGLLSLAPLLPPLERLTLGGGGGSGGGGSGGGGSGGGGSQLHLPQVLADAVSRLVLGKFGDLTDNFSS
PHARRKVLAGVVMTTGTDVKDAKVISVSTGTKCINGEYMSDRGLALNDCHAEIISRRSLLRFLYTQLELYLNNKDDQKRSIFQKSE
RGGFRLKENVQFHLYISTSPCGDARIFSPHEPILEEPADRHPNRKARGQLRTKIESG£GTIPVRSNASIQTWDGVLQGERLLTMSC
SDKIARWNVVGIQGSLLSIFVEPIYFSSIILGSLYHGDHLSRAMYQRISNIEDLPPLYTLNKPLLSGISNAEARQPGKAPNFSVNW
TVGDSAIEVINATTGKDELGRASRLCKHALYCRWMRVHGKVPSHLLRSKITKPNVYHESKLAAKEYQAAKARLFTAFIKAGLGAWV
EKPTEQDQFSLT (SEQ ID NO: 45)
>EcCas6e-H20L-NES-Linker-hADAR2_{DD}-E488Q/T375G (ADARv2)
MYLSKVIIARAWSRDLYQLLQGLWHLFPNRPDAARDFLFHVEKRNTPEGCHVLLQSAQMPVSTAVATVIKTKQVEFQLQVGVPLYF
RLRANPIKTILDNQKRLDSKGNIKRCRVPLIKEAEQIAWLQRKLGNAARVEDVHPISERPQYFSGDGKSGKIQTVCFEGVLTINDA
PALIDLVQQGIGPAKSMGCGLLSLAPLLPPLERLTLGGGGGSGGGGSGGGGSGGGGSQLHLPQVLADAVSRLVLGKFGDLTDNFSS
PHARRKVLAGVVMTTGTDVKDAKVISVSTGGKCINGEYMSDRGLALNDCHAEIISRRSLLRFLYTQLELYLNNKDDQKRSIFQKSE
RGGFRLKENVQFHLYISTSPCGDARIFSPHEPILEEPADRHPNRKARGQLRTKIESGQGTIPVRSNASIQTWDGVLQGERLLTMSC
SDKIARWNVVGIQGSLLSIFVEPIYFSSIILGSLYHGDHLSRAMYQRISNIEDLPPLYTLNKPLLSGISNAEARQPGKAPNFSVNW
TVGDSAIEVINATTGKDELGRASRLCKHALYCRWMRVHGKVPSHLLRSKITKPNVYHESKLAAKEYQAAKARLFTAFIKAGLGAWV
EKPTEQDQFSLT (SEQ ID NO: 46)
>EcCas6e DR coding sequence
GAGTTCCCCGCGCCAGCGGGGATAAACCG (SEQ ID NO: 47)
>DMD Exon54X (G > A) target sequence
agaaaagtccacatgataacagagaatatcaatgcctctTagagaagcattcataaaaggtatgaattacattatttc (SEQ ID
NO: 49)
>Rpe65 Q64X mini-gene
ATGattgaacaccctgctggtggctacaagaaactatttgaaactgtggaggaactgtcctcaccactaacagctcatgtcacagg
ttggtctcattgatcttagcttgcttattgattgtacattctaaactggcatcttgcagtatagcctaggttgatataaaccatga
cacctttcttgctttatcctgagtactgggttcaagctaacagaacctaactcttcagaaagctttcttttctgtcaagatcatcc
tgtatcagacatcaagtaagcagtagacaggagccactgttagtatcatcaactctgtccaaagacctcatgtgactcatgctttg
ctatgtgtaacaggatgctatgtgaaatgtcagcagttgggcaaatcttttctctgttttctgctctataaaaggcaaggagagca
gtgaacactcttcaactagatggtggtttagtatcaaacattagctttcagtcaaagcaaatattgttttctaatttttatttgat
gtctgcttgatagttgaacttaatatgtaggcaacaatcgatttgttttaaaataaaaacctggtaaggattgagcaaacatgaac
ttttctggggatgagaggactgatcaaacctcattgtgaaaaatggaagtccccaatgacaatcttggtgccattcatttttcttt
ttttatgaggaaacaaaagctcttagagaaagcaggaggacattctacatatgaatccaggtcctgacacttatccctgtatctta
tctgccacaaagggtgaatttcataaagcgtgctcatttatatcccaagataaatgggagttttagattttaaaaactcagccaac
attcttgctgcttcatataaaaggttctttctacatgagtagagtagtgcacatctttaatttcagcattcaaaacacagaggcag
aagtgtgaggccagcttggtctgtttaactgagtatagaccatcaaaaaaccctatcttaaaaaatgcattttttaaaaaattgac
acctagttttaatattttgatccatgctcctaaagtctatccttgtactttttaaaccaccaaattgcataagaaaaactgatgtt
ctgtctataggtaagctgacaaataacaaataggcacatagaaaatctagtaagtagtaccacctatagctctagattttaaagag
ccaggattcctgttttttcatttaaatagtcttttttctatagacacatctttttaaaatacatttattctcactcttaatccata
cttctactcctcactccactaaaatatatcaagaaatttgaaaatcttcttaaaaatgagatggcaaaaggcaaatggattggtgt
ttggggaaatctattaggtggagagtcccaatcctctcaagatgcttctccctcactccttccaaccctttatccacactctgggg
catctccacatttcccaaggtgtgatctcctagtgaaggtgagtcaagtcagcagacattttctaagtgctgtccagcgcgagggc
tggaaatgaaaatcactgccagctctatgaggaagaagctgcccaatcaggctgctgatatactctgccttaccaaggacaagcct
agcccaaggcagggataagaagcaatgttctgtctccccttcatcacaggcaggatccccctctggctcaccggcagtctccttcg
atgtgggccaggactctttgaagttggatctgagccattttaccacctgtttgatgggTaagccctcctgcacaagtttgacttta
aagaaggacatgtcacataccacagaaggtaaagcagcactccatgccactcagagtagagcctatctcagttcttctcctgtttg
aactttcaccttcatgaacccagagaaaacctctaaacacatttctttcttctttcttcatggtctagccatgtctgtatatttgc
acagccacctgagaatgttaaaatgagataatggggttcaagagctctgagacaaacatggctatcttcaaatcctgaataaacca
tgcaccattttgtcttaaatgatggcatctggtagggaattattggcctctaattactttgtcaactgtagaatgaaagaaatggt
agaatctactctatagagtctacccatgactaaatgagaataaaatatgtcactcattgtaataatgcagtaaaaacaaaaactgc
tagtacatctgtgattactagactattgggttatctaaatttctttcattttttatcatcagaaaggtctaaaaaagaagctggta
aaatgactcagaaggtgtgggtacatgccattaagccttaccctaatccaatccacatataaactacctctagcaagttttcctct
gaactctgtatgcattccatagctcctgcacatgtgtgtgtgtgtgcgcgcacacacacacacactaatgtttaaaaaattatagg
gacttgaagagagttccacaaaagggggggagggacaaaatatgtcacagaaacactagatacagatgtgaagaagagtgagagaa
gggagaaagacagagggatacaaagagaaaatttttagccagttatttgctaaagggttagagcaatgactcagcatccagagaac
ccaggattcaattctgagcaccatggcagctgctgctcacaactgtctacaactctagaccaaggggatccgatgtactttctagc
ctctatgggtaacaggcacacatgtagtacacagacatatgtgcaagcagaacacacatacatataatattcgtaataataatttt
attaatagtaacaacaataaagagttattactaaagccatcattccaagaagcccccataacaacacagtaaataccctgattcct
atttgaatattcaggcttgttagtggtatgattaccataaatttcaagcattttgaagggacatcaaaggaagagtaaggggaaac
tggaaataggatttgaactgtgacaaaagagaggaagggactgacaagcataagtttttctaggaaagcaattttggatttcagac
acagattttatataacttctaatgtgtactttcatatacattcctttttataaaagcacaaaatcaaattttactctgccttccat
atggcaagcctcaagtagcattttccaggtggtctaggaagtgactcacagataaatcagcctgtgaaagagataaaattcaataa
aaactaaattttaaaatatataatacattttagaattaaagagaaacacaaggtcaaacagatgaaaaaaatataatcaatcacac
tatgtcctataatgtgatgagctacatgtgtgacatagccacatgttctagtttgttttctacttctgtgataaacatcatgacca
agaacaagttgagtaggaaagatgtttatttcatctttacaattcatcaccaggggaagccaaggcaggaaggaatcagaacatgg
aacacagaccatgaaggaatattgtttactgcctaatggtttgtgtcttcttcttcttcttcttcttcttcttcttcttcttcttc
ttcttcttcttcttcttcttcttcttcttcttcttcttcttcttcttcttcttctccttttcttcttcttcttcttcttcttcttt
cttcttctttcttcttctttcttcttcttctttcttctttcttcttcttcttcatcttcttctttcttcttcttcttcaccttcct
cctcctcctcctcttccttctcctccactgctgccactgctgccaccacctccttcttctctttacctctctctcctttcctaacc
ctttcttttaaaaatgttttggaaatatttcatgtatatgttgcctgcatgtatatctgtgcaccacattcatgcagtacctgcag
aaactggaagatcatgtcacatctcctggaagtggagttatagatagatggttgtgagcccaaaacatggatgctgggaattgaat
ccaagtcttctggaagagcagtcaatgatctcaatggctgagccaccacttcagccttcagcctgttttctgaagtggtacctgtc
cagtggtaccatacacagtgggctggtcttgcctgtatcaatcattaatttgttaaaatgtcctcatggacttacctacagaaaat
ctgatggaggcatcttttcaatgaaagttttcttttcctccaaagaattctagtttgtgtcaagttgaaaaaatctacacaccatg
acgtggggcactttcctgttttctaactctgatcaatgtcacaatcacatatatctactcatctttcggagcctcagtctctctat
caccggcttggggagattcatgattccaatttgatgaaaacacactgactctcagtttcatgtgtctagaatgtatagcctacttg
tatcctttatccatttctcaatattcttcagattcatccgcactgatgcttatgttcgagcaatgactgagaagaggattgtcata
acagaatttggcacctgtgctttcccagacccctgcaagaatatattttccag (SEQ ID NO: 50)
>d2EGFP
VSKGEELFTGVVPILVELDGDVNGHKFSVSGEGEGDATYGKLTLKFICTTGKLPVPWPTLVTTLTYGVQCFSRYPDHMKQHDFFKS
AMPEGYVQERTIFFKDDGNYKTRAEVKFEGDTLVNRIELKGIDFKEDGNILGHKLEYNYNSHNVYIMADKQKNGIKVNFKIRHNIE
DGSVQLADHYQQNTPIGDGPVLLPDNHYLSTQSALSKDPNEKRDHMVLLEFVTAAGITLGMDELYKKLSHGFPPEVEEQDDGTLPM
SCAQESGMDRHPAACASARINV (SEQ ID NO: 52)
>mCherry*
MVSKGEEDNMAIIKEFMRFKVHMEGSVNGHEFEIEGEGEGRPYEGTQTAKLKVTKGGPLPFAWDILSPQFMYGSKAYVKHPADIPD
YLKLSFPEGFKWERVMNFEDGGWTVTQDSSLQDGEFIYKVKLRGTNFPSDGPVMQKKTMG*EASSERMYPEDGALKGEIKQRLKL
KDGGHYDAEVKTTYKAKKPVQLPGAYNVNIKLDITSHNEDYTIVEQYERAEGRHSTGGMDELYK (SEQ ID NO: 53 and SEQ
ID NO: 92)
>spacer coding sequence targeting mCherry*
ggaggaggcctccCagcccatggtcttctt (SEQ ID NO: 54)
>EcCas6e
YLSKVIIARAWSRDLYQLHQGLWHLFPNRPDAARDFLFHVEKRNTPEGCHVLLQSAQMPVSTAVATVIKTKQVEFQLQVGVPLYFR
LRANPIKTILDNQKRLDSKGNIKRCRVPLIKEAEQIAWLQRKLGNAARVEDVHPISERPQYFSGDGKSGKIQTVCFEGVLTINDAP
ALIDLVQQGIGPAKSMGCGLLSLAPL (SEQ ID NO: 55)
>RescueS
QLHLPQVLADAVSRLVIGKFGDLTDNFSSPHARRIGLAGVVMTTGTDVKDAKVICVSTGAKCINGEYLSDRGLALNDCHAEIVSRR
SLLRFLYTQLELYLNNEDDQKRSIFQKSERGGFRLKENIQFHLYISTSPCGDARIFSPHEAILEEPADRHPNRKARGQLRTKIEAG
QGTIPVRNNASIQTWDGVLQGERLLTMSCSDKIARWNVVGIQGSLLSIFVEPIYFSSIILGSLYHGDHLSRAMYQRISNIEDLPPL
YTLNKPLLTGISNAEARQPGKAPIFSVNWTVGDSAIEVINATTGKGELGRASRLCKHALYCRWMRVHGKVPSHLLRSKITKPNVYH
ETKLAAKEYQAAKARLFTAFIKAGLGAWVEKPTEQDQFSLT (SEQ ID NO: 56)
NES (SEQ ID NO: 48)
LPPLERLTL
nucleoplasmin bipartite NLS of KRPAATKKAGQAKKKK (SEQ ID NO: 64)
c-myc NLS of PAAKRVKLD (SEQ ID NO: 65) or RQRRNELKRSP (SEQ ID NO: 66)
hRNPA1 M9 NLS of NQSSNFGPMKGGNFGGRSSGPYGGGGQYFAKPRNQGGY (SEQ ID NO: 67)
sequence RMRIZFKNKGKDTAELRRRRVEVSVELRKAKKDEQILKRRNV (SEQ ID NO: 68) of IBB domain from
importin-alpha
sequences VSRKRPRP (SEQ ID NO: 69) and PPKKARED (SEQ ID NO: 70) of myoma T protein
sequence PQPKKKPL (SEQ ID NO: 71) of human p53
sequence SALIKKKKKMAP (SEQ ID NO: 72) of mouse c-abl IV
sequences DRLRR (SEQ ID NO: 73) and PKQKKRK (SEQ ID NO: 74) of influenza virus NS1
sequence RKLKKKIKKL (SEQ ID NO: 75) of Hepatitis virus delta antigen
sequence REKKKFLKRR (SEQ ID NO: 76) of mouse Mx1 protein
sequence KRKGDEVDGVDEVAKKKSKK (SEQ ID NO: 77) of human poly(ADP-ribose) polymerase
sequence RKCLQAGMNLEARKTKK (SEQ ID NO: 78)
MS2 binding loop (5′-ggcccAACAUGAGGAUCACCCAUGUCUGCAGgggcc-3′, SEQ ID NO: 79)
MS2 (MS2 coat protein)
MASNFTQFVLVDNGGTGDVTVAPSNFANGVAEWISSNSRSQAYKVTCSVRQSSAQKRKYTIKVEVPKVATQTVGGVELPVAAWRSY
LNMELTIPIFATNSDCELIVKAMQGLLKDGNPIPSAIAANSGIY (SEQ ID NO: 82)
QBeta binding loop (5′-ggcccAUGCUGUCUAAGACAGCAUgggcc-3′, SEQ ID NO: 80)
Qbeta (Qbeta coat protein)
MAKLETVTLGNIGKDGKQTLVLNPRGVNPTNGVASLSQAGAVPALEKRVTVSVSQPSRNRKNYKVQVKIQNPTACTANGSCDPSVT
RQAYADVTFSFTQYSTDEERAFVRTELAALLASPLLIDAIDQLNPAY (SEQ ID NO: 83)
PP7 binding loop (5′-ggcccUAAGGGUUUAUAUGGAAACCCUUAgggcc-3′ (SEQ ID NO: 81)
PP7 (PP7 coat protein)
MSKTIVLSVGEATRTLTEIQSTADRQIFEEKVGPLVGRLRLTASLRQNGAKTAYRVNLKLDQADVVDCSTSVCGELPKVRYTQVWS
HDVTIVANSTEASRKSLYDLTKSLVVQATSEDLVVNLVPLGR (SEQ ID NO: 84)

| | |
|---|---|
| Cas13e.3 | MAVDYSLKNEWYREINKSCFTVALNVAYDNCKAKGHENLLREAQRSKGGITNEQIKNVQTEIKTRLEDIR |
| amino acid | SHFSHFYHDEKSLIFEKDNIVKDFLESAYEKAQSSVIGSTRQSDYKGVVPPLFEPHDGMITAAGVVFLAS |
| sequence | FFCHRSNVYRMLGAVKGFKHTGKEELSDGAKRDYGFTRRLMAHYSLRDSYVIKAEETKSFRDLLGYLSRV |
| | PQQAVDWLNEHNQLSEDEKKEFLNQKPSDEESQEQSKTENTDRQADRMPRRSLRKTDKFILFAAKFIEDW |
| | AQKEKMDVTFARYQKTVTEDENKNQDGKQVRDVQLKYEKDTKKLNPDFDYKWTYYIRNNHAIIQIKPDEY |
| | KQAVSARISENELKYLVLLIFQGKGWEAIKKIGDYIFHIGNKIKIGRFDHNEERRMPSFLKNPPADIIGE |
| | MVENRLKYIRDELNKVIETIKKEEPQNNKWLLYKGKKISIILKFISDSISDIKKRPDVNEYNTLRDMLQK |
| | LDFDNFYERLKSYVSEGRIEQTLYDEIKGIKDISTLCIKICELRLAALEELEKEGGDDLNKYIGLAVQEK |
| | HKNYDDSNTPQKKAERFLESQFSVGKNFLRETFYDEYIKNRKSLYEIIKEKITGITPLNENRWYLMDKNP |
| | KEFESKDSKIIRGLCNIYIQDILCMKIALWYYENLSPSYKNKLKWDFIGQGFGYDRYKLSYKTDCGITIE |
| | FKLADLNRLDIIEKPKMIENICHSFILEKDVKKQTISWHEFRQDGIAKYRKLQKEVVEAVFEFENSLKIP |
| | DKNWLTQGYVPFNKNKRFEDKGFSTFILEEAVRKGKIKSDDKEPLRKVRTDFFHEQFDSTDAERRIFDKY |
| | MPAKHDGKNKGGKMQEKQEKSYTRRI (SEQ ID NO: 111) |
| Cas13e.4 | MEKEQGLYSIDRYQGAGKWCFAIGANRAWDNYNERPKLFSESLLRYEKATRRDWFDEETRGLIKKSDVRQ |
| amino acid | RLRKIRCYFSHYCHDNTCLGFDPDDDLRKIMEKAYERAIFEQRKHLSTETDIETPALFEPHGRITAAGVV |
| sequence | FFCSFFVERRILNRLMGRIPGFKKTEGEYGATRQMFSKYCLRDSYSIRASDSNAVLFRDILGYLSRAPSQ |
| | YYRHNKDQCDKDGHPERKKDKFINLALRYLESFVPARLRNHTLSVGRKEVVRMETNAVAEGEGEYRPYPP |
| | KAKVKVVFTEDDPERPYYITHNTVILQTAKKEEDIHHCKVGVNELKYLVLLCLQGKAEKAVAGIEGYVRR |
| | IQGRFADHTNKVARDDDERLVRGLPEFVRVASGIETPDEVRELKSRLDHIRKKWQTKKAESAEAQLHRKA |
| | RDVLRHINWESQRPLGIEQYNRLLELLVNRDLESFAAEMKELKRRGLISEELLKSVEGIRNLNTLHVKVC |
| | NLVLTRLEHLVENDPEELKRHIGIVPREEKEGPSYEEKVRAFVQQPMMYRGFLRNSFFKGSGKSFAKLVE |
| | EELHKKGCPDVPLGTDYYLVRDLERDERKNRFHNDNAALYETLALDRLCVLMARDCLVRLNRNLEKHATR |
| | ISWEATDAGDTICLELPRRDRDHESFRLSFGVRDYPKLYVMDDPVFLCGLMKHFFPDNQAIQYHELYSEG |
| | INKYTAMQAEGIAATLKLEEKTIKEKNMQIPATGYIRFCEIVSQSDFAPGEKRVLKNVRNGLLHYHLEFE |
| | PTEWAEFREIMKREGFDTAKKRKSTRKK (SEQ ID NO: 112) |
| Cas13e.5 | MKIKNENTDKKTELYSIDKYKGRDKWCFAIVLNKAQTNLTENPDLFEQTITKYDRIRKEGWFDEETKKLI |
| amino acid | YIQENEHKIKGEIKTLAREVLKNLRNYFSHHFYKQDCLIFPKDNIVRIIMGRAYERSEYEIKKNLKEDIS |
| sequence | IELPALFEPEGKITTAGIVFFTSFFVERRFLHRLMGSVQGFTKTEGEYKITRDVFAKYCLRDSYSVKAQD |
| | NDAVMFRDIIGYLSRVPTESFQHIKNPKKQNESQLSERKTDKFISFALKYLKDYGFEDLKGHYTAFFARS |
| | EIKKEKEDIEIKDDKKHKPHRMKSKIEIHFDKTKEDRFYIERNNVILKIQRKGGRANILRMGIYELKYLV |
| | LLCLSGKAREAINRIDDYLNDLRNKIPHIENMNKEGIGEQIRSLPGFVRSQLGFVQIDDEKKKENRLDYV |
| | EKKWEKKRAESKELKLNRKGRDILRYINERCKKPLTIDRYNRILELLVEKNIEGFYHELEELRKTGRIEK |
| | NITQALVGEKNINALHIKICKLVQDELKSLEKEDLKKYIGLTPKEEKVVSFEEKLGRILDKPVIYKGFLR |
| | YQFFKNDKKSFARLVEEIIKEKTGGLDVPIETEYYSISTLGRFDKANKTLYETLAMDRLCMMMARRYFLS |
| | LNKILAKRAQNIEWKKESGKEFIVFKFNMPQDTGKSISIRFSPKDYTKLYVKNDSEFLARLCQYFFPNEK |
| | AIDYHKLYSHGINKYTNFQKEGIEAILELEEKIIKKRKINSPENYLSFEEILNQSIYNDEEKNTLIQIRH |
| | SLLHYQILFSKNDLTKFYNVMKREGIEKIWSLVI (SEQ ID NO: 113) |
| Cas13e.6 | MAVNYSLREKWYRGVNKCCFTVALNIAVDNCKSKGCETLLKEAEHSKGGITDEQIQQSYTEVEKRLNDIR |
| amino acid | NYFSHFYHGDECLIFKKDDIVKRFMESVFATAVSNVVGGTKESDYKGVVPPLFEQSNEDYMITAAGVIFL |
| sequence | ASFFCHRSNVYRMLGAVKGFKHTGKEELSDGQKRDHGFTHRLLAHYSLRDSYSVKIEETKSFRDLLGYLS |
| | RVPQQAVDWLNERNELSEDEKKEFLNQKSSEEESPEQPEPENAEWRTEKTSRRSLRKTEKFILFAAKFIE |
| | DRAEKEKQDVTFARYQKTVTKEENKNQDGKQARVVRLKYEEDKKDDEKPREHFNLEWMYYIRNEHAIIQI |
| | KPKDKEAVAARISENELKYLVLLIFEGKGGDAFNKLSDYIFRMTQKIKSGQINPNEARLPSFLKNPVKNI |
| | TDKMVRNRLDYIRGQIKDVLEKINMEEPQNNKWLIYKGKKISLVLKFISDGISDIKKRPNVKEYDTLRDT |
| | LQKLDFNRFYERLKSYVSDGRLAAALYDKIKGIDDISELCKKVCELMFARLAELEKKGGFELYRYIGMEV |
| | QEKDEKYDEWNSPQKKAERFLESQFSIGENFLRESFYSEYCQKQECIDKEISLNTSVKNRKSLVYIVKEK |
| | LKDIMPLHNDRWYLIDRNPKDFERKDSKVIKGLCNTYVQDVLCMKMARWYYGQLNPALKNNIKWDETGQG |
| | HGYDRYKLSYRTNFGITIEFKLADFTRLDIIEKSDMIENICRSFIKPNRTISWYDFKQDGVEEYRKRQYK |
| | AVRAVFAFEESLIIPGRDWLSQGFVPFIKNEEYVKKGFSLFVLDEAVRQLKIKGSDKDAMRQVRNDFFHE |
| | QFQAKDEQWKVFEGYLSCFMIDRPKGEKNKKRYNGNKK (SEQ ID NO: 114) |
| Cas13e.7 | MEKYLIKNFEGINKSKFTVALNIANDNCKNKGIQELLKEAQRSKGGITDTQITEVQEHIKERLNSVRNYF |
| amino acid | SHCYHEKKPLYFEANDPVKIFLEETFAKAVENLQGRFLSDKYKLTVPPLFEPNQNNTITAAGVIFLASFF |
| sequence | CHRSYVYRMLGGIPGFKRSDKKKWGDGQKIDYGFTRKLMSFYSLRDSYSVNVQENKELTAFRDILGYLAR |
| | VPGQAIDWLIEKGKLTKEEGKQFYLGEQSEEREEKAKKEEIKYALRKTDKFMLFAVRFIEDWAEQERIKV |
| | EFARYEKMTIVNENKKQDEKEERKVKFVSDEPTAAGWTYYIRNNHAIIKIIPDDKKKKAVSARISENELK |
| | YLVLTIIDGNGKNAIAYIGDYIFRTARQIENKSYNAESEKYAPAFVRGGQKKSVDKRIKYIRDEIQQVIN |
| | DIEAEQEKQKNEQDAPAENRTWLIYKGKKISIILRYVNDNIAEYKKRLSVTEYNELRGYLQQLDFINFHR |
| | KLAEYQHHGRLPNGFAESINKFQDLSKLCIEVCERQKKKLQEMAAKGGIELEQYIGLAPKEENQEQNKYA |
| | TKANNFIKVWLSIPENFLRQKFYDKFCKQQECKNKGSDKPDNTSVPQRKYFIAIIREKNIRPIHADKYYL |
| | LGQNPKDYERPDGKIIRQLCDVYCKDGLCMAMAKWYYENRLGKFKDLIEWQTGDDKQQHGYAGHTLEYQA |
| | TEKIKIRFKLADFTRLDIIEPPERVKNICRQWETELLKKTRDGTISWYDFKLNGLEPYRQWQGYAVADIF |
| | WFEESLKINETQWQGRTHMPFNFEKDKPLWCNILDEAVKQNKIEKQDTQALRRVRHDCFHEEFLANYEQL |
| | KIFKNLISDKAKDAKPKDKKSRKNEQKYGKR (SEQ ID NO: 115) |
| Cas13e.8 | METTEEKKEIYSIDKYQGAGKWCFAIILNRARDNYEGNPHLLSESLLRFEAVNRRDWFDEETRELIIQMD |
| amino acid | AETKTDSNLKPSTPAYKILIGIRNYFSHFYHTEECLYFKEDDPIRIILENAYKKAKQHHIERLEKETDIE |
| sequence | FPALFESNDRITSAGIIFFSSLFVERRILNRLMGYVGGFKKTKGEYNITREIFSTYCLRDSYSIMAADSN |
| | AVIFRDILGYLSRVPSEYYQHNKEKCEKENKPKRKTKKFIYFALRYLEKFVLKEIKNQKVSIARMEVVRE |
| | KSKEAEGEDEQHKPYPDKAKVKIVFDSIGKELPYYINHKTVIMKIQRNGEAVNFCKIGINELKYLVLLCL |
| | QGKSVDAIAKINGYIDRIKKRLENPKTRIDIDSERDNEFIKGLPEFVKIQSGRTPDEEREKKSRIDYIRK |
| | KWEKKKEESPKTELHRKGRDILRYINWHCEPPLGSEEYNHLLSLLVNKDLDGFENELKELKRTEQISKKL |
| | LEMLKGFSNLNELHLKVCSIVPVELVYLEKNDPEKLAEYIGLIPQKTDKEPPSYEEKVKNFIKQPMIYKG |
| | FLRDTFFSSGKTFAKLVEETFLHKYPHSDVPLGRDYYHVTTLDRFHKDNSILYETLALDRLCVVMARKFH |
| | ETLNQELAKESKQIVWENNTIILELPRSKTSSSDTFQICFDIKHYMKLYVMDDVEFLGGLMRHFFQKEKT |
| | IEYYNLYSFGINKYTEMQRNGIEAILRLEEKIIREKNIKRKPDKNYISFYEIMEASNYPDNDKKTLNKVR |
| | RALLHYHLKFEPADYNKFVAIMKREGLERKKKTGKATRRWVKK (SEQ ID NO: 116) |
| Cas13f.6 | MENIKLEKQKAAFYFNQAELNLKAIEGNIFDKGRRKTLFDNPKILSKVENFIFNFKDVTKNAKGEIDCLL |
| amino acid | SKLMELRNFYSHYVHKPDVKELSKGEKPLLERYYQIAIEATGSENVKLEIIENDKWLTDAGVLLFLCMFL |
| sequence | KKSQANKLISGISGFKRNDTFGQPRRNLFNYFSVRERYKVVPDMQKHFLLFVLVNHLSEQDDYIEKAQQP |
| | YNIGEGLFFHRIASTFLNVSGILRNMEFYTYQSKRLKEQRGELKREKDIFTWEEPFQGNSYFEINGHKGV |
| | IGEDELKELCYALLSYNKSKYAVEQIEKFLKGFGEVKSEQEIRDSDILNESYFPTNYFAESNIGSIKEKI |
| | LNRLGKTDDSYKKTGTKIKPYDMMKEVMEFINNSLPADEKLKRKDYRRYLKMVRIWDSEKDNIKREFESK |
| | EWSKYFSSNFWMAKNLERVYGLAREKNAELFNKLKAVVEKMDEREFEKYRQINSAEDLASLRRLANDYGV |
| | KWEEKDWQEYSGQIKKQISDRQKLTIMKQRITAELKKKHGIENLNLRITIDSNKSRKAVLNRIAVPRGFV |
| | KEHILGWQGSEKVSKKTREAKCKILLSKEYEELSKQFFQTRNYDKMTQVNSLYEKNKLIAFMAVYLMGQL |
| | NIRFDKPTRLNELEKAEVDFKISDKVTAKIPFSQYPSLVYAMSSKYADSVGSYKFENDEKNKPFLGKIDI |
| | IEKQRMEFIKEVLGFEEYLFEKKIIDKSKFADTATHISFREICDELIQKGWDENKLTNLKDARNAALHGE |
| | IPAETSFREAKPLINGLKK (SEQ ID NO: 117) |
| Cas13f.7 | MINIELKKEEAAFYFNQANLNISGLDEVIEKQLPHIGSKKENAKKAIDKIFDNITVLKKVENFVFYFKDV |
| amino acid | AKNERVELDALLLKLIDLRNFYSHYVHNDNVKILSDGEETLLEKYYQIAIEATGSKDVKLEIIDNEKKLT |
| sequence | DAGILFLLCMFLKKSQANKLISSISGFKRNDKEWQPRRNLFTYYSLREGYKVVPDMEKHFLLFTLVNHLS |
| | TQDENIENTQPSDDIGRGLFFHRIASTFLNISGIFNNMEFYPYQSNRLKERRGDIAPDKDSFAWIEPFQG |
| | NSYFKINGYKGVIGENELKELCFAVLLHNKSKYAVEQIEKFLKCFKEVQSKQEIIECDILDECYFPANYL |
| | NQPETKSLKEKLLSRITGKINYSFDTAEKAFDKMKDVMEFINGCLPSDEKLKRKDYSRYLKMVRFWGGEK |
| | DNIKREFEGKKWTRFFPSELWHKRTLEDVYKFALKKNKKRLEELKVKIEGLNEDDLLKYQKVNNIKNLEN |
| | LRLLAHDLDLSWREKDWGEYSGQIKKQISDNQKLTIMKQRVIAELKKKHGIENINLRISLDSNKSIQAVL |
| | NRIAIPKGFIKRHVLHLQENEKTSRKIREAKCKILLSKKYEYLSRKFLDEKNFDKLTQINGLYEKNRLIA |
| | FMVIYLLKQLGLELKNETKLIELKKTRVKYKISDKVAEDIPLSHYPSLVYAMSRKYVDNIDNYEFPDEYA |
| | KKAILDKVDIIENQRMEFIKQVLGFEKYLFDNNIIDKSKFTDVETHISFVKIHDELIEKGWDTEKLSKLK |
| | HARNKALHGEIPGGTSFEKAKLLINELKK (SEQ ID NO: 118) |
| Cas13d.1 | MKKQKSKKTVSKTSGLKEALSVQGTVIMTSFGKGNMANLSYKIPSSQKPQNLNSSAGLKNVEVSGKKIKF |
| amino acid | QGRHPKIATTDNPLFKPQPGMDLLCLKDKLEMHYFGKTFDDNIHIQLIYQILDIEKILAVHVNNIVFTLD |
| sequence | NVLHPQKEELTEDFIGAGGWRINLDYQTLRGQTNKYDRFKNYIKRKELLYFGEAFYHENERRYEEDIFAI |
| | LTLLSALRQFCFHSDLSSDESDHVNSFWLYQLEDQLSDEFKETLSILWEEVTERIDSEFLKTNTVNLHIL |
| | CHVFPKESKETIVRAYYEFLIKKSFKNMGFSIKKLREIMLEQSDLKSFKEDKYNSVRAKLYKLFDFIITY |
| | YYDHHAFEKEALVSSLRSSLTEENKEEIYIKTARTLASALGADFKKAAADVNAKNIRDYQKKANDYRISF |
| | EDIKIGNTGIGYFSELIYMLTLLLDGKEINDLLTTLINKFDNIISFIDILKKLNLEFKFKPEYADFFNMT |
| | NCRYTLEELRVINSIARMQKPSADARKIMYRDALRILGMDNRPDEEIDRELERTMPVGADGKFIKGKQGF |
| | RNFIASNVIESSRFHYLVRYNNPHKTRTLVKNPNWKFVLEGIPETQIKRYFDVCKGQEIPPTSDKSAQI |
| | DVLARIISSVDYKIFEDVPQSAKINKDDPSRNFSDALKKQRYQAIVSLYLTVMYLITKNLVYVNSRYVIA |
| | FHCLERDAFLHGVTLPKMNKKIVYSQLTTHLLTDKNYTTYGHLKNQKGHRKWYVLVKNNLQNSDITAVSS |
| | FRNIVAHISVVRNSNEYISGIGELHSYFELYHYLVQSMIAKNNWYDTSHQPKTAEYLNNLKKHHTYCKDF |
| | VKAYCIPFGYVVPRYKNLTINELFDRNNPNPEPKEEV (SEQ ID NO: 119) |
| Cas13d.2 | MKKSKVKLNGVKAVYHISPDVRVIAAFGRGNNSVLDKHIENGSVEELQNHSDIEVNISRKTYSFRKKSLK |
| amino acid | KAAGQFSVPDNTNDQLGIREELEKEIFGRKFDDNIHIQAAYAVNDIIKMLAVAANLAETAINGLDRENTE |
| sequence | NDMIGFCIIPHITYQTYADDKRSKFEGFIEKVKTQGTFSYFPDILPKFKKEESEEESDNEKLYYIMCIIS |
| | LIRNSATHSKSSNSDTTDYIFGEFNSVNKEALTTTADNLIKSKIDFINKEGFSKNQKNNIYRLLKAKADT |
| | PENTARLIRRLYAFTIRKQDKNLGFSLKKLRECAIRSIDRSIEYMKYLPSKKYDTVRSKLYTLMDFVVYS |
| | YLKYHKDGKKFSKEMVEQLRAAESDKVKDDIYRDEAEKLYNIEIISRTINALISDIKSDFDQPKHGNECY |
| | QPINDGMKEAEKDFITTDQLSLFTKFIYVLCQFLDGKNINILLSSLISKFQQIEAFNGDIRKLNLNIRDD |
| | GKIGYDSKKYSIFEKSGQIADDLDKLRGVIKMDINDLNAYETMIKDALRVIGVDESDIESIYQTHFKTQD |
| | KKDSVSGFFRNNIINSRRFRYIIKYINPSDAYRIIQNENVRNYVLGRMNDAIIDRYAHSVGIEDKVHDKR |
| | KVLSDILSKVKFDNFTKLTYINPKDKNKGEKAKEREKPKAILGLYLTIVYLVVKSLVRINSQYVMAVYHL |
| | ERDSRLCPGVSSNNLLSMTNHYCDRDNHLLKEKHIVKLERYKKTQKTQKTICTAYRNAIAHLSAVRKGVK |
| | YIGDIQKADSYFGIYHYCMQKLIYSANGQPFAEFVRSIFGDEKELDKLRNGSYSQAILRALNYPFGYNPA |
| | RYKNLSYEKIFIRAWQDEDTNKKT (SEQ ID NO: 120) |
| Cas13d.3 | MNKIHKKQGKTTAKSLGLKSVLKIENDLVVTTFGKKDNPMVVEQSINKASGEKELYVDEDQVKFDSSLIK |
| amino acid | EKNILSLDSIQHSNHQIIVNIDQKDASEIGMDYLRLKPELEKEFFGKTFYDNVHIQIAYNLLDLKKIIGL |
| sequence | HIGNAIQALENLGRDGSDLVGICDATKPLNYLDDVKQKADIGFMNRLKPYFMYFDGVLKLDNSKNKNGEL |
| | NQLDIENWDVIRILSLIRQGCAHAGAYSSLLYTAQNNKVYADLINKALSIFSDDLDKFNKSFLKQSKMNL |
| | FILFDLYNCRFDRSLQEKIIKEYYRYVLYKDNKNLGFSLKNVRNLIIEGKYDEQERSGKLQTIRSKLNTL |
| | LDFYLYGYYQKNPTFVENIVAKLRESKNDEDKEKVYEEEYHRLLSENNYLVDKKCSDIVYRINEAVKNRK |
| | IFVNANINAVVEKVSCSCFPSLIYVLCKFLDGKEVNELTTAIINKLENIASLINALVTLKSYGGFSEQYK |
| | IFDYPNINGLIDDFRMVKNLTSTKRKLKKASGGEDRIGRQLYADAINIFKEDSFVSANDEKGTGLDQYVN |
| | KFFSKDDLGARKVRNLLLNNIIKNRRFVYLIKYIDPKDCYKLVHNEKIVRFALGQYDESQMPLNQLQKYY |
| | DAVIENREGFRKCNDRKKIIDTLVSEINRVSIDGILDIGNRLVNRGNNDYINHQKQIISLYLTIAYLIVK |
| | GVVHTNSLYFIAWHAYERDNNFKFGNDGKDYLALTKEYLTNKKKRVKQLLDHNIEEANNSLDSKYFSAYR |
| | NKVVHLNFCNIFVNYLDGIGDIHSYYDIYQYVIQKWSIAERSKDFIDPQYLTKLSNDLKQYRTYQRNFLK |
| | IINLPFAYNLARYKNLTIGDLFNDKYPLPKETVKEFYNEE (SEQ ID NO: 121) |
| Cas13d . 4 | |
| amino acid | SSFGKGNNSVLEKRIIGDVSSDVNKDSHMYVNPKYTKKNYEIKNGFSSGSSLTTHPNKPDKNSGMDALCL |
| sequence | KTYFEKEIFKDKFNDNMHIQAIYNIFDIEKTLAKHITNIIYAVNSLDRSYIQSGNDTIGFGLNFNIPYAE |
| | YGGGKDSNGKPENKSAWEKRESFIKFYNNAKDRFGYFESVFYQNGKQISEEKFYIYLNILNFVRNSTFHY |
| | NNTSSHLYKERYCKINPKNNLKTDFEFVSYLNEFVKNKFKNVNKNFISNEKNNLYIILNAYGEDIEDVEV |
| | VKKYSKELYKLSVLKTNKNLGVNVKKLRESAIEYGYCPLPYDKEKEVAKLSSIKHKLYKTYDFVITHYLN |
| | SNDKLLLEIVEALRLSKNDDKKENVYKIYAEKIFKAEYVINPIKTISNLFAEKGDKLFNEKVSISEEYVE |
| | DIRIDKNIHNFTKVIFFLTCFLDGKEINDLLTNIISKLQVIEDHNNVIKAIANNNDAVYKDYSDKYAVFK |
| | NSGKIATELEAIKSIARMENKINKAFKEPLLKDAMLALGVSPNDLDEKYEKYFKTDVDADKDHQKVSTFL |
| | MNNVINNSRFKYVVKYINPADINRLAKNKHLVKFVLDQIPHKQIDSYYNSVSTVEEPSYKGKIQLLTKKI |
| | TGLNFYSLFENCKIPNVEKEKKKAVITLYFTIIYILVKNLVNINGLYTLALYFVERDGFFYKKICEKKDK |
| | KKTNKDVDYLLLPEIFSGSKYREETKNLKLPKEKDREIMKKYLPNDEDRKEYNKFFKQYRNNIVHLNIIA |
| | NLSKLTSTIDKEINSYFEIFHYCAQRVMFDYCKNNNKVVL (SEQ ID NO: 122) |
| Cas13d.5 | MKKNSNDKTNAKRMGIKSFIKNGDERFITTSIKNEFPVELKLDVIKKTCEPAHEPVSFDYDPKKIDFEKP |
| amino acid | VLKEKLTSGQSGQKLSTRLFIQKDRDICGIRRKYLEKIFNSNFIEEKKDSNLPMQIVAKVLSTEKVFSNA |
| sequence | LNKIISQFLSMPRGGVTDNHGEYEIIGNIINHKSLQELNKEKKTKRIKKYLQSVIKNQSYLYNKQFLLSL |
| | DESKGSRNDIDENELYDYIRFLAILRNGIAHVFYEKNEPETAKESLFRLVDFIKNDKKLEGAFAKIKIQV |
| | NTLYKCRKEEYIKKSGKNFEIIRKIYQNDKPDEKVKDWIRYDFDKSYKYIGLSVAKLGNYTSWAKDIDNL |
| | RDKSNPDSGYAGIMHRLNEFSVYLKVKALSTEEKDKYLKNLISKENCEEKDKYYKNIAQFFCSSDLKFAN |
| | VLQMVKEIKKNKGCTSEDKNCKLCVDERKFNDLSVIVYFISCFLDNKDQNIFLSDLINRFGALSDLLRIQ |
| | NKILGAGNKYNENYSFLKNERYVTEIKMELETIFALVKVSYKKEDKAFNRLLEDGLVMFGFSKDEAGMKV |
| | AGLKEIKEKKEGHYKNKSRSFLINSIVNSRKFAYLAKSIDPQKVPAIIKNEHIVRYILGRINKTNPGQIG |
| | RYWRYIMSQNHAGTDKVDDLTNEIIKINIKNILNDAGGWQKSKLNDNNNKKKLKYQQLIGLYLTVAYIFV |
| | KNMLECNARYFSAFAQIEKDYLIYTNSDEFYYIDKNKKNLVTERYLKLVKDIIEKNKNTVRKDKIFRKKR |
| | QRKHLADISKSIIEFEKLPCCIFTLLRNITEHLNVASNIDIIEGYGKRAGKYHKNAPASYFIFYHYIIQK |
| | ILADKICTRNLLNIINTYGEPSISFIKIIYVPFAYNLPRYLNLTDARIFCNMDDK (SEQ ID NO: |
| | 123) |
| Cas13c.1 | MKKLKNPSNRNSLPSIIISKFDSSKIYEIKVKYEKLARLDRLEIGDMSLDENLNILFKKVNFNGIDLEIL |
| amino acid | NPLLLDFDSYTISGKLQKNSTNKTILTLKKDGKIIKYNVLEKDNKYFKNGKEFVIPKDVKEEGKRLVNDK |
| sequence | FLLTIEDKKREENSLPKKRKKETQRDILKDETIEIYKRISSNSNIKSEDIYRIKRYMLFRSDMMFFYTFI |
| | DNFFYCLYKNKNEQLWNTNFKEKENLGKFIEFTLNDTLKNPRNGILKSYSKDLKVVQEDFVKIKDIFEKI |
| | RHALAHFDFTFIDNLLSNNIEFDFNIKLLNIVIEDSQDLYYEAKKEFIEDEKMDILDEKDISIKKLYTFY |
| | SKIDIKKPAFNKLINSFLIKDGVENSKLKEYIKEKYNCHYFIDIHDNKEYKKIYNEHKKLISENQNLQLN |
| | SKENGQKIKINNDRLEELKGKMNELTKANSLKRLEFKLRLAFGFIKVEYNIFKDFKNNFSEDIKKDMNID |
| | LEKIKSYLDTSYSNNQFFNYKVYNKKTKQKDIDKDIFDDIEKETLKELVENDSLLKIILLFYIFTPKELK |
| | GEFLGFIKKFYHDTKNIDKDTKDKEEPLEQIKQEVPLKLKILEKNLTILTIFNYSISLNIEYDKNNNSFY |
| | ERGNKFKKIYKDLKISHNQEEFDKSLLAPLLKYYMNLYKLLNDFEIYLLLKYKNKDNLNKESLNKLINDE |
| | QLKHNDHYNFTTLLSEYFNFDPKKNKKYETLTILRNSISHQKIDNLIYNLDKNKILEQRVKIVELIKEQR |
| | DIKETLKFDPINDFTMKTVQLLKSLENQSEKRDKIEEILKQQDLSANDFYNIYKLKGVESIKKELFIRLG |
| | KTKIEEKIQEDIAKGSI (SEQ ID NO: 124) |
| Cas13c.2 | MNSIEKIKKPSNRNSIPSIIISDYDENKIKEIKVKYLKLARLDKITIQDMEIRDNIVEFKKILLNGIEHT |
| amino acid | IKDNQKIEFDNYEITAYVRASKQRRDGKITQAKYVVTITDKYLRDNEKEKRFKSTERELPNDTLLMRYKQ |
| sequence | ISGFDTLTSKDIYKIKRYIDFKNEMLFYFQFIEEFFSPLLPKGTNFYSLNIEQNKDKVVKYIVYRLNDDF |
| | KNQSLNQFIKKTDTIKYDFLKIQKILSDFRHALAHFDFDFIQKFFDDELDKNRFDISTISLIKTMLQEKE |
| | EKYYQEKNNYIEDSDTLTLFDEKESNFSKIHNFYIKISQKKPAFNKLINSFLSKDGVPNEELKSYLATKK |
| | IDFFEDIHSNKEYKKIYIKHKNLVVEKQKEESQEKPNGQKLKNYNDELQKLKDEMNKITKQNSLNRLEVK |
| | LRLAFGFIANEYNYNFKNFNDKFTLDVKKEQKIKVFKNSSNEKLKEYFESTFIEKRFFHFCVKFFNKKTK |
| | KEETKQKNIFNLIENETLEELVKESPLLQIITLLYLFIPKELQGEFVGFILKIYHHTKNITNDTKEDEKS |
| | IEDTQNSFSLKLKILAKNLRGLQLFNYSLSHNTLYNTKEHFFYEKGNRWQSVYKSLEISHNQDEFDIHLV |
| | IPVIKYYINLNKLIGDFEIYALLTYADKNSITEKLSDITKRDDLKFRGYYNFSTLLFKTFMINTNYEQNQ |
| | KSTQYIKQTRNDIAHQNIENMLKAFENNEIFAQREEIVNYLQKEHKMQEILHYNPINDFTMKTVQYLKSL |
| | NIHSQKESKIADIHKKESLVPNDYYLIYKLKVIELLKQKVIEAIGETKDEEKIKNAIAKEEQIKKGYNK |
| | (SEQ ID NO: 125) |

| | |
|---|---|
| Cas13e.3 DR | GCTGGAGCAGCCCTCGATTTGCTGGGTAATCACAGC (SEQ ID NO: 126) |
| coding sequence | |
| Cas13e.4 DR | GCTGAAGCAACCCTGGTTTTGCGGGGTGATTACAGC (SEQ ID NO: 127) |
| coding sequence | |
| Cas13e.5 DR | GCTGTAGAAGCCTCCGATTTGTGAGGTGATGACAGC (SEQ ID NO: 128) |
| coding sequence | |
| Cas13e.6 DR | GCTGGAGCAGCCCTCGATTTGCAGGGTAATCACAGC (SEQ ID NO: 129) |
| coding sequence | |
| Cas13e.7 DR | GCTGGAGCAGCCCTCGATTTGCAGGGTTATCACAGC (SEQ ID NO: 130) |
| coding sequence | |
| Cas13e.8 DR | GTTGGAGTAGCCCCGGATTTGCGGGGTGATTACAGC (SEQ ID NO: 131) |
| coding sequence | |
| Cas13f.6 DR | GCTGTGATAGACCTCGATTTGTGGGGTAGTAACAGC (SEQ ID NO: 132) |
| coding sequence | |
| Cas13f.7 DR | GCTGTGATGGACCTCGATTTGTGGGGTAGTAACAGC (SEQ ID NO: 133) |
| coding sequence | |
| Cas13d.1 DR | CAACTACAACCCCGTAAAAATACGGGGTTCTGAAAC (SEQ ID NO: 134) |
| coding sequence | |
| Cas13d.2 DR | GTTAAATACCACCTAAGAATGAGGAGGTTCTATAAC (SEQ ID NO: 135) |
| coding sequence | |
| Cas13d.3 DR | GAACGATAGCCTGCTGAAATATGCAGGTTCTAAGAC (SEQ ID NO: 136) |
| coding sequence | |
| Cas13d.4 DR | GATTGAAAGCTATGCGAATTTGCACAGTCTTAAAAC (SEQ ID NO: 137) |
| coding sequence | |
| Cas13d.5 DR | GAGATAGACCCTTGTTAACTCGTAAGGTTCTGTGAC (SEQ ID NO: 138) |
| coding sequence | |
| Cas13c.1 DR | ATTGGATATACCCCTAATTTGAGAGGGGAATAAAAC (SEQ ID NO: 139) |
| coding sequence | |
| Cas13c.2 DR | GTTGGACTATACCCTCGTTTGTAGGGGGAATAAAAC (SEQ ID NO: 140) |
| coding sequence | |

| | |
|---|---|
| SsoCas6 amino acid | MPLIFKIGYNVIPLQDVILPTPSSKVLKYLIQSGKLIPSLKDLITSRDKYKPIFISHLGFN |
| sequence | QRRIFQTNGNLKTITKGSRLSSIIAFSTQANVLSEVADEGIFETVYGKFHIMIESIEIVEV |
| | EKLKEEVEKHMNDNIRVRFVSPTLLSSKVLLPPSLSERYKKIHAGYSTLPSVGLIVAYAYN |
| | VYCNLIGKKEVEVRAFKFGILSNALSRIIGYDLHPVTVAIGEDSKGNLRKARGVMGWIEFD |
| | IPDERLKRRALNYLLTSSYLGIGRSRGIGFGEIRLEFRKIEEKEG (SEQ ID NO: 141) |
| MmCas6 amino acid | MDLEYMHISYPNILLNMRDGSKLRGYFAKKYIDEEIVHNHRDNAFVYKYPQIQFKIIDRSP |
| sequence | LIIGIGSLGINFLESKRIFFEKELIISNDTNDITEVNVHKDMDHFGTTDKILKYQFKTPWM |
| | ALNAKNSEIYKNSDEIDREEFLKRVLIGNILSMSKSLGYTIEEKLKVKINLKEVPVKFKNQ |
| | NMVGFRGEFYINFDIPQYLGIGRNVSRGFGTWKV (SEQ ID NO: 142) |
| SpCas5d amino acid | MYRSRDFYVRVSGQRALFTMPATKGGSERSSYSVPTRQALNGIVDAIYYKPTFTNIVTEVK |
| sequence | VINQIQTELQGVRALLHDYSADLSYVSYLSDVVYLIKFHFVWNEDRKDLNSDRLPAKHEAI |
| | MERSIRKGGRRDVFLGTRECLGLLDDISQEEYETTVSYYNGVNIDLGIMFHSFAYPKDKKT |
| | PLKSYFTKTVMKNGVITFKAQSECDIVNTLSSYAFKAPEEIKSVNDECMEYDAMEKGEN |
| | (SEQ ID NO: 143) |
| BhCas5d amino acid | MRNEVQFELFGDYALFTDPLTKIGGEKLSYSVPTYQALKGIAESIYWKPTIVFVIDELRVM |
| sequence | KPIQMESKGVRPIEYGGGNTLAHYTYLKDVHYQVKAHFEFNLHRPDLAFDRNEGKHYSILQ |
| | RSLKAGGRRDIFLGARECQGYVAPCEFGSGDGFYDGQGKYHLGTMVHGFNYPDETGQHQLD |
| | VRLWSAVMENGYIQFPRPEDCPIVRPVKEMEPKIFNPDNVQSAEQLLHDLGGE (SEQ ID |
| | NO: 144) |
| SaCas6 amino acid | MPNDPYSLYSIVIELGAAEKGFPTGILGRSLHSQVLQWFKQDNPFLATELHQSQISPFSIS |
| sequence | PLMGKRHAKLTKAGDRLFFRICLLRGDLLQPLLNGIEQTVNQSVCLDKFRFRLCQTHILPG |
| | SHPLAGASHYSLISQTPVSSKITLDFKSSTSFKVDRKIIQVFPLGEHVFNSLLRRWNNFAP |
| | EDLHFSQVDWSIPIAAFDVKTIPIHLKKVEIGAQGWVTYIFPNTEQAKIASVLSEFAFFSG |
| | VGRKTTMGMGQVQVRS (SEQ ID NO: 145) |
| EcCas6e amino acid | MYLSKVIIARAWSRDLYQLHQGLWHLFPNRPDAARDFLFHVEKRNTPEGCHVLLQSAQMPV |
| sequence | STAVATVIKTKQVEFQLQVGVPLYFRLRANPIKTILDNQKRLDSKGNIKRCRVPLIKEAEQ |
| | IAWLQRKLGNAARVEDVHPISERPQYFSGDGKSGKIQTVCFEGVLTINDAPALIDLVQQGI |
| | GPAKSMGCGLLSLAPL (SEQ ID NO: 146) |
| PaCasof amino acid | MDHYLDIRLRPDPEFPPAQLMSVLFGKLHQALVAQGGDRIGVSFPDLDESRSRLGERLRIH |
| sequence | ASADDLRALLARPWLEGLRDHLQFGEPAVVPHPTPYRQVSRVQAKSNPERLRRRLMRRHDL |
| | SEEEARKRIPDTVARALDLPFVTLRSQSTGQHFRLFIRHGPLQVTAEEGGFTCYGLSKGGF |
| | VPWF (SEQ ID NO: 147) |
| MtCas6 amino acid | MAARRGGIRRTDLLRRSGQPRGRHRASAAESGLTWISPTLILVGFSHRGDRRMTEHLSRLT |
| sequence | LTLEVDAPLERARVATLGPHLHGVLMESIPADYVQTLHTVPVNPYSQYALARSTTSLEWKI |
| | STLTNEARQQIVGPINDAAFAGFRLRASGIATQVTSRSLEQNPLSQFARIFYARPETRKFR |
| | VEFLTPTAFKQSGEYVFWPDPRLVFQSLAQKYGAIVDGEEPDPGLIAEFGQSVRLSAFRVA |
| | SAPFAVGAARVPGFTGSATFTVRGVDTFASYIAALLWFGEFSGCGIKASMGMGAIRVQPLA |
| | PREKCVPKP (SEQ ID NO: 148) |
| PfCas6 amino acid | MRFLIRLVPEDKDRAFKVPYNHQYYLQGLIYNAIKSSNPKLATYLHEVKGPKLFTYSLFMA |
| sequence | EKREHPKGLPYFLGYKKGFFYFSTCVPEIAEALVNGLLMNPEVRLWDERFYLHEIKVLREP |
| | KKFNGSTFVTLSPIAVTVVRKGKSYDVPPMEKEFYSIIKDDLQDKYVMAYGDKPPSEFEME |
| | VLIAKPKRFRIKPGIYQTAWHLVFRAYGNDDLLKVGYEVGFGEKNSLGFGMVKVEGNKTTK |
| | EAEEQEKITFNSREELKTGV (SEQ ID NO: 149) |
| PaCsf5 amino acid | MFVTQVIFNIGERTYPDRARAMVAELMDGVQPGLVATLMNYIPGTSTSRTEFPTVQFGGAS |
| sequence | DGFCLLGFGDGGGAIVRDAVPLIHAALARRMPDRIIQVEHKEHSLSAEARPYVLSYTVPRM |
| | VVQKKQRHAERLLHEAEGKAHLEGLFLRSLQRQAAAVGLPLPENLEVEFKGAVGDFAAKHN |
| | PNSKVAYRGLRGAVFDVNARLGGIWTAGFMLSKGYGQFNATHQLSGAVNALSE (SEQ ID |
| | NO: 150) |
| MtCsf5 amino acid | MHQTLIRINWPKGFKCPPAEFREKLAKSEMFPPEFFHYGTELAVWDKQTAEVEGKIKTVSK |
| sequence | EKIIKTFDKPIPLNGRAPVRVIGGQAWAGVIADPEMEGMLIPHLGSILKVASSAAGCAVKI |
| | ELEQRKFGISYTEYPVKYNLRELVLKRRCEDARSTDIESLIADRIWGGVSGESYYGIDGTC |
| | AKFGFEPPSREQLELRIFPMKNIGLHMKSSDGLSKEYMSLIDAEVWMNAKLEGVWQVGNLI |
| | SRGYGRFIKSIGAQS (SEQ ID NO: 151) |
| SsoCas6 DR coding | SEQ ID NO: 152 |
| sequence | GATAATCTCTTATAGAATTGAAAG |
| MmCas6 DR coding | SEQ ID NO: 153 |
| sequence | CTAAAAGAATAACTTGCAAAATAACAAGCATTGAAAC |
| SpCas5d DR coding | SEQ ID NO: 154 |
| sequence | GTCTCACCCTTCATGGGTGAGTGGATTGAAAT |
| BhCas5d DR coding | SEQ ID NO: 155 |
| sequence | GTCGCACTCTTCATGGGTGCGTGGATTGAAAT |
| SaCas6 DR coding | SEQ ID NO: 156 |
| sequence | GTTTCAGTCCCGTAGTCGGGATTTAGTGGTTGGAAAG |
| EcCas6e DR coding | SEQ ID NO: 157 |
| sequence | GAGTTCCCCGCGCCAGCGGGGATAAACCG |
| PaCas6f DR coding | SEQ ID NO: 158 |
| sequence | GTTCACTGCCGTATAGGCAGCTAAGAAA |
| MtCas6 DR coding | SEQ ID NO: 159 |
| sequence | GTCGTCAGACCCAAAACCCCGAGAGGGGACGGAAAC |
| PfCas6 DR coding | SEQ ID NO: 160 |
| sequence | GTTACAATAAGACTAAAATAGAATTGAAAG |
| PaCsf5 DR coding | SEQ ID NO: 1616 |
| sequence | GTATTTCCCGCGTGCGCGGGGGTGAGCGG |
| MtCsf5 DR coding | SEQ ID NO: 162 |
| sequence | TATTGGATACACCCACTCATTGGTGGGTGGTTAGAAC |

hADAR2_{DD-}E488Q/T375G (ADARv2) (SEQ ID NO: 163)
QLHLPQVLADAVSRLVLGKFGDLTDNFSSPHARRKVLAGVVMTTGTDVKDAKVISVSTGGKCINGEYMSDRGLALNDCHAEIISRR
SLLRFLYTQLELYLNNKDDQKRSIFQKSERGGFRLKENVQFHLYISTSPCGDARIFSPHEPILEEPADRHPNRKARGQLRTKIESG
QGTIPVRSNASIQTWDGVLQGERLLTMSCSDKIARWNVVGIQGSLLSIFVEPIYFSSIILGSLYHGDHLSRAMYQRISNIEDLPPL
YTLNKPLLSGISNAEARQPGKAPNFSVNWTVGDSAIEVINATTGKDELGRASRLCKHALYCRWMRVHGKVPSHLLRSKITKPNVYH
ESKLAAKEYQAAKARLFTAFIKAGLGAWVEKPTEQDQFSLT
>spacer coding sequence-5mis/30 nt targeting mCherry* (SEQ ID NO: 164)
ctccCagcccatggttttcttctgcattac
>The reporter plasmid (pCMV-BFP-P2A-mCherry(W148X)-ployA) (SEQ ID NO: 165):
CGTTACATAACTTACGGTAAATGGCCCGCCTGGCTGACCGCCCAACGACCCCCGCCCATTGACGTCAATAATGACGTATGTTCCCA
TAGTAACGCCAATAGGGACTTTCCATTGACGTCAATGGGTGGAGTATTTACGGTAAACTGCCCACTTGGCAGTACATCAAGTGTAT
CATATGCCAAGTACGCCCCCTATTGACGTCAATGACGGTAAATGGCCCGCCTGGCATTATGCCCAGTACATGACCTTATGGGACTT
TCCTACTTGGCAGTACATCTACGTATTAGTCATCGCTATTACCATGGTGATGCGGTTTTGGCAGTACATCAATGGGCGTGGATAGC
GGTTTGACTCACGGGGATTTCCAAGTCTCCACCCCATTGACGTCAATGGGAGTTTGTTTTGGCACCAAAATCAACGGGACTTTCCA
AAATGTCGTAACAACTCCGCCCCATTGACGCAAATGGGCGGTAGGCGTGTACGGTGGGAGGTCTATATAAGCAGAGCTGGTTTAGT
GAACCGTCAGATCCGCTAGCATGAGCGAGCTGATTAAGGAGAACATGCACATGAAGCTGTATATGGAGGGCACCGTGGACAACCAT
CACTTCAAGTGCACATCCGAGGGCGAAGGCAAGCCCTACGAGGGCACCCAGACCATGAGAATCAAGGTGGTCGAGGGCGGCCCTCT
CCCCTTCGCCTTCGACATCCTGGCTACTAGCTTCCTCTACGGCAGCAAGACCTTCATCAACCACACCCAGGGCATCCCCGACTTCT
TCAAGCAGTCCTTCCCTGAGGGCTTCACATGGGAGAGAGTCACCACATACGAGGACGGGGGCGTGCTGACCGCTACCCAGGACACC
AGCCTCCAGGACGGCTGCCTCATCTACAACGTCAAGATCAGAGGGGTGAACTTCACATCCAACGGCCCTGTGATGCAGAAGAAAAC
ACTCGGCTGGGAGGCCTTCACCGAGACACTGTACCCCGCTGACGGCGGCCTGGAAGGCAGAAACGACATGGCCCTGAAGCTCGTGG
GCGGGAGCCATCTGATCGCAAACATCAAGACCACATATAGATCCAAGAAACCCGCTAAGAACCTCAAGATGCCTGGCGTCTACTAT
GTGGACTACAGACTGGAAAGAATCAAGGAGGCCAACAACGAGACATACGTCGAGCAGCACGAGGTGGCAGTGGCCAGATACTGCGA
CCTCCCTAGCAAACTGGGGCACAAGCTGAATGGCGCCACTAACTTCTCCCTGTTGAAACAAGCAGGGGATGTCGAAGAGAATCCCG
GGCCAATGGTGAGCAAGGGCGAGGAGGATAACATGGCCATCATCAAGGAGTTCATGCGCTTCAAGGTGCACATGGAGGGCTCCGTG
AACGGCCACGAGTTCGAGATCGAGGGCGAGGGCGAGGGCCGCCCCTACGAGGGCACCCAGACCGCCAAGCTGAAGGTGACCAAGGG
TGGCCCCCTGCCCTTCGCCTGGGACATCCTGTCCCCTCAGTTCATGTACGGCTCCAAGGCCTACGTGAAGCACCCCGCCGACATCC
CCGACTACTTGAAGCTGTCCTTCCCCGAGGGCTTCAAGTGGGAGCGCGTGATGAACTTCGAGGACGGCGGCGTGGTGACCGTGACC
CAGGACTCCTCCCTGCAGGACGGCGAGTTCATCTACAAGGTGAAGCTGCGCGGCACCAACTTCCCCTCCGACGGCCCCGTAATGCA
GAAGAAAACCATGGGCTAGGAGGCCTCCTCCGAGCGGATGTACCCCGAGGACGGCGCCCTGAAGGGCGAGATCAAGCAGAGGCTGA
AGCTGAAGGACGGCGGCCACTACGACGCTGAGGTCAAGACCACCTACAAGGCCAAGAAGCCCGTGCAGCTGCCCGGCGCCTACAAC
GTCAACATCAAGTTGGACATCACCTCCCACAACGAGGACTACACCATCGTGGAACAGTACGAACGCGCCGAGGGCCGCCACTCCAC
CGGCGGCATGGACGAGCTGTACAAGTAAATCCTGTGCCTTCTAGTTGCCAGCCATCTGTTGTTTGCCCCTCCCCCGTGCCTTCCTT
GACCCTGGAAGGTGCCACTCCCACTGTCCTTTCCTAATAAAATGAGGAAATTGCATCGCATTGTCTGAGTAGGTGTCATTCTATTC
TGGGGGGTGGGGTGGGGCAGGACAGCAAGGGGGAGGATTGGGAAGAGAATAGCAGGCATGCTGGGGA
>spacer coding sequence targeting mCherry (W148X) (SEQ ID NO: 166):
TACATCCGCTCGGAGGAGGCCTCCCAGCCCATGGTTTTCTTCTGCATTAC
>NT-spacer coding sequence (SEQ ID NO: 167):
GGTCTTCGATATTCAAGCGTCGGAAGACCT
>dCas13e.2-N150 + C150 amino acid sequence (SEQ ID NO: 167):
MSGWITPAGILLLASFFVERGILHRLMGNIGGFKDNRGEYGLTHDIETTYCLKGSYSIRAQDHDAVMFRDILGYLSRVPTESE
QRIKQPQIRKEGQLSERKTDKFITFALNYLEDYGLKDLEGCKACFARSKIVREQENVESINDKEYKPHENKKKVEIHFDQSKEDRF
YINRNNVILKIQKKDGHSNIVRMGVYELKYLVLMSLVGKAKEAVEKIDNYIQDLRDQLPYIEGKNKEEIKEYVRFFPRFIRSHLGL
LQINDEEKIKARLDYVKTKWLDKKEKSKELELHKKGRDILRYINERCDRELNRNVYNRILELLVSKDLTGFYRELEELKRTRRIDK
NIVQNLSGQKTINALHEKVCDLVLKEIESLDTENLRKYLGLIPKEEKEVTFKEKVDRILKQPVIYKGFLRYQFFKDDKKSFVLLVE
DALKEKGGGCDVPLGKEYYKIVSLDKYDKENKTLCETLAMDRLCLMMARQYYLSLNAKLAQEAQQIEWKKEDSIELII
>dCas13e.2-N180 + C180 amino acid sequence (SEQ ID NO: 169):
MGFKDNRGEYGLTHDIFTTYCLKGSYSIRAQDHDAVMFRDILGYLSRVPTESFQRIKQPQIRKEGQLSERKTDKFITFALNYL
EDYGLKDLEGCKACFARSKIVREQENVESINDKEYKPHENKKKVEIHFDQSKEDRFYINRNNVILKIQKKDGHSNIVRMGVYELKY
LVLMSLVGKAKEAVEKIDNYIQDLRDQLPYIEGKNKEEIKEYVRFFPRFIRSHLGLLQINDEEKIKARLDYVKTKWLDKKEKSKEL
ELHKKGRDILRYINERCDRELNRNVYNRILELLVSKDLTGFYRELEELKRTRRIDKNIVQNLSGQKTINALHEKVCDLVLKEIESL
DTENLRKYLGLIPKEEKEVTFKEKVDRILKQPVIYKGFLRYQFFKDDKKSFVLLVEDALKEKGGGCDVPLGKEYYKIVSLDKYDKE
NKTLCETLAMDRLCLMMA
>dCas13e.3-N180 + C180 amino acid sequence (SEQ ID NO: 170):
MAHYSLRDSYVIKAEETKSFRDLLGYLSRVPQQAVDWLNEHNQLSEDEKKEFLNQKPSDEESQEQSKTENTDRQADRMPRRSL
RKTDKFILFAAKFIEDWAQKEKMDVTFARYQKTVTEDENKNQDGKQVRDVQLKYEKDTKKLNPDFDYKWTYYIRNNHAIIQIKPDE
YKQAVSARISENELKYLVLLIFQGKGWEAIKKIGDYIFHIGNKIKIGRFDHNEERRMPSFLKNPPADIIGEMVENRLKYIRDELNK
VIETIKKEEPQNNKWLLYKGKKISIILKFISDSISDIKKRPDVNEYNTLRDMLQKLDFDNFYERLKSYVSEGRIEQTLYDEIKGIK
DISTLCIKICELRLAALEELEKEGGDDLNKYIGLAVQEKHKNYDDSNTPQKKAERFLESQFSVGKNFLRETFYDEYIKNRKSLYEI
IKEKITGITPLNENRWYLMDKNPKEFESKDSKIIRGLCNIYIQDILCMKIALWYYENLSPSYKNKLKWDFIGQGFGYDR
>dCas13e.7-N150 + C150 amino acid sequence (SEQ ID NO: 171):
MGIPGFKRSDKKKWGDGQKIDYGFTRKLMSFYSLRDSYSVNVQENKELTAFRDILGYLARVPGQAIDWLIEKGKLTKEEGKQF
YLGEQSEEREEKAKKEEIKYALRKTDKFMLFAVRFIEDWAEQERIKVEFARYEKMTIVNENKKQDEKEERKVKFVSDEPTAAGWTY
YIRNNHAIIKIIPDDKKKKAVSARISENELKYLVLTIIDGNGKNAIAYIGDYIFRTARQIENKSYNAESEKYAPAFVRGGQKKSVD
KRIKYIRDEIQQVINDIEAEQEKQKNEQDAPAENRTWLIYKGKKISIILRYVNDNIAEYKKRLSVTEYNELRGYLQQLDFINFHRK
LAEYQHHGRLPNGFAESINKFQDLSKLCIEVCERQKKKLQEMAAKGGIELEQYIGLAPKEENQEQNKYATKANNFIKVWLSIPENF
LRQKFYDKFCKQQECKNKGSDKPDNTSVPQRKYFIAIIREKNIRPIHADKYYLLGQNPKDYERPDGKIIRQLCDVYCKDGLCMAMA
KWYYENRLGKFKDLIEWQTGDDKQQHGYAGHTLEYQATEKIKIRFKLADFTRLDIIEP
>dCas13f.2-N150 + c150 amino acid sequence (SEQ ID NO: 172):
MLISGISGFKRNDPTGQPRRNLFTYFSAREGYKALPDMQKHFLLFTLVNYLSNQDEYISELKQYGEIGQGAFFNRIASTFLNI
SGISGNTKFYSYQSKRIKEQRGELNSEKDSFEWIEPFQGNSYFEINGHKGVIGEDELKELCYALLVAKQDINAVEGKIMQFLKKFR
NTGNLQQVKDDEMLEIEYFPASYFNESKKEDIKKEILGRLDKKIRSCSAKAEKAYDKMKEVMEFINNSLPAEEKLKRKDYRRYLKM
VRFWSREKGNIEREFRTKEWSKYFSSDFWRKNNLEDVYKLATQKNAELFKNLKAAAEKMGETEFEKYQQINDVKDLASLRRLTQDF
GLKWEEKDWEEYSEQIKKQITDRQKLTIMKQRVTAELKKKHGIENLNLRITIDSNKSRKAVLNRIAIPRGFVKKHILGWQGSEKIS
KNIREAECKILLSKKYEELSRQFFEAGNFDKLTQINGLYEKNKLTAFMSVYLMGRLNIQLNKHTE
>PspCas13b-DR, SEQ ID NO: 173
GTTGTGGAAGGTCCAGTTTTGAGGGGCTATTACAAC
>dPspCas13b, SEQ ID NO: 174
MNIPALVENQKKYFGTYSVMAMLNAQTVLDHIQKVADIEGEQNENNENLWFHPVMSHLYNAKNGYDKQPEKTMFIIERLQSYF
PFLKIMAENQREYSNGKYKQNRVEVNSNDIFEVLKRAFGVLKMYRDLTNAYKTYEEKLNDGCEFLTSTEQPLSGMINNYYTVALRN
MNERYGYKTEDLAFIQDKRFKFVKDAYGKKKSQVNTGFFLSLQDYNGDTQKKLHLSGVGIALLICLFLDKQYINIFLSRLPIFSSY
NAQSEERRIIIRSFGINSIKLPKDRIHSEKSNKSVAMDMLNEVKRCPDELFTTLSAEKQSRFRIISDDHNEVLMKRSSDRFVPLLL
QYIDYGKLFDHIRFHVNMGKLRYLLKADKTCIDGQTRVRVIEQPLNGFGRLEEAETMRKQENGTFGNSGIRIRDFENMKRDDANPA
NYPYIVDTYTHYILENNKVEMFINDKEDSAPLLPVIEDDRYVVKTIPSCRMSTLEIPAMAFHMFLFGSKKTEKLIVDVHNRYKRLF
QAMQKEEVTAENIASFGIAESDLPQKILDLISGNAHGKDVDAFIRLTVDDMLTDTERRIKRFKDDRKSIRSADNKMGKRGFKQIST
GKLADFLAKDIVLFQPSVNDGENKITGLNYRIMQSAIAVYDSGDDYEAKQQFKLMFEKARLIGKGTTEPHPFLYKVFARSIPANAV
EFYERYLIERKFYLTGLSNEIKKGNRVDVPFIRRDQNKWKTPaMKTLGRIYSEDLPVELPRQMFDNEIKSHLKSLPQMEGIDFNNA
NVTYLIAEYMKRVLDDDFQTFYQWNRNYRYMDMLKGEYDRKGSLQHCFTSVEEREGLWKERASRTERYRKQASNKIRSNRQMRNAS
SEEIETILDKRLSNSRNEYQKSEKVIRRYRVQDALLFLLAKKTLTELADFDGERFKLKEIMPDAEKGILSEIMPMSFTFEKGGKKY
TITSEGMKLKNYGDFFVLASDKRIGNLLELVGSDIVSKED
>dPspCas13b-linker-NES-GS linker-hADARv1, SEQ ID NO: 175
MNIPALVENQKKYFGTYSVMAMLNAQTVLDHIQKVADIEGEQNENNENLWFHPVMSHLYNAKNGYDKQPEKTMFIIERLQSYF
PFLKIMAENQREYSNGKYKQNRVEVNSNDIFEVLKRAFGVLKMYRDLTNAYKTYEEKLNDGCEFLTSTEQPLSGMINNYYTVALRN
MNERYGYKTEDLAFIQDKRFKFVKDAYGKKKSQVNTGFFLSLQDYNGDTQKKLHLSGVGIALLICLFLDKQYINIFLSRLPIFSSY
NAQSEERRIIIRSFGINSIKLPKDRIHSEKSNKSVAMDMLNEVKRCPDELFTTLSAEKQSRFRIISDDHNEVLMKRSSDRFVPLLL
QYIDYGKLFDHIRFHVNMGKLRYLLKADKTCIDGQTRVRVIEQPLNGFGRLEEAETMRKQENGTFGNSGIRIRDFENMKRDDANPA
NYPYIVDTYTHYILENNKVEMFINDKEDSAPLLPVIEDDRYVVKTIPSCRMSTLEIPAMAFHMFLFGSKKTEKLIVDVHNRYKRLF
QAMQKEEVTAENIASFGIAESDLPQKILDLISGNAHGKDVDAFIRLTVDDMLTDTERRIKRFKDDRKSIRSADNKMGKRGFKQIST
GKLADFLAKDIVLFQPSVNDGENKITGLNYRIMQSAIAVYDSGDDYEAKQQFKLMFEKARLIGKGTTEPHPFLYKVFARSIPANAV
EFYERYLIERKFYLTGLSNEIKKGNRVDVPFIRRDQNKWKTPAMKTLGRIYSEDLPVELPRQMFDNEIKSHLKSLPQMEGIDFNNA
NVTYLIAEYMKRVLDDDFQTFYQWNRNYRYMDMLKGEYDRKGSLQHCFTSVEEREGLWKERASRTERYRKQASNKIRSNRQMRNAS
SEEIETILDKRLSNSRNEYQKSEKVIRRYRVQDALLFLLAKKTLTELADFDGERFKLKEIMPDAEKGILSEIMPMSFTFEKGGKKY
TITSEGMKLKNYGDFFVLASDKRIGNLLELVGSDIVSKEDGSLQLPPLERLTLGGGGGSGGGGSGGGGSGGGGSQLHLPQVLADAV
SRLVLGKFGDLTDNFSSPHARRKVLAGVVMTTGTDVKDAKVISVSTGTKCINGEYMSDRGLALNDCHAEIISRRSLLRFLYTOLEL
YLNNKDDQKRSIFQKSERGGFRLKENVQFHLYISTSPCGDARIFSPHEPILEEPADRHPNRKARGQLRTKIESGQGTIPVRSNASI
QTWDGVLQGERLLTMSCSDKIARWNVVGIQGSLLSIFVEPIYFSSIILGSLYHGDHLSRAMYQRISNIEDLPPLYTLNKPLLSGIS
NAEARQPGKAPNFSVNWTVGDSAIEVINATTGKDELGRASRLCKHALYCRWMRVHGKVPSHLLRSKITKPNVYHESKLAAKEYQAA
KARLFTAFIKAGLGAWVEKPTEQDQFSLT
>ddPspCas13b-D369A, K370A, R380A, SEQ ID NO: 176
MNIPALVENQKKYFGTYSVMAMLNAQTVLDHIQKVADIEGEQNENNENLWFHPVMSHLYNAKNGYDKQPEKTMFIIERLQSYF
PFLKIMAENQREYSNGKYKQNRVEVNSNDIFEVLKRAFGVLKMYRDLTNAYKTYEEKLNDGCEFLTSTEQPLSGMINNYYTVALRN
MNERYGYKTEDLAFIQDKRFKFVKDAYGKKKSQVNTGFFLSLQDYNGDTQKKLHLSGVGIALLICLFLDKQYINIFLSRLPIFSSY
NAQSEERRIIIRSFGINSIKLPKDRIHSEKSNKSVAMDMLNEVKRCPDELFTTLSAEKQSRFRIISDDHNEVLMKRSSDRFVPLLL
QYIDYGKLFDHIRFHVNMGKLRYLLKAAATCIDGQTRVAVIEQPLNGFGRLEEAETMRKQENGTFGNSGIRIRDFENMKRDDANPA
NYPYIVDTYTHYILENNKVEMFINDKEDSAPLLPVIEDDRYVVKTIPSCRMSTLEIPAMAFHMFLFGSKKTEKLIVDVHNRYKRLF
QAMQKEEVTAENIASFGIAESDLPQKILDLISGNAHGKDVDAFIRLTVDDMLTDTERRIKRFKDDRKSIRSADNKMGKRGFKQIST
GKLADFLAKDIVLFQPSVNDGENKITGLNYRIMQSAIAVYDSGDDYEAKQQFKLMFEKARLIGKGTTEPHPFLYKVFARSIPANAV
EFYERYLIERKFYLTGLSNEIKKGNRVDVPFIRRDQNKWKTPAMKTLGRIYSEDLPVELPRQMFDNEIKSHLKSLPQMEGIDFNNA
NVTYLIAEYMKRVLDDDFQTFYQWNRNYRYMDMLKGEYDRKGSLQHCFTSVEEREGLWKERASRTERYRKQASNKIRSNRQMRNAS
SEEIETILDKRLSNSRNEYQKSEKVIRRYRVQDALLFLLAKKTLTELADFDGERFKLKEIMPDAEKGILSEIMPMSFTFEKGGKKY
TITSEGMKLKNYGDFFVLASDKRIGNLLELVGSDIVSKED
>ddPspCas13b-D369A,K370A,R38OA-linker-NES-GS linker-hADARvl, SEQ ID NO: 177
MNIPALVENQKKYFGTYSVMAMLNAQTVLDHIQKVADIEGEQNENNENLWFHPVMSHLYNAKNGYDKQPEKTMFIIERLQSYF
PFLKIMAENQREYSNGKYKQNRVEVNSNDIFEVLKRAFGVLKMYRDLTNAYKTYEEKLNDGCEFLTSTEQPLSGMINNYYTVALRN
MNERYGYKTEDLAFIQDKRFKFVKDAYGKKKSQVNTGFFLSLQDYNGDTQKKLHLSGVGIALLICLFLDKQYINIFLSRLPIFSSY
NAQSEERRIIIRSFGINSIKLPKDRIHSEKSNKSVAMDMLNEVKRCPDELFTTLSAEKQSRFRIISDDHNEVLMKRSSDRFVPLLL
QYIDYGKLFDHIRFHVNMGKLRYLLKAAATCIDGQTRVAVIEQPLNGFGRLEEAETMRKQENGTFGNSGIRIRDFENMKRDDANPA
NYPYIVDTYTHYILENNKVEMFINDKEDSAPLLPVIEDDRYVVKTIPSCRMSTLEIPAMAFHMFLFGSKKTEKLIVDVHNRYKRLF
QAMQKEEVTAENIASFGIAESDLPQKILDLISGNAHGKDVDAFIRLTVDDMLTDTERRIKRFKDDRKSIRSADNKMGKRGFKQIST
GKLADFLAKDIVLFQPSVNDGENKITGLNYRIMQSAIAVYDSGDDYEAKQQFKLMFEKARLIGKGTTEPHPFLYKVFARSIPANAV
EFYERYLIERKFYLTGLSNEIKKGNRVDVPFIRRDQNKWKTPAMKTLGRIYSEDLPVELPRQMFDNEIKSHLKSLPQMEGIDFNNA
NVTYLIAEYMKRVLDDDFQTFYQWNRNYRYMDMLKGEYDRKGSLQHCFTSVEEREGLWKERASRTERYRKQASNKIRSNRQMRNAS
SEEIETILDKRLSNSRNEYQKSEKVIRRYRVQDALLFLLAKKTLTELADFDGERFKLKEIMPDAEKGILSEIMPMSFTFEKGGKKY
TITSEGMKLKNYGDFFVLASDKRIGNLLELVGSDIVSKEDGSLQLPPLERLTLGGGGGSGGGGSGGGGSGGGGSQLHLPQVLADAV
SRLVLGKFGDLTDNFSSPHARRKVLAGVVMTTGTDVKDAKVISVSTGTKCINGEYMSDRGLALNDCHAEIISRRSLLRFLYTQLEL
YLNNKDDQKRSIFQKSERGGFRLKENVQFHLYISTSPCGDARIFSPHEPILEEPADRHPNRKARGQLRTKIESGQGTIPVRSNASI
QTWDGVLQGERLLTMSCSDKIARWNVVGIQGSLLSIFVEPIYFSSIILGSLYHGDHLSRAMYQRISNIEDLPPLYTLNKPLLSGIS
NAEARQPGKAPNFSVNWTVGDSAIEVINATTGKDELGRASRLCKHALYCRWMRVHGKVPSHLLRSKITKPNVYHESKLAAKEYQAA
KARLFTAFIKAGLGAWVEKPTEQDQFSLT

1. A CRISPR-Cas system, comprising:
(a) a CRISPR RNA (crRNA) binding polypeptide or a polynucleotide coding sequence (e.g., a DNA coding sequence or an RNA coding sequence) thereof, said crRNA binding polypeptide comprising, consisting essentially of, or consisting of a crRNA binding domain of a Cas effector protein,
(b) a heterologous functional domain or a polynucleotide coding sequence thereof (e.g., a DNA coding sequence or an RNA coding sequence), and
(c) a guide RNA (gRNA) or a polynucleotide coding sequence (e.g., a DNA coding sequence or an RNA coding sequence) thereof, said gRNA comprising:
(i) a 5′ direct repeat (DR) sequence and a 3′ direct repeat (DR) sequence, each capable of forming a complex with the crRNA binding domain; and
(ii) a spacer sequence capable of hybridizing to a target RNA, and guiding or recruiting the complex to the target RNA,
wherein the crRNA binding polypeptide:
(1) is linked (e.g., fused) to the heterologous functional domain, and
(2) substantially lacks the ability (e.g., having no more than 50%, 40%, 30%, 20%, 10%, 5%, 2%, or 1% of that of the Cas effector protein) to process or cleave DR sequence on the gRNA, and
wherein said spacer sequence is flanked by said 5′ and 3′ DR sequences at the 5′ end and the 3′ end of said spacer sequence, respectively; optionally, said 5′ and 3′ DR sequences are identical.

2. A guide RNA (gRNA) comprising:
a) a 5′ direct repeat (DR) sequence and a 3′ direct repeat (DR) sequence, each capable of forming a complex with a CRISPR RNA (crRNA) binding polypeptide comprising, consisting essentially of, or consisting of a crRNA binding domain of a Cas effector protein; and
b) a spacer sequence capable of hybridizing to a target RNA, and guiding or recruiting the complex to the target RNA,
wherein said spacer sequence is flanked by said 5′ and 3′ DR sequences at the 5′ end and the 3′ end of said spacer sequence, respectively; optionally, said 5′ and 3′ DR sequences are identical.

3-4. (canceled)

5. A modified Cas13 protein with both HEPN1 and HEPN2 domains substantially removed from a parental or wild-type Cas13 effector protein (e.g., substantially lacking both the HEPN1 and HEPN2 domains of the parental or wild-type Cas13 effector protein), with the proviso that the modified Cas13 protein is not minidCas13e.1-N180+C150.

6-7. (canceled)

8. A fusion protein comprising:
a) the modified Cas13 protein of claim 5; and,
b) a heterologous functional domain, e.g., a deaminase domain.

9. A CRISPR-Cas13 system comprising:
a) the modified Cas13 protein of claim 5 or a polynucleotide coding sequence (e.g., a DNA coding sequence or an RNA coding sequence) thereof; and
b) a guide RNA (gRNA) or a polynucleotide coding sequence (e.g., a DNA coding sequence or an RNA coding sequence) thereof, said gRNA comprising:
i. a direct repeat (DR) sequence capable of forming a complex with the modified Cas13 protein of the fusion protein; and,
ii. a spacer sequence capable of hybridizing to a target RNA, and guiding or recruiting the complex to the target RNA.

10. (canceled)

11. The CRISPR-Cas system of claim 1, wherein the Cas effector protein is a Class 2, Type VI (Cas13) effector protein.

12. (canceled)

13. The CRISPR-Cas system of claim 11, wherein said crRNA binding domain substantially lacks both the HEPN1 and HEPN2 domains of the Cas effector protein;
optionally wherein the crRNA binding domain has a first deletion of or comprising the HEPN1 domain, and a second deletion of or comprising the HEPN2 domain;
optionally, (1) the first deletion is no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues larger than the HEPN1 domain of the Cas13 effector protein, and is no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues smaller than the HEPN1 domain of the Cas13 effector protein; and (2) the second deletion is no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues larger than the HEPN2 domain of the Cas13 effector protein, and is no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues smaller than the HEPN2 domain of the Cas13 effector protein.

14-15. (canceled)

16. The CRISPR-Cas13 system of claim 1, wherein the DR sequence or the 5′ and/or the 3′ DR sequences each
(1) has substantially the same secondary structure as the secondary structure of any one of SEQ ID NOs: 8-14 and 126-140, and/or is encoded by or comprises any one of SEQ ID NOs: 8-14 and 126-140;
(2) has substantially the same secondary structure as the secondary structure of any one of SEQ ID NOs: 47 and 152-162; and/or is encoded by or comprises any one of SEQ ID NOs: 47 and 152-162.

17-24. (canceled)

25. The CRISPR-Cas system of claim 1,
wherein the gRNA comprises, from 5′ to 3′, a first DR sequence, a first spacer sequence, a second DR sequence, a second spacer sequence, and a third DR sequence, whereby said first spacer sequence is flanked by said first and second DR sequences at the 5′ end and the 3′ end of said first spacer sequence, respectively, and said second spacer sequence is flanked by said second and third DR sequences at the 5′ end and the 3′ end of said second spacer sequence, respectively;
wherein the first spacer sequence and the second spacer sequence are each capable of hybridizing to a first target RNA and a second target RNA, respectively, and guiding or recruiting the complex to the first target RNA and the second target RNA, respectively, and
wherein the first and the second target RNA are the same or different.

26-27. (canceled)

28. The CRISPR-Cas system of claim 1, wherein the target RNA is an mRNA.

29-30. (canceled)

31. The CRISPR-Cas system of claim 1, wherein the heterologous functional domain comprises:
a reporter protein or a detection label (e.g., GST, HRP, CAT, GFP, HcRed, DsRed, CFP, YFP, BFP), a protein targeting moiety, a DNA binding domain (e.g., MBP, Lex A DBD, Gal4 DBD), an epitope tag (e.g., His, myc, V5, FLAG, HA, VSV-G, Trx, etc), a transcription activation domain (e.g., VP64 or VPR), a transcription inhibition domain (e.g., KRAB moiety or SID moiety), a nuclease domain (e.g., FokI), a deaminase domain (e.g., ADAR1, ADAR2, APOBEC, AID, or TAD) or a functional fragment thereof, a methylation domain, a demethylation domain (e.g., FTO, ALKBH5), a methyltransferase domain, a methylation reader domain, a transcription release factor, an HDAC, a moiety having ssRNA cleavage activity, a moiety having dsRNA cleavage activity, a moiety having ssDNA cleavage activity, a moiety having dsDNA cleavage activity, a DNA or RNA ligase domain, or any combination thereof; or
a m6A-associated regulation domain, such as, a m6A-associated methyltransferase domain (e.g., METTL3, METTL14, WTAP, KIAA1429, or a functional fragment thereof), a m6A-associated demethylation domain (e.g., Fat mass and obesity-associated protein (FTO), ALKBH5, or a functional fragment thereof), or a combination thereof.

32-34. (canceled)

35. The CRISPR-Cas system of claim 1, wherein the spacer sequence comprises a cystine (C) mismatch opposite to the adenosine (A) in the target RNA and/or an adenosine (A) mismatch opposite to the cytidine (C) in the target RNA.

36-41. (canceled)

42. The CRISPR-Cas system of claim 1, wherein the crRNA binding polypeptide and/or the heterologous functional domain are/is linked to a nuclear localization signal (NLS) sequence or a nuclear export signal (NES).

43-44. (canceled)

45. A polynucleotide comprising a first and a second polynucleotides encoding the protein component and the gRNA component of the CRISPR-Cas system of claim 1, respectively.

46-50. (canceled)

51. A vector comprising the polynucleotide of claim 45, which is optionally a retroviral vector, a phage vector, an adenoviral vector, a herpes simplex viral (HSV) vector, an AAV vector, or a lentiviral vector.

52-58. (canceled)

59. A recombinant AAV (rAAV) viral particle comprising the AAV vector of claim 51, encapsidated within a capsid of the serotype of AAV1, AAV2, AAV3A, AAV3B, AAV4, AAV5, AAV6, AAV7, AAVrh74, AAV8, AAV9, AAV10, AAV11, AAV12, AAV13, AAV.DJ, AAV.PHP.eB, or a mutant thereof.

60. A delivery system comprising (1) a delivery vehicle, and (2) the CRISPR-Cas system of claim 1.

61. (canceled)

62. A cell or a progeny thereof, comprising the CRISPR-Cas system of claim 1.

63-65. (canceled)

66. A pharmaceutical composition comprising:
(i) the CRISPR-Cas13 system of claim 1; and
(ii) a pharmaceutically acceptable excipient.

67. A kit comprising
(i) the CRISPR-Cas system claim 1; and
(ii) an instruction for using component (i).

68. A method of modifying a target RNA, the method comprising contacting the target RNA with the CRISPR-Cas13 system of claim 1, wherein the spacer sequence is substantially complementary to at least 15 contiguous nucleotides of the target RNA;
wherein the crRNA binding polypeptide associates with the gRNA to form a complex;
wherein the complex binds to the target RNA; and wherein upon binding of the complex to the target RNA, the complex modifies the target RNA (e.g., deaminates a target ribonucleotide base (e.g., A or C) in the target RNA).

69-71. (canceled)

72. A method of treating a condition or disease in a subject in need thereof, the method comprising administering to the subject the CRISPR-Cas13 system of claim 1, wherein the spacer sequence is substantially complementary to at least 15 contiguous nucleotides of a target RNA associated with the condition or disease; wherein the crRNA binding polypeptide associates with the gRNA to form a complex; wherein the complex binds to the target RNA; and wherein upon binding of the complex to the target RNA, the complex modifies the target RNA (e.g., deaminates a target ribonucleotide base (e.g., A or C) in the target RNA), thereby treating the condition or disease in the subject.

73-74. (canceled)
